(19) United States
(12) Patent Application Publication
(10) Pub. No.: US 2010/0173888 A1
(43) Pub. Date: Jul. 8, 2010

(54) Nicotinamide Derivatives

(75) Inventors: Atli Thorarensen, Dardenne Prairie, MO (US); John I. Trujillo, Saint Charles, MO (US); Wei Huang, Wildwood, MO (US); Steve R. Turner, Labadie, MO (US); Simon Mantell, Sandwich (GB); Ross Strang, Sandwich (GB); Siew Kuen Yeap, Sandwich (GB)

Correspondence Address: PFIZER INC.;PATENT DEPARTMENT, Bld 114 M/S 114, EASTERN POINT ROAD, GROTON, CT, 06340, US

(73) Assignee: Pfizer Inc

(21) Appl. No.: 12/486,443

(22) Filed: Jun. 17, 2009

Related U.S. Application Data
(60) Provisional application No. 61/073,873, filed on Jun. 19, 2008.

(30) Foreign Application Priority Data
Jun. 18, 2008 (EP) 08158522.6

(51) Int. Cl. A61K 31/397; C07D 213/56; C07D 401/12; A61K 31/444; A61K 31/44; A61K 31/496; C07D 413/12; A61K 31/5377; A61K 31/553; A61P 11/06

(52) U.S. Cl. 514/210.18; 546/316; 546/262; 514/332; 514/355; 544/365; 514/253.13; 544/131; 514/235.5; 540/544; 514/211.15; 514/210.2

(57) ABSTRACT

The present invention relates to compounds of the formula (I)

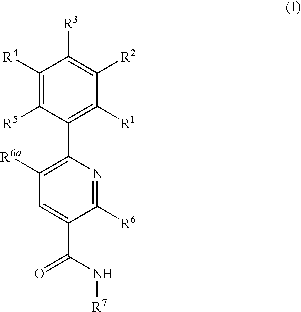

and pharmaceutically acceptable salts and solvates thereof, wherein the substituents are defined herein, to compositions containing such compounds and to the uses of such compounds for the treatment of allergic and respiratory conditions.

The present invention relates to nictonamide derivatives, pharmaceutical compositions comprising such derivatives and their use as medicaments. More particularly, the present invention provides N-cycloalkyl-3-phenylnicotinamide derivatives which are hematopoietic prostaglandin D₂ synthase inhibitors and useful for the treatment of a number of disease, particularly allergic and respiratory diseases.

Prostaglandin D₂ (PGD₂) is a metabolite of arachidonic acid. PGD₂ promotes sleep, inhibits platelet aggregation, relaxes smooth muscle contraction, induces bronchoconstriction and attracts inflammatory cells including Th2 cells, eosinophils and basophils. Both lipocalin-type PGD synthase (L-PGDS) and hematopoietic PGDS (H-PGDS) convert PGH₂ to PGD₂.

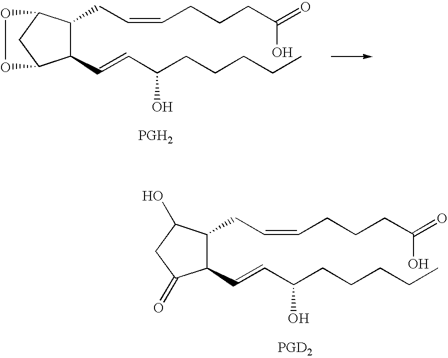

L-PGDS, also known as glutathione-independent PGDS or brain PGDS, is a 26 kDa secretory protein that is expressed by meningeal cells, epithelial cells of the choroid plexus and oligodendrocytes in the brain. L-PGDS secreted into cerebrospinal fluid is thought to be the source of PGD₂ in the central nervous system. In addition, epithelial cells in the epididymis and Leydig cells in the testis express L-PGDS and are thought to be the source of PGD₂ found in the seminal fluid. L-PGDS belongs to the lipocalin superfamily that consists of lipophilic ligand carrier proteins such as retinol- and retinoic acid-binding proteins.

In contrast, H-PGDS is a 26 kDa cytosolic protein that is responsible for the synthesis of PGD₂ in immune and inflammatory cells including mast cells, antigen-presenting cells and Th2 cells. H-PGDS is the only vertebrate member of the sigma class of glutathione S-transferases (GSTs). While both H- and L-PGDS convert PGH₂ to PGD₂, the mechanism of catalysis and specific activity of the enzymes are quite different.

The production of PGD₂ by H-PGDS is thought to play a pivotal role in airway allergic and inflammatory processes and induces vasodilatation, bronchoconstriction, pulmonary eosinophil and lymphocyte infiltration, and cytokine release in asthmatics. PGD₂ levels increase dramatically in bronchoalveolar lavage fluid following allergen challenge and the observation that patients with asthma exhibit bronchoconstriction upon inhalation of PGD₂ underscores the pathologic consequences of high levels of PGD₂ in the lung. Treatment with PGD₂ produces significant nasal congestion and fluid secretion in man and dogs, and PGD₂ is 10 times more potent than histamine and 100 times more potent than bradykinin in producing nasal blockage in humans, demonstrating a role for PGD₂ in allergic rhinitis.

Several lines of evidence suggest that PGDS is an excellent target for allergic and respiratory diseases or conditions. H-PGDS overexpressing transgenic mice show increased allergic reactivity accompanied by elevated levels of Th2 cytokines and chemokines as well as enhanced accumulation of eosinophils and lymphocytes in the lung. In addition, PGD₂ binds to two GPCR receptors, DP1 and CRTH2. Antigen-induced airway and inflammatory responses are strongly decreased in DP1-receptor null mice and recent evidence shows that PGD₂ binding to CRTH2 mediates cell migration and the activation of Th2 cells, eosinophils, and basophils in vitro and likely promotes allergic disease in vivo. Finally, several published reports link H-PGDS gene polymorphisms with atopic asthma. For example, Aritake et al., Structural and Functional Characterization of HQL-79, and Orally Selective inhibitor of Human Hematopoietic Prostaglandin D Synthase, Journal of Biological Chemistry 2006, 281(22), pp. 15277-15286, provides a rational basis for believing that inhibition of H-PGDS is an effective way of treating several allergic and non-allergic diseases.

There is a need to provide new inhibitors of H-PDGS that are suitable as drug candidates. Such compounds should be potent, selective inhibitors of H-PGDS with appropriate metabolic stability and pharmacokinetic properties. Compounds have now been found that are inhibitors of H-PGDS, and at expected efficacious doses, do not significantly inhibit L-PGDS or kinases.

The invention therefore provides, as embodiment E1, a compound of formula (I):

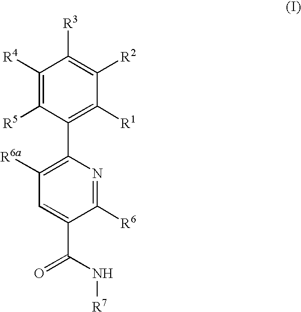

or a pharmaceutically acceptable salt thereof, or a pharmaceutically acceptable solvate of said compound or salt, wherein:

R¹, R², R³, R⁴ and R⁵ are each independently H, F, Cl, —CN, —NH₂, —CH₃, —CH₂F, —CHF₂, —CF₃, —OH, —OCH₃, —OCH₂F, —OCHF₂ or —OCF₃;

R⁶ is H, —NH₂, —OH or —CH₃;

R^{6a} is H, F or Cl;

R⁷ is C₃-C₈ cycloalkyl or C₅-C₁₂ bicycloalkyl, said C₃-C₈ cycloalkyl being optionally fused to a phenyl ring or a 5- or 6-membered aromatic heterocyclic ring; said group R⁷ being (a) optionally substituted by 1-3 substituents selected from R^{a}, —OR^{b}, —S(O)ₙR^{b}, —COR^{b}, —NRₓR^{b}, —OCOR^{b}, —COOR^{b}, —NR^{x}COR^{b}, —CONRₓR^{b}, —NR^{x}SO₂R^{b}, —SO₂NR^{x}R^{b}, —NR^{x}SO₂NR^{x}R^{b}, —NR^{x}COOR^{b}, —NR^{x}CONRₓR^{b}, —OCONR^{x}R^{b}, —OCOOR^{b}, —CONR^{x}SO₂R^{b}, oxo and —CN, and (b) optionally substituted by one or more halo atoms;

R^{a} is in each instance independently selected from C₁-C₆ alkyl, C₃-C₈ cycloalkyl, C₆-C₁₂ bicycloalkyl, Aryl¹, Het¹, Het², Het³ and Het⁴, said C₁-C₆ alkyl, C₃-C₈ cycloalkyl, C₆-C₁₂ bicycloalkyl, Aryl¹, Het¹, Het², Het³ and Het⁴ each being optionally substituted by 1-3 substituents selected from R^{c}, —OR^{d}, —S(O)ₙR^{d}, —COR^{d}, —NR^{x}R^{d}, —OCONR^{d}, —COOR^{d}, —NR^{x}COR^{d}, —CONR^{x}R^{d} —NR^{x}SO₂R^{d}, —SO₂NR^{x}R^{d}, —NR^{x}SO₂NR^{x}R^{d}, —NR^{x}COOR^{d}, —NR^{x}CONR^{x}R^{d}, —OCONR^{x}R^{d}, —OCOOR^{d}, —CONR^{x}SO₂R^{d}, oxo and —CN and one or more halo atoms;

R^{b} is in each instance independently selected from H, C₁-C₈ alkyl, C₃-C₈ cycloalkyl, C₆-C₁₂ bicycloalkyl, Aryl¹, Het¹, Het², Het³ and Het⁴, said C₁-C₆ alkyl, C₃-C₈ cycloalkyl, C₆-C₁₂ bicycloalkyl, Aryl¹, Het¹, Het², Het³ and Het⁴ each being optionally substituted by 1-3 substituents selected from R^{c}, —OR^{d}, —S(O)ₙR^{d}, —COR^{d}, —NR^{x}R^{d}, —OCOR^{d}, —COOR^{d}, —NR^{x}COR^{d}, —CONR^{x}R^{d} —NR^{x}SO₂R^{d}, —SO₂NR^{x}R^{d}, —NR^{x}SO₂NR^{x}R^{d}, —NR^{x}COOR^{d}, —NR^{x}CONR^{x}R^{d}, —OCONR^{x}R^{d}, —OCOOR^{d}, —CONR^{x}SO₂R^{d}, oxo and —CN and one or more halo atoms;

n is 0, 1 or 2;

R^{x} is in each instance independently H, C₁-C₆ alkyl or C₃-C₈ cycloalkyl, said C₁-C₆ alkyl or C₃-C₈ cycloalkyl being optionally substituted by one or more halo atoms;

Aryl¹ is phenyl or naphthyl;

Het¹ is a 3 to 8-membered saturated or partially unsaturated monocyclic heterocycle, containing 1 or 2 heteroatoms selected from O and N;

Het² is a 6 to 12-membered saturated or partially unsaturated multicyclic heterocycle containing 1 or 2 heteroatoms selected from O and N;

Het³ is (i) a 6-membered aromatic heterocycle containing 1-3 N atoms or (ii) a 5-membered aromatic heterocycle containing either (a) 1-4 N atoms or (b) 1 O or S atom and 0-3 N atoms;

Het⁴ is (i) a 10-membered bicyclic aromatic heterocycle containing 1-4 N atoms or (ii) a 9-membered bicyclic aromatic heterocycle containing either (a) 1-4 N atoms or (b) 1 O or S atom and 0-3 N atoms;

R^{c} is in each instance independently selected from C₁-C₆ alkyl, C₃-C₈ cycloalkyl, C₆-C₁₂ bicycloalkyl, Aryl², Het⁵, Het⁶, Het⁷ and Het⁸, said C₁-C₆ alkyl, C₃-C₈ cycloalkyl, C₆-C₁₂ bicycloalkyl, Aryl², Het⁵, Het⁶, Het⁷ and Het⁸ each being optionally substituted by 1-3 substituents selected from R^{e} and one or more halo atoms;

R^{d} is in each instance independently selected from H, C₁-C₆ alkyl, C₃-C₈ cycloalkyl, C₆-C₁₂ bicycloalkyl, Aryl², Het⁵, Het⁶, Het⁷ and Het⁸, said C₁-C₆ alkyl, C₃-C₈ cycloalkyl, C₆-C₁₂ bicycloalkyl, Aryl², Het⁵, Het⁶, Het⁷ and Het⁸ each being optionally substituted by 1-3 substituents selected from R^{e} and one or more halo atoms;

Aryl² is phenyl or naphthyl;

Het⁵ is a 3 to 8-membered saturated or partially unsaturated monocyclic heterocycle, containing 1 or 2 heteroatoms selected from O and N;

Het⁶ is a 6 to 12-membered saturated or partially unsaturated multicyclic heterocycle containing 1 or 2 heteroatoms selected from O and N;

Het⁷ is (i) a 6-membered aromatic heterocycle containing 1-3 N atoms or (ii) a 5-membered aromatic heterocycle containing either (a) 1-4 N atoms or (b) 1 O or S atom and 0-3 N atoms;

Het⁸ is (i) a 10-membered bicylic aromatic heterocycle containing 1-4 N atoms or (ii) a 9-membered bicylic aromatic heterocycle containing either (a) 1-4 N atoms or (b) 1 O or S atom and 0-3 N atoms; and

R^{e} is —OR^{x}, —S(O)ₙR^{x}, —COR^{x}, —NR^{x}R^{x}, —OCOR^{x}, —COOR^{x}, —NR^{x}COR^{x}, —CONR^{x}R^{x} , —NR^{x}SO₂R^{x}, —SO₂NR^{x}R^{x}, —NR^{x}SO₂NR^{x}NR^{x}, —NR^{x}COOR^{x}, —NR^{x}CONR^{x}R^{x}, —OCONR^{x}R^{x}, —OCOOR^{x}, —CONR^{x}SO₂R^{x}, oxo or —CN;

with the proviso that the compound of formula (I) is not:
N-cyclohexyl-2-methyl-6-phenyl-3-pyridinecarboxamide,
N-(2-methylcyclohexyl)-2-methyl-6-(3-bromophenyl)-3-pyridinecarboxamide,
N-{2-[(hydroxyamino)carbonyl]cyclopentyl}-6-(2-methylphenyl)-3-pyridinecarboxamide,
N-{2-[(hydroxyamino)carbonyl]cyclopentyl}-6-(2-methoxyphenyl)-3-pyridinecarboxamide,
N-cyclopropyl-2-phenylnicotinamide,
N-cyclopropyl-2-phenyl-6-chloronicotinamide,
N-cyclopropyl-2-phenyl-6-bromonicotinamide,
N-cyclopropyl-2-(2-chlorophenyl)nicotinamide,
N-cyclopropyl-2-(4-chlorophenyl)nicotinamide, or
N-cyclopropyl-2-(4-methoxyphenyl)nicotinamide.

In a preferred embodiment E2, R¹, R², R³, R⁴ and R⁵ are each independently H, F, —CH₃, or —OCH₃ and R⁶, R^{6a} and R⁷ are as defined in embodiment E1 above.

In a preferred embodiment E3, R¹ and R⁵ are H, R², R³ and R⁴ are each independently H, F, —CH₃, or —OCH₃ and R⁶, R^{6a} and R⁷ are as defined in embodiment E1 above.

In a preferred embodiment E4, R¹, R³, R⁴ and R⁵ are H and R² is F; or R¹, R³, R⁴ and R⁵ are H and R² is —CH₃; or R¹, R³, R⁴ and R⁵ are H and R² is —OCH₃; or R¹, R², R⁴ and R⁵ are H and R³ is F; or R¹, R³ and R⁵ are H and R² and R⁴ are both F; or R¹, R², R³, R⁴ and R⁵ are each H; and R⁶, R^{6a} and R⁷ are as defined in embodiment E1 above.

In a preferred embodiment E5, R¹, R³, R⁴ and R⁵ are H, R² is F and R⁶, R^{6a} and R⁷ are as defined in embodiment E1 above.

In a preferred embodiment E6, R⁶ is H and R¹, R², R³, R⁴, R⁵, R^{6a} and R⁷ are as defined in embodiment E1 above.

In a preferred embodiment E7, R^{6a} is H or Cl and R¹, R², R³, R⁴, R⁵, R⁶ and R⁷ are as defined in embodiment E1 above.

In a preferred embodiment E8, R^{6a} is H and R¹, R², R³, R⁴, R⁵, R⁶ and R⁷ are as defined in embodiment E1 above.

In a preferred embodiment E9, R⁷ is C₃-C₆ cycloalkyl, said C₃-C₆ cycloalkyl being optionally fused to a phenyl ring or a 5- or 6-membered aromatic heterocyclic ring; said group R⁷ being optionally substituted by 1-3 substituents selected from R^{a}, —OR^{b}, —COR^{b}, —NR^{x}R^{b}, —COOR^{b}, —NR^{x}COR^{b}, —CONR^{x}R^{b}, oxo and one or more halo atoms; and R¹, R², R³, R⁴, R⁵, R⁶ and R^{6a} are as defined in embodiment E1 above.

In a preferred embodiment E10, R⁷ is C₃-C₆ cycloalkyl, said C₃-C₆ cycloalkyl being optionally fused to a phenyl ring or a 5- or 6-membered aromatic heterocyclic ring; said group R⁷ being optionally substituted by 1-3 substituents selected from —COOR^{b}, Het³, —COHet¹, Het¹, —OHet³, —OR^{b}, C₁-C₆ alkyl, —CONR^{x}R^{b}, —NR^{x}R^{b}, —NR^{x}COR^{b}, —O(C₁-C₆ alkyl), oxo or one or more halo atoms, said C₁-C₆ alkyl, Het¹ and Het³ each being optionally substituted by 1-3 substituents selected from R^{c}, —OR^{d}, —S(O)ₙR^{d}, —COR^{d}, —NR^{x}R^{d}, —OCOR^{d}, —COOR^{d}, —NR^{x}COR^{d}, —CONR^{x}R^{d} —NR^{x}S(O)ₙR^{d}, —S(O)ₙNR^{x}R^{d} and —CN and one or more halo atoms; and R¹, R², R³, R⁴, R⁵, R⁶ and R^{6a} are as defined in embodiment E1 above.

In a preferred embodiment E11, R⁷ is C₃-C₆ cycloalkyl, said C₃-C₆ cycloalkyl being optionally fused to a phenyl ring or a 5- or 6-membered aromatic heterocyclic ring; said group R⁷ being optionally substituted by 1-2 substituents selected from —COOH, —COO(C₁-C₆ alkyl), Het³, —(C₁-C₆ alkylene)Het¹, —COHet¹, Het¹, —OHet³, —NR^{x}Het¹, —OH, —O(C₁-C₆ alkyl), —O(C₁-C₆ alkylene)OH, —O(C₁-C₆ alkylene)OR^{x}, —(C₁-C₆ alkylene)OH, C₁-C₆ alkyl, —(C₁-C₆ alkylene)CONR^{x}R^{x}, —(C₁-C₆ alkylene)NR^{x}R^{x}, —O(C₁-C₆ alkylene)CONR^{x}R^{x}, —CONR^{x}R^{x}, —CONR^{x}(C₁-C₆ alkylene)Ph, —CONR^{x}(C₁-C₆ alkylene)NR^{x}R^{x}, —NR^{x}R^{x}, —NR^{x}COR^{x}, —O(C₁-C₆alkyl), oxo or one or more halo atoms, each C₁-C₆ alkyl being optionally substituted by one or more halo atoms and said Het³, —(C₁-C₆ alkylene)Het¹, —COHet¹, Het¹, —NR^{x}Het¹ and —OHet³ being optionally substituted by 1-2 substituents selected from C₁-C₆ alkyl, C₃-C₆ cycloalkyl, —OR^{x}, —NR^{x}R^{x}, —COO(C₁-C₆ alkyl) and S(C₁-C₆ alkyl); and R¹, R², R³, R⁴, R⁵, R⁶ and R^{6a} are as defined in embodiment E1 above.

In a preferred embodiment E12, R⁷ is C₃-C₆ cycloalkyl, said C₃-C₆ cycloalkyl being optionally fused to a phenyl, imidazolyl, pyridyl or pyrazolyl ring; said group R⁷ being optionally substituted by 1-2 substituents selected from pyridyl, imidazolyl, (C₁-C₆ alkyl)imidazolyl, (C₁-C₆ alkyl)thioimidazolyl, (C₁-C₆ alkyl)tetrazolyloxy, piperazinylcarbonyl, (C₁-C₆ alkyl)piperazinylcarbonyl, (C₁-C₆ cycloalkyl)piperazinylcarbonyl, (C₁-C₆ alkyl)piperazinyl, [(C₁-C₆ alkyl)-OCO][C₁-C₆ alkyl]piperazinylcarbonyl, aminoazetidinylcarbonyl, pyrrolidinylcarbonyl, hydroxypyrrolidinylcarbonyl, hydroxypyrrolidinyl, aminopyrrolidinylcarbonyl, hydroxypiperidinylcarbonyl, hydroxypiperidinyl, morpholinyl, morpholinylcarbonyl, morpholinyl(C₁-C₆ alkyl), (C₁-C₆ alkyl)piperazinyl(C₁-C₆ alkyl)carboxy, amino, (C₁-C₆ alkyl)amino, furanylamino, (C₁-C₆ haloalkyl)carbonylamino, hydroxy, hydroxy(C₁-C₆ alkyl), hydroxyl(C₁-C₆ alkoxy), C₁-C₆ alkoxy, (C₁-C₆ alkoxy)C₁-C₆ alkoxy, [(C₁-C₆ alkoxy)C₁-C₆ alkyl]amino, [(C₁-C₆ alkoxy)C₁-C₆ alkyl][C₁-C₆ alkyl]amino phenyl(C₁-C₆ alkyl)aminocarbonyl, (phenyl(C₁-C₆ alkyl))(C₁-C₆ alkyl)aminocarbonyl, di-(C₁-C₆ alkyl)aminocarbonyl, (di-(C₁-C₆ alkyl)aminocarbonyl)C₁-C₆ alkoxy, oxo, (di-(C₁-C₆ alkyl)aminocarbonyl)C₁-C₆ alkyl, (di-(C₁-C₆ alkyl)amino)C₁-C₆ alkyl, (C₁-C₆ alkyl)oxycarbonyl, carboxy, oxazepinyl, C₁-C₆ alkyl, (C₃-C₈ cycloalkyl)aminocarbonyl, ((C₁-C₆ alkylamino)C₁-C₆ alkyl)(C₁-C₆ alkyl)aminocarbonyl, (C₁-C₆ alkyl)carbonylamino and fluoro; and R¹, R², R³, R⁴, R⁵, R⁶ and R^{6a} are as defined in embodiment E1 above.

In a preferred embodiment E13, R⁷ is C₃-C₆ cycloalkyl, said C₃-C₆ cycloalkyl being optionally fused to a phenyl, imidazolyl, pyridyl or pyrazolyl ring; said group R⁷ being optionally substituted by 1 substituent selected from (2-methylpiperazin-4-yl)carbonyl, 1-cyclopropylpiperazin-4-ylcarbonyl, (3-methylpiperazine-4-yl)carbonyl, 1-tert-butyloxycarbonyl-3-methylpiperazin-4-ylcarbonyl, 3-hydroxypyrrolidinyl, 4-hydroxypiperidinyl, morpholin-4-ylmethyl, (1-methylpiperazin-4-yl)methyl, (3-aminoazetidin-1-yl)carbonyl, (3-aminopyrrolidin-1-yl)carbonyl, pyrid-2-yl, methoxycarbonyl, carboxy, (1-methylpiperazin-4-yl)carbonyl, piperazin-4-ylcarbonyl, (4-hydroxypiperidin-1-yl)carbonyl, (3-hydroxypyrrolidin-1-yl)carbonyl, hydroxyl, hydroxymethyl, pyrrolidin-1-ylcarbonyl, amino, oxazepinyl, methyl, 2-methoxyethoxy, benzylaminocarbonyl, dimethylaminocarbonyl, (methyl)(ethyl)aminocarbonyl, cyclopentylaminocarbonyl, isopropylaminocarbonyl, morpholin-4-ylcarbonyl, (2-methylaminoethyl)(methyl)aminocarbonyl, tert-butylaminocarbonyl, (benzyl)(methyl)aminocarbonyl, (dimethylamino)methyl, diethylaminocarbonyl, (1-ethylpiperazin-4-yl)carbonyl, 2-hydroxyethoxy, methylamino, methoxy, (dimethylaminocarbonyl)methyl, 2-methylthioimidazol-3-yl, methylcarbonylamino, 2-methoxyethylamino, (2-methoxyethyl)(methyl)amino, furan-3-ylamino, trifluoromethylcarbonylamino, oxo, ethyl, isopropyl, imidazol-1-yl, 1-hydroxy-1-methylethyl, (dimethylaminocarbonyl)methyl, morpholin-4-yl, 1-methylpiperazin-4-yl, imidazol-2-yl, 2-methylimidazol-1-ylmethyl, 2-methylimidazol-1-yl, 2-isopropylimidazol-1-yl and 1-methyltetrazol-5-yloxy or 2 substituents selected from fluoro, methyl, hydroxyl, carboxy and (1-methylpiperazin-4-yl)carbonyl; and R¹, R², R³, R⁴, R⁵, R⁶ and R^{ha} are as defined in embodiment E1 above.

In a preferred embodiment E14, R⁷ is a cyclopropyl group, with the optional substitution defined in any one of embodiments E9, E10, E11, E12 or E13; and R¹, R², R³, R⁴, R⁵, R⁶ and R^{6a} are as defined in embodiment E1 above.

In a preferred embodiment E15, R⁷ is a cyclopentyl group, with the optional substitution defined in any one of embodiments E9, E10, E11, E12 or E13; and R¹, R², R³, R⁴, R⁵, R⁶ and R^{6a} are as defined in embodiment E1 above.

In a preferred embodiment E16, R⁷ is a cyclohexyl group, with the optional substitution defined in any one of embodiments E9, E10, E11, E12 or E13; and R¹, R², R³, R⁴, R⁵, R⁶ and R^{6a} are as defined in embodiment E1 above.

In a preferred embodiment E17, R⁷ is 1,2,3,4-tetrahydronaphthalenyl, 4,5,6,7-tetrahydro-1H-benzimidazolyl, 5,6,7,8-tetrahydroquinolinyl, 4,5,6,7-tetrahydro-1H-indazolyl or 2,3-dihydro-1H-indenyl, said 1,2,3,4-tetrahydronaphthalenyl, 4,5,6,7-tetrahydro-1H-benzimidazolyl, 5,6,7,8-tetrahydroquinolinyl, 4,5,6,7-tetrahydro-1H-indazolyl and 2,3-dihydro-1H-indenyl being optionally substituted by one group selected from C₁-C₆ alkyl group and hydroxyl; and R¹, R², R³, R⁴, R⁵, R⁶ and R^{6a} are as defined in embodiment E1 above.

In a preferred embodiment E18, R⁷ is C₅-C₁₂ bicycloalkyl, particularly bicyclopentyl; and R¹, R², R³, R⁴, R⁵, R⁶ and R^{6a} are as defined in embodiment E1 above.

In a preferred embodiment E19, the compound of formula (I) is a compound of formula (Ia):

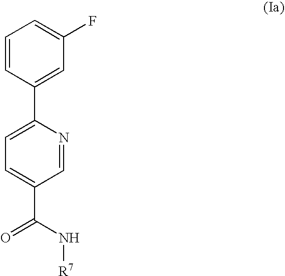

or a pharmaceutically acceptable salt thereof, or a pharmaceutically acceptable solvate of said compound or salt, wherein R⁷ is as defined above in any one of embodiments E1, E9, E10, E11, E12, E13, E14, E15, E16, E17 or E18.

Further preferred embodiments of the invention are created by combining the definitions given for R¹-R⁵ in any one of embodiments E1, E2, E3, E4 or E5 with the definition given for R⁶ in embodiment E1 or E6, the definition given for R^{6a} in any one of embodiments E1, E7 or E8 and the definition given for R⁷ in any one of embodiments E1, E9, E10, E11, E12, E13, E14, E15, E16, E17 or E18.

In preferred embodiment E20, the invention provides a compound selected from:
6-(3-fluorophenyl)-N-{cis-3-[(4-hydroxypiperidin-1-yl)carbonyl]cyclohexyl}nicotinamide;
N-[trans-4-(dimethylcarbamoyl)cyclohexyl]-6-(3-fluorophenyl)nicotinamide;
N-[4-trans-(cyclopropylhydroxymethyl)cyclohexyl]-6-(3-fluorophenyl)nicotinamide; and
N-{trans-4-[acetamidoethyl]cyclohexyl}-6-(3-fluorophenyl)nicotinamide;
   or a pharmaceutically acceptable salt or solvate thereof.

Particularly preferred is 6-(3-fluorophenyl)-N-{cis-3-[(4-hydroxypiperidin-1-yl)carbonyl]cyclohexyl}nicotinamide, or a pharmaceutically acceptable salt or solvate thereof, as well as each of its enantiomers, 6-(3-fluorophenyl)-N-{(1R,3S)-3-[(4-hydroxypiperidin-1-yl)carbonyl]cyclohexyl}nicotinamide and 6-(3-fluorophenyl)-N-{(1S,3R)-3-[(4-hydroxypiperidin-1-yl)carbonyl]cyclohexyl}nicotinamide, or a pharmaceutically acceptable salt or solvate of either. Most preferred is 6-(3-fluorophenyl)-N-{(1S,3R)-3-[(4-hydroxypiperidin-1-yl)carbonyl]cyclohexyl}nicotinamide or a pharmaceutically acceptable salt or solvate thereof.

The present invention also provides: a method of treating a disease or condition mediated at least in part by prostaglandin D₂ produced by H-PGDS, in a subject in need of such treatment, comprising administering to the subject a therapeutically effective amount of a compound of formula (I), or a pharmaceutically acceptable salt or solvate thereof; the use of a compound of formula (I), or a pharmaceutically acceptable salt or solvate thereof, for the manufacture of a medicament for treating a disease or condition mediated at least in part by prostaglandin D₂ produced by H-PGDS; a compound of formula (I), or a pharmaceutically acceptable salt or solvate thereof, for use as a medicament; a compound of formula (I), or a pharmaceutically acceptable salt or solvate thereof, for use in the treatment of a disease or condition mediated at least in part by prostaglandin D₂ produced by H-PGDS; a pharmaceutical composition comprising a compound of formula (I), or a pharmaceutically acceptable salt or solvate thereof, and a pharmaceutically acceptable excipient; a pharmaceutical composition for the treatment of a disease or condition mediated at least in part by prostaglandin D₂ produced by H-PGDS comprising a compound of formula (I), or a pharmaceutically acceptable salt or solvate thereof.

The disease or condition mediated at least in part by prostaglandin D₂ produced by H-PGDS is preferably an allergic or respiratory condition such as allergic rhinitis, nasal congestion, rhinorrhea, perennial rhinitis, nasal inflammation, asthma of all types, chronic obstructive pulmonary disease (COPD), chronic or acute bronchoconstriction, chronic bronchitis, small airways obstruction, emphysema, chronic eosinophilic pneumonia, adult respiratory distress syndrome, exacerbation of airways hyper-reactivity consequent to other drug therapy, airways disease that is associated with pulmonary hypertension, acute lung injury, bronchiectasis, sinusitis, allergic conjunctivitis or atopic dermatitis, particularly asthma or chronic obstructive pulmonary disease, most particularly asthma.

Other diseases and conditions of interest are inflammation (including neuroinflammation), arthritis (including rheumatoid arthritis, spondyloarthropathies, systemic lupus erythematous arthritis, osteoarthritis and gouty arthritis), pain, fever, pulmonary sarcoisosis, silicosis, cardiovascular disease (including atherosclerosis, myocardial infarction, thrombosis, congestive heart failure and cardiac reperfusion injury), cardiomyopathy, stroke, ischaemia, reperfusion injury, brain edema, brain trauma, neurodegeneration, liver disease, inflammatory bowel disease (including Crohn's disease and ulcerative colitis), nephritis, retinitis, retinopathy, macular degeneration, glaucoma, diabetes (including type 1 and type 2 diabetes), diabetic neurorpathy, viral and bacterial infection, myalgia, endotoxic shock, toxic shock syndrome, autoimmune disease, osteoporosis, multiple sclerosis, endometriosis, menstrual cramps, vaginitis, candidiasis, cancer, fibrosis, obesity, muscular dystrophy, polymyositis, Alzheimer's disease, skin flushing, eczema, psoriasis, atopic dermatitis and sunburn.

Types of asthma include atopic asthma, non-atopic asthma, allergic asthma, atopic bronchial IgE-mediated asthma, bronchial asthma, essential asthma, true asthma, intrinsic asthma caused by pathophysiologic disturbances, extrinsic asthma caused by environmental factors, essential asthma of unknown or inapparent cause, bronchitic asthma, emphysematous asthma, exercise-induced asthma, allergen induced asthma, cold air induced asthma, occupational asthma, infective asthma caused by bacterial, fungal, protozoal, or viral infection, non-allergic asthma, incipient asthma, wheezy infant syndrome and bronchiolytis.

Included in the use of the compounds of formula (I) for the treatment of asthma, is palliative treatment for the symptoms and conditions of asthma such as wheezing, coughing, shortness of breath, tightness in the chest, shallow or fast breathing, nasal flaring (nostril size increases with breathing), retractions (neck area and between or below the ribs moves inward with breathing), cyanosis (gray or bluish tint to skin, beginning around the mouth), runny or stuffy nose, and headache.

The present invention also provides any of the uses, methods or compositions as defined above wherein the compound of formula (I), or pharmaceutically acceptable salt or solvate thereof, is used in combination with another pharmacologically active compound, particularly one of the compounds listed in Table 1 below. Specific combinations useful according to the present invention include combinations comprising a compound of formula (I), or a pharmaceutically acceptable salt or solvate thereof, and (i) a glucocorticosteroid or DAGR (dissociated agonist of the corticoid receptor); (ii) a β₂ agonist, an example of which is a long-acting β₂ agonist; (iii) a muscarinic M3 receptor antagonist or an anticholinergic agent; (iv) a histamine receptor antagonist, which may be an H1 or an H3 antagonist; (v) a 5-lipoxygenase inhibitor; (vi) a thromboxane inhibitor; or (vii) an LTD₄ inhibitor. Generally, the compounds of the combination will be administered together as a formulation in association with one or more pharmaceutically acceptable excipients.

### Table I

(a) 5-lipoxygenase activating protein (FLAP) antagonists;
(b) Leukotriene antagonists (LTRAs) including antagonists of LTB₄, LTC₄, LTD₄, and LTE₄;
(c) Histamine receptor antagonists including H1 and H3 antagonists;
(d) α₁- and α₂-adrenoceptor agonist vasoconstrictor sympathomimetic agents for decongestant use;
(e) muscarinic M3 receptor antagonists or anticholinergic agents;
(f) PDE inhibitors, e.g. PDE3, PDE4 and PDE5 inhibitors, such as theophylline;
(g) Sodium cromoglycate;
(h) COX inhibitors both non-selective and selective COX-1 or COX-2 inhibitors (such as NSAIDs);
(i) glucocorticosteroids or DAGR (dissociated agonists of the corticoid receptor);
(j) Monoclonal antibodies active against endogenous inflammatory entities;
(k) β2 agonists, including long-acting β2 agonists;
(l) Integrin antagonists;
(m) Adhesion molecule inhibitors including VLA-4 antagonists;
(n) Kinin-B₁- and B₂-receptor antagonists;
(o) Immunosuppressive agents, including inhibitors of the IgE pathway, and cyclosporin;
(p) Inhibitors of matrix metalloproteases (MMPs), such as, MMP9, and MMP12;
(q) Tachykinin NK₁, NK₂ and NK₃ receptor antagonists;
(r) Protease inhibitors, such as elastase inhibitors, chymase and cathepsin G;
(s) Adenosine A2a receptor agonists and A2b antagonists;
(t) Inhibitors of urokinase;
(u) Compounds that act on dopamine receptors, such as D2 agonists;
(v) Modulators of the NFκB pathway, such as IKK inhibitors;
(w) modulators of cytokine signaling pathways such as syk kinase, JAK kinase inhibitors, p38 kinase, SPHK-1 kinase, Rho kinase, EGF-R or MK-2;
(x) Agents that can be classed as mucolytics or anti-tussive, and mucokinetics;
(y) Antibiotics;
(z) Antivirals;
(aa) Vaccines;
(bb) Chemokines;
(cc) Epithelial sodium channel (ENaC) blockers or Epithelial sodium channel (ENaC) inhibitors;
(dd) P2Y2 Agonists and other Nucleotide receptor agonists;
(ee) Inhibitors of thromboxane;
(ff) Niacin;
(gg) Inhibitors of 5-lypoxygenase (5-LO); and
(hh) Adhesion factors including VLAM, ICAM, and ELAM.

Besides being useful for human treatment, compounds of formula (I) are also useful for veterinary treatment of companion animals, exotic animals and farm animals.

When used in the present application, the following abbreviations have the meanings set out below:

APCI (in relation to mass spectrometry) is atmospheric pressure chemical ionization;

BOC or Boc is tert-butyloxycarbonyl;

BOP is (benzotriazol-1-yloxy)tris(dimethylamino)phosphonium hexafluorophosphate;

CDl is 1,1-carbonyldiimidazole;

CH₂Cl₂ is dichloromethane;

CO₂Et is ethyl carboxylate;

DCC is N,N′-dicyclohexylcarbodiimide;

DCM is dichloromethane;

CDCl₃ is deuterochloroform;

DEA is diethylamine;

DIEA is diisopropylethylamine;

DIPEA is N,N-diisopropylethylamine;

DMA is N,N-dimethylacetamide;

DMAP is 4-dimethylaminopyridine

DMF is dimethylformamide;

DMSO is dimethyl sulphoxide;

DMSO-d₆ is fully deuterated dimethyl sulphoxide;

EDC/EDAC is N-(3-Dimethylaminopropyl)-N′-ethylcarbodiimide hydrochloride;

ES (in relation to mass spectrometry) is electrospray;

Et is ethyl;

EtOAc is ethyl acetate;

GCMS is gas chromatography mass spectrometry;

h is hour(s);

HATU is N,N,N′,N′-tetramethyl-O-(7-azabenzotriazol-1-yl)uronium hexafluorophosphate;

HBTU is N,N,N′,N′-tetramethyl-O-(1H-benzotriazol-1-yl)uronium hexafluorophosphate;

1H NMR or ¹H NMR is proton nuclear magnetic resonance;

HOAt is 1-hydroxy-7-azabenzotriazole;

HOBt is 1-hydroxybenzotriazole;

HPLC is high performance liquid chromatography;

HRMS is high resolution mass spectrometry;

IPA is isopropyl alcohol;

iPr is isopropyl;

LCMS is liquid chromatography mass spectrometry;

LRMS is low resolution mass spectrometry;

Me is methyl;

MeCN is acetonitrile;

MeOH is methanol;

MeOD-d₄ is fully deuterated methanol;

MgSO₄ is magnesium sulphate;

min is minute(s);

NH₄Cl is ammonium chloride;

NH₄OH is a solution of ammonia in water;

MS is mass spectroscopy;

NMM is 4-methylmorpholine;

NMP is N-methylpyrrolidinone;
RT is retention time;

TBTU is O-(benzotriazol-1-yl)-N,N,N′,N′-tetramethyluronium tetrafluoroborate; TEA is triethylamine;

TFA is trifluoroacetic acid; and

THF is tetrahydrofuran.

Unless otherwise defined herein, scientific and technical terms used in connection with the present invention shall have the meanings that are commonly understood by those of ordinary skill in the art.

The phrase “therapeutically effective” is intended to qualify the amount of compound or pharmaceutical composition, or the combined amount of active ingredients in the case of combination therapy. This amount or combined amount will achieve the goal of treating the relevant condition.

The term “treatment,” as used herein to describe the present invention and unless otherwise qualified, means administration of the compound, pharmaceutical composition or combination to effect preventative, palliative, supportive, restorative or curative treatment. The term treatment encompasses any objective or subjective improvement in a subject with respect to a relevant condition or disease.

The term “preventive treatment,” as used herein to describe the present invention, means that the compound, pharmaceutical composition or combination is administered to a subject to inhibit or stop the relevant condition from occurring in a subject, particularly in a subject or member of a population that is significantly predisposed to the relevant condition.

The term “palliative treatment,” as used herein to describe the present invention, means that the compound, pharmaceutical composition or combination is administered to a subject to remedy signs and/or symptoms of a condition, without necessarily modifying the progression of, or underlying etiology of, the relevant condition.

The term “supportive treatment,” as used herein to describe the present invention, means that the compound, pharmaceutical composition or combination is administered to a subject as a part of a regimen of therapy, but that such therapy is not limited to administration of the compound, pharmaceutical composition or combination. Unless otherwise expressly stated, supportive treatment may embrace preventive, palliative, restorative or curative treatment, particularly when the compounds or pharmaceutical compositions are combined with another component of supportive therapy.

The term “restorative treatment,” as used herein to describe the present invention, means that the compound, pharmaceutical composition or combination is administered to a subject to modify the underlying progression or etiology of a condition. Non-limiting examples include an increase in forced expiratory volume in one second (FEV 1) for lung disorders, inhibition of progressive nerve destruction, reduction of biomarkers associated and correlated with diseases or disorders, a reduction in relapses, improvement in quality of life and the like.

The term “curative treatment,” as used herein to describe the present invention, means that compound, pharmaceutical composition or combination is administered to a subject for the purpose of bringing the disease or disorder into complete remission, or that the disease or disorder is undetectable after such treatment.

The term “alkyl”, alone or in combination, means an acyclic, saturated hydrocarbon group of the formula CₙH₂ₙ₊₁ which may be linear or branched. Examples of such groups include methyl, ethyl, n-propyl, isopropyl, n-butyl, isobutyl, sec-butyl, tert-butyl, pentyl, iso-amyl and hexyl. Unless otherwise specified, an alkyl group comprises from 1 to 6 carbon atoms.

The term “alkylene” means a bivalent acyclic, saturated hydrocarbon group of the formula CₙH₂ₙ which may be linear or branched. Example of such groups include —CH₂—, —CH(CH₃)—, —CH₂CH₂—, —CH(CH₃)CH₂—, —CH(CH₃)CH(CH₃)— and —CH₂CH₂CH₂—. Unless otherwise specified, an alkylene group comprises from 1 to 6 carbon atoms.

The carbon atom content of alkyl and various other hydrocarbon-containing moieties is indicated by a prefix designating a lower and upper number of carbon atoms in the moiety, that is, the prefix Cᵢ-Cⱼ indicates a moiety of the integer “i” to the integer “j” carbon atoms, inclusive. Thus, for example, C₁-C₆ alkyl refers to alkyl of one to six carbon atoms, inclusive.

The term “hydroxy,” as used herein, means an OH radical.

Het¹ and Het⁵ are saturated or partially saturated (i.e. non aromatic) heterocycles and may be attached via a ring nitrogen atom or a ring carbon atom. Equally, when substituted, the substituent may be located on a ring nitrogen atom or a ring carbon atom. Specific examples include oxiranyl, aziridinyl, oxetanyl, azetidinyl, tetrahydrofuranyl, pyrrolidinyl, tetrahydropyranyl, piperidinyl, 1,4-dioxanyl, morpholinyl, piperazinyl, azepanyl, oxepanyl, oxazepanyl and diazepinyl.

Het² and Het⁶ are saturated or partially saturated heterocycles and may be attached via a ring nitrogen atom or a ring carbon atom. Equally, when substituted, the substituent may be located on a ring nitrogen atom or a ring carbon atom. Het² and Het⁶ are multicyclic heterocyclic groups, containing two or more rings. Such rings may be joined so as to create a bridged, fused or spirofused ring system, as illustrated with two six-membered rings below (heteroatoms not shown):

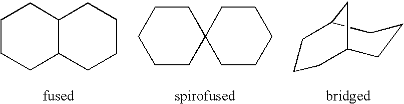

Het² and Het⁶ may be fully saturated or partially unsaturated, i.e. they may have one or more degrees of unsaturation but may not be fully aromatic. In the case of a fused ring system, one of the rings may be aromatic but not both of them.

Het³ and Het⁷ are aromatic heterocycles and may be attached via a ring carbon atom or a ring nitrogen atom with an appropriate valency. Equally, when substituted, the substituent may be located on a ring carbon atom or a ring nitrogen atom with an appropriate valency. Specific examples include thienyl, furanyl, pyrrolyl, pyrazolyl, imidazoyl, oxazolyl, isoxazolyl, thiazolyl, isothiazolyl, triazolyl, oxadiazolyl, thiadiazolyl, tetrazolyl, pyridyl, pyridazinyl, pyrimidinyl and pyrazinyl.

Het⁴ and Het⁸ are aromatic heterocycles and may be attached via a ring carbon atom or a ring nitrogen atom with an appropriate valency. Equally, when substituted, the substituent may be located on a ring carbon atom or a ring nitrogen atom with an appropriate valency. Het⁴ and Het⁸ are aromatic and are therefore necessarily fused bicycles. Specific examples include benzofuranyl, benzothienyl, indolyl, benzimidazolyl, indazolyl, benzotriazolyl, pyrrolo[2,3-b]pyridyl, pyrrolo[2,3-c]pyridyl, pyrrolo[3,2-c]pyridyl, pyrrolo[3,2-b]pyridyl, imidazo[4,5-b]pyridyl, imidazo[4,5-c]pyridyl, pyrazolo[4,3-d]pyridyl, pyrazolo[4,3-c]pyridyl, pyrazolo[3,4-c]pyridyl, pyrazolo[3,4-b]pyridyl, isoindolyl, indazolyl, purinyl, indolizinyl, imidazo[1,2-a]pyridyl, imidazo[1,5-a]pyridyl, pyrazolo[1,5-a]pyridyl, pyrrolo[1,2-b]pyridazinyl, imidazo[1,2-c]pyrimidinyl, quinolinyl, isoquinolinyl, cinnolinyl, quinazolinyl, quinoxalinyl, phthalazinyl, 1,6-naphthyridinyl, 1,7-naphthyridinyl, 1,8-naphthyridinyl, 1,5-naphthyridinyl, 2,6-naphthyridinyl, 2,7-naphthyridinyl, pyrido[3,2-d]pyrimidinyl, pyrido[4,3-d]pyrimidinyl, pyrido[3,4-d]pyrimidinyl, pyrido[2,3-d]pyrimidinyl, pyrido[2,3-d]pyrazinyl, pyrido[3,4-b]pyrazinyl, pyrimido[5,4-d]pyrimidinyl, pyrazino[2,3-b]pyrazinyl and pyrimido[4,5-d]pyrimidine.

The term “cycloalkyl” means a means a monocyclic, saturated hydrocarbon group of the formula CₙH₂ₙ₋₁. Examples include cyclopropyl, cyclobutyl, cyclopentyl, cyclohexyl, and cycloheptyl. Unless otherwise specified, a cycloalkyl group comprises from 3 to 8 carbon atoms.

The term bicycloalkyl means a bicyclic, saturated hydrocarbon group of the formula CₙH₂ₙ₋₃ in which the two rings are joined in a fused, spiro-fused or bridged manner (see above). The following groups are illustrative of C₅-C₁₂ bicycloalkyl (note that as drawn, these groups have an extra hydrogen atom where the linking bond would be):

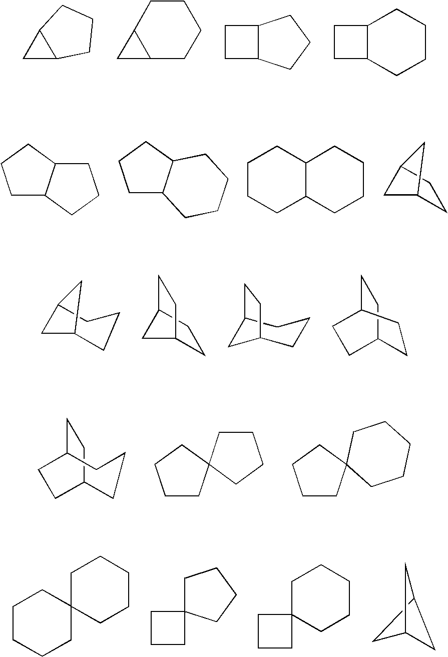

In the definition of R⁷, the C₃-C₈ cycloalkyl ring may be fused to a phenyl ring or a 5- or 6-membered aromatic heterocylic ring. In the case of such fusion, the R⁷ group may be attached to the amide nitrogen through the cycloalkyl ring or through the fused ring but is preferably attached through the cycloalkyl ring. Equally, in the case where the R⁷ group is substituted, such substitution may occur on the cycloalkyl ring, the fused ring or both. The 5- or 6-membered aromatic heterocyclic ring is preferably (i) a 6-membered aromatic heterocycle containing 1-3 N atoms or (ii) a 5-membered aromatic heterocycle containing either (a) 1-4 N atoms or (b) 1 O or S atom and 0-3 N atoms. Specific examples of preferred 5- or 6-membered aromatic heterocyclic rings are given above in relation to Het³/Het⁷. Where the C₃-C₈ cycloalkyl ring of R⁷ is fused, it is particularly preferred that it is fused to a phenyl, imidazolyl, pyridyl or pyrazolyl ring.

The term “oxo” means a doubly bonded oxygen.

The term “alkoxy” means a radical comprising an alkyl radical that is bonded to an oxygen atom, such as a methoxy radical. Examples of such radicals include methoxy, ethoxy, propoxy, isopropoxy, butoxy and tert-butoxy.

As used herein, the terms “co-administration”, “co-administered” and “in combination with”, referring to a combination of a compound of formula (I) and one or more other therapeutic agents include the following:
simultaneous administration of such a combination of a compound of formula (I) and a further therapeutic agent to a patient in need of treatment, when such components are formulated together into a single dosage form which releases said components at substantially the same time to said patient,
substantially simultaneous administration of such a combination of a compound of formula (I) and a further therapeutic agent to a patient in need of treatment, when such components are formulated apart from each other into separate dosage forms which are taken at substantially the same time by said patient, whereupon said components are released at substantially the same time to said patient, and
sequential administration of such a combination of a compound of formula (I) and a further therapeutic agent to a patient in need of treatment, when such components are formulated apart from each other into separate dosage forms which are taken at consecutive times by said patient with a significant time interval between each administration, whereupon said components are released at substantially different times to said patient.

The term ‘excipient’ is used herein to describe any ingredient other than a compound of formula (I). The choice of excipient will to a large extent depend on factors such as the particular mode of administration, the effect of the excipient on solubility and stability, and the nature of the dosage form. The term “excipient” encompasses diluent, carrier or adjuvant.

Pharmaceutically acceptable salts of the compounds of formula (I) include the acid addition and base salts thereof.

Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate, naphatlene-1,5-disulfonic acid and xinofoate salts.

Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts. For a review on suitable salts, see Handbook of Pharmaceutical Salts: Properties, Selection, and Use by Stahl and Wermuth (Wiley-VCH, 2002).

Pharmaceutically acceptable salts of compounds of formula (I) may be prepared by one or more of three methods:
(i) by reacting the compound of formula (I) with the desired acid or base;
(ii) by removing an acid- or base-labile protecting group from a suitable precursor of the compound of formula (I) or by ring-opening a suitable cyclic precursor, for example, a lactone or lactam, using the desired acid or base; or
(iii) by converting one salt of the compound of formula (I) to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

The compounds of formula (I) may also exist in unsolvated and solvated forms. The term ‘solvate’ is used herein to describe a molecular complex comprising the compound of formula (I), or a pharmaceutically acceptable salt thereof, and one or more pharmaceutically acceptable solvent molecules, for example, ethanol. The term ‘hydrate’ is employed when said solvent is water.

A currently accepted classification system for organic hydrates is one that defines isolated site, channel, or metal-ion coordinated hydrates—see Polymorphism in Pharmaceutical Solids by K. R. Morris (Ed. H. G. Brittain, Marcel Dekker, 1995). Isolated site hydrates are ones in which the water molecules are isolated from direct contact with each other by intervening organic molecules. In channel hydrates, the water molecules lie in lattice channels where they are next to other water molecules. In metal-ion coordinated hydrates, the water molecules are bonded to the metal ion.

When the solvent or water is tightly bound, the complex will have a well-defined stoichiometry independent of humidity. When, however, the solvent or water is weakly bound, as in channel solvates and hygroscopic compounds, the water/solvent content will be dependent on humidity and drying conditions. In such cases, non-stoichiometry will be the norm.

Also included within the scope of the invention are multi-component complexes (other than salts and solvates) wherein the drug and at least one other component are present in stoichiometric or non-stoichiometric amounts. Complexes of this type include clathrates (drug-host inclusion complexes) and co-crystals. The latter are typically defined as crystalline complexes of neutral molecular constituents which are bound together through non-covalent interactions, but could also be a complex of a neutral molecule with a salt. Co-crystals may be prepared by melt crystallisation, by recrystallisation from solvents, or by physically grinding the components together—see Chem Commun, 17, 1889-1896, by O. Almarsson and M. J. Zaworotko (2004). For a general review of multi-component complexes, see J Pharm Sci, 64 (8), 1269-1288, by Haleblian (August 1975).

The compounds of the invention may exist in a continuum of solid states ranging from fully amorphous to fully crystalline. The term ‘amorphous’ refers to a state in which the material lacks long range order at the molecular level and, depending upon temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterised by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterised by a phase change, typically first order (‘melting point’).

The compounds of formula (I) may also exist in a mesomorphic state (mesophase or liquid crystal) when subjected to suitable conditions. The mesomorphic state is intermediate between the true crystalline state and the true liquid state (either melt or solution). Mesomorphism arising as the result of a change in temperature is described as ‘thermotropic’ and that resulting from the addition of a second component, such as water or another solvent, is described as ‘lyotropic’. Compounds that have the potential to form lyotropic mesophases are described as ‘amphiphilic’ and consist of molecules which possess an ionic (such as —COO⁻Na⁺, —COO⁻K⁺, or —SO₃⁻Na⁺) or non-ionic (such as —N⁻N⁺(CH₃)₃) polar head group. For more information, see Crystals and the Polarizing Microscope by N. H. Hartshorne and A. Stuart, 4^{th} Edition (Edward Arnold, 1970).

Hereinafter all references to compounds of formula (I) (also referred to as compounds of the invention) include references to salts, solvates, multi-component complexes and liquid crystals thereof and to solvates, multi-component complexes and liquid crystals of salts thereof.

Also included within the scope of the invention are all polymorphs and crystal habits of compounds of formula (I), prodrugs and isomers thereof (including optical, geometric and tautomeric isomers) as hereinafter defined and isotopically-labeled forms thereof.

As indicated, so-called ‘prodrugs’ of the compounds of formula (I) are also within the scope of the invention. Thus certain derivatives of a compound of formula (I) which may have little or no pharmacological activity themselves can, when administered into or onto the body, be converted into a compound of formula (I) having the desired activity, for example, by hydrolytic cleavage. Such derivatives are referred to as ‘prodrugs’. Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (Ed. E. B. Roche, American Pharmaceutical Association).

Prodrugs in accordance with the invention can, for example, be produced by replacing appropriate functionalities present in the compounds of formula (I) with certain moieties known to those skilled in the art as ‘pro-moieties’ as described, for example, in Design of Prodrugs by H. Bundgaard (Elsevier, 1985).

Some examples of prodrugs in accordance with the invention include:
(i) where the compound of formula (I) contains a carboxylic acid functionality (—COOH), an ester thereof, for example, a compound wherein the hydrogen of the carboxylic acid functionality of the compound of formula (I) is replaced by (C₁-C₈)alkyl;
(ii) where the compound of formula (I) contains an alcohol functionality (—OH), an ether thereof, for example, a compound wherein the hydrogen of the alcohol functionality of the compound of formula (I) is replaced by (C₁-C₆)alkanoyloxymethyl; and
(iii) where the compound of formula (I) contains a primary or secondary amino functionality (—NH₂ or —NHR where R≠H), an amide thereof, for example, a compound wherein, as the case may be, one or both hydrogens of the amino functionality of the compound of formula (I) is/are replaced by (C₁-C₁₀)alkanoyl.

Further examples of replacement groups in accordance with the foregoing examples and examples of other prodrug types may be found in the aforementioned references.

Moreover, certain compounds of formula (I) may themselves act as prodrugs of other compounds of formula (I).

Compounds of formula (I) containing one or more asymmetric carbon atoms can exist as two or more stereoisomers. Where a compound of formula (I) contains an alkenyl or alkenylene group, geometric cis/trans (or Z/E) isomers are possible. Where structural isomers are interconvertible via a low energy barrier, tautomeric isomerism (‘tautomerism’) can occur. This can take the form of proton tautomerism in compounds of formula (I) containing, for example, an imino, keto, or oxime group, or so-called valence tautomerism in compounds which contain an aromatic moiety. It follows that a single compound may exhibit more than one type of isomerism.

Included within the scope of the present invention are all stereoisomers, geometric isomers and tautomeric forms of the compounds of formula (I), including compounds exhibiting more than one type of isomerism, and mixtures of one or more thereof. Also included are acid addition or base salts wherein the counterion is optically active, for example, d-lactate or 1-lysine, or racemic, for example, dl-tartrate or dl-arginine.

Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). Alternatively, the racemate (or a racemic precursor) may be reacted with a suitable optically active compound, for example, an alcohol, or, in the case where the compound of formula (I) contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person. Chiral compounds of formula (I) (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine. Concentration of the eluate affords the enriched mixture. Chiral chromatography using sub-and supercritical fluids may be employed. Methods for chiral chromatography useful in some embodiments of the present invention are known in the art (see, for example, Smith, Roger M., Loughborough University, Loughborough, UK; Chromatographic Science Series (1998), 75 (Supercritical Fluid Chromatography with Packed Columns), pp. 223-249 and references cited therein). In some relevant examples herein, columns were obtained from Chiral Technologies, Inc, West Chester, Pa., USA, a subsidiary of Daicel® Chemical Industries, Ltd., Tokyo, Japan.

When any racemate crystallises, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer. While both of the crystal forms present in a racemic mixture have identical physical properties, they may have different physical properties compared to the true racemate. Racemic mixtures may be separated by conventional techniques known to those skilled in the art—see, for example, Stereochemistry of Organic Compounds by E. L. Eliel and S. H. Wilen (Wiley, 1994).

The present invention includes all pharmaceutically acceptable isotopically-labelled compounds of formula (I) wherein one or more atoms are replaced by atoms having the same atomic number, but an atomic mass or mass number different from the atomic mass or mass number which predominates in nature. Isotopically-labelled compounds of formula (I) can generally be prepared by conventional techniques known to those skilled in the art or by processes analogous to those described in the accompanying Examples and Preparations using an appropriate isotopically-labelled reagent in place of the non-labelled reagent previously employed.

Also included within the scope of the invention are metabolites of compounds of formula (I), that is, compounds formed in vivo upon administration of the drug. Some examples of metabolites in accordance with the invention include
(i) where the compound of formula (I) contains a methyl group, an hydroxymethyl derivative thereof (—CH₃->—CH₂OH):
(ii) where the compound of formula (I) contains an alkoxy group, an hydroxy derivative thereof (—OR->—OH);
(iii) where the compound of formula (I) contains a tertiary amino group, a secondary amino derivative thereof (—NR¹R²—>—NHR¹ or —NHR²);
(iv) where the compound of formula (I) contains a secondary amino group, a primary derivative thereof (—NHR¹—>—NH₂);
(v) where the compound of formula (I) contains a phenyl moiety, a phenol derivative thereof (-Ph->-PhOH); and
(vi) where the compound of formula (I) contains an amide group, a carboxylic acid derivative thereof (—CONH₂->COOH).

For administration to human patients, the total daily dose of a compound of formula (I) is typically in the range of 0.01 mg to 500 mg depending, of course, on the mode of administration. In another embodiment of the present invention, the total daily dose of a compound of formula (I) is typically in the range of 0.1 mg to 300 mg. In yet another embodiment of the present invention, the total daily dose of a compound of formula (I) is typically in the range of 1 mg to 30 mg. The total daily dose may be administered in single or divided doses and may, at the physician's discretion, fall outside of the typical range given herein. These dosages are based on an average human subject having a weight of about 65 kg to 70 kg. The physician will readily be able to determine doses for subjects whose weight falls outside this range, such as infants and the elderly.

In the case of dry powder inhalers and aerosols, the dosage unit is determined by means of a prefilled capsule, blister or pocket or by a system that utilises a gravimetrically fed dosing chamber. Units in accordance with the invention are typically arranged to administer a metered dose or “puff” containing from 1 to 5000 μg of drug. The overall daily dose will typically be in the range 1 μg to 20 mg which may be administered in a single dose or, more usually, as divided doses throughout the day.

A compound of formula (I) can be administered per se, or in the form of a pharmaceutical composition, which, as active constituent contains an efficacious dose of at least one compound of the invention, in addition to customary pharmaceutically innocuous excipients and/or additives.

Pharmaceutical compositions suitable for the delivery of compounds of the present invention and methods for their preparation will be readily apparent to those skilled in the art. Such compositions and methods for their preparation may be found, for example, in Remington's Pharmaceutical Sciences, 19th Edition (Mack Publishing Company, 1995).

Compounds of formula (I) may be administered orally. Oral administration may involve swallowing, so that the compound enters the gastrointestinal tract, or buccal or sublingual administration may be employed by which the compound enters the blood stream directly from the mouth. Formulations suitable for oral administration include solid formulations such as tablets, capsules containing particulates, liquids, or powders, lozenges (including liquid-filled), chews, multi- and nano-particulates, gels, solid solution, liposome, films, ovules, sprays and liquid formulations. Oral administration is preferred, especially in the form of a tablet.

Liquid formulations include suspensions, solutions, syrups and elixirs. Such formulations may be employed as fillers in soft or hard capsules and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

Compounds of formula (I) may also be used in fast-dissolving, fast-disintegrating dosage forms such as those described in Expert Opinion in Therapeutic Patents, 11 (6), 981-986, by Liang and Chen (2001).

For tablet dosage forms, depending on dose, the drug may make up from 1 weight % to 80 weight % of the dosage form, more typically from 5 weight % to 60 weight % of the dosage form.

In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %. In one embodiment of the present invention, the disintegrant will comprise from 5 weight % to 20 weight % of the dosage form. Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate. Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet. Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %. In one embodiment of the present invention, lubricants comprise from 0.5 weight % to 3 weight % of the tablet. Other possible ingredients include anti-oxidants, colourants, flavouring agents, preservatives and taste-masking agents.

Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated. Formulations of tablets are discussed in Pharmaceutical Dosage Forms: Tablets, Vol. 1, by H. Lieberman and L. Lachman (Marcel Dekker, New York, 1980).

Consumable oral films for human or veterinary use are typically pliable water-soluble or water-swellable thin film dosage forms which may be rapidly dissolving or mucoadhesive and typically comprise a compound of formula (I), a film-forming polymer, a binder, a solvent, a humectant, a plasticiser, a stabiliser or emulsifier, a viscosity-modifying agent and a solvent. Some components of the formulation may perform more than one function. The film-forming polymer may be selected from natural polysaccharides, proteins, or synthetic hydrocolloids and is typically present in the range 0.01 to 99 weight %, more typically in the range 30 to 80 weight %. Other possible ingredients include anti-oxidants, colorants, flavourings and flavour enhancers, preservatives, salivary stimulating agents, cooling agents, co-solvents (including oils), emollients, bulking agents, anti-foaming agents, surfactants and taste-masking agents. Films in accordance with the invention are typically prepared by evaporative drying of thin aqueous films coated onto a peelable backing support or paper. This may be done in a drying oven or tunnel, typically a combined coater dryer, or by freeze-drying or vacuuming.

Solid formulations for oral administration may be formulated to be immediate and/or modified release. Modified release includes delayed, sustained, pulsed, controlled, targeted and programmed release. Suitable modified release formulations for the purposes of the invention are described in U.S. Pat. No. 6,106,864. Details of other suitable release technologies such as high energy dispersions and osmotic and coated particles are to be found in Pharmaceutical Technology On-line, 25(2), 1-14, by Verma et al (2001). The use of chewing gum to achieve controlled release is described in WO 00/35298.

Compounds of formula (I) may also be administered directly into the blood stream, into muscle, or into an internal organ. Suitable means for parenteral administration include intravenous, intraarterial, intraperitoneal, intrathecal, intraventricular, intraurethral, intrasternal, intracranial, intramuscular and subcutaneous. Suitable devices for parenteral administration include needle (including microneedle) injectors, needle-free injectors and infusion techniques.

Compounds of the invention may also be administered topically to the skin or mucosa, that is, dermally or transdermally.

The compounds of formula (I) can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler, as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane, or as nasal drops. For intranasal use, the powder may comprise a bioadhesive agent, for example, chitosan or cyclodextrin.

The pressurised container, pump, spray, atomizer, or nebuliser contains a solution or suspension of the compound of formula (I) comprising, for example, ethanol, aqueous ethanol, or a suitable alternative agent for dispersing, solubilising, or extending release of the compound, a propellant as solvent and an optional surfactant, such as sorbitan trioleate, oleic acid, or an oligolactic acid.

Prior to use in a dry powder or suspension formulation, the drug product is micronised to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

Capsules (made, for example, from gelatin or hydroxypropylmethylcellulose), blisters and cartridges for use in an inhaler or insufflator may be formulated to contain a powder mix of the compound of the invention, a suitable powder base such as lactose or starch and a performance modifier such as l-leucine, mannitol, or magnesium stearate. The lactose may be anhydrous or in the form of the monohydrate, preferably the latter. Other suitable excipients include dextran, glucose, maltose, sorbitol, xylitol, fructose, sucrose and trehalose.

A suitable solution formulation for use in an atomiser using electrohydrodynamics to produce a fine mist may contain from 1 μg to 20 mg of the compound of the invention per actuation and the actuation volume may vary from 1 μl to 100 μl. A typical formulation may comprise a compound of formula (I), propylene glycol, sterile water, ethanol and sodium chloride. Alternative solvents which may be used instead of propylene glycol include glycerol and polyethylene glycol.

Suitable flavours, such as menthol and levomenthol, or sweeteners, such as saccharin or saccharin sodium, may be added to those formulations of the invention intended for intranasal administration. Formulations for intranasal administration may be formulated to be immediate and/or modified release using, for example, PGLA. Modified release includes delayed, sustained, pulsed, controlled, targeted and programmed release.

Compounds of formula (I) may also be administered directly to the eye or ear, typically in the form of drops of a micronised suspension or solution in isotonic, pH-adjusted, sterile saline.

Compounds of formula (I) may be combined with soluble macromolecular entities, such as cyclodextrin and suitable derivatives thereof or polyethylene glycol-containing polymers, in order to improve their solubility, dissolution rate, taste-masking, bioavailability and/or stability for use in any of the aforementioned modes of administration. Drug-cyclodextrin complexes, for example, are found to be generally useful for most dosage forms and administration routes. Both inclusion and non-inclusion complexes may be used. As an alternative to direct complexation with the drug, the cyclodextrin may be used as an auxiliary additive, i.e. as a carrier, diluent, or solubiliser. Most commonly used for these purposes are alpha-, beta- and gamma-cyclodextrins, examples of which may be found in international patent publications WO-A-91/11172, WO-A-94/02518 and WO-A-98/55148.

Inasmuch as it may desirable to administer a combination of active compounds, for example, for the purpose of treating a particular disease or condition, it is within the scope of the present invention that two or more pharmaceutical compositions, at least one of which contains a compound of formula (I), may conveniently be combined in the form of a kit suitable for coadministration of the compositions. Thus, a kit of the invention comprises two or more separate pharmaceutical compositions, at least one of which contains a compound of formula (I), and means for separately retaining said compositions, such as a container, divided bottle, or divided foil packet. An example of such a kit is the familiar blister pack used for the packaging of tablets, capsules and the like. Such a kit is particularly suitable for administering different dosage forms, for example, oral and parenteral, for administering separate compositions at different dosage intervals, or for titrating the separate compositions against one another. To assist compliance, the kit typically comprises directions for administration and may be provided with a so-called memory aid.

All the compound of formula (I) can be made by the specific and general experimental procedures described below in combination with the common general knowledge of one skilled in the art (see, for example, Comprehensive Organic Chemistry, Ed. Barton and Ollis, Elsevier; Comprehensive Organic Transformations: A Guide to Functional Group Preparations, Larock, John Wiley and Sons).

The compounds of formula (I), being amides, are conveniently prepared by coupling an amine of formula (III) and an acid of formula (II) in accordance with Scheme 1.

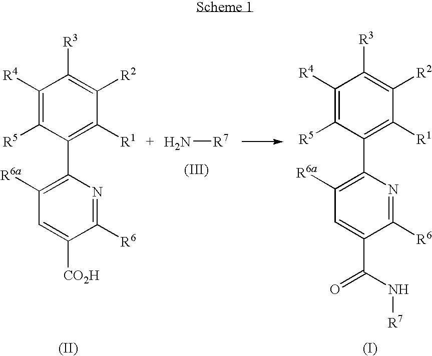

Those skilled in the art will appreciate that there are many known ways of preparing amides. For example, see Montalbetti, C. A. G. N and Falque, V., Amide bond formation and peptide coupling, Tetrahedron, 2005, 61(46), pp. 10827-10852 and references cited therein. The examples provided herein are thus not intended to be exhaustive, but merely illustrative.

The following general methods i, ii and iii have been used.
(i) To the carboxylic acid (0.15 mmol) and 1-hydroxybenzotriazole (0.3 mmol) in DMF (1.0 mL) was added 0.3.mmol of PS-Carbodiimide resin (Argonaut, 1.3 mmol/g). The mixture was shaken for 10 min and then the amine (0.1 mmol) in DMF (1 mL) was added. The mixture was allowed to agitate overnight at room temperature and subsequently treated with 0.60 mmole of PS-trisamine (Argonaut, 3.8 mmol/g). The reaction mixture was filtered, concentrated in vacuo and purified by reverse phase chromatography.
(ii) To the carboxylic acid (0.15 mmol) and HBTU (0.175 mmol) in DMF (1.0 mL) was added 0.45 mmol triethylamine. The mixture was stirred for 30 minutes and then the amine (0.2 mmol) in DMF (1.0 mL) was added. The mixture was allowed to stir overnight at room temperature and subsequently partitioned between water and a suitable organic solvent. The organic phase was separated, concentrated in vacuo and purified by either by reverse phase chromatography, normal phase chromatography or crystallisation.
(iii) To the carboxylic acid (0.15 mmol) in DMF was added N,N-carbonyldiimidazole (0.18 mmol) in DMF (1.0 mL). The mixture was stirred for 30 min and then the amine (0.18 mmol) in DMF (1.0 mL) was added. The mixture was allowed to stir overnight at room temperature and subsequently partitioned between water and a suitable organic solvent. The organic layer was separated, concentrated in vacuo and purified by reverse phase chromatography, normal phase chromatography or crystallisation.

Where it is stated that compounds were prepared in the manner described for an earlier Example, the skilled person will appreciate that reaction times, number of equivalents of reagents and reaction temperatures may be modified for each specific reaction, and that it may nevertheless be necessary or desirable to employ different work-up or purification conditions.

Those skilled in the art will appreciate that there are many known ways of preparing aryl pyridines of formula (II). Such methods are disclosed in patent textbooks and laboratory handbooks which constitute the common general knowledge of the skilled person, including the textbooks referenced above and references cited therein. Typically, an aryl (or heteroaryl) halide (Cl, Br, I) or trifluoromethanesulphonate is stirred with an organometallic species such as a stannane, organomagnesium derivative or a boronate ester or boronic acid in the presence of a catalyst, usually a palladium derivative between 0° C. and 120° C. in solvents including tetrahydrofuran, toluene, DMF and water for 1 to 24 hours. For example, an aryl (or heteroaryl) bromide may be heated to 100° C. in a mixture of water/toluene with a base such as sodium carbonate or sodium hydroxide, a palladium catalyst such as tetrakis(triphenylphosphine)palladium (0), a phase transfer catalyst such as tetra-n-butyl ammonium bromide and an aryl (or heteroaryl) boronic acid or ester. As a second example, an aryl (or heteroaryl) boronic ester an aryl (or heteroaryl) halide (Cl, Br, I) or aryl (or heteroaryl) trifluoromethanesulphonate and a fluoride source such as KF or CsF in a non-aqueous reaction medium such as 1,4-dioxane may be employed. It may be necessary to protect the acid functionality in the compound of formula (II) during such a coupling reaction—suitable protecting groups and their use are well known to the skilled person (see, e.g., ‘Protective Groups in Organic Synthesis’ by Theorora Greene and Peter Wuts (third edition, 1999, John Wiley and Sons).

Amines of formula (III) are in many cases commercially available and may otherwise be prepared by standard methodology well known the skilled person—see, for example, ‘Comprehensive Organic Transformations’ by Richard Larock (1999, VCH Publishers Inc.).

The following tabulated compounds have been prepared using the methodology described above. Data relating to purification and characterization are provided in the tables and relevant HPLC and LCMS methods are described in detail below the tables, along with more specific details relating to the preparation and charactersisation of selected compounds.

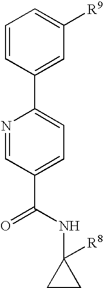

| Ex | R⁸ | R⁹ | Name | Purification and Characterisation |
|---|---|---|---|---|
| 1 | 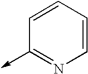 | F | 6-(3-Fluorophenyl)-N-(1- pyridin-2- ylcyclopropyl)nicotinamide | LCMS Method (C) RT 1.39 min m/z Obs [M + 1] 334.35 calc [M + 1] 334.1 |
| 2 | —CO₂CH₃ | F | Methyl 1-({[6-(3- fluorophenyl)pyridin-3- yl]carbonyl}amino) cyclopropanecarboxylate | Purifed by HPLC Method (E) LCMS Method (F) RT 4.13 min m/z Obs [M + 1] 315.1118 calc [M + 1] 315.32 |
| 3 | —CO₂CH₃ | —CH₃ | Methyl 1-({[6-(3- methylphenyl)pyridin-3- yl]carbonyl}amino) cyclopropanecarboxylate | Purifed by HPLC Method (E) LCMS Method (F) RT 4.21 min m/z Obs [M + 1] 311.1395 calc [M + 1] 311.36 |
| 4 | —CO₂CH₃ | —OCH₃ | Methyl 1-({[6-(3- methoxyphenyl)pyridin-3- yl]carbonyl}amino) cyclopropanecarboxylate | Purifed by HPLC Method (E) LCMS Method (F) RT 4.82 min m/z Obs [M + 1] 311.1761 calc [M + 1] 311.40 |

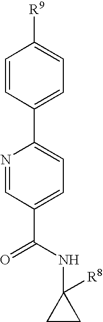

| | | | | Purification and |
|---|---|---|---|---|
| Ex | R⁸ | R⁹ | Name | Characterisation |
| 5 | —CO₂CH₃ | F | Methyl 1-({[6-(4- | Purifed by HPLC Method (E) |
| | | | fluorophenyl) | LCMS Method (F) RT 4.13 |
| | | | pyridin-3- | min m/z Obs [M + 1] |
| | | | yl]carbonyl} | 315.1 calc [M + 1] 315.11 |
| | | | amino)cyclopro | |
| | | | panecarboxylate | |

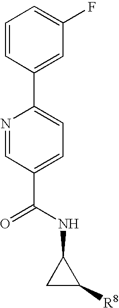

| | | | Purification and |
|---|---|---|---|
| Ex | R⁸ | Name | Characterisation |
| 6 | H | N-Cyclopropyl-6-(3- | LRMS [M + 1] 257 obs, [M + 1] 257 |
| | | fluoro-phenyl)- | calc. |
| | | nicotinamide | ¹H NMR (CDCl₃, 400 MHz) ppm |
| | | | δ 0.66-0.70 (m, 2H), 0.91- |
| | | | 0.95 (m, 2H), 2.92-2.97 (m, 1H), |
| | | | 6.25-6.35 br m, 1H), 7.13- |
| | | | 7.18 (m, 1H), 7.43-7.49 (m, 1H), |
| | | | 7.77-7.81 (m, 3H), 8.17-8.19 |
| | | | (m, 1H), 8.98-8.98 (m, 1H). |

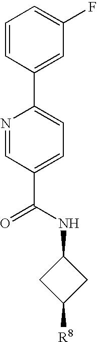

| | | | Purification and |
|---|---|---|---|
| Ex | R⁸ | Name | Characterisation |
| 7 | —CH₂N(CH₃)₂ | N-{cis-3- | Purified by HPLC Method (B) |
| | | [(Dimethylamino) | LCMS Method (A) RT 2.07 |
| | | methyl]cyclobutyl}- | min (100%) 328.2 m/z |
| | | 6-(3-fluorophenyl) | [M + 1] |
| | | nicotinamide | |

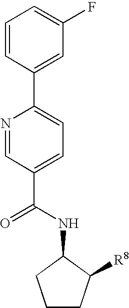

| | | | Purification and |
|---|---|---|---|
| Ex | R⁸ | Name | Characterisation |
| 8 | —CO₂H | cis-2-({[6-(3- | ¹H NMR (400 MHz, CDCl₃) 1.713-1.779 (m, 1H), |
| | | Fluorophenyl) | 1.824-1.920 (m, 2H), 2.111-2.214 (m, 3H), 3.087- |
| | | pyridin-3- | 3.138 (m, 1H), 4.670-4.707 (m, 1H), 7.171-7.196 (m, |
| | | yl]carbonyl}amino) | 1H), 7.460-7.629 (m, 3H,), 7.779-7.780 (m, 1H), 8.311- |
| | | cyclopentanecar | 8.331 (m, 1H), 8.488-8.514 (m, 1H), 9.344-9.348 (m, |
| | | boxylic acid | 1H). |
| 9 | H | N-Cyclopentyl-6- | ¹H NMR (400 MHz, DMSO-d₆) δ ppm 1.49-1.61 (m, 4 |
| | | (3- | H) 1.64-1.77 (m, 2 H) 1.84-1.97 (m, 2 H) 4.20-4.31 |
| | | fluorophenyl) | (m, 1 H) 7.29-7.36 (m, 1 H) 7.53-7.60 (m, 1 H) 7.94- |
| | | nicotinamide | 7.99 (m, 1 H) 8.01 (d, J = 8.1 Hz, 1 H) 8.14 (d, J = 8.1 |
| | | | Hz, 1 H) 8.29 (dd, J = 8.4, 2.6 Hz, 1 H) 8.54 (d, J = 7.3 |
| | | | Hz, 1 H) 9.07 (d, J = 2.2 Hz, 1 H). |
| | | | MS calc [M + 1] 285.3, obs [M + 1] 285.1. |
| 10 | —CH₂OH | 6-(3- | LCMS Method (C) RT 2.23 min MS Obs [M + 1] 329.25 |
| | | Fluorophenyl)-N- | calc [M + 1] 329.16 |
| | | [(1R,2S)-2- | |
| | | (methoxymethyl) | |
| | | cyclopentyl] | |
| | | nicotinamide | |

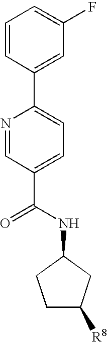

| | | | Purification and |
|---|---|---|---|
| Ex | R⁸ | Name | Characterisation |
| 11a | —CO₂H | (1S,3R)-3-({[6-(3- | ¹H NMR (400 MHz, MeOD-d₄) ppm 1.82-1.85 (m, 1H) |
| | | Fluorophenyl) | 1.94-1.99 (m, 1H) 2.06-2.12 (m, 3H) 2.36-2.42 (m, 1H) |
| | | pyridin-3- | 2.90-3.00 (m, 1H) 4.45-4.48 (m, 1H) 7.23-7.28 (m, 1H) |
| | | yl]carbonyl}amino) | 7.54-7.59 (m, 1H) 7.85-7.88 (m, 1H) 7.89-7.93 (m, 1H) |
| | | cyclopentanecar | 8.02-8.04 (m, 1H) 8.30-8.33 (m, 1H) 9.09 (s, 1H) |
| | | boxylic acid | |
| 11b | —CO₂H | (1R,3S)-3-({[6-(3- | ¹H NMR (400 MHz, DMSO-d₆) δ ppm 1.61-1.69 (m, |
| | | Fluorophenyl) | 1H) 1.78-1.96 (m, 4H) 2.16-2.32 (m, 1H) 2.71-2.82 (m, |
| | | pyridin-3- | 1H) 4.26-4.32 (m, 1H) 7.31-7.36 (m, 1H) 7.55-7.60 (m, |
| | | yl]carbonyl}amino) | 1H) 7.96-8.03 (m, 1H) 8.14-8.16 (m, 1H) 8.27-8.31 (m, |
| | | cyclopentanecar | 1H) 8.74-8.76 (m, 1H) 9.08 (s, 1H) |
| | | boxylic acid | |
| 12 | 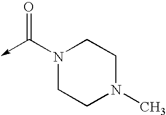 | 6-(3- Fluorophenyl)-N- {(1R,3S)-3-[(4- methylpiperazin- 1-yl)carbonyl] cyclopentyl} nicotinamide | Purified by HPLC method (A) LCMS method (B) RT 2.82 min (100%) ES⁺ m/z 411.1 [M + 1] |
| 13 | 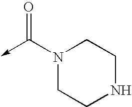 | 6-(3- Fluorophenyl)-N- [(1R,3S)-3- (piperazin-1- ylcarbonyl) cyclopentyl] nicotinamide | Purified by HPLC method (B) LCMS method (B) RT 2.14 min (100%) ES⁺ m/z 397.1 [M + 1] |
| 14 | 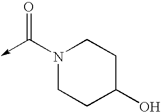 | 6-(3- Fluorophenyl)-N- {(1R,3S)-3-[(4- hydroxypiperidin- 1-yl)carbonyl] cyclopentyl} nicotinamide | Purified by HPLC Method (B) LCMS Method (B) RT 2.84 min (100%) m/z 412.1 [M + 1] |
| 15 | 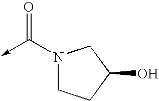 | 6-(3- Fluorophenyl)-N- [(1R,3S)-3-{[(3S)- 3- hydroxypyrrolidin- 1-yl]carbonyl} cyclopentyl] nicotinamide | Purified by HPLC method (A) LCMS method (B) RT 2.62 min (100%) ES⁺ m/z 399.1 [M + 1] |
| 16 | 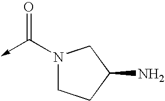 | N-[(1R,3S)-3- {[(3S)-3- Aminopyrrolidin- 1- yl]carbonyl} cyclopentyl]-6-(3- fluorophenyl) nicotinamide | Purified by HPLC method (A) LCMS method (A) RT 2.41 min (100%) ES⁺ m/z 397.1 [M + 1] |

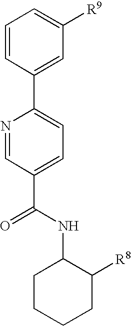

| | | | | Purification and |
|---|---|---|---|---|
| Ex | R⁸ | R⁹ | Name | Characterisation |
| 17 | —OH | F | 6-(3- | ¹H NMR (400 MHz, DMSO-d₆) δ ppm 1.20-1.39 (m, 2 |
| | | | Fluorophenyl)- | H) 1.41-1.85 (m, 6 H) 3.83-3.93 (m, 2 H) 4.70 (d, |
| | | | N-(2- | J = 4.4 Hz, 1 H) 7.28-7.37 (m, 1 H) 7.53-7.61 (m, 1 |
| | | | hydroxycyclohexyl) | H) 7.94-7.99 (m, 1 H) 8.02 (d, J = 8.1 Hz, 1 H) 8.13 (d, |
| | | | nicotinamide | J = 8.1 Hz, 1 H) 8.18 (d, J = 7.3 Hz, 1 H) 8.32 (dd, J = |
| | | | | 8.1, 2.2 Hz, 1 H) 9.10 (d, J = 2.2 Hz, 1 H). |
| | | | | MS calc [M + 1] 315.4, obs [M + 1] 315.1. |
| 18 | H | F | N-Cyclohexyl-6- | Purifed by HPLC Method (E) |
| | | | (3- | LCMS Method (F) RT 5.01 min m/z Obs [M + 1] |
| | | | fluorophenyl) | 299.1558 calc [M + 1] 299.36 |
| | | | nicotinamide | |
| 19 | H | —CH₃ | N-Cyclohexyl-6- | Purifed by HPLC Method (E) |
| | | | (3- | LCMS Method (F) RT 5.13 min m/z Obs [M + 1] 295.18 |
| | | | methylphenyl) | calc [M + 1] 295.40 |
| | | | nicotinamide | |
| 20 | H | —OCH₃ | N-Cyclohexyl-6- | Purifed by HPLC Method (E) |
| | | | (3- | LCMS Method (F) RT 4.82 min m/z Obs [M + 1] |
| | | | methoxyphenyl) | 311.1761 calc [M + 1] 311.40 |
| | | | nicotinamide | |
| 21 | H | H | N-Cyclohexyl-6- | ¹H NMR (400 MHz, DMSO-d₆) δ ppm 2.14-2.32 (m, 1 |
| | | | phenylnicotin- | H) 2.35-2.52 (m, 2 H) 2.92-3.14 (m, 4 H) 3.49 (dd, |
| | | | amide | J = 13.18, 7.69 Hz, 2 H) 4.71 (d, J = 6.59 Hz, 1 H) 7.62 |
| | | | | (t, J = 7.32 Hz, 1 H) 7.68-7.82 (m, 2 H) 7.84 (d, J = 5.49 |
| | | | | Hz, 1 H) 7.94 (d, J = 8.05 Hz, 1 H) 8.02 (t, J = 8.60 Hz, |
| | | | | 2 H) 8.57 (d, J = 5.49 Hz, 1 H) 8.82 (d, J = 6.59 Hz, 1 |
| | | | | H). |
| | | | | MS calc [M + 1] 281.1654, obs [M + 1] 281.1685. |

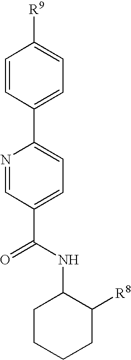

| | | | | Purification and |
|---|---|---|---|---|
| Ex | R⁸ | R⁹ | Name | Characterisation |
| 22 | H | F | N-Cyclohexyl-6-(4- | Purifed by HPLC Method (E) |
| | | | fluorophenyl) | LCMS Method (F) RT 4.91 min m/z Obs [M + 1] |
| | | | nicotinamide | 299.1549 calc [M + 1] 299.36 |

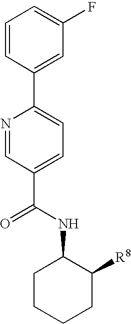

| | | | Purification and |
|---|---|---|---|
| Ex | R⁸ | Name | Characterisation |
| 23 | —OH | 6-(3-Fluorophenyl)-N- | Purified by HPLC Method A |
| | | [cis-2- | LCMS Method (B) RT 3.00 minutes (100% area), |
| | | hydroxycyclohexyl] | ES m/z [MH] 315.1 |
| | | nicotinamide | |
| 24 | —CH₂OH | 6-(3-Fluorophenyl)-N- | LCMS Method (C) RT 2.01 min MS Obs [M + 1] 329.25 |
| | | [(1R,2S)-2- | calc [M + 1] 329.16 |
| | | (hydroxymethyl) | |
| | | cyclohexyl] | |
| | | nicotinamide | |

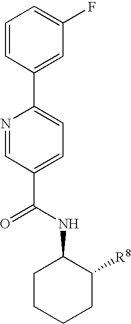

| | | | Purification and |
|---|---|---|---|
| Ex | R⁸ | Name | Characterisation |
| 25 | —OH | 6-(3-fluorophenyl)-N- | LCMS Method (C) RT 1.74 min m/z Obs [M + 1] |
| | | [(1S,2S)-2- | 315.25 calc [M + 1] 315.1 |
| | | hydroxycyclohexyl] | |
| | | nicotinamide | |

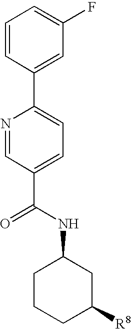

| | | | Purification and |
|---|---|---|---|
| Ex | R⁸ | Name | Characterisation |
| 26 | —OH | 6-(3- | Purified by HPLC Method (B) |
| | | Fluorophenyl)-N- | LCMS Method B RT 2.68 minutes (100% area), |
| | | [cis-3- | ES m/z [M + 1] 315.1 |
| | | hydroxycyclohexyl] | |
| | | nicotinamide | |
| 27 | —CO₂H | cis-3-({[6-(3- | ¹H NMR (400 MHz, DMSO-d₆) δ ppm 1.14-1.44 (m, |
| | | Fluorophenyl) | 4H) 1.78-1.80 (1H, m) 1.86-1.90 (2H, m) 2.04-2.11 |
| | | pyridin-3- | (m, 1H) 2.32-2.39 (m, 1H) 3.80-3.89 (m, 1H) 7.29- |
| | | yl]carbonyl} | 7.34 (m, 1H), 7.54-7.60 (m, 1H) 7.94-7.98 (m, 1H), |
| | | amino) | 8.00-8.02 (m, 1H) 8.12-8.14 (m, 1H) 8.27-8.30 (m, 1H) |
| | | cyclohexanecarboxylic | 8.50-8.52 (m, 1H) 9.07 (s, 1H) 12.10 (s broad, 1H) |
| | | acid | |
| 28 | 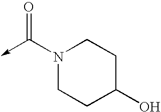 | 6-(3- Fluorophenyl)-N- {cis-3-[(4- hydroxypiperidin- 1- yl)carbonyl] cyclohexyl} nicotinamide | Purified by HPLC Method (B) LCMS Method (A) RT 2.67 minutes (100%) area, ES m/z [M + H] 426.1 |
| 29 | 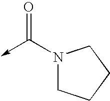 | 6-(3- Fluorophenyl)-N- [cis-3-(pyrrolidin- 1-ylcarbonyl) cyclohexyl] nicotinamide | Purified by HPLC Method (B) LCMS Method (A) RT 2.91 minutes (100%) area, ES m/z [M + 1] 396.1 |
| 30 | —CONHCH₂Ph | N-[cis-3- | Purified by HPLC Method (A) |
| | | (Benzylcarbamoyl) | LCMS Method (B) RT 3.13 minutes (100%) area, |
| | | cyclohexyl]-6-(3- | ES m/z [M + 1] 432.1 |
| | | fluorophenyl) | |
| | | nicotinamide | |
| 31 | —CON(CH₃)₂ | N-[cis-3- | Purified by HPLC Method (B) |
| | | (Dimethyicarbamoyl) | LCMS Method (A) RT 3.12 (100 %) area, |
| | | cyclohexyl]-6- | ES m/z [M + 1] 370.2 |
| | | (3- | |
| | | fluorophenyl) | |
| | | nicotinamide | |
| 32 | 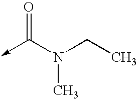 | N-{cis-3- [Ethyl(methyl) carbamoyl] cyclohexyl}-6-(3- fluorophenyl) nicotinamide | Purified by HPLC Method (A) LCMS Method (A) RT 3.14 minutes (100%) area, ES m/z [M + H] 384.2 |
| 33 | 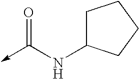 | N-[cis-3- (Cyclopentylcarbamoyl) cyclohexyl]- 6-(3-fluorophenyl) nicotinamide | LCMS (ES+) 410 [M + 1] ¹H NMR (400 MHz, DMSO-d₆) δ ppm 1.20-140 (m, 5H), 1.42-1.53 (m, 3H), 1.56-1.73 (m, 3H), 1.71-1.90 (m, 5H), 2.19-2.28 (m, 1H), 3.-3.90 (m, 1H), 3.91-4.01 (m, 1H), 7.28-7.35 (m, 1H), 7.58- 7.61 (m, 1H), 7.70-7.76 (m, 1H), 7.93-8.04 (m, 2H), 8.10-8.16 (m, 1H), 8.26-8.31 (m, 1H), 8.49-8.56 (m, 1H), 9.05-9.10 (m, 1H). |
| 34 | —CONHⁱPr | 6-(3- | LCMS (ES+) 384 [M + 1] ¹H NMR |
| | | Fluorophenyl)-N- | (400 MHz, DMSO-d₆) |
| | | [cis-3- | δ ppm 0.98-1.06 (m, 6H), 1.20-1.35 (m, 3H),1.38-1.52 |
| | | (isopropylcarbamoyl) | (m, 1H), 1.60-1.68 (m, 1H), 1.76-1.91 (m, 3H), 2.16- |
| | | cyclohexyl] | 2.26 (m, 1H), 3.75-3.90 (m, 2H), 7.28-7.36 (m, 1H), |
| | | nicotinamide | 7.52-7.67 (m, 2H), 7.92-8.04 (m, 2H), 8.10-8.16 (m, |
| | | | 1H), 8.25-8.31 (m, 1H), 8.49-8.55 (m, 1H), 9.05-9.10 |
| | | | (m, 1H). |
| 35 | 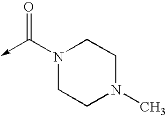 | 6-(3- Fluorophenyl)-N- {cis-3-[(4- methylpiperazin- 1-yl)carbonyl] cyclohexyl} nicotinamide | Purified by HPLC Method (A) LCMS Method (B) RT 2.84 minutes (100%) area, ES m/z [M + 1] 425.2 |
| 36 | 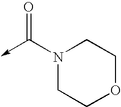 | 6-(3- Fluorophenyl)-N- [cis-3-(morpholin- 4-ylcarbonyl) cyclohexyl] nicotinamide | Purified by HPLC Method (A) LCMS Method (B) RT 2.85 minutes (100%) area, ES m/z [M + 1] 412.2 |
| 37 | 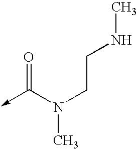 | 6-(3- Fluorophenyl)-N- [cis-3-{methyl[2- (methylamino)ethyl] carbamoyl} cyclohexyl] nicotinamide | Purified by HPLC Method (B) LCMS Method (B) RT 2.92 minutes (100%) area, ES m/z [M + H] 413.2 |
| 38 | —CONH^{t}Bu | N-[cis-3-(tert- | Purified by HPLC Method (A) |
| | | Butylcarbamoyl) | LCMS Method (B) RT 3.22 minutes (100%) area, |
| | | cyclohexyl]-6-(3- | ES m/z [M + 1] 398.1 |
| | | fluorophenyl) | |
| | | nicotinamide | |
| 39 | 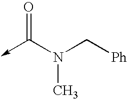 | N-{cis-3- [Benzyl(methyl) carbamoyl] cyclohexyl}- 6-(3- fluorophenyl) nicotinamide | Purified by HPLC Method (A) LCMS Method (B) RT 3.20 minutes (100%) area, ES m/z [M + 1] 446.2 |
| 40 | 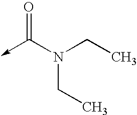 | N-[cis-3- (Diethylcarbamoyl) cyclohexyl]-6-(3- fluorophenyl) nicotinamide | Purified by HPLC Method (A) LCMS Method (A) RT 3.05 minutes (100%) area, ES m/z MH+ 398.2 |
| 41 | 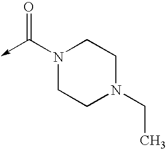 | N-{cis-3-[(4- Ethylpiperazin-1- yl)carbonyl] cyclohexyl}-6-(3- fluorophenyl) nicotinamide | ¹H NMR (400MHz, DMSO-d₆) ppm 1.0-1.02 (m, 3H) 1.19-1.28 (m, 2H) 1.41-1.60 (m, 2H) 1.61-1.64 (m, 1H) 1.72-1.83 (m, 2H) 1.89-1.91 (m, 1H) 2.29-2.35 (m, 6H) 2.75-2.86 (m, 1H) 3.46-3.55 (m, 4H) 3.93-3.96 (m, 1H) 7.32-7.37 (m, 1H) 7.56-7.62 (m, 1H) 7.97-7.99 (m, 2H) 8.02-8.04 (m, 1H) 8.30-8.33 (m, 1H) 8.52-8.54 (m, 1H) 9.10 (s, 1H) |
| 41a | 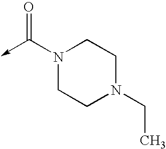 | Enantiomer 1 | Peak 1 Isolated by chiral HPLC (see experiemental section below) |
| 41b | 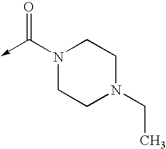 | Enantiomer 2 | Peak 2 Isolated by chiral HPLC (see experimental section below) |
| 42 | 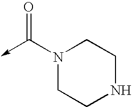 | 6-(3- Fluorophenyl)-N- [cis-3-(piperazin- 1- ylcarbonyl) cyclohexyl] nicotinamide | LCMS (ES−) 411 [M + 1] ¹H NMR (400 MHz, DMSO-d₆) δ ppm 1.18-1.36 (m, 2H), 1.38-1.58 (m, 2H), 1.63-1.70 (m, 1H), 1.76-1.93 (m, 3H), 2.78-2.89 (m, 1H), 2.99- 3.17 (m, 4H), 3.63-3.81 (m, 4H), 3.88-3.99 (m, 1H), 7.28-7.36 (m, 1H), 7.52-7.61 (m, 1H), 7.92-8.03 (m, 2H), 8.10-8.15 (m, 1H), 8.28-8.34 (m, 1H), 8.52-8.57 (m, 1H), 9.06-9.10 (m, 1H), 9.18-9.29 (br. s. 2H) |
| 43 | 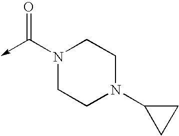 | N-{cis-3-[(4- Cyclopropylpiperazin- 1-yl)carbonyl] cyclohexyl}-6-(3- fluorophenyl) nicotinamide | Purified by HPLC Method (B) LCMS Method (A) RT 2.38 minutes (100%) area, ES m/z [M + 1] 451.1 |

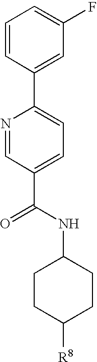

| | | | Purification and |
|---|---|---|---|
| Ex | R⁸ | Name | Characterisation |
| 44 | —NH₂ | N-(4- | LCMS (ES) Obs m/z 312 [M − 1] calc 312.38 [M − 1] |
| | | Aminocyclohexyl)- | ¹H NMR (400 MHz CDCl₃) δppm 1.40-1.93 (m, 8H), |
| | | 6-(3- | 2.98-3.07 (m, 1H), 4.15-4.25 (m, 1H), 6.22-6.34 (m, |
| | | fluorophenyl) | 1H), 7.09-7.19 (m, 1H), 7.40-7.50 (m, 1H), 7.73-7.84 |
| | | nicotinamide | (m, 3H), 8.13-8.20 (m, 1H), 8.98-9.05 (m, 1H). |
| 45 | 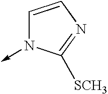 | 6-(3- Fluorophenyl)-N- {4-[2-(methylthio)- 1H-imidazol-1- yl]cyclohexyl} nicotinamide | Purified using HPLC method (A) LCMS method (B) RT 3.05 min (100% area) ES m/z 411.1 [M + 1] |
| 46 | —NHCOCH₃ | N-(4- | LCMS (ES−) m/z 354 (M − 1) calc 354.4 [M − 1] |
| | | Acetamido- | ¹H NMR (400 MHz CDCl₃) δ ppm 1.54-2.06 (11H, m), |
| | | cyclohexyl)-6-(3- | 3.88-3.98 (m, 1H), 4.06-4.20 (m, 1H), 5.63-5.74 (m, |
| | | fluorophenyl) | 1H), 6.34-6.43 (m, 1H), 7.08-7.18 (m, 1H), 7.38-7.49 |
| | | nicotinamide | (m, 1H), 7.68-7.80 (m, 3H), 8.11-8.21 (m, 1H), 8.94- |
| | | | 9.02 (m, 1H) |
| 47 | 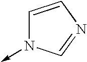 | 6-(3- Fluorophenyl)-N- [4-(1H-imidazol-1- yl)cyclohexyl] nicotinamide | LCMS (ES) Obs 363.2 (M − 1) calc 363.4 [M − 1] ¹H NMR (400 MHz CD₃OD) δ ppm 1.81-2.07 (m, 6H), 2.12-2.28 (m, 2H), 4.18-4.29 (m, 2H), 6.94-7.01 (m, 1H), 7.14-7.23 (m, 1H), 7.45-7.56 (m, 1H), 7.75-7.89 (m, 3H), 7.94-8.00 (m, 1H), 8.23-8.29 (m, 1H), 9.00- 9.06 (m, 1H). |
| 48 | —C(CH₃)₂OH | 6-(3- | ¹H NMR (400 MHz, DMSO-d₆) δ ppm 1.03 (s, 6 H) |
| | | Fluorophenyl)-N- | 1.09-1.19 (m, 2 H) 1.23-1.39 (m, 2 H) 1.81 (dd, 2 H) |
| | | [4-(1-hydroxy-1- | 1.92 (dd, 2 H) 3.64-3.79 (m, 1 H) 3.97 (s, 1 H) 7.24- |
| | | methylethyl) | 7.35 (m, 1 H) 7.49-7.60 (m, 1 H) 7.91 (d, 1 H) 7.99 (d, |
| | | cyclohexyl] | 1 H) 8.10 (d, 1 H) 8.26 (d, 1 H) 8.38 (d, 1 H) 9.04 (s, 1 |
| | | nicotinamide | H). |
| | | | MS calc [M + 1] 357.20, obs [M + H] 357.21. |
| 49 | 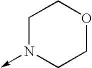 | Diastereomer 1 6-(3- Fluorophenyl)-N- (4-morpholin-4- ylcyclohexyl) nicotinamide | LCMS (APCl) m/z 384 [M + 1] Calc 384.47 [M + 1] ¹H NMR (400 MHz CDCl₃) δ ppm 1.57-1.93 (m, 8H), 2.15-2.25 (m, 1H), 2.50-2.58 (m, 4H), 3.70-3.76 (m, 4H), 4.20-4.29 (m, 1H), 6.18-6.26 (m, 1H), 7.12-7.18 (m, 1H), 7.43-7.50 (m, 1H), 7.75-7.82 (m, 3H), 8.12- 8.20 (m, 1H), 8.98-9.04 (m, 1H) |
| 50 | 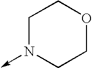 | Diastereoiner 2 6-(3- Fluorophenyl)-N- (4-morpholin-4- ylcyclohexyl) nicotinamide | LCMS (ES) m/z 384 [M + 1] Calc 384.47 [M + 1] ¹H NMR (400 MHz DMSO-d₆) δ ppm 1.21-1.41 (4H, m), 1.80-1.95 (m, 4H), 2.12-2.22 (m, 1H), 2.42-2.51 (m, 4H), 3.51-3.56 (m, 4H), 3.67-3.78 (m, 1H), 7.27- 7.34 (m, 1H), 7.52-7.58 (m, 1H), 7.91-7.97 (m, 1H), 7.98-8.01 (m, 1H), 8.10-8.14 (m, 1H), 8.24-8.29 (m, 1H), 8.41-8.46 (m, 1H), 9.04-9.06 (m, 1H) |
| 51 | 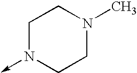 | Diastereomer 1 6-(3- Fluorophenyl)-N- [-4-(4- methylpiperazin- 1-yl)cyclohexyl] nicotinamide | LCMS (ES) m/z 398 [M + 1] Calc 397.51 [M + 1] ¹H NMR (400 MHz CDCl₃) δ ppm 0.70-0.92 (m, 4H), 0.94-1.97 (9H, m), 2.22-2.34 (m, 3H), 2.41-2.72 (m, 4H), 4.22-4.33 (m, 1H), 6.22-6.28 (m, 1H), 7.12-7.16 (m, 1H), 7.42-7.53 (m, 1H), 7.76-7.83 (m, 3H), 8.15- 8.21 (m, 1H), 9.00-9.05 (m, 1H) |
| 52 | 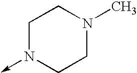 | Diastereomer 2 6-(3- Fluorophenyl)-N- [-4-(4- methylpiperazin- 1-yl)cyclohexyl] nicotinamide | LCMS (ES) m/z 398 [M + 1] calc 397.51 [M + 1] ¹H NMR (400 MHz DMSO-d₆) δ ppm 1.18-1.44 (m, 4H), 1.75-1.97 (m, 4H), 2.12 (s, 3H), 2.16-2.57 (m, 1H), 3.62-3.79 (m, 1H), 7.47-7.61 (m, 1H), 7.90-8.04 (m, 2H), 8.08-8.15 (m, 1H), 8.25-8.32 (m, 1H), 8.37- 8.48 (m, 1H), 9.01-9.08 (m, 1H) |
| 53 | 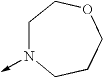 | 6-(3- Fluorophenyl)-N- [4-(1,4-oxazepan- 4-yl)cyclohexyl] nicotinamide | LCMS (APCl) m/z 398 [M + 1] calc 398.49 [M + 1] ¹H NMR (400 MHz CDCl₃) δ ppm 1.23-1.36 (m, 2H), 1.40-1.52 (m, 2H), 1.81-1.94 (m, 4H), 2.16-2.23 (m, 2H), 2.51-2.63 (m, 1H), 2.72-2.80 (m, 4H), 3.68- 3.73 (m, 2H), 3.75-3.84 (m, 2H), 3.88-3.97 (m, 1H), 5.88-5.98 (m, 1H), 7.09-7.19 (m, 1H), 7.40-7.52 (m, 1H), 7.73-7.84 (m, 3H), 8.11-8.20 (m, 1H), 8.96-9.01 (m, 1H) |
| 54 | 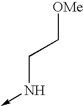 | Diastereomer 1 6-(3- Fluorophenyl)-N- {4-[(2- methoxyethyl) amino]cyclohexyl} nicotinamide | LCMS (ES) m/z 370.15 [M − 1] Calc 370.46 [M + 1] ¹H NMR (400 MHz CDCl₃) ppm 1.44-1.68 (m, 2H), 1.70-1.92 (m, 6H), 2.64-2.74 (m, 1H), 2.76-2.84 (m, 2H), 3.32-3.40 (m, 3H), 3.44-3.56 (m, 2H), 4.16-4.29 (m, 1H), 6.17-6.27 (m, 1H), 7.08-7.20 (m, 1H), 7.40- 7.51 (m, 1H), 7.73-7.84 (m, 3H), 8.08-8.20 (m, 1H), 8.96-9.04 (m, 1H) |
| 55 | 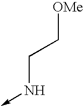 | Diastereomer 2 6-(3- Fluorophenyl)-N- {4-[(2- methoxyethyl) amino]cyclohexyl} nicotinamide | LCMS (ES) m/z 372 [M + 1] Calc 372.46 [M + 1] ¹H NMR (400 MHz CD₃OD) δ ppm 1.19-1.37 (m, 2H), 1.38-1.55 (m, 2H), 1.93-2.11 (m, 4H), 2.42-2.60 (m, 1H), 2.71-2.84 (m, 2H), 3.36 (s, 3H), 3.45-3.55 (m, 2H), 3.82-3.94 (m, 1H), 7.14-7.25 (m, 1H), 7.43-7.56 (m, 1H), 7.76-7.91 (m, 2H), 7.94-8.03 (m, 1H), 8.21- 8.31 (m, 1H), 9.00-9.07 (m, 1H) |
| 56 | 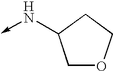 | Diastereomer 1 6-(3- Fluorophenyl)-N- [-4- (tetrahydrofuran- 3- ylamino) cyclohexyl] nicotinamide | ¹H NMR (400 MHz CDCl₃) δ ppm 1.25-1.40 (m, 4H), 1.65-1.72 (m, 1H), 1.90-2.05 (m, 2H), 2.05-2.25 (m, 3H), 2.40-2.55 (m, 1H), 3.45-3.55 (m, 2H), 3.75-4.05 (m, 4H), 5.90-6.00 (m, 1H), 7.10-7.20 (m, 1H),7.40- 7.50 (m, 1H), 7.70-7.85 (m, 3H), 8.10-8.20 (m, 1H), 8.95-9.05 (m, 1H) |
| 57 | 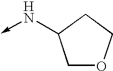 | Diastereomer 2 6-(3- Fluorophenyl)-N- [-4- (tetrahydrofuran- 3-ylamino) cyclohexyl] nicotinamide | ¹H NMR (400 MHz CDCl₃) δ ppm 1.37-1.91 (m, 10H), 2.07-2.20 (m, 1H), 2.73-2.80 (m, 1H), 3.48-3.57 (m, 2H), 3.73-3.86 (m, 2H), 3.89-3.98 (m, 1H), 4.20-4.26 (m, 1H), 6.13-6.21 (m, 1H), 7.09-7.19 (m, 1H), 7.42- 7.49 (m, 1H), 7.75-7.83 (m, 3H), 8.12-8.19 (m, 1H), 8.99-9.04 (m, 1H) |
| 58 | 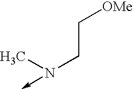 | Diastereomer 1 6-(3- Fluorophenyl)-N- {4-[(2- methoxyethyl) (methyl)amino] cyclohexyl} nicotinamide | LCMS (ES) m/z 386 [M + 1] Calc 386.48 [M + 1] ¹H NMR (400 MHz CD₃OD) δ ppm 1.60-1.76 (m, 6H), 1.97-2.06 (m, 2H), 2.33 (3H, s) 2.45-2.55 (m, 1H), 2.68-2.74 (m, 2H), 3.33-3.34 (m, 3H), 3.50-3.54 (m, 2H), 4.10-4.16 (m, 1H), 7.17-7.24 (m, 1H), 7.48-7.57 (m, 1H), 7.78-7.91 (m, 2H), 7.95-7.99 (m, 1H), 8.23- 8.27 (m, 1H), 9.00-9.04 (m, 1H). |
| 59 | 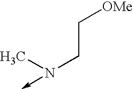 | Diastereomer 2 6-(3- Fluorophenyl)-N- {4-[(2- methoxyethyl) (methyl)amino] cyclohexyl} nicotinamide | LCMS (ES) m/z 386 [M + 1] Calc 385.48 [M + 1] ¹H NMR (400 MHz CD₃OD) δ ppm 1.39-1.51 (m, 4H), 1.90-2.00 (m, 2H), 2.05-2.12 (m, 2H), 2.31 (3H, s), 2.46-2.56 (m, 1H), 2.66-2.72 (m, 2H), 3.35 (3H, s), 3.48-3.52 (m, 2H), 3.80-3.91 (m, 1H), 7.17-7.24 (m, 1H), 7.47-7.56 (m, 1H), 7.77-7.89 (m, 2H), 7.95-7.99 (m, 1H), 8.21-8.28 (m, 1H), 9.00-9.05 (m, 1H) |
| 60 | 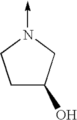 | Diastereomer 1 6-(3- Fluorophenyl)-N- {4-[(3R)-3- hydroxypyrrolidin- 1- yl]cyclohexyl} nicotinamide | LCMS (ES) m/z 384 [M + 1] Calc 383.47 [M + 1] ¹H NMR (400 MHz CD₃OD) ppm 1.12-1.16 (m, 1H), 1.24-1.34 (m, 1H), 1.64-1.77 (m, 5H), 1.87-1.98 (m, 2H), 2.05-2.25 (m, 2H), 2.53-2.63 (m, 2H), 2.73-2.88 (m, 2H), 4.01-4.10 (m, 1H), 4.29-4.37 (m, 1H), 7.16- 7.25 (m, 1H), 7.48-7.57 (m, 1H), 7.79-7.89 (m, 2H), 7.93-8.00 (m, 1H), 8.23-8.30 (m, 1H), 9.01-9.06 (m, 1H) |
| 61 | 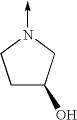 | Diastereoiner 2 6-(3- Fluorophenyl)-N- {4-[(3R)-3- hydroxypyrrolidin- 1- yl]cyclohexyl} nicotinamide | LCMS (ES) m/z 384 [M + 1] Calc 383.47 [M + 1] ¹H NMR (400 MHz CD₃OD) δ ppm 1.35-1.51 (m, 4H), 1.69-1.80 (m, 2H), 2.02-2.17 (m, 4H), 2.17-2.27 (m, 1H), 2.54-2.62 (m, 1H), 2.67-2.77 (m, 1H), 2.77-2.89 (m, 1H), 2.95-3.02 (m, 1H), 3.85-3.95 (m, 1H), 4.31- 4.39 (m, 1H), 7.17-7.27 (m, 1H), 7.46-7.56 (m, 1H), 7.80-7.90 (m, 2H), 7.97-8.02 (m, 1H), 8.25-8.29 (m, 1H), 9.00-9.04 (m, 1H) |
| 62 | 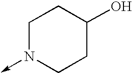 | 6-(3- Fluorophenyl)-N- [4-(4- hydroxypiperidin- 1-yl)cyclohexyl] nicotinamide | Only one diastereoisomer isolated, stereochemistry unconfirmed ¹H NMR (400 MHz CD₃OD) δ ppm 1.48-1.82 (m, 8H), 1.84-1.95 (m, 2H), 1.95-2.11 (m, 2H), 2.22-2.43 (m, 3H), 2.87-3.00 (m, 2H), 3.56-3.66 (m, 1H), 4.09-4.18 (m, 1H), 7.15-7.25 (m, 1H), 7.45-7.57 (m, 1H), 7.77- 7.89 (m, 2H), 7.94-8.01 (m, 1H), 8.22-8.29 (m, 1H), 8.99-9.05 (m, 1H). LCMS m/z 398.1 [M + 1] Calc 398.5 [M + 1] |
| 63 | 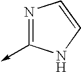 | Diastereomer 1 6-(3- Fluorophenyl)-N- [4-(1H-imidazol-2- yl)cyclohexyl] nicotinamide | Purified by HPLC Method (A) LCMS Method (A) RT 2.21 min (100%) ES+ 365.1 m/z [M + 1] |
| 64 | 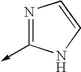 | Diasteromer 2 6-(3- Fluorophenyl)-N- [4-(1H-imidazol-2- yl)cyclohexyl] nicotinamide | LCMS (ES) Obs 363 [M − 1] Calc 363.4 [M + 1] ¹H NMR (400 MHz CD₃OD) ppm 1.48-1.64 (m, 2H), 1.64-1.76 (m, 2H), 2.06-2.19 (m, 4H), 2.72-2.82 (m, 1H), 3.93-4.04 (m, 1H), 6.86-6.92 (m, 2H), 7.13-7.23 (m, 1H), 7.48-7.54 (m, 1H), 7.81-7.89 (m, 2H), 7.96- 8.03 (m, 1H), 8.25-8.30 (m, 1H), 9.03-9.08 (m, 1H) |
| 64A | 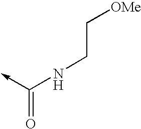 | 6-(3- fluorophenyl)-N- {(1S,3R)-3-[(2- methoxyethyl) carbamoyl] cyclohexyl} nicotinamide | Purified by HPLC Method (B) LCMS Method (A) RT 2.90 minutes (100%) area, ES m/z [M + H] 400.2 |
| 64B | 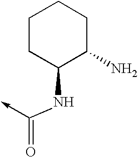 | N-[cis-3- {[(1S,2S)-2- aminocyclohexyl] carbamoyl} cyclohexyl]-6-(3- fluorophenyl) nicotinamide | Purified by HPLC Method (B) LCMS Method (B) RT 2.90 minutes (100%) area, ES m/z [M + H] 439.2 |
| 64C | 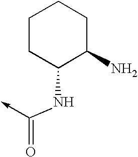 | N-[cis-3- {[(1R,2R)-2- aminocyclohexyl] carbamoyl} cyclohexyl]-6-(3- fluorophenyl) nicotinamide | Purified by HPLC Method (B) LCMS Method (B) RT 2.31 minutes (100%) area, ES m/z [M + H] 439.2 |
| 64D | 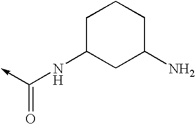 | N-{cis-3-[(3- aminocyclohexyl) carbamoyl] cyclohexyl}-6-(3- fluorophenyl) nicotinamide N-{trans-3-[(3- aminocyclohexyl) carbamoyl]cyclohexyl}-6-(3- fluorophenyl) nicotinamide | Purified by HPLC Method (A) LCMS Method (B) RT 2.96 minutes (100%) area, ES m/z [M + H] 439.2 |
| 64E | 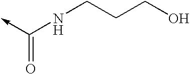 | 6-(3- fluorophenyl)-N- {cis-3-[(3- hydroxypropyl) carbamoyl]cyclohexyl} nicotinamide | Purified by HPLC Method (A) LCMS Method (B) RT 2.72 minutes (100%) area, ES m/z [M + H] 400.2 |
| 64F | 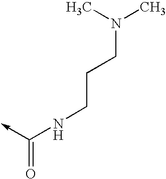 | N-[cis-3-{[3- (dimethylamino) propyl] carbamoyl} cyclohexyl]-6-(3- fluorophenyl) nicotinamide | Purified by HPLC Method (A) LCMS Method (B) RT 3.00 minutes (100%) area, ES m/z [M + H] 427.2 |
| 64G | 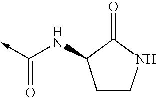 | 6-(3- fluorophenyl)-N- [cis-3-{[(3R)-2- oxopyrrolidin-3- yl]carbamoyl} cyclohexyl] nicotinamide | Purified by HPLC Method (A) LCMS Method (B) RT 2.62 minutes (100%) area, ES m/z [M + H] 425.2 |
| 64H | 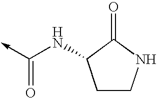 | 6-(3- fluorophenyl)-N- [cis-3-{[(3S)-2- oxopyrrolidin-3- yl]carbamoyl} cyclohexyl] nicotinamide | Purified by HPLC Method (A) LCMS Method (B) RT 2.63 minutes (100%) area, ES m/z [M + H] 425.2 |
| 64I | 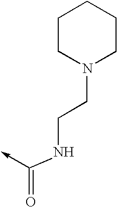 | 6-(3- fluorophenyl)-N- {cis-3-[(2- piperidin-1- ylethyl)carbamoyl] cyclohexyl} nicotinamide | Purified by HPLC Method (B) LCMS Method (B) RT 3.05 minutes (100%) area, ES m/z [M + H] 453.3 |
| 64J | 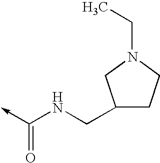 | N-[cis-3-{[(1- ethylpyrrolidin-3- yl)methyl] carbamoyl} cyclohexyl]-6- (3-fluorophenyl) nicotinamide | Purified by HPLC Method (A) LCMS Method (B) RT 3.17 minutes (100%) area, ES m/z [M + H] 453.3 |
| 64K | 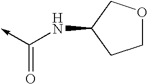 | 6-(3- fluorophenyl)-N- {cis-3-[(3R)- tetrahydrofuran-3- ylcarbamoyl] cyclohexyl} nicotinamide | Purified by HPLC Method (B) LCMS Method (B) RT 2.94 minutes (100%) area, ES m/z [M + H] 412.2 |
| 64L | 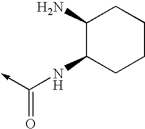 | N-[(1S,3R)-3- {[cis-2- aminocyclohexyl] carbamoyl} cyclohexyl]-6-(3- fluorophenyl) nicotinamide | Purified by HPLC Method (B) LCMS Method (B) RT 2.91 minutes (100%) area, ES m/z [M + H] 439.2 |
| 64M | 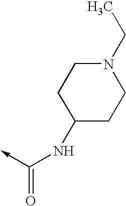 | N-{cis-3-[(1- ethylpiperidin- 4-yl)carbamoyl] cyclohexyl}-6- (3-fluorophenyl) nicotinamide | Purified by HPLC Method (B) LCMS Method (B) RT 2.22 minutes (100%) area, ES m/z [M + H] 453.3 |

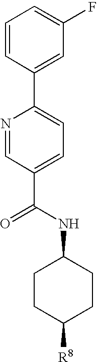

| | | | Purification and |
|---|---|---|---|
| Ex | R⁸ | Name | Characterisation |
| 65 | —OH | 6-(3- | Purified by HPLC Method (A) |
| | | Fluorophenyl)-N- | LCMS Method (B) RT 2.69 minutes (100% area), ES |
| | | (cis-4- | m/z [M + 1] 315.1 |
| | | hydroxycyclohexyl) | |
| | | nicotinamide | |
| 66 | —CO₂H | cis-4-({[6-(3- | ¹H NMR (400 MHz, DMSO-d₆) δ ppm 1.59-1.66 (m, |
| | | Fluorophenyl) | 4H) 2.01-2.04 (m, 2H) 3.32-3.38 (m, 2H) 3.90-3.93 (m, |
| | | pyridin-3- | 1H) 7.29-7.37 (m, 1H) 7.56-7.62 (m, 1H) 7.96-7.99 (m, |
| | | yl]carbonyl} | 1H) 8.02-8.04 (m, 1H) 8.13-8.15 (m, 1H) 8.30-8.32 |
| | | amino) | (m, 1H) 8.46-8.48 (m, 1H) 9.09 (s, 1H) 12.14 (s broad, |
| | | cyclohexanecarboxylic | 1 H) |
| | | acid | |
| 67 | —CH₂N(CH₃)₂ | N-{cis-4- | Purified by HPLC method (B) |
| | | [(Dimethylamino) | LCMS method (A) RT 2.23 min (100% area) ES m/z |
| | | methyl]cyclohexyl}- | 356 [M + 1] |
| | | 6-(3- | |
| | | fluorophenyl) | |
| | | nicotinamide | |
| 68 | 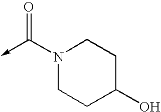 | 6-(3- Fluorophenyl)-N- {cis-4-[(4- hydroxypiperidin- 1-yl)carbonyl] cyclohexyl} nicotinamide | Purified by HPLC Method (A) LCMS Method (B) RT 2.74 minutes (100%) area, ES m/z [M + 1] 426.5 |
| 69 | 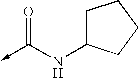 | N-[cis-4- (Cyclopentylcarbmoyl) cyclohexyl]-6-(3- fluorophenyl) nicotinamide | Purified by HPLC Method (A) LCMS Method (B) RT 3.19 minutes (100%) area, ES m/z [M + 1] 410.5 |
| 70 | 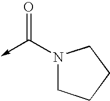 | 6-(3- Fluorophenyl)-N- [cis-4-(pyrrolidin- 1- ylcarbonyl) cyclohexyl]nicotinamide | Purified by HPLC Method (B) LCMS Method (B) RT 2.92 minutes (100%) area, ES m/z [M + 1] 396.5 |
| 71 | —CONHCH₂Ph | N-[cis-4- | Purified by HPLC Method (B) |
| | | (Benzylcarbamoyl) | LCMS Method (B) RT 3.14 minutes (100%) area, |
| | | cyclohexyl]-6-(3- | ES m/z [M + 1] 432.4 |
| | | fluorophenyl) | |
| | | nicotinamide | |
| 72 | 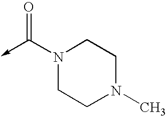 | 6-(3- Fluorophenyl)-N- {cis-4-[(4- methylpiperazin- 1-yl)carbonyl] cyclohexyl} nicotinamide | Purified by HPLC Method (A) LCMS Method (B) RT 2.83 minutes (100%) area, ES m/z [M + 1] 425.5 |
| 73 | 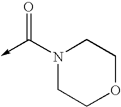 | 6-(3- Fluorophenyl)-N- [cis-4-(morpholin- 4-ylcarbonyl) cyclohexyl] nicotinamide | Purified by HPLC Method (A) LCMS Method (B) RT 2.90 minutes (100%) area, ES m/z [M + 1] 412.5 |
| 74 | —CON(CH₃)₂ | N-[cis-4- | Purified by HPLC Method (A) |
| | | (Dimethylcarbamoyl) | LCMS Method (B) RT 2.98 minutes (100%) area, |
| | | cyclohexyl]-6-(3- | ES m/z [M + 1] 370.4 |
| | | fluorophenyl) | |
| | | nicotinamide | |
| 75 | —CONH^{t}Bu | N-[cis-4-(tert- | Purified by HPLC Method (A) |
| | | Butylcarbamoyl) | LCMS Method (B) RT 3.20 minutes (100%) area, |
| | | cyclohexyl]-6-(3- | ES m/z [M + 1] 398.5 |
| | | fluorophenyl) | |
| | | nicotinamide | |
| 76 | 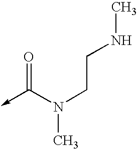 | 6-(3- Fluorophenyl)-N- (cis-4-{methyl[2- (methylamino) ethyl]carbamoyl} cyclohexyl) nicotinamide | Purified by HPLC Method (B) LCMS Method (A) RT 3.07 minutes (100%) area, ES m/z [M + 1] 413.6 |
| 77 | 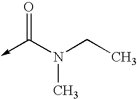 | N-{cis-4- [Ethyl(methyl) carbamoyl] cyclohexyl}-6-(3- fluorophenyl) nicotinamide | Purified by HPLC Method (B) LCMS Method (A) RT 3.15 minutes (100%) area, ES m/z [M + 1] 384.2 |
| 78 | 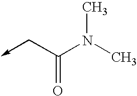 | N-{cis-4-[2- (Dimethylamino- 2-oxoethyl] cyclohexyl}-6-(3- fluorophenyl) nicotinamide | Purified by HPLC Method (A) LCMS Method (A) RT 3.00 min (100%) ES+ 384.1 m/z [M + 1] |
| 79 | 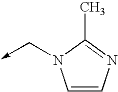 | 6-(3- Fluorophenyl)-N- {cis-4-[(2-methyl- 1H-imidazol-1- yl)methyl] cyclohexyl} nicotinamide | Purified by HPLC method (B) LCMS method (A) RT 2.36 min (100% area) ES m/z 393 [M + 1] |
| 80 | 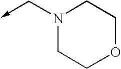 | 6-(3- Fluorophenyl)-N- [cis-4-(morpholin- 4-ylmethyl) cyclohexyl] nicotinamide | Purified by HPLC method (B) LCMS method (A) RT 2.24 min (100% area) ES m/z 398 [M + 1] |
| 81 | 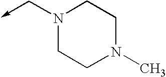 | 6-(3- Fluorophenyl)-N- {cis-4-[(4- methylpiperazin- 1-yl)methyl] cyclohexyl} nicotinamide | Purified by HPLC method (B) LCMS method (A) RT 2.07 min (100% area) ES m/z 411 [M + 1] |

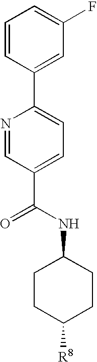

| Ex | R⁸ | Name | Purification and Characterisation |
|---|---|---|---|
| 82 | 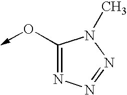 | 6-(3-Fluorophenyl)-N-{trans-4-[(1-methyl-1H- tetrazol-5-yl)oxy]cyclohexyl}nicotinamide | Purified by HPLC Method (A) LCMS method (B) RT 2.92 min (100% area) ES m/z 397 [M + 1] |
| 83 | —OCH₃ | 6-(3-Fluorophenyl)-N-(trans-4- | Purified by HPLC Method (A) |
| | | methoxycyclohexyl)nicotinamide | LCMS Method (B) RT 3.04 |
| | | | minutes (100%) area, ES m/z |
| | | | [M + 1] 329.5 |
| 84 | —CO₂H | trans-4-({[6-(3-Fluorophenyl)pyridin-3- | ¹H NMR (400 MHz, DMSO-d₆) δ |
| | | yl]carbonyl}amino)cyclohexanecarboxylic acid | ppm 1.57-1.75 (m, 4H) 2.01-2.04 |
| | | | (m, 2H) 3.29-3.40 (m, 2H) |
| | | | 3.89-3.96 (m, 1H) 7.32-7.37 (m, |
| | | | 1H) 7.56-7.62 (m, 1H) 7.97-7.99 |
| | | | (m, 1H) 8.02-8.04 (m, 1H) |
| | | | 8.13-8.15 (m, 1H) 8.30-8.32 (m, |
| | | | 1H) 8.46-8.48 (m, 1H) 9.09 (s, 1H) |
| | | | 12.14 (s broad, 1H) |
| 85 | 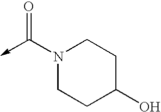 | 6-(3-Fluorophenyl)-N-{trans-4-[(4- hydroxypiperidin-1- yl)carbonyl]cyclohexyl}nicotinamide | Purified by HPLC Method (A) LCMS Method (B) RT 2.68 min (100%) area, ES m/z [M + 1] 426.2 |
| 86 | 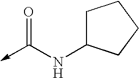 | N-[trans-4-(Cyclopentylcarbamoyl)cyclohexyl]- 6-(3-fluorophenyl)nicotinamide | LCMS (ES+) 410 [M + 1] 1H NMR (400 MHz, DMSO-d₆) δ ppm 1.28-1.54 (m, 8H) 1.57-1.68 (m, 2H), 1.70-1.82 (m, 4H), 1.87-1.97 (m, 2H), 2.02-2.11 (m, 1H), 3.72-3.82 (m, 1H), 3.92-4.02 (m, 1H), 7.28-7.35 (m, 1H), 7.52-7.65 (m, 2H), 7.91-8.04 (m, 2H), 8.10-8.15 (m, 1H), 8.25-8.30 (m, 1H), 8.41-8.48 (m, 1H), 9.04-9.10 (m, 1H). |
| 87 | 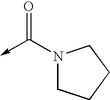 | 6-(3-Fluorophenyl)-N-[trans-4-(pyrrolidin-1- ylcarbonyl)cyclohexyl]nicotinamide | Purified by HPLC Method (A) LCMS Method (B) RT 2.97 min (100%) area, ES m/z [M + 1] 396.2 |
| 88 | —CONHⁱPr | 6-(3-Fluorophenyl)-N-[trans-4- | LCMS (ES+) 384 [M + 1] 1H |
| | | (isopropylcarbamoyl)cyclohexyl]nicotinamide | NMR (400 MHz, DMSO-d₆) δ |
| | | | ppm 0.99-1.03 (m, 6H) 1.32-1.51 |
| | | | (m, 4H), 1.70-1.80 (m, 2H), |
| | | | 1.87-1.97 (m, 2H), 1.99-2.09 (m, |
| | | | 1H), 3.70-3.87 (m, 2H), 7.27-7.36 |
| | | | (m, 1H), 7.49-7.62 (m, 2H), |
| | | | 7.91-8.04 (m, 2H), 8.10-8.15 (m, |
| | | | 1H), 8.25-8.30 (m, 1H), 8.39-8.48 |
| | | | (m, 1H), 9.04-9.10 (m, 1H). |
| 89 | —OCH₂CH₂OH | 6-(3-Fluorophenyl)-N-[trans-4-(2- | Purified by HPLC Method (B) |
| | | hydroxyethoxy)cyclohexyl]nicotinamide | LCMS method (A) RT 2.84 min |
| | | | (100%) ES⁺ 359.2 m/z [M + 1] |
| 90 | 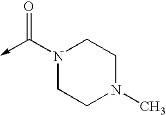 | 6-(3-Fluorophenyl)-N-{trans-4-[(4- methylpiperazin-1- yl)carbonyl]cyclohexyl}nicotinamide | Purified by HPLC Method (A) LCMS Method (B) RT 2.74 minutes (100%) area, ES m/z [M + 1] 425.2 |
| 91 | 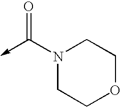 | 6-(3-Fluorophenyl)-N-[trans-4-(morpholin-4- ylcarbonyl)cyclohexyl]nicotinamide | Purified by HPLC Method (A) LCMS Method (B) RT 2.75 minutes (100%) area, ES m/z [M + 1] 412.2 |
| 92 | —CON(CH₃)₂ | N-[trans-4-(Dimethylcarbamoyl)cyclohexyl]-6- | Purified by HPLC Method (A) |
| | | (3-fluorophenyl)nicotinamide | LCMS Method (B) RT 2.76 |
| | | | minutes (100%) area, ES m/z |
| | | | [M + 1] 370.2 |
| 93 | 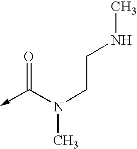 | 6-(3-Fluorophenyl)-N-(trans-4-{methyl[2- (methylamino)ethyl]carbamoyl}cyclohexyl) nicotinamide | Purified by HPLC Method (A) LCMS Method (A) RT 2.23 minutes (100%) area, ES m/z [M + 1] 413.2 |
| 94 | —NHCH₃ | 6-(3-Fluorophenyl)-N-[trans-4- | ¹H NMR (400 MHz, DMSO-d₆) δ |
| | | (methylamino)cyclohexyl]nicotinamide | ppm 1.31-1.41 (m, 2H) 1.58-1.68 |
| | | | (m, 2H) 2.12-2.24 (m, 4H) |
| | | | 2.48-2.54 (m, 1H) 2.77 (s, 3H) |
| | | | 3.97-4.04 (m, 1H) 7.55-7.60 (m, |
| | | | 1H) 7.78-7.86 (m, 1H) 8.18-8.21 |
| | | | (m, 1H) 8.24-8.26 (m, 1H) |
| | | | 8.35-8.37 (m, 1H) 8.51-8.54 (m, |
| | | | 1H) 9.31 (s, 1H) |
| 95 | 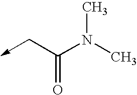 | N-{trans-4-[2-(Dimethylamino)-2- oxoethyl]cyclohexyl}-6-(3- fluorophenyl)nicotinamide | ¹H NMR (400 MHz MeOD-d₄) δ ppm 1.19-1.29 (m, 2H) 1.42-1.54 (m, 2H) 1.52-1.96 (m, 3H) 2.03-2.10 (m, 2H) 2.35-2.38 (m, 2H) 2.99 (s, 3H) 3.12 (s, 3H) 3.89-3.96 (m, 1H) 7.23-7.27 (m, 1H) 7.53-7.59 (m, 1H) 7.85-7.93 (m, 2H) 8.01-8.03 (m, 1H) 8.29-8.32 (m, 1H) 9.08 (s, 1H) |
| 96 | 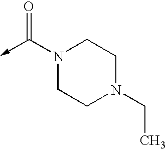 | N-{trans-4-[(4-Ethylpiperazin-1- yl)carbonyl]cyclohexyl}-6-(3- fluorophenyl)nicotinamide | Purified by HPLC Method (A) LCMS Method (A) RT 2.46 minutes (100%) area, ES m/z [M + 1] 439.1 |
| 97 | —C(CH₃)₂OH | 6-(3-Fluorophenyl)-N-[trans-4-(1-hydroxy-1- | LCMS (ES+) 357 (M + 1) |
| | | methylethyl)cyclohexyl]nicotinamide | ¹H NMR (400 MHz MeOD-d₄) δ |
| | | | ppm 1.2 (s, 6H) 1.24-1.5 (m, 5H) |
| | | | 1.96-2.04 (m, 2H) 2.09-2.16 (m, |
| | | | 2H) 3.84-3.94 (m, 1H) 7.22-7.29 |
| | | | (m, 1H) 7.53-7.60 (m, 1H), |
| | | | 7.84-7.94 (m, 2H), 8.00-8.04 (m, |
| | | | 1H), 8.29-8.33 (m, 1H), 9.06-9.09 |
| | | | (m, 1H) |
| 98 | 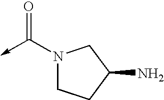 | N-(trans-4-{[(3S)-3-Aminopyrrolidin-1- yl]carbonyl}cyclohexyl)-6-(3- fluorophenyl)nicotinamide | LCMS m/z 411 [M + 1] Calc 411.5 [M + 1] ¹H NMR (400 MHz MeOD-d₄) δ ppm 1.42-1.57 (m, 2H), 1.59-1.74 (m, 2H), 1.89-1.98 (m, 2H), 2.07-2.16 (m, 3H), 2.30-2.58 (m, 2H), 3.49-3.70 (m, 2H), 3.71-3.84 (m, 2H), 3.87-4.04 (m, 2H), 7.17-7.28 (m, 1H), 7.49-7.56 (m, 1H), 7.80-7.90 (m, 2H), 7.96-8.02 (m, 1H), 8.24-8.31 (m, 1H), 8.52-8.58 (m, 1H), 9.02-9.06 (m, 1H) |
| 99 | 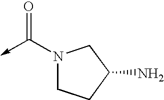 | N-(trans-4-{[(3R)-3-Aminopyrrolidin-1- yl]carbonyl}cyclohexyl)-6-(3- fluorophenyl)nicotinamide | LCMS m/z 411 [M + 1] Calc 411.5 [M + 1] |
| 100 | 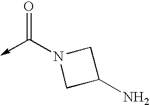 | N-{trans-4-[(3-Aminoazetidin-1- yl)carbonyl]cyclohexyl}-6-(3- fluorophenyl)nicotinamide | LCMS m/z 397 [M + 1] Calc 397.1 [M + 1] ¹H NMR (400 MHz, DMSO-d₆) δ ppm 1.36-1.51 (m, 4H), 1.67-1.80 (m, 2H), 1.85-1.99 (m, 2H), 2.10-2.22 (m, 1H), 3.70-3.87 (2H), 3.94-4.03 (m, 1H), 4.05-4.17 (m, 2H), 4.40-4.49 (m, 1H), 7.28-7.34 (m, 1H), 7.52-7.61 (m, 1H), 7.88-8.00 (m, 2H), 8.06-8.15 (m, 1H), 8.24-8.30 (m, 1H), 9.02-9.08 (m, 1H) |
| 101 | 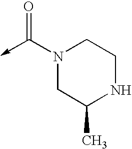 | 6-(3-Fluorophenyl)-N-(trans-4-{[(3S)-3- methylpiperazin-1- yl]carbonyl}cyclohexyl)nicotinamide | Purified by HPLC method (B) LCMS method (B) RT 2.68 min (100% area) ES m/z 425 [M + 1] |
| 102 | 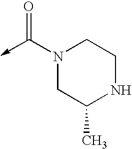 | 6-(3-Fluorophenyl)-N-(trans-4-{[(3R)-3- methylpiperazin-1- yl]carbonyl}cyclohexyl)nicotinamide | Purified by HPLC method (B) LCMS method (B) RT 2.68 min (100% area) ES m/z 425 [M + 1] |
| 103 | —CH₂OH | 6-(3-Fluorophenyl)-N-[trans-4- | ¹H NMR (400 MHz, DMSO-d₆) δ |
| | | (hydroxymethyl)cyclohexyl]nicotinamide | ppm 0.92-1.05 (m, 2H) 1.26-1.40 |
| | | | (m, 3H) 1.75-1.84 (m, 2H) |
| | | | 1.85-1.94 (m, 2H) 3.23 (t, J = 5.9 |
| | | | Hz, 2H) 4.43 (t, J = 5.5 Hz, 1H) |
| | | | 7.29-7.36 (m, 1H) 7.53-7.60 (m, |
| | | | 1H) 7.93-7.99 (m, 1H) 8.01 (d, |
| | | | J = 8.1 Hz, 1H) 8.13 (d, J = 8.1 |
| | | | Hz, 1H) 8.29 (dd, J = 8.8, 2.2 Hz, |
| | | | 1H) 8.47 (d, J = 8.1 Hz, 1H) 9.07 |
| | | | (d, J = 1.5 Hz, 1H) |
| | | | MS calc [M + 1] 329.4, obs |
| | | | [M + H] 329.3 |
| 104 | —OH | 6-(3-Fluorophenyl)-N-(trans-4- | ¹H NMR (400 MHz, DMSO-d₆) δ |
| | | hydroxycyclohexyl)nicotinamide | ppm 1.18-1.31 (m, 2H) 1.31-1.44 |
| | | | (m, 2H) 1.80-1.91 (m, 4H) |
| | | | 3.69-3.80 (m, 1H) 4.59 (d, J = 4.4 |
| | | | Hz, 1H) 7.29-7.36 (m, 1H) |
| | | | 7.53-7.61 (m, 1H) 7.93-7.98 (m, |
| | | | 1H) 8.01 (d, J = 7.3 Hz, 1H) 8.13 |
| | | | (d, J = 8.1 Hz, 1H) 8.27 (dd, |
| | | | J = 8.8, 2.2 Hz, 1H) 8.44 (d, |
| | | | J = 8.1 Hz, 1H) 9.06 (d, J = 2.2 |
| | | | Hz, 1H) |
| | | | MS calc [M + H] 315.4, Obs |
| | | | [M + H] 315.1 |
| 105 | 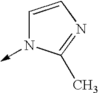 | 6-(3-Fluorophenyl)-N-[trans-4-(2-methyl-1H- imidazol-1-yl)cyclohexyl]nicotinamide | Purified by HPLC method (B) LCMS method (B) RT 2.46 min (100% area) ES m/z 379 [M + 1] |
| 106 | 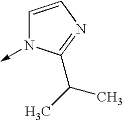 | 6-(3-Fluorophenyl)-N-[trans-4-(2-isopropyl-1H- imidazol-1-yl)cyclohexyl]nicotinamide | Purified by HPLC method (A) LCMS method (A) RT 2.39 min (100% area) ES m/z 407 [M + 1] |
| 107 | 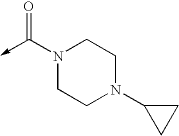 | N-{trans-4-[(4-Cyclopropylpiperazin-1- yl)carbonyl]cyclohexyl}-6-(3- fluorophenyl)nicotinamide | LCMS (ES−) 451 [M + 1] ¹H NMR (400 MHz, DMSO-d₆) δ ppm 0.27-0.48 (m, 4H), 1.35-1.54 (m, 4H), 1.57-1.78 (m, 3H), 1.86-2.00 (m, 2H), 3.20-3.49 (m, 9H), 3.70-3.81 (m, 1H), 7.27-7.37 (m, 1H), 7.51-7.62 (m, 1H), 7.91-8.05 (m, 2H), 8.10-8.18 (m, 1H), 8.25-8.32 (m, 1H), 8.45-8.54 (m, 1H), 9.02-9.10 (br. s. 1H) |
| 108 | 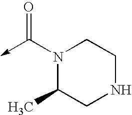 | 6-(3-Fluorophenyl)-N-(trans-4-{[(2R)-2- methylpiperazin-1- yl]carbonyl}cyclohexyl)nicotinamide | Purified by HPLC method (B) LCMS method (B) RT 2.68 min (100% area) ES m/z 425 [M + 1] |
| 109 | 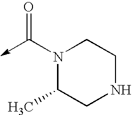 | 6-(3-Fluorophenyl)-N-(trans-4-{[(2S)-2- methylpiperazin-1- yl]carbonyl}cyclohexyl)nicotinamide | Purified by HPLC method (B) LCMS method (B) RT 2.8 min (100% area) ES m/z 425 [M + 1] |
| 110 | 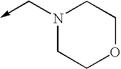 | 6-(3-Fluorophenyl)-N-[trans-4-(morpholin-4- ylmethyl)cyclohexyl]nicotinamide | Purified by HPLC method (B) LCMS method (A) RT 2.31 min (100% area) ES m/z 398 [M + 1] |
| 111 | 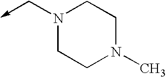 | 6-(3-Fluorophenyl)-N-{trans-4-[(4- methylpiperazin-1- yl)methyl]cyclohexyl}nicotinamide | Purified by HPLC method (B) LCMS method (A) RT 2.07 min (100% area) ES m/z 411 [M + 1] |
| 112 | —CH₂N(CH₃)₂ | N-{trans-4- | Purified by HPLC method (B) |
| | | [(Dimethylamino)methyl]cyclohexyl}-6-(3- | LCMS method (A) RT 2.3 min |
| | | fluorophenyl)nicotinamide | (100% area) ES m/z 356 [M + 1] |
| 113 | 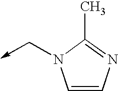 | 6-(3-Fluorophenyl)-N-{trans-4-[(2-methyl-1H- imidazol-1-yl)methyl]cyclohexyl}nicotinamide | Purified by HPLC method (A) LCMS method (A) RT 2.36 min (100% area) ES m/z 393 [M + 1] |
| 113A | 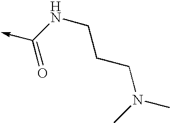 | N-(trans-4-{[3- (dimethylamino)propyl]carbamoyl}cyclohexyl)- 6-(3-fluorophenyl)nicotinamide | Purified by HPLC Method (A) LCMS Method (A) RT 2.20 minutes (100%) area, ES m/z [M + H] 427.2 |
| 113B | 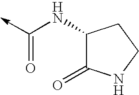 | 6-(3-fluorophenyl)-N-(trans-4-{[(3R)-2- oxopyrrolidin-3- yl]carbamoyl}cyclohexyl)nicotinamide | Purified by HPLC Method (A) LCMS Method (B) RT 2.63 minutes (100%) area, ES m/z [M + H] 425.2 |
| 113C | 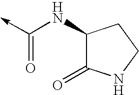 | 6-(3-fluorophenyl)-N-(trans-4-{[(3S)-2- oxopyrrolidin-3- yl]carbamoyl}cyclohexyl)nicotinamide | Purified by HPLC Method (A) LCMS Method (B) RT 2.57 minutes (100%) area, ES m/z [M + H] 425.2 |
| 113D | 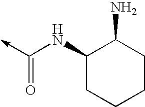 | N-(trans-4-{[(1R,2S)-2- aminocyclohexyl]carbamoyl}cyclohexyl)-6-(3- fluorophenyl)nicotinamide | Purified by HPLC Method (A) LCMS Method (B) RT 2.81 minutes (100%) area, ES m/z [M + H] 439.2 |
| 113E | 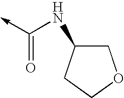 | 6-(3-fluorophenyl)-N-{trans-4-[(3R)- tetrahydrofuran-3- ylcarbamoyl]cyclohexyl}nicotinamide | Purified by HPLC Method (A) LCMS Method (B) RT 2.71 minutes (100%) area, ES m/z [M + H] 412.2 |
| 113F | 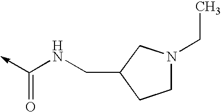 | N-(trans-4-{[(1-ethylpyrrolidin-3- yl)methyl]carbamoyl}cyclohexyl)-6-(3- fluorophenyl)nicotinamide | Purified by HPLC Method (A) LCMS Method (B) RT 3.06 minutes (100%) area, ES m/z [M + H] 453.3 |
| 113G | 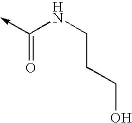 | 6-(3-fluorophenyl)-N-{trans-4-[(3- hydroxypropyl)carbamoyl]cyclohexyl} nicotinamide | Purified by HPLC Method (A) LCMS Method (B) RT 2.62 minutes (100%) area, ES m/z [M + H] 400.2 |
| 113H | 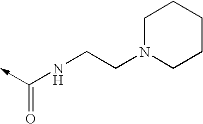 | 6-(3-fluorophenyl)-N-{trans-4-[(2-piperidin-1- ylethyl)carbamoyl]cyclohexyl}nicotinamide | Purified by HPLC Method (B) LCMS Method (B) RT 2.98 minutes (100%) area, ES m/z [M + H] 453.3 |
| 113I | 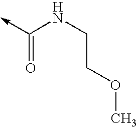 | 6-(3-fluorophenyl)-N-{trans-4-[(2- methoxyethyl)carbamoyl]cyclohexyl} nicotinamide | Purified by HPLC Method (A) LCMS Method (B) RT 2.79 minutes (100%) area, ES m/z [M + H] 400.2 |
| 113J | 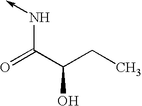 | 6-(3-fluorophenyl)-N-(trans-4-{[(2R)-2- hydroxybutanoyl]amino}cyclohexyl)nicotinamide | Purified by HPLC Method (B) LCMS Method (A) RT 2.95 minutes (100%) area, ES m/z [M + H] 400.2 |
| 113K | 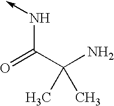 | 6-(3-fluorophenyl)-N-{trans-4-[(2- methylalanyl)amino]cyclohexyl}nicotinamide | Purified by HPLC Method (B) LCMS Method (A) RT 2.85 minutes (100%) area, ES m/z [M + H] 399.2 |
| 113L | 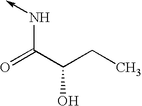 | 6-(3-fluorophenyl)-N-(trans-4-{[(2S)-2- hydroxybutanoyl]amino}cyclohexyl)nicotinamide | Purified by HPLC Method (B) LCMS Method (A) RT 2.85 minutes (100%) area, ES m/z [M + H] 400.2 |
| 113M | 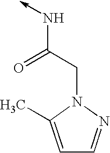 | 6-(3-fluorophenyl)-N-(trans-4-{[(5-methyl-1H- pyrazol-1- yl)acetyl]amino}cyclohexyl)nicotinamide | LCMS (ES+) 436 [M + 1] ¹H NMR (400 MHz DMSO-d₆) δ ppm 1.21-1.48 (m, 4H), 1.81-1.96 (m, 4H), 2.20 (s, 3H), 3.42-3.57 (m, 1H), 3.72-3.83 (m, 1H), 4.66 (s, 2H), 6.00 (s, 1H), 7.22 (s, 1H), 7.22-7.32 (m, 1H), 7.52-7.60 (m, 1H), 7.92-8.06 (m, 3H), 8.12 (d, 1H), 8.24 (d, 1H), 8.43 (d, 1H), 9.03 (s, 1H) |
| 113N | 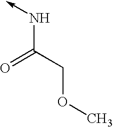 | 6-(3-fluorophenyl)-N-{trans-4- [(methoxyacetyl)amino]cyclohexyl}nicotinamide | LCMS (ES+) 386 [M + 1] ¹H NMR (400 MHz DMSO-d₆) δ ppm 1.36-1.47 (m, 4H), 1.73-1.92 (m, 4H), 3.26 (s, 3H), 3.54-3.66 (m, 1H), 3.69-3.79 (m, 1H), 3.79 (s, 2H), 7.26-7.34 (m, 1H), 7.52-7.60 (m, 2H), 7.82-8.06 (m, 2H), 8.11 (d, 1H), 8.24 (d, 1H), 8.45 (d, 1H), 9.06 (s, 1H) |
| 113O | 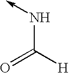 | 6-(3-fluorophenyl)-N-(trans-4- formamidocyclohexyl)nicotinamide | Purified by HPLC Method (B) LCMS Method (B) RT 2.76 minutes (100%) area, ES m/z [M + H] 342.2 |
| 113P | 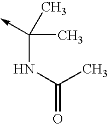 | N-[trans-4-(1-acetamido-1- methylethyl)cyclohexyl]-6-(3- fluorophenyl)nicotinamide | Purified by HPLC Method (A) LCMS Method (B) RT 3.03 minutes (100%) area, ES m/z [M + H] 398.2 |
| 113Q | 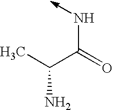 | N-[trans-4-(D-alanylamino)cyclohexyl]-6-(3- fluorophenyl)nicotinamide | Purified by HPLC Method (B) LCMS Method (A) RT 2.22 minutes (100%) area, ES m/z [M + H] 385.2 |
| 113R | 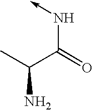 | N-[trans-4-(L-alanylamino)cyclohexyl]-6-(3- fluorophenyl)nicotinamide | Purified by HPLC Method (B) LCMS Method (A) RT 2.22 minutes (100%) area, ES m/z [M + H] 385.2 |

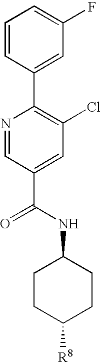

| | | | Purification |
|---|---|---|---|
| | | | and Charac- |
| Ex | R⁸ | Name | terisation |
| 114 | —CH₂OH | 5-Chloro-6-(3-fluorophenyl)-N-(trans-4- | ¹H NMR |
| | | hydroxymethylcyclohexyl)nicotinamide | (DMSO-d₆) |
| | | | δ 0.97-1.06 |
| | | | (m, 2H) |
| | | | 1.30-1.40 |
| | | | (m, 3H) |
| | | | 1.77-1.84 |
| | | | (m, 2H) |
| | | | 1.84-1.95 |
| | | | (m, 2H) |
| | | | 3.73-3.79 |
| | | | (m, 1H) |
| | | | 4.38-4.40 |
| | | | (m, 1H) |
| | | | 7.35-7.38 |
| | | | (m, 1H) |
| | | | 7.53-7.60 |
| | | | (m, 2H) 8.44 |
| | | | (s, 1H) |
| | | | 8.55-8.57 |
| | | | (m, 1H) 9.03 |
| | | | (s, 1H) |

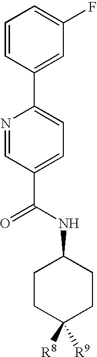

| | | | | Purification and |
|---|---|---|---|---|
| Ex | R⁸ | R⁹ | Name | Characterisation |
| 115 | —CH₃ | —OH | 6-(3-Fluorophenyl)-N-(trans-4- | Purified by HPLC Method (A) |
| | | | hydroxy-4- | LCMS Method (B) T 2.82 |
| | | | methylcyclohexyl)nicotinamide | minutes (100% area), |
| | | | | ES m/z [M + H] 329.2 |
| 116 | —CO₂H | —CH₃ | cis-4-({[6-(3-Fluorophenyl)pyridin-3- | Purified by HPLC Method (B) |
| | | | yl]carbonyl}amino)-1- | LCMS method (A) RT 3.17 |
| | | | methylcyclohexanecarboxylic acid | (100% area) 357.1 [M + H]⁺ |
| 117 | —CH₃ | —CO₂H | trans-4-({[6-(3-Fluorophenyl)pyridin- | Purified using HPLC |
| | | | 3-yl]carbonyl}amino)-1- | Method (A) |
| | | | methylcyclohexanecarboxylic acid | LCMS method (B) RT 2.17 |
| | | | | min (100% area) ES m/z |
| | | | | 357.1 [M + 1] |
| 118 | 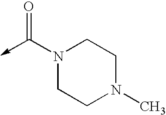 | —CH₃ | 6-(3-Fluorophenyl)-N-{cis-4-methyl- 4-[(4-methylpiperazin-1- yl)carbonyl]cyclohexyl}nicotinamide | Purified by HPLC Method (A), LCMS Method (A) RT 2.29 min (ES) m/z 314.1 [M + 1] |
| 119 | F | F | N-(4,4-Difluorocyclohexyl)-6-(3- | LRMS obs (ES) 335.13 |
| | | | fluorophenyl)nicotinamide | [M + 1] calc 335.1 [M + 1] ¹H NMR (400 MHz CDCl₃) δ |
| | | | | ppm 1.57-1.78 (m, 2H), |
| | | | | 1.84-2.09 (m, 2H), 2.12-2.27 |
| | | | | (m, 4H), 4.08-4.25 (m, 1H), |
| | | | | 6.04-6.17 (m, 1H), 7.12-7.23 |
| | | | | (m, 1H), 7.40-7.53 (m, 1H), |
| | | | | 7.75-7.86 (m, 3H), 8.13-8.23 |
| | | | | (m, 1H), 8.97-9.07 (m, 1H). |

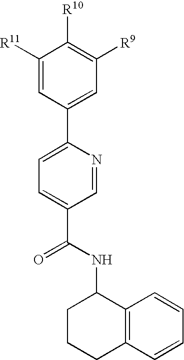

| | | | | | Purification and |
|---|---|---|---|---|---|
| Ex | R⁹ | R¹⁰ | R¹¹ | Name | Characterisation |
| 120 | H | H | H | 6-Phenyl-N-(1,2,3,4- | Purifed by HPLC |
| | | | | tetrahydronaphthalen- | Method (E) |
| | | | | 1-yl)nicotinamide | LCMS Method |
| | | | | | (F) RT 5.09 min |
| | | | | | m/z Obs [M + 1] |
| | | | | | 329.2 calc |
| | | | | | [M + 1] 329.17 |
| 121 | OCH₃ | H | H | 6-(3-Methoxyphenyl)- | Purifed by HPLC |
| | | | | N-(1,2,3,4- | Method (E) |
| | | | | tetrahydronaphthalen- | LCMS Method |
| | | | | 1-yl)nicotinamide | (F) RT 5.20 min |
| | | | | | m/z Obs [M + 1] |
| | | | | | 359.2 calc |
| | | | | | [M + 1] 359.18 |
| 122 | F | H | H | 6-(3-Fluorophenyl)-N- | Purifed by HPLC |
| | | | | (1,2,3,4- | Method (E) |
| | | | | tetrahydronaphthalen- | LCMS Method |
| | | | | 1-yl)nicotinamide | (F) RT 5.28 min |
| | | | | | m/z Obs [M + 1] |
| | | | | | 347.2 calc |
| | | | | | [M + 1] 347.16 |
| 123 | H | F | H | 6-(4-Fluorophenyl)-N- | Purifed by HPLC |
| | | | | (1,2,3,4- | Method (E) |
| | | | | tetrahydronaphthalen- | LCMS Method |
| | | | | 1-yl)nicotinamide | (F) RT 5.26 min |
| | | | | | m/z Obs [M + 1] |
| | | | | | 347.2 calc |
| | | | | | [M + 1] 347.16 |
| 124 | F | H | F | 6-(3,5- | Purifed by HPLC |
| | | | | Difluorophenyl)-N- | Method (E) |
| | | | | (1,2,3,4- | LCMS Method |
| | | | | tetrahydronaphthalen- | (F) RT 5.20 min |
| | | | | 1-yl)nicotinamide | m/z Obs [M + 1] |
| | | | | | 364.1387 calc |
| | | | | | [M + 1] 364.39 |

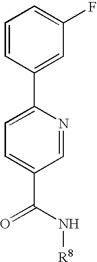

| | | | Purification and |
|---|---|---|---|
| Ex | R⁸ | Name | Characterisation |
| 125 | 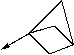 | N-Bicyclo[1.1.1]pent-1-yl-6-(3- fluorophenyl)nicotinamide | LCMS Method (C) RT 2.21 min m/z Obs [M + 1] 283.25 calc [M + 1] 283.12 |
| 126 | 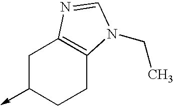 | N-(1-Ethyl-4,5,6,7-tetrahydro-1H- benzimidazol-5-yl)-6-(3- fluorophenyl)nicotinamide | LRMS (ES+) 365 [M + 1] calc 365.43 [M + 1] ¹H NMR (400 MHz MeOD-d₄) ppm 1.35-1.43 (m, 3H) 1.88-2.01 (m, 1H) 2.19-2.28 (m, 1H) 2.61-2.78 (m, 3H) 2.92-3.01 (m, 1H) 3.90-4.00 (m, 2H) 4.31-4.42 (m, 1H) 7.17-7.25 (m, 1H) 7.48-7.56 (m, 1H) 7.80-7.91 (m, 2H) 7.96-8.02 (m, 1H) 8.26-8.32 (m, 1H) |
| 127 | 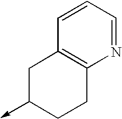 | 6-(3-Fluorophenyl)-N-(5,6,7,8- tetrahydroquinolin-6- yl)nicotinamide | Purified by HPLC Method (B) LCMS Method (B) RT 2.99 min (100%) 348.2 m/z [M + H]+ |
| 128 | 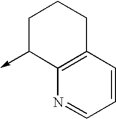 | 6-(3-Fluorophenyl)-N-(5,6,7,8- tetrahydroquinolin-8- yl)nicotinamide | Purified by HPLC Method (A) LCMS Method (B) RT 3.13 min (100%) 348.1 m/z [M + H]+ |
| 129 | 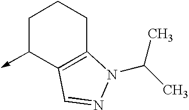 | 6-(3-Fluorophenyl)-N-(1-isopropyl- 4,5,6,7-tetrahydro-1H-indazol-4- yl)nicotinamide | LRMS m/z Obs 378 [M]⁺ calc 378.2 |
| 130 | 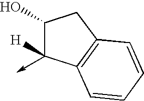 | 6-(3-Fluorophenyl)-N-(cis-2- hydroxy-2,3-dihydro-1H-inden- 1yl]nicotinamide | LCMS Method (C) RT 1.82 min m/z Obs 315.12 [M + 1] calc 315.14 [M + 1] |
| 130A | 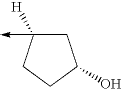 | 6-(3-fluorophenyl)-N-[(1R,3R)-3- hydroxycyclopentyl]nicotinamide | Purified by HPLC Method (A) LCMS Method (B) RT 2.64 minutes (100%) area, ES m/z [M + H] 301.1 |

Details of purification methods referenced in the tables above along with further details concerning the preparation and characterization of selected tabulated Examples are provided in the following section.

Method A

| HPLC | LCMS Method A | | HPLC Method A | |
|---|---|---|---|---|
| conditions | (analytical) | | (preparative) | |
| Column | Sunfire C18 | | Sunfire Prep C18 | |
| | 5 μm 4.6 × 50 mm | | 5 μm 19 × 100 mm | |
| Temperature | Ambient | | Ambient | |
| Detection | UV 225 nm - ELSD-MS | | ELSD-MS | |
| System/Data file | CTC-MUX1 | | Fractionlynx 1 | |
| Injection volume | 5 μL | | 1000 μL | |
| Flow rate | 1.5 mL/min | | 18 mL/min | |
| Mobile phase | A: H₂O + 0.1% formic | | A: H₂O + 0.1% formic | |
| | acid | | acid | |
| | B: MeCN + 0.1% formic | | B: MeCN + 0.1% formic | |
| | acid | | acid | |
| | Time | | Time | |
| | (min) | % B | (min) | % B |
| Gradient | 0 | 5 | 0-1.0 | 5 |
| | 0-3.0 | 5-95 | 1.0-7.0 | 5-98 |
| | 3.0-4.0 | 95 | 7.0-9.0 | 98 |
| | 4.0-4.1 | 95-5 | 9.0-9.10 | 98-5 |
| | 4.1-5.0 | 5 | 9.10-10 | 5 |

Method B

| HPLC | LCMS Method B | HPLC Method B |
|---|---|---|
| conditions | (analytical) | (preparative) |
| Column | XTerra C18 | XTerra C18 |
| | 5 μm 4.6 × 50 mm | 5 μm 19 × 100 mm |
| Temperature | Ambient | Ambient |
| Detection | UV 225 nm - ELSD-MS | ELSD-MS |
| System/Data file | CTC-MUX1 | Fractionlynx 1 |
| Injection volume | 5 μL | 1000 μL |
| Flow rate | 1.5 mL/min | 18 mL/min |
| Mobile phase | A: H₂O + 0.1% ammonia | A: H₂O + 0.1% DEA |
| | B: MeCN + 0.1% | B: MeCN + 0.1% DEA |
| | ammonia | |

| | | | | |
|---|---|---|---|---|
| | Time | | Time | |
| | (min) | % B | (min) | % B |
| Gradient | 0 | 5 | 0-1.0 | 5 |
| | 0-3.0 | 5-95 | 1.0-7.0 | 5-98 |
| | 3.0-4.0 | 95 | 7.0-9.0 | 98 |
| | 4.0-4.1 | 95-5 | 9.0-9.10 | 98-5 |
| | 4.1-5.0 | 5 | 9.10-10 | 5 |

Methods X and Y

| HPLC | LCMS Method X | | LCMS Method Y | |
|---|---|---|---|---|
| conditions | (analytical) | | (analytical) | |
| Column | Waters Xbridge | | Waters Xbridge | |
| | C18 50 × 2.1 mm, | | C18 50 × 2.1 mm, | |
| | 3.5 μm | | 3.5 μm | |
| Temperature | 30° C. | | 30° C. | |
| Detection | DAD 220-320 nm - MSD | | DAD 220-320 nm - MSD | |
| | (es-positive/negative) | | (es-positive/negative) | |
| System/Data file | Agilent 1200 | | Agilent 1100 | |
| Injection volume | 1.5 μL | | 5 μL | |
| Flow rate | 0.8 mL/min | | 0.8 mL/min | |
| Mobile phase | A: MeCN + 0.1% formic | | A: MeCN + 10 mM | |
| | acid | | Ammonia | |
| | B: H₂O + 0.1% formic | | B: H₂O + 10 mM | |
| | acid | | Ammonia | |
| | Time | | Time | |
| | (min) | % A | (min) | % A |
| Linear Gradient | 0 | 2 | 0 | 2 |
| | 3.5 | 98 | 3.5 | 98 |
| | 6 | 98 | 6 | 98 |

### EXAMPLE 6

### N-Cyclopropyl-6-(3-fluoro-phenyl)-nicotinamide

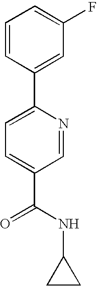

6-(3-Fluorophenyl)nicotinic acid (0.15 g, 0.691 mmol) was dissolved in dichloromethane (3 mL). To this stirred solution were added 1-ethyl-3-(3-dimethylaminopropyl)carbodiimide (0.146 g, 0.760 mmol) and 1-hydroxy-7-azabenzotriazole (0.094 g, 0.691 mmol), followed by cyclopropylamine (0.0394 g, 0.691 mmol). After 18 hours stirring at room temperature water (3 mL) was added and the phases were separated. The solvents were evaporated, and the product purified using flash column chromatography using a DCM to DCM/MeOH 85/15 gradient to give 44 mg of the title product.

### EXAMPLE 9

### N-Cyclopentyl-6-(3-fluorophenyl)nicotinamide

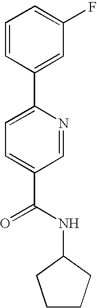

6-(3-Fluorophenyl)nicotinic acid (10.8 mg, 50 mmol), HATU (19 mg, 50 mmol) and triethylamine (5.1 mg, 50 mmol) were dissolved in DMF. Cyclopentylamine (4.3 mg, 50 mmol) was added and the solution was agitated at room temperature for 16 hours. The solvent was evaporated and the compound was purified by HPLC to give the title compound (6 mg). HPLC Method C shows the analytical conditions used. HPLC Method D shows preparative conditions used.

Examples 1, 10, 24, 25, 104, 125 and 130 were similarly prepared by substituting cyclopentylamine with the appropriate amine.

LCMS Method C (analytical)

| HPLC | | | |
|---|---|---|---|
| conditions | LCMS (QC) | | |
| Column | Analytical S&P Advantage Armor C18 | | |
| | 5 μm 4.6 × 50 mm | | |
| Temperature | Ambient | | |
| Detection | UV 220-400 nm - ELSD-MS | | |
| Injection | 12 μL | | |
| volume | | | |
| Flow rate | 4.0 mL/min | | |
| Mobile | A: H₂O + 0.5% trifluoroacetic acid | | |
| phase | B: MeCN | | |
| | Time (min) | % A | % B |
| Gradient | 0 | 95 | 5 |
| | 0.50 | 95 | 5 |
| | 3.60 | 5 | 95 |
| | 3.95 | 95 | 5 |
| | 4.00 | 95 | 5 |

HPLC Method D (preparative)

| HPLC | | | |
|---|---|---|---|
| conditions | Preparative | | |
| Column | Phenomenex Luna C18(2) 5 μm 21.2 × 50 mm | | |
| Temperature | Ambient | | |
| Detection | ELSD | | |
| Injection | 2000 μL | | |
| volume | | | |
| Flow rate | 45.0 mL/min | | |
| Mobile | A: H₂O + 0.5% trifluoroacetic acid | | |
| phase | B: MeCN + 0.5% trifluoroacetic acid | | |
| | Time (min) | % A | % B |
| Gradient | 0 | 90 | 10 |
| | 0.10 | 90 | 10 |
| | 2.30 | 30 | 70 |
| | 2.70 | 5 | 95 |
| | 3.70 | 5 | 95 |
| | 3.90 | 90 | 10 |
| | 4.00 | 90 | 10 |

### EXAMPLE 11A

### (1S,3R)-3-{([6-(3-Fluorophenyl)pyridin-3-yl]carbonyl}amino)cyclopentanecarboxylic acid

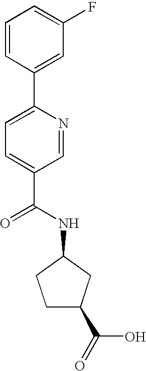

This compound was prepared in the same way as Example 27 starting from (1S,3R)-3-({[6-(3-fluorophenyl)pyridin-3-yl]carbonyl}amino)cyclopentanecarboxylic acid ethyl ester.

### EXAMPLE 11B

### (1R,3S)-3-({[6-(3-Fluorophenyl)pyridin-3-yl]carbonyl}amino)cyclopentanecarboxylic acid

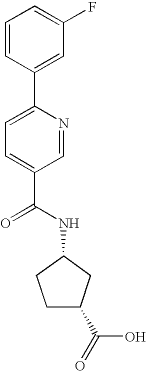

This compound was prepared in the same way as Example 11a starting from (1R,3S)-3-({[6-(3-fluorophenyl)pyridin-3-yl]carbonyl}amino)cyclopentanecarboxylic acid methyl ester.

¹H NMR (400 MHz, DMSO-d₆) δ ppm 1.56-1.69 (m, 1H) 1.73-1.97 (m, 4H) 2.17-2.26 (m, 1H) 2.70-2.83 (m, 1H) 4.25-4.32 (m, 1H) 7.25-7.35 (m, 1H) 7.52-7.61 (m, 1H) 7.89-8.03 (m, 2H) 8.10-8.16 (m, 1H) 8.26-8.30 (m, 1H) 8.57-8.61 (m, 1H) 9.08-9.09 (m, 1H) 12.08 (br s, 1H).

### EXAMPLE 22

### N-Cyclohexyl-6-(4-fluorophenyl)nicotinamide

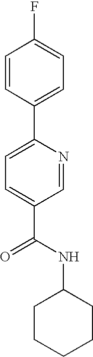

6-(3-Fluorophenyl)nicotinic acid (33 mg, 0.15 mmol), HOBT (46 mg, 0.3 mmol) and cyclohexylamine (15 mg, 0.15 mmol) were added to a suspension of polymer suspended carbodiimide (0.2 mmol) in DMF (1 mL). The reaction was stirred at room temperature for 18 hours. The solvent was removed under reduced pressure and the residue was purified by reverse phase HPLC chromatography (Method E). The products were analysed using LCMS (Method F). This gave (50 mg) of the title compound.

### HPLC Method E (Preparative)

Purification was achieved using a Waters Sunfire C18 Column 20×50 mm×5 μm eluting with a water/acetonitrile/0.1% formic acid gradient, typically from 85% water to 5% water over 8 minutes. The flow rate was 30 ml/min and the trigger was by mass spectrometry.

### LCMS Method F (Analytical)

Analysis was conducted using a Sunfire C18 Column, 2.1×50 mm×5 μm. Gradient elution was carried out with water/acetonitrile/0.1% formic acid, gradient 95-5% water over 8 minutes, 1 min hold at the end of the run., flow rate 1 mL/min, purity assessment by UV (215 nM).

| HPLC | LCMS Method (G) |
|---|---|
| conditions | (analytical) |
| Column | Fortis Pace |
| | C18 20 × 2.1 mm, 3.0 μm |
| Temperature | 75° C. |
| Detection | DAD 210-450 nm |
| Injection volume | 1.5 μL |
| Flow rate | 1.8 mL/min |
| Mobile phase | A: H2O |
| | B: MeCN |
| | C: 2% Formic acid(aq) |
| Linear Gradient | 70-2% A over 1.8 min, |
| | 0.2 min hold, |

| HPLC | LCMS Method (H) | | LCMS Method (I) | |
|---|---|---|---|---|
| conditions | (analytical) | | (analytical) | |
| Column | Waters Xbridge | | Waters Xbridge | |
| | C18 50 × 2.1 mm, | | C18 50 × 2.1 mm, | |
| | 3.5 μm | | 3.5 μm | |
| Temperature | 30° C. | | 30° C. | |
| Detection | DAD 220-320 nm - MSD | | DAD 220-320 nm - MSD | |
| | (es-positive/negative) | | (es-positive/negative) | |
| System/Data file | Agilent 1200 | | Agilent 1100 | |
| Injection volume | 1.5 μL | | 5 μL | |
| Flow rate | 0.8 mL/min | | 0.8 mL/min | |
| Mobile phase | A: MeCN + 0.1% formic | | A: MeCN + 10 mM | |
| | acid | | Ammonia | |
| | B: H₂O + 0.1% formic | | B: H₂O + 10 mM | |
| | acid | | Ammonia | |
| | Time | | Time | |
| | (min) | % A | (min) | % A |
| Linear Gradient | 0 | 2 | 0 | 2 |
| | 3.5 | 98 | 3.5 | 98 |
| | 6 | 98 | 6 | 98 |

Examples 2, 3, 4, 5 and 18, 19, 20, 21, 22 were prepared in a similar manner.

### EXAMPLE 27

### cis-3-({[6-(3-Fluorophenyl)pyridin-3-yl]carbonyl}amino)cyclohexanecarboxylic acid

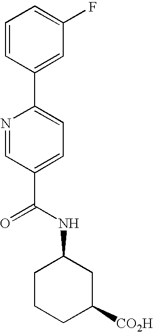

Sodium hydroxide solution (1 M, 45 mL) was added dropwise to a solution of methyl cis-3-({[6-(3-fluorophenyl)pyridin-3-yl]carbonyl}amino)cyclohexanecarboxylate (Example 133, 0.815 g, 2.29 mmol) in methanol (45 mL) at 55° C. The resulting solution was removed from the heat and stirred at room temperature for 5 minutes. The reaction was found to be complete and so aqueous hydrochloric acid (2 M) was added dropwise until the reaction mixture was pH 2. The resulting mixture was partitioned between ethyl acetate (150 mL) and water (100 mL) and the aqueous layer was extracted further with ethyl acetate (50 mL). The combined organic extracts were dried (MgSO₄), filtered and concentrated to give 747 mg of cis-3-({[6-(3-fluorophenyl)pyridin-3-yl]carbonyl}amino)cyclohexanecarboxylic acid as a white solid.

### EXAMPLE 41

### N-cis-3-[(4-Ethylpiperazin-1-yl)carbonyl]cyclohexyl}-6-(3-fluorophenyl)nicotinamide

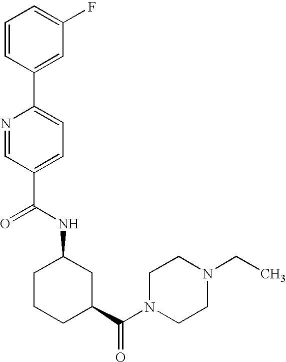

The title compound was prepared using N,N-carbonyldiimidazole as the coupling agent as described in the general methods section.

The enantiomers of the compound were separated using the HPLC conditions below to give the enantiomer A (peak 1) example 41a and enantiomer B (peak 2) example 41b.

### HPLC Conditions:

| | |
|---|---|
| Column analytical (250 * 46 mm id) | Chiralpak AD-H |
| Column prep (250 * 21.2 mm id) | |
| Mobile phase: | heptane:IPA:DEA (70:30:0.1) |
| Flow rate (ml/min) | 1.0 (analytical) 15 (prep) |
| Detection (nm) | 225 nm and 254 nm |
| Temperature: | Ambient |
| Sample dissolution(mg/ml): | 46 mg in 1 ml EtOH + 1.5 ml |
| | MeOH = 18.4 mg/ml |
| Maximum injection volume (μl): | 1500 μl |

| | Enantiomer 1 | | Enantiomer 2 | | |
|---|---|---|---|---|---|
| | RT (min.) | Area % | RT (min.) | Area % | % ee |
| Mixture | 12.065 | — | 17.068 | — | |
| Pk 1 | 12.029 | 100 | — | — | >99.5 |
| Pk 2 | — | — | 17.130 | 100 | >99.5 |

### EXAMPLE 44

### N-(4-Aminocyclohexyl)-6-(3-fluorophenyl)nicotinamide

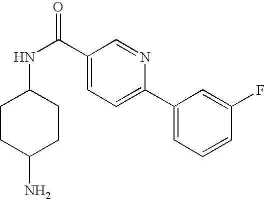

tert-Butyl [4-({[6-(3-fluorophenyl)pyridin-3-yl]carbonyl}amino)cyclohexyl]carbamate, preparation 140 (680 mg, 1.64 mmol) was dissolved in a 4M solution of hydrogen chloride in dioxane (5 mL) and then stirred at room temperature overnight. The solvent was removed under reduced pressure and the residue was partitioned between dichloromethane (20 mL) and saturated aqueous sodium hydrogen carbonate solution (20 mL). The organic layer was filtered through a phase separation tube and evaporated to give a brown gum (450 mg). The product appears to be just one stereoisomer with stereochemistry unknown.

### EXAMPLES 45 AND 46

### N-(4-Acetamidocyclohexyl)-6-(3-fluorophenyl)nicotinamide and 6-(3-fluorophenyl)-N-{4-[2-(methylthio)-1H-imidazol-1-yl]cyclohexyl}nicotinamide

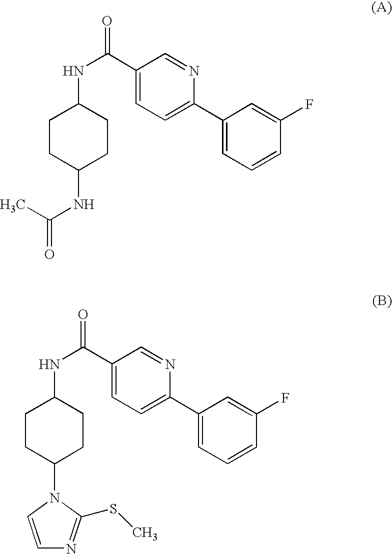

Dimethyl (2,2-diethoxyethyl)dithioimidocarbonate (249 mg, 1.05 mmol) was added to a stirred solution of tert-butyl [4-({[6-(3-fluorophenyl)pyridin-3-yl]carbonyl}amino)cyclohexyl]carbamate, preparation 140, (395 mg, 1.26 mmol) in acetic acid (5 mL). The reaction mixture was heated to reflux for 8 h and then allowed to cool. The solvent was removed under reduced pressure and the residue was partitioned between ethyl acetate (20 mL) and saturated aqueous sodium hydrogen carbonate. The ethyl acetate was removed and the aqueous layer extracted once again with dichloromethane. The organic layers were combined, dried using anhydrous MgSO₄, filtered and evaporated under reduced pressure. The residue was purified by chromatography on silica eluting with dichloromethane:methanol:0.88 aqueous ammonia 100:0:0 to 95:5:0.25 to give compound (B) as a brown gum (250 mg). Further elution of the column with dichloromethane:methanol:0.88 aqueous ammonia 80:20:2 gave compound (A) (40 mg).

### EXAMPLE 47

### 6-(3-Fluorophenyl)-N-[4-(1H-imidazol-1-yl)cyclohexyl]nicotinamide

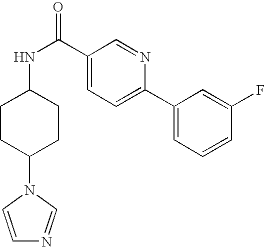

Raney nickel (1680 mg, 19.6 mmol) was added to a solution of 6-(3-fluorophenyl)-N-{-4-[2-(methylthio)-1H-imidazol-1-yl]cyclohexyl}nicotinamide (Example 45) (225 mg, 0.55 mmol) in a mixture of water (5 mL) and ethanol (20 mL). The reaction mixture was stirred for 90 min at room temperature. More Raney nickel (500 mg, 5.83 mmol) was added after 1 hour and 2 hours. The reaction mixture was filtered through Celite®, washing with 1M solution of ammonia in methanol (30 mL) and dichloromethane (20 mL) and the combined liquors on evaporation gave a brown gum (100 mg). The filter pad was suspended in dichloromethane/1M solution of ammonia in methanol (2:1 ratio, 50 mL) for 48 hours. The Celite® was filtered off and the solvents evaporated to give another 30 mg of brown gum. The batches of residue were combined and purified by chromatography on silica eluting with ethyl acetate:methanol:0.88 aqueous ammonia 100:0:0 to 75:25:2.5 to give the title compound as a light brown solid.

### EXAMPLES 49 AND 50

### 6-(3-Fluorophenyl)-N-(4-morpholin-4-ylcyclohexyl)nicotinamide

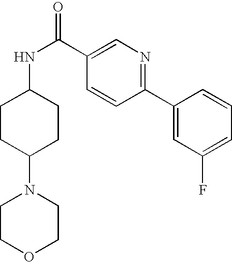

The title compound was prepared in analogous manner to Example 62 using morpholine (104 mg, 1.20 mmol) instead of piperidin-4-ol. After work-up the residue was purified on silica eluting with EtOAc/EtOAc:MeOH:NH₃ (95:5:0.5), 100/0 to 0/100 then eluted with CH₂Cl₂:MeOH:NH₃, 90:10:1 to 80:20:22 to afford two products, the cis and trans isomers of the title compound. The first product which eluted was obtained as a colourless gum (22 mg) and the second product which eluted was obtained as a colourless solid (44 mg).

### EXAMPLES 51 AND 52

### 6-(3-Fluorophenyl)-N-[4-(4-methylpiperazin-1-yl)cyclohexyl]nicotinamide

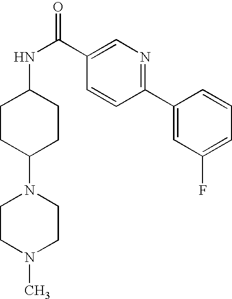

The title compound was prepared in analogous manner to Example 62 using N-methylpiperazine (120 mg, 1.20 mmol) instead of piperidin-4-ol. After work-up the residue was purified on silica eluting with dichloromethane/dichloromethane:MeOH:NH₃ (70:30:3), 100/0 to 0/100 to afford two products the cis and trans isomers of the title compound. The first product which eluted was obtained as a brown gum (6 mg) and the second product which eluted was obtained as a colourless solid (44 mg).

### EXAMPLE 53

### 6-(3-Fluorophenyl)-N-[4-(1,4-oxazepan-4-yl)cyclohexyl]nicotinamide

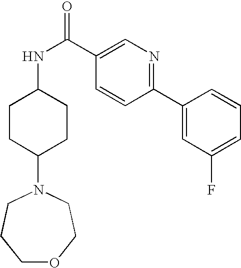

The title compound was prepared in analogous manner to Example 62 using 1,4-oxazepane hydrochloride salt (99 mg, 0.80 mmol) instead of piperidin-4-ol. After work-up the compound (58 mg) crystallised out from DMSO (1 mL).

### EXAMPLES 54 AND 55

### 6-(3-Fluorophenyl)-N-{4-[(2-methoxyethyl)amino]cyclohexyl}nicotinamide

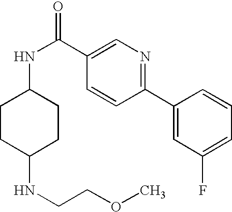

The title compound was prepared in analogous manner to Example 62 using 2-methoxyethanamine (60 mg, 0.80 mmol) instead of piperidin-4-ol and heptane:IPA:DEA (90:10:0.1) as eluant from the HPLC. This gave two compounds (29 mg) and (15 mg), cis and trans isomers of 6-(3-fluorophenyl)-N-{4-[(2-methoxyethyl)amino]cyclohexyl}nicotinamide.

### EXAMPLES 56 AND 57

### 6-(3-Fluorophenyl)-N-[4-(tetrahydrofuran-3-ylamino)cyclohexyl]nicotinamide

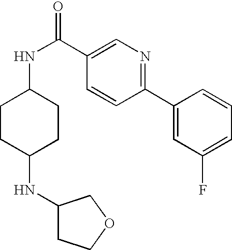

The title compound was prepared in analogous manner to Example 62 using tetrahydrofuran-3-amine hydrochloride salt (99 mg, 0.80 mmol) instead of piperidin-4-ol and heptane:IPA:DEA (83:17:0.1) as eluant from the HPLC. This gave two compounds (25 mg) and (5 mg), cis and trans isomers of 6-(3-fluorophenyl)-N-[4-(tetrahydrofuran-3-ylamino)cyclohexyl]nicotinamide.

### EXAMPLES 58 AND 59

### 6-(3-Fluorophenyl)-N-{4-[(2-methoxyethyl)(methyl)amino]cyclohexyl}nicotinamide

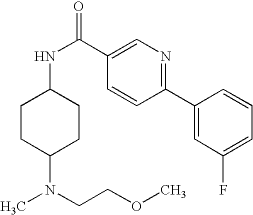

The title compound was prepared in analogous manner to Example 62 using (2-methoxy-ethyl)methylamine instead of piperidin-4-ol and heptane:IPA:DEA (80:20:0.1) as eluant from the HPLC. This gave two compounds (20 mg) and (30 mg), cis and trans isomers of 6-(3-fluorophenyl)-N-{4-[(2-methoxyethyl)(methyl)amino]cyclohexyl}nicotinamide.

### EXAMPLES 60 AND 61

### 6-(3-Fluorophenyl)-N-{4-[(3S)-3-hydroxypyrrolidin-1-yl]cyclohexyl}nicotinamide

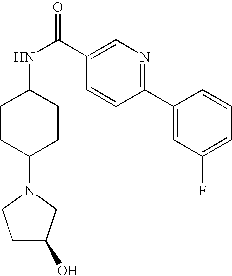

The title compound was prepared in analogous manner to Example 62 using (3S)-pyrrolidin-3-ol hydrochloride salt (99 mg, 0.8 mmol) instead of piperidin-4-ol. The product was purified by HPLC using the same conditions as for Example 62, eluant heptane:IPA:DEA 80:20:1, to give the two isomers (20 mg and 4 mg).

### EXAMPLE 62

### 6-(3-Fluorophenyl)-N-[4-(4-hydroxypiperidin-1-yl)cyclohexyl]nicotinamide PF—

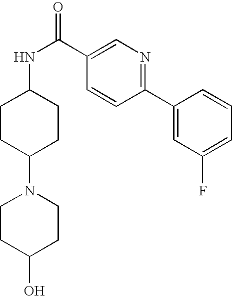

6-(3-Fluorophenyl)-N-(4-oxocyclohexyl)nicotinamide (250 mg, 0.80 mmol) was dissolved in a 1:1 mixture of tetrahydrofuran and DMSO (3 mL). Piperidin-4-ol (81 mg, 0.80 mmol) and acetic acid (72 mg, 1.2 mmol) were added. The reaction mixture was left to stir at room temperature for 10 minutes and then sodium triacetoxyborohydride (424 mg, 2.0 mmol) was added. The reaction was stirred for 15 hours at room temperature. The pH of the solution was adjusted to 12 with 3M aqueous sodium hydroxide solution. Water (5 mL) and dichloromethane (20 mL) were added. The resultant biphasic layer was stirred rapidly for 20 min and the organic layer was removed and filtered through a phase separation tube. The solvent was evaporated and the residue purified by HPLC using a Chiralpak AD-H (250×21.2 mm id) column eluting with heptane:IPA:DEA (80:20:0.1). This gave one compound of unknown cis/trans stereochemical assignment (25 mg).

### EXAMPLES 63 AND 64

### 6-(3-Fluorophenyl)-N-[4-(1H-imidazol-2-yl)cyclohexyl]nicotinamide (cis and trans diastereomers)

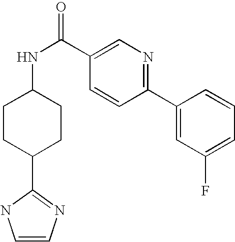

Starting material (A) from Preparation 19 (29 mg, 0.18 mmol) and 6-(3-fluorophenyl)nicotinic acid (38 mg, 0.18 mmol) were dissolved in DMF (5 mL). Triethylamine (89 mg, 0.88 mmol) and HBTU (84 mg, 0.22 mmol) were added and the reaction mixture was stirred at 50° C. for 16 hours. The solvent was removed and the residue purified by chromatography on silica eluting with ethyl acetate:methanol:0.88 aqueous ammonia 100:0:0 to 75:25:0.75 to give the title compound 63 as a light brown gum (57 mg).

Starting material (B) from Preparation 19 (29 mg, 0.18 mmol) and 6-(3-fluorophenyl)nicotinic acid (38 mg, 0.18 mmol) were dissolved in DMF (5 ml). Triethylamine (89 mg, 0.88 mmol) and HBTU (84 mg, 0.22 mmol) were added and the reaction mixture was stirred at 50° C. for 16 hours. The solvent was removed under reduced pressure and the residue was purified by chromatography on silica eluting with ethyl acetate:methanol:0.88 aqueous ammonia 100:0:0 to 75:25:0.75 to give the title compound 64 as a light brown gum. The gum was triturated from hot acetonitrile/methanol to give a beige solid (5 mg)

### EXAMPLE 66

### cis-4-({[6-(3-Fluorophenyl)pyridin-3-yl]carbonyl}amino)cyclohexanecarboxylic acid

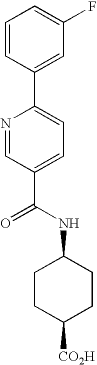

Sodium hydroxide solution (1 M, 14 mL) was added dropwise to a solution of methyl cis-4-({[6-(3-fluorophenyl)pyridin-3-yl]carbonyl}amino)cyclohexanecarboxylate (Example 132, 0.25 g, 0.70 mmol) in methanol (14 mL) at 55° C. The resulting solution was removed from the heat and stirred at room temperature for 5 minutes. The reaction was found to be complete and so aqueous hydrochloric acid (2 M) was added dropwise until the reaction mixture was pH 2. The resulting mixture was partitioned between ethyl acetate (50 mL) and water (50 mL) and the aqueous layer was extracted further with ethyl acetate (25 mL). The combined organic extracts were dried (MgSO4), filtered and concentrated to give 240 mg of cis-4-({[6-(3-fluorophenyl)pyridin-3-yl]carbonyl}amino)cyclohexanecarboxylic acid as a colourless gum. After several days the gum had crystallised on standing and was triturated with hot toluene and cooled to room temperature. The resulting solid was filtered off, washed with diethyl ether and dried to give 101 mg of the title compound containing approximately 8% of the trans isomer.

### EXAMPLE 82

### 6-(3-Fluorophenyl)-N-{trans-4-[(1-methyl-1H-tetrazol-5-yl)oxy]cyclohexyl}nicotinamide

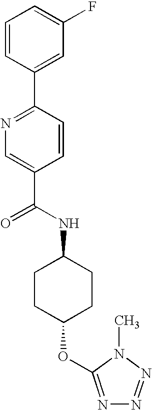

To a suspension of 6-(3-fluorophenyl)-N-(trans-4-hydroxycyclohexyl)nicotinamide (60 mg, 0.19 mmol), example 104 in anhydrous THF (10 mL) was added sodium hydride (60% dispersion in oil, 5.0 mg, 0.125 mmol) and the reaction mixture was stirred at room temperature for 30 minutes. 5-Chloro-1-methyl-1H-tetrazole (25 mg, 0.210 mmol) was added and the mixture was stirred at room temperature for 20 hours and then at reflux for 18 hours. The cooled reaction mixture was partitioned between water (20 mL) and dichloromethane (15 mL) and the organic layer was separated, dried over anhydrous MgSO₄, filtered and evaporated. The product was purified by reverse phase HPLC.

### EXAMPLE 84

### trans-4-({[6-(3-Fluorophenyl)pyridin-3yl]carbonyl}amino)cyclohexanecarboxylic acid

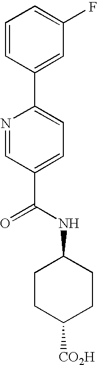

Sodium hydroxide solution (1 M, 100 mL) was added dropwise to a solution of methyl trans-4-({[6-(3-fluorophenyl)pyridin-3-yl]carbonyl}amino) cyclohexanecarboxylate (Example 131, 1.80 g, 5.05 mmol) in methanol (130 mL) at 55° C. The resulting solution was removed from the heat and allowed to cool to room temperature. The reaction was found to be complete and so aqueous hydrochloric acid (2 M) was added dropwise until the reaction mixture was pH 2. The resulting mixture was cooled in an ice bath and stirred for 15 min. The precipitated product was filtered off, washed with water and diethyl ether and dried under vacuum at 50° C. to give 1.6 g (93%) of trans-4-({[6-(3-fluorophenyl)pyridin-3 yl]carbonyl}amino)cyclohexanecarboxylic acid.

### EXAMPLE 89

### 6-(3-Fluorophenyl)-N-[trans-4-(2-hydroxyethoxy)cyclohexyl]nicotinamide

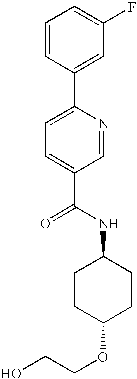

The compound from Preparation 9 (100 mg, 0.223 mmol) was dissolved in ethanol (2 mL) and to this solution was added ammonium formate (141 mg, 2.23 mmol) and 20% palladium hydroxide on carbon (10 mg). The reaction was refluxed for 2 hours, stirred at room temperature for 18 hours and then refluxed for a further 4 hours. The reaction mixture was cooled to room temperature, filtered through Arbocel and evaporated. The residue was purified on reverse phase HPLC.

### EXAMPLE 92A

### N-[trans-4-(Dimethylcarbamoyl)cyclohexyl]-6-(3-fluorophenyl)nicotinamide

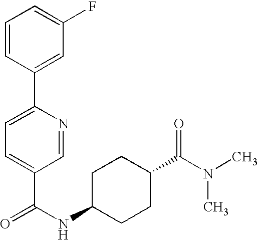

1,1-Carbonyldiimidazole (CDI, 15.69 g, 96.75 mmol) was added to a solution of 6-(3-fluorophenyl)nicotinic acid (Preparation 1) (21.01 g, 96.75 mmol) in acetonitrile (520 mL) and the reaction mixture was stirred at room temperature for 2 days. Additional CDI (3.81 g, 23.5 mmol) was added and stirring was continued for 1 hour followed by the addition of further CDI (1.9 g, 11.7 mmol) and an additional 30 minutes stirring. 4-Aminocyclohexylcarboxylic acid dimethylamide (WO-2008/068171, 20.0 g, 96.75 mmol) was added and the reaction mixture was heated under reflux for 18 hours. After cooling, the product was isolated by filtration from the reaction mixture, washed with acetonitrile and dried in vacuo at 40° C. to give 40.50 g (113%) of a white solid. Purification of 24 g was achieved by reverse phase column chromatography on Phenomenex Luna C18 (2) 3 μm particle size, eluting with a gradient from 9010 to 10:90 (by volume) 0.1% aqueous formic acid:methanol to give 14.2 g of the title compound.

### EXAMPLE 94

### 6-(3-Fluorophenyl)-N-[trans-4-(methylamino)cyclohexyl]nicotinamide

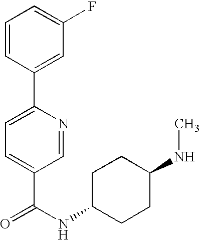

Aqueous sodium hydroxide solution (1 M, 5 mL) was added to a solution of the compound of Example 137 (83.0 mg, 0.20 mmol) in methanol (10 mL) at 55° C. Further methanol (5 mL) was added and the reaction mixture was heated at 55° C. for 5 min and then cooled to room temperature. The resulting mixture was partitioned between ethyl acetate (100 mL) and water (75 mL) and the aqueous layer was extracted further with ethyl acetate (2×50 mL). The combined organic extracts were washed with aqueous sodium hydroxide solution (0.2 M, 50 mL), dried (MgSO₄), filtered and evaporated to give 43 mg of the title compound as a white solid.

### EXAMPLE 97

### 6-(3-Fluorophenyl)-N-[trans-4-(1-hydroxy-1-methylethyl)cyclohexyl]nicotinamide

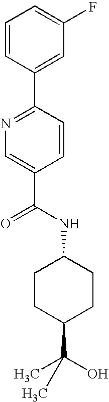

To a solution of 6-(3-Fluorophenyl)nicotinic acid (5.52 g, 25.4 mmol) in N,N-dimethylformamide (50 mL) at room temperature was added 1,1′-carbonyldiimidazole (4.67 g, 25.4 mmol) and the mixture was stirred for 3 hours before the addition of trans-2-(4-aminocyclohexyl)propan-2-ol (4.00 g, 28.0 mmol) and triethylamine (8.9 mL, 64.0 mmol). Stirring was continued for 18 hours. The reaction mixture was concentrated and the residue was partitioned between ethyl acetate (200 ml) and water (100 ml). The organic phase was separated, washed with brine (2×100 ml), dried over anhydrous magnesium sulphate and concentrated in vacuo to give a white solid. Recrystallisation from acetonitrile gave 6-(3-fluorophenyl)-N-[trans-4-(1-hydroxy-1-methylethyl)cyclohexyl]nicotinamide as a white solid (6.7 g).

### EXAMPLE 105

### 6-(3-Fluorophenyl)-N-[trans-4-(2-methyl-1H-imidazol-1-yl)cyclohexyl]nicotinamide

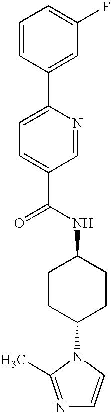

6-(3-Fluorophenyl)-N-[trans-4-(2-methyl-1H-imidazol-1-yl)cyclohexyl]nicotinamide, was prepared using the method described in Tetrahedron, 62, 2006, 8199-8206. Thus, a solution of N-(trans-4-amino-cyclohexyl)-6-(3-fluoro-phenyl)-nicotinamide, example 142B (140 mg, 0.477 mmol) in methanol (6 mL) was heated at reflux for 5 hours with 40% glyoxal in water (102 μL, 0.894 mmol), ammonium acetate (68.9 mg, 0.894 mmol) and acetaldehyde (50 μL, 0.894 mmol). The reaction was cooled to room temperature, concentrated in vacuo and then partitioned between dichloromethane (15 mL) and 2M sodium hydroxide (5 mL). The organic layer was separated and evaporated and the residue was purified on silica eluting with dichloromethane:methanol:ammonia in a ratio of 90:10:1. The fractions containing product were evaporated and then purified by reverse phase HPLC.

### EXAMPLE 106

### 6-(3-Fluorophenyl)-N-[trans-4-(2-isopropyl-1H-imidazol-1-yl)cyclohexyl]nicotinamide

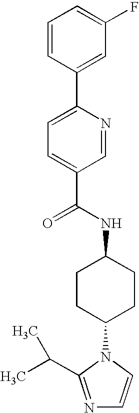

The title compound was prepared analogously to Example 105 by using isobutyraldehyde rather than acetaldehyde.

### EXAMPLE 111

### 6-(3-Fluorophenyl)-N-{trans-4-[(4-methylpiperazin-1-yl)methyl]cyclohexyl}nicotinamide

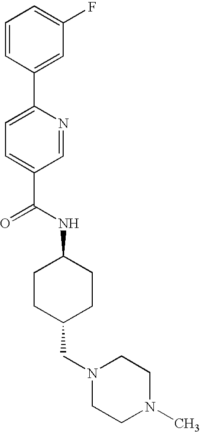

To a solution of 6-(3-fluoro-phenyl)-N-[trans-4-hydroxymethyl-cyclohexyl)nicotinamide, example 103, (730 mg, 2.22 mmol) and pyridine (7 mL) in tetrahydrofuran (7 mL) was added methanesulphonic anhydride (599 mg, 3.33 mmol). The reaction was stirred at room temperature for 3 hours and then an appropriate portion of the reaction was removed and reacted with N-methyl piperazine (556 mg, 5.56 mmol) at 80° C. overnight. The reaction was evaporated to dryness and purified by reverse phase HPLC.

Examples 110, 112, and 113 were analogously prepared.

Similarly, Examples 67, 79, 80 and 81 were prepared using the same method starting from 6-(3-fluoro-phenyl)-N-[cis-4-hydroxymethyl-cyclohexyl)-nicotinamide.

### EXAMPLE 114

### 5-Chloro-6-(3-fluorophenyl)-N-[trans-4-(hydroxymethyl)cyclohexyl]nicotinamide

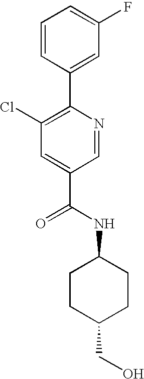

The 5-chloro-6-(3-fluorophenyl)nicotinic acid (50 mg, 0.20 mmol), (trans-4-aminocyclohexyl)methanol, preparation 2, (33 mg, 0.20 mmol), EDC (42.2 mg, 0.22 mmol), and HOBT (27.0 mg, 0.20 mmol) were added to dimethylacetamide (2 mL). N-Methylmorpholine (0.055 mL, 0.50 mmol) was added and the reaction was stirred overnight at room temperature. The reaction was diluted with ethyl acetate (20 mL) and water (20 mL). The organic layer was removed, dried with anhydrous MgSO₄ and evaporated to give the title compound.

### EXAMPLE 116

### cis-4-({[6-(3-Fluorophenyl)pyridin-3-yl]carbonyl}amino)-1-methylcyclohexanecarboxylic acid

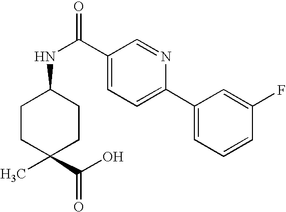

Methyl cis-4-({[6-(3-fluorophenyl)pyridin-3-yl]carbonyl}amino)-1-methylcyclohexanecarboxylate (86 mg, 0.23 mmol, Example 135) was dissolved in 1,4 dioxane (3 mL). 1M aqueous sodium hydroxide (3 mL) was added and the reaction mixture was stirred for 60 hours at room temperature. The dioxane was evaporated under reduced pressure and the pH of the remaining liquors was adjusted to pH 1. The resulting aqueous solution was extracted with dichloromethane (3 mL). The dichloromethane was filtered through a phase separation tube and evaporated under reduced pressure. This gave 31 mg of product

### EXAMPLE 117

### trans-4-({[6-(3-Fluorophenyl)pyridin-3-yl]carbonyl}amino)-1-methylcyclohexanecarboxylic acid

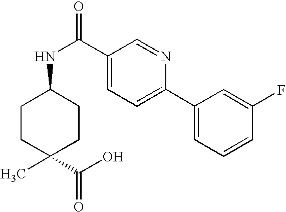

Methyl cis-4-({[6-(3-fluorophenyl)pyridin-3-yl]carbonyl}amino)-1-methylcyclohexanecarboxylate (59 mg, 0.16 mmol, Example 136) was hydrolysed with 1M aqueous sodium hydroxide solution (3 mL) using the same conditions and purification procedure as for Example 116, giving 11 mg of the title compound.

### EXAMPLE 119

### N-(4,4-Difluorocyclohexyl)-6-(3-fluorophenyl)nicotinamide

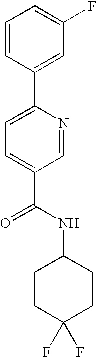

6-(3-Fluorophenyl)nicotinic acid (490 mg, 2.26 mmol) and HATU (944 mg, 2.48 mmol) were dissolved in anhydrous DMF (10 mL). Diisopropylethylamine (437 mg, 3.38 mmol) was added. The mixture was stirred at room temperature under nitrogen for 15 minutes and then 4,4-difluorocyclohexylamine (490 mg, 1.27 mmol) was added. After stirring at room temperature for 6 hours, the solvent was removed under reduced pressure. The residue was partitioned between ethyl acetate (50 mL) and saturated aqueous sodium bicarbonate (50 mL). The organic layer was washed with brine, dried over anhydrous MgSO₄, filtered and evaporated. The residue was partly re-dissolved into dichloromethane (5 mL). The remaining solid was filtered off and was found to be the title compound (140 mg).

### EXAMPLE 126

### N-(1-Ethyl-4,5,6,7-tetrahydro-1H-benzoimidazol-5-yl)-6-(3-fluoro-phenyl)-nicotinamide

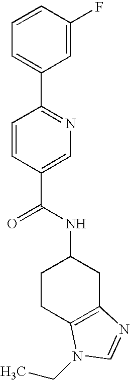

To a solution of 6-(3-fluorophenyl)nicotinic acid (37 mg, 0.169 mmol) in dimethylformamide (1 mL) was added 1-ethyl-4,5,6,7-tetrahydro-1H-benzoimidazol-5-ylamine (Preparation 30, 28 mg, 0.170 mmol), HBTU (28 mg, 0.170 mmol) and triethylamine (51 mg, 0.507 mmol) and the reaction mixture was stirred at room temperature for 48 hours under nitrogen. The reaction mixture was concentrated to dryness and partitioned between dicholoromethane (10 mL) and water (10 mL). The aqueous phase was re-extracted with dichloromethane (3×5 mL). The combined organic phases were dried over anhydrous sodium sulfate, filtered and concentrated in vacuo. The crude product was purified using chromatography on silica eluting with a mixture of dichloromethane:methanol:aqueous ammonia 100:0:0 to 90:10:1. The product fractions were combined and evaporated to give the desired product as a white solid (5 mg).

The following additional Examples have been prepared using the specific methods described below.

### EXAMPLE 131

### Methyl trans-4-({[6-(3-fluorophenyl)pyridin-3-yl]carbonyl}amino) cyclohexanecarboxylate

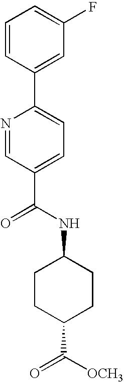

N,N-Carbonyldiimidazole (1.17 g, 7.22 mmol) was added to a stirred solution of 6-(3-fluorophenyl)nicotinic acid (1.44 g, 6.56 mmol) in dry dimethylformamide (20 mL) and the resulting solution was stirred at room temperature for 2 hours. N-Ethyl-diisopropylamine (1.06 g, 8.20 mmol) was then added followed by trans-1,4-aminocyclohexane carboxylic acid methyl ester hydrochloride salt (1.39 g, 7.18 mmol) in small batches and the resulting solution was stirred at room temperature for 17 hours. The mixture was concentrated in vacuo and the solid residue was partitioned between ethyl acetate (250 mL) and water (150 mL). Aqueous hydrochloric acid (2 M) was added to the aqueous layer to adjust the pH to 2. The organic layer was separated and washed with further aqueous hydrochloric acid (2M, 75 mL), sodium carbonate aqueous (2%, 75 mL) and water (75 mL). The organic layer was dried over anhydrous MgSO₄, filtered and evaporated to give 1.81 g of the title compound as a white solid.

¹H NMR (400 MHz, DMSO-d₆) δ ppm 1.32-1.48 (m, 4H) 1.89-1.99 (m, 4H) 2.25-2.32 (m, 1H) 3.59 (s, 3H) 3.72-3.81 (m, 1H) 7.28-7.32 (m, 1H) 7.52-7.56 (m, 1H) 7.93-7.96 (m, 1H) 7.98-8.00 (m, 1H) 8.10-8.12 (m, 1H) 8.26-8.27 (m, 1H) 8.46-8.48 (m, 1H) 9.06-9.07 (m, 1H).

### EXAMPLE 132

### Methyl cis-4-({[6-(3-fluorophenyl)pyridin-3-yl]carbonyl}amino)cyclohexanecarboxylate

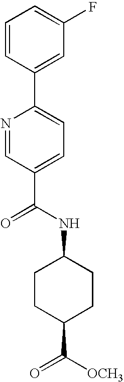

HBTU (1.54 g, 4.05 mmol) and triethylamine (1.49 g, 14.7 mmol) were added to a stirred solution of 6-(3-fluorophenyl)nicotinic acid (0.80 g, 3.68 mmol) in dry dimethylformamide (20 mL) and the resulting solution was stirred at room temperature for 30 minutes. cis-1,4-Aminocyclohexane carboxylic acid methyl ester hydrochloride salt (0.82 g, 4.24 mmol, prepared by the method of J. Med. Chem., 20(2), 1997, 279-290) was added in small batches and the solution was stirred at room temperature for 17 hours. The mixture was concentrated in vacuo and the oily residue was partitioned between ethyl acetate (70 mL) and water (70 mL). The organic layer was separated and washed with more water (2×70 mL). The organic layer was dried over anhydrous MgSO₄, filtered and evaporated to give a red/brown oil. The crude product was chromatographed on silica eluting with heptane:ethyl acetate, 4:1, 2:1, 1:1 volume mixtures. The fractions containing product were combined and evaporated to give 820 mg of the title product as a pale brown oil. On standing the product crystallised and was triturated with diethyl ether, filtered and dried in vacuo to give the title product as a white solid. ¹H NMR (400 MHz, DMSO-d₅) δ ppm 1.50-1.75 (m, 6H) 2.01-2.07 (m, 2H) 2.56-2.62 (m, 1H) 3.66 (s, 3H) 3.92-3.97 (m, 1H) 7.31-7.36 (m, 1H) 7.56-7.62 (m, 1H) 7.96-7.99 (m, 1H) 8.02-8.04 (m, 1H) 8.13-8.15 (d, 1H), 8.29-8.32 (m, 1H) 8.43-8.45 (m, 1H) 9.09 (s, 1H).

LRMS: m/z (APCI) 357 [MH]+.

### EXAMPLE 133

### Methyl cis-3-({[6-(3-fluorophenyl)pyridin-3-yl]carbonyl}amino)cyclohexanecarboxylate

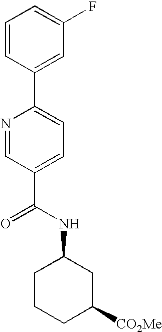

Using the method described for the preparation of Example 132, the title compound was prepared starting from cis-3-amino-cyclohexanecarboxylic acid methyl ester.

¹H NMR (400 MHz, methanol-d₄) δ ppm 1.28-1.39 (m, 2H) 1.44-1.56 (m, 2H) 1.94-2.02 (m, 3H) 2.26-2.31 (m, 1H) 2.53-2.61 (m, 1H) 3.72 (s, 3H) 3.97-4.04 (m, 1H) 7.23-7.28 (m, 1H) 7.54-7.58 (m, 1H) 7.85-7.89 (m, 1H) 7.91-7.93 (m, 1H) 8.02-8.04 (m, 1H) 8.29-8.33 (m, 1H) 9.09 (m, 1H).

### EXAMPLE 134

### Ethyl (1S,3R)-3-({[6-(3-fluorophenyl)pyridin-3-yl]carbonyl}amino)cyclopentanecarboxylate

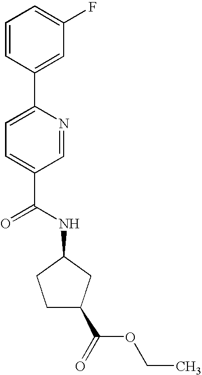

The title compound was prepared in the same way as Example 132 starting from ethyl (1S,3R)-3-aminocyclopentane carboxylate.

¹H NMR (400 MHz, MeOD-d₄) δ ppm 1.26-1.32 (m, 3H) 1.82-1.86 (m, 1H) 1.92-1.99 (m, 1H) 2.05-2.11 (m, 3H) 2.36-2.43 (m, 1H) 2.97-3.04 (m, 1H) 4.11-4.23 (m, 2H) 4.45-4.48 (m, 1H) 7.23-7.27 (m, 1H) 7.54-7.59 (m, 1H) 7.85-7.79 (m, 1H) 7.91-7.93 (m, 1H) 8.02-8.04 (m, 1H) 8.30-8.33 (m, 1H) 9.08-9.09 (m, 1H).

### EXAMPLE 135

### Methyl cis-4-({[6-(3-fluorophenyl)pyridin-3-yl]carbonyl}amino)-1-methyl cyclohexanecarboxylate

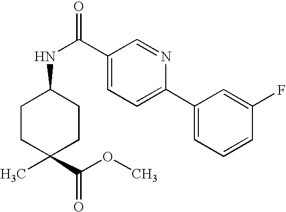

Methyl cis-4-amino-1-methylcyclohexanecarboxylate hydrochloride salt (41 mg, 0.2 mmol, Preparation 15) and 6-(3-fluorophenyl)nicotinic acid (43 mg, 0.2 mmol) were dissolved into DMF (0.75 mL). The reaction was stirred and triethylamine (100 mg, 0.99 mmol) and HBTU (93 mg, 0.25 mmol) were added. The reaction mixture was stirred for 16 hours at 50° C. The solvent was then removed under reduced pressure. The residue was purified by chromatography on silica eluting with a mixture of heptane:ethyl acetate 95:5 and 75:25 to give the title compound (68 mg) as an off white solid.

LRMS (ES): observed 371 (M+1), calculated 371.17 [M+1].

¹H NMR (400 MHz DMSO-d₆) δ ppm 1.16-1.42 (m, 7H) 1.95-2.10 (m, 2H) 2.20-2.32 (m, 2H) 3.72 (s, 3H) 3.92-4.06 (m, 1H) 5.90-6.00 (m, 1H) 7.09-7.19 (m, 1H) 7.41-7.49 (m, 1H) 7.72-7.84 (m, 3H) 8.10-8.19 (m, 1H) 8.94-9.01 (m, 1H).

### EXAMPLE 136

### Methyl trans-4-({[6-(3-fluorophenyl)pyridin-3-yl]carbonyl}amino)-1-methyl cyclohexanecarboxylate

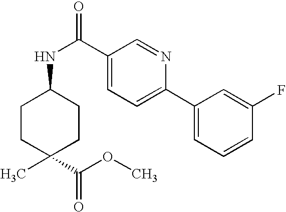

The title compound was prepared using a 1:1 mixture of methyl trans-4-amino-1-methylcyclohexanecarboxylate hydrochloride salt and methyl 4-aminocyclohexanecarboxylate (preparation 14) together with the reagents used in Example 135 on the same molar scale. This method gave the title compound (59 mg) as a 1:1 mixture with methyl 4-({[6-(3-fluorophenyl)pyridin-3-yl]carbonyl}amino)methylcyclohexanecarboxylate. LRMS (ES): observed 371 (M+1), calculated 371.17 [M+1].

### EXAMPLE 137

### 6-(3-Fluoro-phenyl)-N-trans-{4-[methyl-(2,2,-trifluoro-acetyl)-amino]cyclohexyl}-nicotinamide

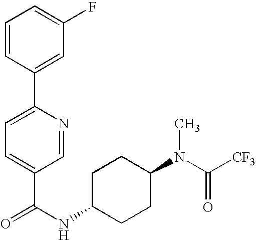

6-(3-Fluoro-phenyl)-N-trans-{4-[methyl-(2,2,-trifluoro-acetyl)-amino]cyclohexyl}-nicotinamide was prepared using the standard amide coupling method using HBTU starting from 6-(3-fluorophenyl)nicotinic acid and the compound from Preparation 5.

LRMS (ES): observed 424 [M+1], calculated 424.41 [M+1].

### EXAMPLE 138

### tert-Butyl (3S)-4-{[trans-4-({[(6-(3-fluorophenyl)pyridine-3-yl]carbonyl}amino)cyclohexyl]carbonyl}-3-methylpiperazine-1-carboxylate

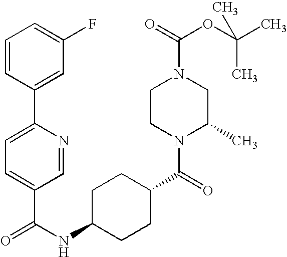

The title compound was prepared from 6-(3-fluorophenyl)nicotinic acid and tert-butyl (3S)-3-methylpiperazine-1-carboxylate according to the general amide coupling method with HBTU.

LRMS: observed 523 [M−1], calculated 523.63 [M−1].

### EXAMPLE 139

### tert-Butyl (3R)-4-{[trans-4-({[6-(3-fluorophenyl)pyridine-3-yl]carbonyl}amino)cyclohexyl]carbonyl}-3-methylpiperazine-1-carboxylate

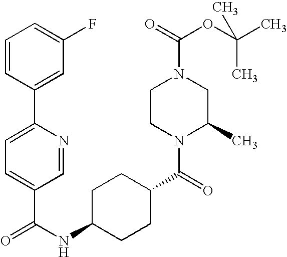

The title compound was prepared using the method of Example 138.

LRMS: observed 523 [M−1], calculated 523.63 [M−1].

### EXAMPLE 140

### tert-Butyl [4-({[6-(3-fluorophenyl)pyridin-3-yl]carbonyl}amino)cyclohexyl]carbamate

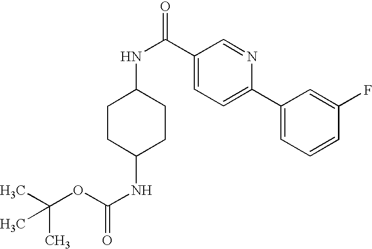

6-(3-Fluorophenyl)nicotinic acid (1.01 g, 4.67 mmol) and tert-butyl (4-aminocyclohexyl) carbamate (1.0 g, 4.67 mmol) was dissolved in DMF (5 mL). The solution was stirred and triethylamine (2.36 g, 23.3 mmol) and HBTU (2.21 mg, 5.83 mmol) were added. The reaction mixture was stirred for 16 hours at 50° C. and then left to stand at room temperature for 60 hours.

The solvent was then removed under reduced pressure and the residue was partitioned between dichloromethane (30 mL) and semi-saturated aqueous sodium hydrogen carbonate solution (20 mL). The organic layer was separated by filtration through a phase separation tube and then evaporated under reduced pressure. The residue was purified by chromatography on silica gel eluting with a mixture of heptane:ethyl acetate 100:0 and 20:80 to give the title compound (680 mg) as a brown solid. LRMS (ES): observed 412 (M−1), calculated 412.21 [M−1].

¹H NMR (400 MHz, DMSO-d₆) δ ppm 1.31-1.41 (s, 9H), 1.49-1.61 (m, 4H), 1.66-1.81 (m, 4H), 3.36-3.44 (m, 1H), 3.79-3.87 (m, 1H), 6.57-6.67 (m, 1H), 7.26-7.35 (m, 1H), 7.51-7.59 (m, 1H), 7.91-8.02 (m, 2H), 8.04-8.14 (m, 1H), 8.24-8.34 (m, 2H), 9.02-9.08 (m, 1H).

### EXAMPLE 141

### 6-(3-Fluorophenyl)-N-(4-oxocyclohexyl)nicotinamide

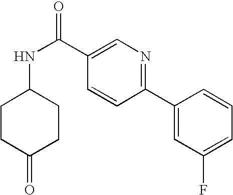

N-1,4-Dioxaspiro[4.5]dec-8-yl-6-(3-fluorophenyl)nicotinamide, preparation 21, (1.45 g, 4.07 mmol) was added to a mixture of water (5 mL) and a 12 M solution of hydrochloric acid in water (5 mL). The suspension was heated under reflux for 1 hour. The pH of the reaction mixture was adjusted to 9 by addition of a 1M aqueous solution of sodium hydroxide. The resulting precipitate was filtered off and dried to give the title compound as a colourless solid (1.09 g).

LRMS (ES): observed 311 [M−1], calculated 311.13 [M−1].

¹H NMR (400 MHz CDCl₆) δ ppm 1.74-1.90 (m, 2H), 2.34-2.64 (m, 6H), 4.40-4.55 (m, 1H), 6.14-6.23 (m, 1H), 7.09-7.20 (m, 1H), 7.39-7.52 (m, 1H), 7.73-7.84 (m, 3H), 8.13-8.23 (m, 1H), 8.98-9.06 (m, 1H).

### EXAMPLE 142A

### Tert-butyl[cis-4-({[6-(3-fluorophenyl)pyridine-3-yl]carbonyl}amino)cyclohexyl]carbamate

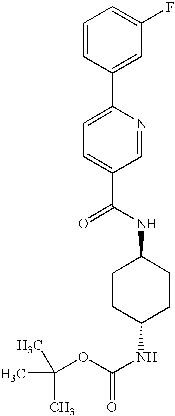

6-(3-Fluorophenyl)nicotinic acid (1015 mg. 4.67 mmol) was dissolved in dimethylformamide (5 mL), 1,1-carbonyl diimidazole (909 mg, 5.61 mmol) was added and the reaction mixture was stirred at room temperature for 1.5 hours. Tert-butyl(trans-3-aminocyclohexyl)carbamate (1000 mg, 4.67 mmol) was added and the reaction mixture was stirred at room temperature overnight. A thick slurry of insoluble material had formed. Further 1,1-carbonyldiimdazole (0.5 g, 3.08 mmol) was added and the reaction was heated to 60° C. for 18 hours with stirring. The dimethylformamide was evaporated, water (20 mL) was added to the residue and the remaining solid was isolated by filtration and drying to give the title compound (900 mg) as a white solid.

LRMS: [M+1] 414 (obs); [M+1] 414.5 (calc). ¹H NMR (400 MHz, DMSO-d₆): δ ppm 1.24-1.33 (m, 1H) 1.37-1.46 (m, 4H) 1.39-1.40 (m, 10H) 1.62-1.78 (m, 6H) 2.40-2.60 (m, 1H) 3.71-3.78 (m, 1H) 6.70-6.75 (m, 1H) 7.31-7.36 (m, 1H) 7.56-7.70 (m, 1H) 7.96-8.00 (m, 1H) 8.01-8.02 (m, 1H) 8.14-8.16 (m, 1H) 8.29-8.32 (m, 1H) 8.46-8.48 (m, 1H) 9.09-9.10 (m, 1H).

### EXAMPLE 142B

### N-(trans-4-Aminocyclohexyl)-6-(3-fluorophenyl)nicotinamide hydrochloride

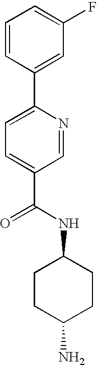

A suspension of tert-butyl[cis-4-({[6-(3-fluorophenyl)pyridine-3-yl]carbonyl}amino)cyclohexyl]carbamate (Example 142A) (900 mg, 2.18 mmol) in 4 M hydrogen chloride in 1,4-dioxane (10 mL) and water (1 mL) was heated at 70° C. for 1.5 hours. The reaction was evaporated and the residue was dried in vacuo, giving the title compound (900 mg) as a white solid. ¹H NMR (400 MHz, DMSO-d₆): δ ppm 1.44-1.51 (m, 4H) 1.93-2.04 (m, 4H) 2.95-3.02 (m, 1H) 3.70-3.77 (m, 1H) 7.32-7.37 (m, 1H) 7.57-7.62 (m, 1H) 7.97-8.04 (m, 2H) 8.15-8.18 (m, 3H) 8.34-8.35 (m, 1H) 8.61-8.63 (m, 1H) 9.11-9.13 (m, 1H).

### EXAMPLE 142

### 6-(3-Fluorophenyl)-N-[trans-4-(glycoloylamino)cyclohexyl]nicotinamide

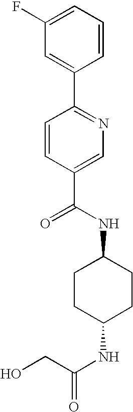

Glycolic acid (15 mg, 0.197 mmol) and 1,1′-carbonyldiimidazole (38.3 mg, 0.236 mmol) were stirred together in dimethylformamide (1 mL) for 1.5 hours. N-(trans-4-Aminocyclohexyl)-6-(3-fluorophenyl)nicotinamide (example 142B), (76.1 mg, 0.197 mmol) was added followed by N,N-diisopropylamine (0.103 mL, 0.591 mmol) and further dimethylformamide (1 mL). The reaction mixture was sonicated and then heated to 55° C. for 18 hours. In a separate vial, glycolic acid (15 mg, 0.197 mmol) and 1,1′-carbonyldiimidazole (38.3 mg, 0.236 mmol) were stirred together in dimethylformamide (0.5 mL) for 1.5 h and then added to the reaction mixture which was subsequently heated at 65° C. for 18 hours. The reaction mixture was partitioned between ethyl acetate (5 mL) and water (3 mL) and the organic layer was separated nd evaporated to give a cream coloured solid which was purified on reverse phase HPLC Method (B) to give 15.8 mg of the title compound. LCMS Method (B) RT 2.69 min 100% area, ES m/z [M+] 371.16.

### EXAMPLE 143

### 6-(3-Fluorophenyl)-N-{trans-4-[(2-hydroxy-2-methylpropanoyl)amino]cyclohexyl}nicotinamide

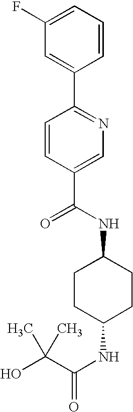

2-Hydroxy-2-methylpropanoic acid (41 mg, 0.394 mmol) and 1,1′-carbonyldiimidazole (70.2 mg, 0.433 mmol) were stirred together in dimethylsulphoxide (1 mL) for 1.5 hours. N-(trans-4-Aminocyclohexyl)-6-(3-fluorophenyl)nicotinamide (Example 142B) (76.1 mg, 0.197 mmol) was added followed by N,N-diisopropylamine (0.103 mL, 0.591 mmol) and the reaction mixture was heated to 75° C. for 18 hours. The reaction mixture was purified using reverse phase HPLC

Method (B) to give 18.0 mg of the title compound. LCMS Method (A) RT 2.76 min 100% area, ES m/z [M+] 1371.16.

### EXAMPLE 144

### 6-(3-Fluorophenyl)-N-(trans-4-{[(2S)-2-hydroxypropanoyl]amino}cyclohexyl)nicotinamide

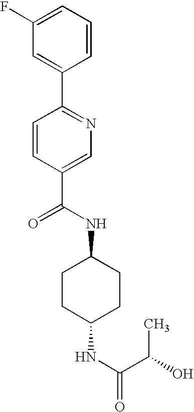

(2S)-2-Hydroxypropanoic acid (7.5 mg, 0.083 mmol) and 1,1′-carbonyldiimidazole (16.2 mg, 0.100 mmol) were stirred together in dimethylsulphoxide (1 mL) for 1.5 hours. N-(trans-4-Aminocyclohexyl)-6-(3-fluorophenyl)nicotinamide (Example 142B, 32.1 mg, 0.083 mmol) was added followed by N,N-diisopropylamine (0.043 mL, 0.249 mmol) and the reaction mixture was stirred at room temperature for 18 hours. In a separate vial, (2S)-2-hydroxypropanoic acid (15 mg, 0.197 mmol) and 1,1′-carbonyldiimidazole (32.0 mg, 0.20 mmol) were stirred together in dimethylsulphoxide (0.2 mL) for 1.5 hours and then added to the reaction mixture which was subsequently heated at 60° C. for 24 hours. The reaction mixture was purified on reverse phase HPLC Method (B) to give 8.5 mg of the title compound. LCMS Method (A) RT 2.61 min 100% area, ES m/z [M+] 385.18.

### EXAMPLE 145

### N-{trans-4-[(N,N-Dimethylglycyl)amino]cyclohexyl}-6-(3-fluorophenyl)nicotinamide

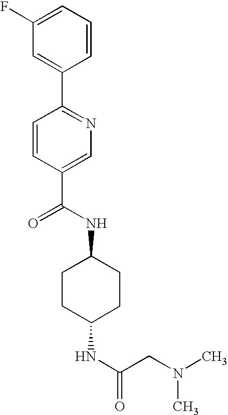

N,N-dimethylglycine (14.5 mg, 0.083 mmol) and 1,1′-carbonyldiimidazole (16.2 mg, 0.100 mmol) were stirred together in dimethylsulphoxide (1 mL) for 1.5 hours. N-(trans-4-Aminocyclohexyl)-6-(3-fluorophenyl)nicotinamide (Example 142B, 32.1 mg, 0.083 mmol) was added followed by N,N-diisopropylamine (0.043 mL, 0.249 mmol) and the reaction mixture was stirred at room temperature for 18 hours. In a separate vial N,N-dimethyglycine (17.2 mg, 0.098 mmol) and 1,1′-carbonyldiimidazole (32.0 mg, 0.20 mmol) were stirred together in dimethylsulphoxide (0.2 mL) for 1.5 hours and then added to the reaction mixture which was subsequently heated at 60° C. for 18 hours. The reaction mixture was purified on reverse phase HPLC Method (A) to give 11.0 mg of the title compound. LCMS Method (B) RT 2.77 min, 100% area, ES m/z [M+] 398.21.

### EXAMPLE 146A

### tert-Butyl(2-{[trans-4-({[6-(3-fluorophenyl)pyridine-3-yl]carbonyl}amino)cyclohexyl]amino}-2-oxoethyl)carbamate

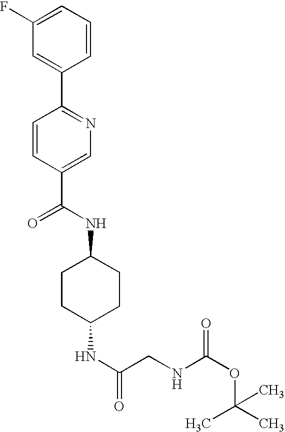

N-(tert-Butoxycarbonyl)glycine (14.5 mg, 0.083 mmol) was dissolved in dimethylsulphoxide (1 mL), 1,1′ carbonyl diimidazole (16.2 mg, 0.10 mmol)) was added and the reaction mixture was stirred at room temperature for 1.5 hours. N-(trans-4-Aminocyclohexyl)-6-(3-fluorophenyl)nicotinamide (Preparation 142B, 32.1 mg, 0.083 mmol) was added followed by N,N-diisopropylethylamine (0.043 mL, 0.249 mmol) and the reaction was stirred at room temperature for 18 hours. The reaction mixture was partitioned between water and ethyl acetate and the organic layer was separated and evaporated in vacuo to give the p[roduct as a gum. LRMS [M+1] 471 (obs) [M+1] 470.54 (calc).

### EXAMPLE 146

### 6-(3-Fluorophenyl)-N-[trans-4-(glycylamino)cyclohexyl]nicotinamide

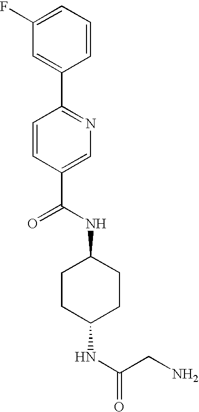

A 4M solution of hydrogen chloride in 1,4-dioxane (1 mL) was added to a solution of tert-butyl(2-{[trans-4-({[6-(3-fluorophenyl)pyridine-3-yl]carbonyl}amino)cyclohexyl]amino}-2-oxoethyl)carbamate (as prepared in Example 146A) in dichloromethane (1 mL) and stirred at room temperature for 2 hours. The solvent was evaporated and the residue was passed through an Isolute™ SCX-2 column eluting with methanol followed by methanolic ammonia. The product fractions were combined, evaporated and purified by reverse phase HPLC Method (B) to give 8.4 mg of the title compound. LCMS Method (B) RT 2.60 min 100% area, ES m/z [M+] 370.18.

### EXAMPLE 147

### N-[trans-4-(1-Acetamidoethyl]cyclohexyl]-6-(3-fluorophenyl)nicotinamide

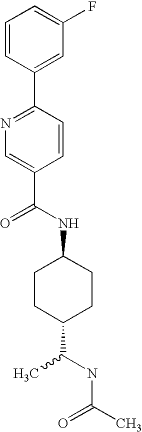

6-(3-Fluorophenyl)nicotinic acid (0.170 g, 0.781 mmol) and N-[1-(trans-4-aminocyclohexyl)ethyl]acetamide (Preparation 76) (0.072 g, 0.391 mmol) were dissolved in dimethylformamide (2 mL). O-Benzotriazol-1-yl-tetramethyluronium hexafluorophosphate (0.222 g, 0.586 mmol) and N,N-diisopropylethylamine (0.136 mL, 0.781 mmol) were then added and the reaction mixture was stirred at room temperature overnight. Water (20 mL) was added and the resulting precipitate was filtered off and purified on silica, eluting with methanol:dichloromethane 5:95, to give the title compound as a white solid (63 mg). LRMS: [M+1] 384 (obs); [M+1] 383.46 (calc). ¹H NMR (400 MHz, DMSO-d₆): δ ppm 0.98-1.00 (m, 3H) 1.03-1.10 (m, 2H) 1.18-1.37 (m, 3H) 1.68-80 (m, 5H) 1.88-1.95 (m, 2H) 3.58-3.68 (m, 2H) 3.69-3.80 (m, 1H) 7.31-7.36 (m, 1H) 7.56-7.58 (m, 1H) 7.63-7.65 (m, 1H) 7.96-8.03 (m, 2H) 8.13-8.15 (m, 1H) 8.28-8.30 (m, 1H) 8.46-8.48 (m, 1H) 9.07 (s, 1H)

### EXAMPLE 148 AND EXAMPLE 149

### N-{trans-4-[(1R)-Acetamidoethyl]cyclohexyl}-6-(3-fluorophenyl)nicotinamide and N-{trans-4[(1S)-acetamidoethyl]cyclohexyl}-6-(3-fluorophenyl)nicotinamide

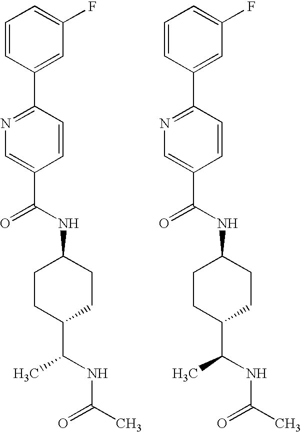

The product from Example 147 was separated into the two enantiomers by chiral preparative HPLC on a Chiralpak IA column with a methanol:ethanol 1:1 mobile phase, and a flow rate of 18 mL per min. Enantiomer (1) is >99.5% pure of the peak eluting at 3.6 min and Enantiomer (2) is 97% pure of the peak eluting at 3.8 min. The fractions were evaporated to give 4.5 mg of each enantiomer as a white solid.

### EXAMPLE 150

### 6-(3-Fluorophenyl)-N-[trans 4(methanesulfonylaminomethyl)cyclohexyl]nicotinamide

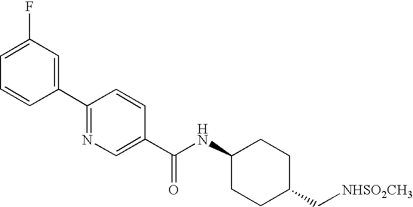

To a solution of 6-(3-fluorophenyl)nicotinic acid (108 mg, 0.49 mmol) in THF (5 mL) was added N-(4-aminocyclohexylmethyl)methanesulfonamide (100 mg, 0.41 mmol, Preparation 89) followed by DIPEA (0.14 mL; 0.825 mmol), DMAP (20.2 mg; 0.165 mmol) and EDC (103 mg; 0.54 mmol). The reaction mixture was stirred at room temperature for 4 hours, concentrated to dryness and partitioned between aqueous 1M NaOH (20 mL) and DCM (40 mL). The organic layer was washed with 1M NaOH aqueous solution and brine and dried over MgSO₄. Filtration and concentration of the organic layer gave a white powder which was purified by HPLC Method (D) to give the title compound. ¹H NMR (400 MHz, CDCl₃): δ ppm 1.22 (m, 5H), 1.95 (m, 2H), 2.21 (m. 2H), 3.03 (m, 3H), 3.97 (m, 1H), 4.31 (m, 1H), 6.00 (m, 1H), 7.15 (m, 1H), 7.45 (m, 1H), 7.80 (m, 3H), 8.15 (dd, 1H), 8.99 (d, 1H), 2 exchangeable protons not seen. LRMS: m/z (AP+) [M+1] 406.

### EXAMPLE 151

### 6-(3-Fluorophenyl)-N-(trans 4-hydroxy-4-trifluoromethylcyclohexyl)nicotinamide and 6-(3-Fluorophenyl)-N-(cis 4-hydroxy-4-trifluoromethylcyclohexyl)nicotinamide

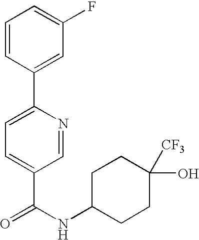

The title compound was prepared using general method (ii) starting from 6-(3-fluorophenyl)nicotinic acid and 4-amino-1-trifluoromethylcyclohexanol (Preparation 85) and the product was purified by HPLC Method A, RT 2.51 min, m/z (ES+) [M+1] 383.

### EXAMPLE 152

### 6-(3-Fluorophenyl)-N-[4-(trans 2-hydroxy-2-methylpropyl)cyclohexyl]nicotinamide

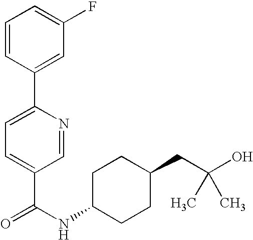

A suspension of Example 261A (706 mg, 1.91 mmol) in THF (6.5 mL) was heated to 40° C. and a few drops of 3.0M methyl magnesium bromide (MeMgBr) in ether was added. The mixture became clear yellow immediately. A further few drops of MeMgBr was added. Reflux was detected. The heat was removed and the remaining MeMgBr (in total 3.18 mL. 9.53 mmol) was added dropwise. The reaction turned from yellow to a strong orange colour. After 30 min the reaction was carefully quenched with aqueous NH₄Cl (10 mL) and the reaction mixture was stirred for 15 minutes at room temperature. The reaction mixture was extracted with EtOAc (3×100 mL). The organic phases were combined, dried over MgSO₄ and concentrated in vacuo to give an orange solid. This solid was suspended in a 2:1 by volume mixture of diethylether and acetonitrile and filtered to give 545 mg of a solid with a pale orange colour (78% yield). A portion of the crude product (100 mg) was purified by silica column chromatography eluting with 4:6 by volume heptane/EtOAc to give the title compound as a white solid 45 mg. LRMS: m/z (AP+) [M+1] 371. ¹H NMR (400 MHz, CD₃OD) δ ppm 1.19 (m, 2H), 1.20 (s, 6H), 1.39-1.47 (m, 5H) 1.96 (m, 4H), 3.84 (m, 1H), 7.20 (m, 1H), 7.51 (m, 1H), 7.83 (m, 2H), 7.98 (m, 1H), 8.24 (m, 1H), 9.01 (d, 1H), 2 exchangeable protons not seen.

### EXAMPLE 153

### 6-(3-Fluorophenyl)-N-(4-hydroxy-piperidin-1-ylmethylcyclohexyl)nicotinamide

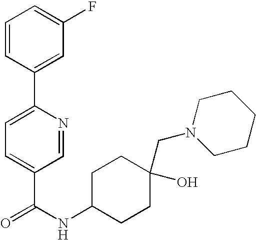

The compound of Preparation 87 (50 mg, 0.15 mmol) and piperidine (431 mg, 5.06 mmol) were combined in toluene (3 mL) and stirred at room temperature for 18 hours. The reaction mixture was concentrated in vacuo and partitioned between DCM and saturated aqueous NaHCO₃ and the organic phase was washed twice with water and concentrated in vacuo. Purification by silica column chromatography eluting with a gradient of DCM (100%) to 95:5:0.5 DCM:MeOH:NH₄OH by volume afforded a yellow solid which on trituration with minimal ethyl acetate afforded the title compound as an off-white solid (20 mg). LCMS Method (G) RT 0.91 min, m/z (ES+) [M+1] 412.

### EXAMPLE 154

### N-[4-(3-Amino-1-hydroxypropyl)cyclohexyl]6-(3-fluorophenyl)nicotinamide hydrochloride

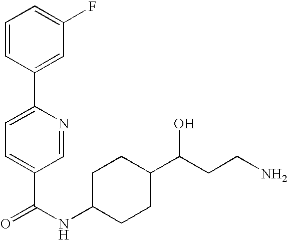

The title compound was prepared in a two step process.

Step (a): Starting with 6-(3-fluorophenyl)nicotinic acid and the product of Preparation 88 using general method (ii).

Step (b): The product of step (a) was treated with 4N HCl in dioxan and heated at 70° C. for 2 hours. Concentration of the mixture in vacuo, re-dissolving in MeOH and evaporating three times afforded the title compound as the hydrochloride salt (160 mg). A portion of this material (30 mg) was further purified by HPLC Method (A) 0.91 min m/z (ES+)[M+1] 372.

### EXAMPLE 155A

### tert-Butyl[cis-4-({[6-(3-fluorophenyl)pyridine-3-yl]carbonyl}amino)cyclohexyl]carbamate

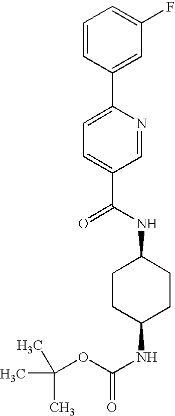

To a solution of 6-(3-fluorophenyl)nicotinic acid (200 mg. 0.921 mmol) in dimethylformamide (5 mL) was added HBTU (367 mg, 0.967 mmol), tert-butyl(cis-4-aminocyclohexyl)carbamate (197 mg, 0.921 mmol) and triethylamine (0.135 mL, 0.967 mmol) and the reaction mixture was stirred at room temperature overnight. The dimethylformamide was evaporated and the reaction mixture was partitioned between dichloromethane (15 mL) and water (15 mL). The organic layer was separated, washed with brine, dried over magnesium sulphate and evaporated in vacuo to give the title compound as a white solid (127 mg). ¹H NMR (400 MHz, CDCl₃): δ ppm 1.44 (s, 9H) 1.63-1.70 (m, 4H) 1.81-1.90 (m, 4H) 3.62-3.72 (m, 1H) 4.10-4.14 (m, 1H) 6.05-6.07 (m, 1H) 7.13-7.17 (m, 1H) 7.43-7.46 (m, 1H) 7.76-7.81 (m, 3H) 8.15-8.18 (m, 1H) 9.00-9.01 (m, 1H).

### EXAMPLE 155

### N-(cis-4-Aminocyclohexyl)-6-(3-fluorophenyl)nicotinamide

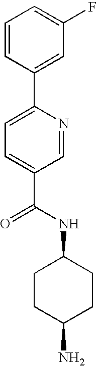

To a solution of tert-butyl[cis-4-({[6-fluorophenyl)pyridine-3-yl]carbonyl}amino)cyclohexyl]carbamate (Example 155A, 127 mg, 0.307 mmol) in 1,4-dioxane (5 mL) was added a 4M solution of hydrogen chloride in 1,4-dioxane (1.54 ml). The reaction mixture was stirred at room temperature overnight. The reaction mixture was washed with dilute sodium hydroxide solution (10 mL) and brine (10 mL) and the organic extracts were evaporated to an almost colourless gum (73 mg). The gum was purified using reverse phase HPLC Method (B) to give 27.7 mg of the title compound. LCMS Method (A), RT 2.93 min 100% area ES m/z [M+] 313.16.

The following seven tabulated compounds were prepared by the previously described general methods (i), (ii) and (iii)

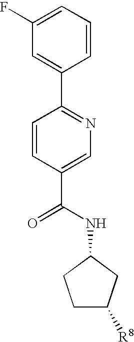

| | | | Purification and |
|---|---|---|---|
| Ex | R⁸ | Name | Characterisation |
| 156 | 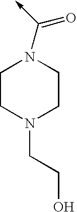 | 6-(3-fluorophenyl)-N-[(1S,3R)-3-{[4-(2- hydroxyethyl)piperazin-1- yl]carbonyl}cyclopentyl]nicotinamide | Purified by HPLC Method (A) LCMS Method (B) RT 2.74 minutes (100%) area, ES m/z [M + H] 441.2 |
| 157 | 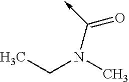 | N-{(1S,3R)-3- [ethyl(methyl)carbamoyl]cyclopentyl}- 6-(3-fluorophenyl)nicotinamide | Purified by HPLC Method (A) LCMS Method (B) RT 3.26 minutes (100%) area, ES m/z [M + H] 370.2 |
| 158 | —OH | 6-(3-fluorophenyl)-N-[cis-3- | Purified by HPLC Method |
| | | hydroxycyclopentyl]nicotinamide | (A) |
| | | | LCMS Method (B) RT 2.67 |
| | | | minutes (100%) area, ES |
| | | | m/z [M + H] 301.2 |
| 159 | 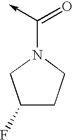 | 6-(3-fluorophenyl)-N-[(1S,3R)-3- {[(3S)-3-fluoropyrrolidin-1- yl]carbonyl}cyclopentyl]nicotinamide | LCMS (ES+) 400 [M + 1] ¹H NMR (400 MHz DMSO-d₆) δ ppm 1.61-2.00 (m, 6H), 2.03-2.25 (m, 2H), 2.91-3.21 (m, 1H), 3.39-3.79 (m, 4H), 4.23-4.40 (m, 1H), 5.20-5.43 (m, 1H), 7.27-7.38 (m, 1H), 7.54-7.59 (m, 1H), 7.93-8.07 (m, 2H), 8.10-8.15 (m, 1H), 8.22-8.28 (m, 1H), 8.76-8.82 (m, 1H), 9.06 (s, 1H). |
| 160 | 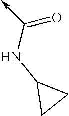 | N-[(1S,3R)-3- (cyclopropylcarbamoyl)cyclopentyl]-6- (3-fluorophenyl)nicotinamide | LCMS (ES+) 368 [M + 1] ¹H NMR (400 MHz DMSO-d₆) δ ppm 0.35-0.40 (m, 2H), 0.57-0.62 (m, 2H), 1.61-1.92 (m, 5H), 1.98-2.06 (m, 1H), 2.59-2.66 (m, 2H), 4.23-4.37 (m, 1H), 7.25-7.37 (m, 1H), 7.51-7.59 (m, 1H), 7.90-8.06 (m, 3H), 8.08-8.13 (m, 1H), 8.20-8.29 (m, 1H), 8.81-8.87 (m, 1H), 9.06 (s, 1H). |
| 161 | 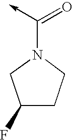 | 6-(3-fluorophenyl)-N-[(1S,3R)-3- {[(3R)-3-fluoropyrrolidin-1- yl]carbonyl}cyclopentyl]nicotinamide | Purified by HPLC Method (B) LCMS Method (B) RT 3.01 minutes (100%) area, ES m/z [M + H] 400.2 |
| 162 | 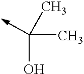 | 6-(3-fluorophenyl)-N-[(1S,3R)-3-(1- hydroxy-1- methylethyl)cyclopentyl]nicotinamide | Purified by HPLC Method (B) LCMS Method (A) RT 3.22 minutes (100%) area, ES m/z [M + H] 343.2 |

### EXAMPLE 163

### N-[(1S,3R)-3-(Dimethylcarbamoyl)cyclopentyl]-6-(3-fluorophenyl)nicotinamide

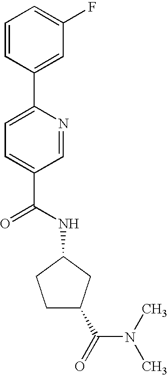

The title compound was prepared using analogous conditions to those described in Example 6 from (1R,3S)-3-({[6-(3-fluorophenyl)pyridin-3-yl]carbonyl}amino)cyclopentanecarboxylic acid (Example 11b) and dimethylamine. The crude product was purified by HPLC method (B). LCMS method (A) RT 3.22 min (100%) ES+m/z 356.17 [M+1].

### EXAMPLE 164

### 6-(3-Fluorophenyl)-N-{(1S,3R)-3-[(2-hydroxy-1-methylethyl)carbamoyl]cyclopentyl}nicotinamide

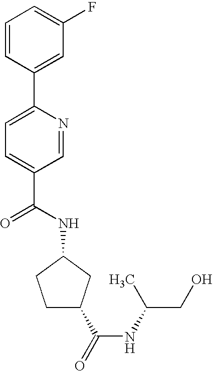

The title compound was prepared using analogous conditions to those described in example 6 from (1R,3S)-3-({[6-(3-fluorophenyl)pyridin-3-yl]carbonyl}amino)cyclopentanecarboxylic acid (Example 11b) and (2R)-2-amino-1-propanol. The crude product was purified by HPLC method (B). LCMS method (A) RT 2.77 min (100%) ES+m/z 386.18 [M+1].

### EXAMPLE 165

### N-[(1R,3S)-3-Dimethylcarbamoyl-cyclopentyl]-6-(3-fluorophenyl)nicotinamide

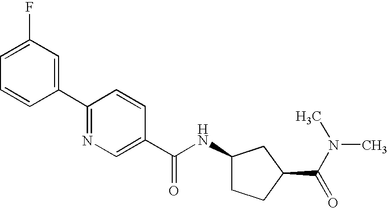

The acid from Example 11a (40 mg, 0.122 mmol) was dissolved in dimethylsulphoxide (0.5 ml) and 1,1′-carbonyldiimidazole (24 mg, 0.146 mmol) was added. The reaction mixture was stirred at room temperature for 1.5 hours, dimethylamine hydrochloride (0.183 mmol) and DIPEA (32 μL, 0.183 mmol) were added and stirring was continued at room temperature for 18 hours. The product was purified by HPLC Method (B) to give 30.6 mg of the title compound (RT 3.12 min m/z [M+1] 355).

### EXAMPLE 166

### 6-(3-Fluorophenyl)-N-[(1R,3S)-3-(1-hydroxy-1-methylethyl)cyclopentyl]nicotinamide

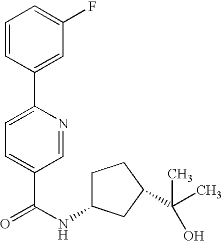

The title compound was prepared using general method (ii) starting from 6-(3-fluorophenyl)nicotinic acid (Preparation 1) and 2-((1R,3S)-3-aminocyclopentyl)propan-2-ol (Preparation 86) and the product was purified by HPLC Method(A) (RT 2.97 min, m/z (ES+) [M+1] 343).

### EXAMPLE 167

### N-[(1S,3R)-3-{[2-(Dimethylamino)ethyl]carbamoyl}cyclopentyl]-6-(3-fluorophenyl)nicotinamide

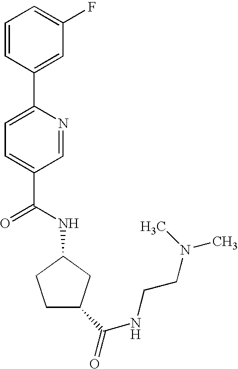

The title compound was prepared using analogous conditions to those described in Example 6 from (1R,3S)-3-({[6-(3-fluorophenyl)pyridin-3-yl]carbonyl}amino)cyclopentanecarboxylic acid (Example 11b) and N,N-dimethyl-1,2-ethanediamine. The crude product was purified by HPLC method (B) (LCMS method (A) RT 2.21 min (100%) ES+m/z 399.21 [M+1]).

### EXAMPLE 168

### 6-(3-Fluorophenyl)-N-[(1S,3R)-3-{(4-(2-hydroxyethyl)piperidin-1-yl]carbonyl}cyclopentyl]nicotinamide

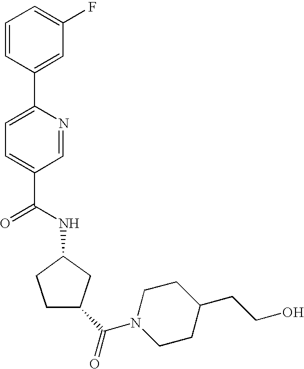

The title compound was prepared using analogous conditions to those described in Example 6 from (1R,3S)-3-({[6-(3-fluorophenyl)pyridin-3-yl]carbonyl}amino)cyclopentanecarboxylic acid (Example 11b) and 4-piperidine ethanol. The crude product was purified by HPLC method (B) (LCMS method (A) RT 3.06 min (100%) ES+m/z 440.22 [M+1]).

### EXAMPLE 169

### 6-(3-Fluorophenyl)-N-{(1S,3R)-3-[(2-methoxyethyl)carbamoyl]cyclopentyl}nicotinamide

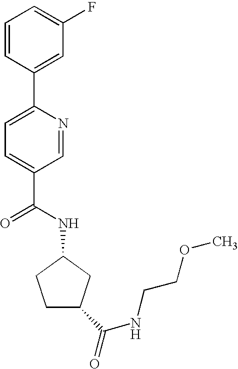

The title compound was prepared using analogous conditions to those described in Example 6 from (1R,3S)-3-({[6-(3-fluorophenyl)pyridin-3-yl]carbonyl}amino)cyclopentanecarboxylic acid (Example 11b) and 1-amino-2-methoxy ethane. The crude product was purified by HPLC method (B) (LCMS method (A) RT 3.07 min (100%) ES+m/z 386.18 [M+1]).

### EXAMPLE 170

### 6-(3-Fluorophenyl)-N-[(1S,3R)-3-(morpholin-4-ylcarbonyl)cyclopentyl]nicotinamide

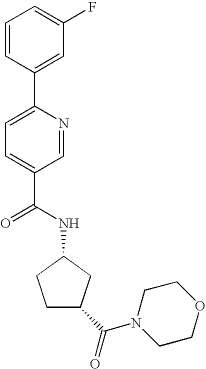

The title compound was prepared using analogous conditions to those described in Example 6 from (1R,3S)-3-({[6-(3-fluorophenyl)pyridin-3-yl]carbonyl}amino)cyclopentanecarboxylic acid (Example 11b) and morpholine. The crude product was purified by HPLC method (B) (LCMS method (A) RT 3.06 min (100%) ES+m/z 398.16 [M+1]).

### EXAMPLE 171

### 6-(3-Fluorophenyl)-N-{(1S,3R)-3-[(2-hydroxy-2-methylpropyl)carbamoyl]cyclopentyl}nicotinamide

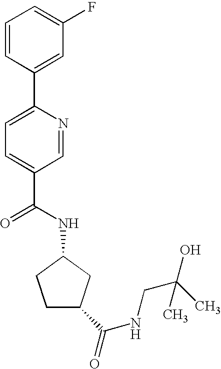

The title compound was prepared using analogous conditions to those described in Example 6 from (1R,3S)-3-({[6-(3-fluorophenyl)pyridin-3-yl]carbonyl}amino)cyclopentanecarboxylic acid (Example 11b) and 1-amino-2-methyl-2-propanol. The crude product was 12.2 purified by HPLC method (B) (LCMS method (A) RT 2.90 min (100%) ES+m/z 400.19 [M+1]).

### EXAMPLE 172

### 6-(3-Fluorophenyl)-N-{(1S,3R)-3-[(3-hydroxy-1,1-dimethylpropyl)carbamoyl]cyclopentyl}nicotinamide

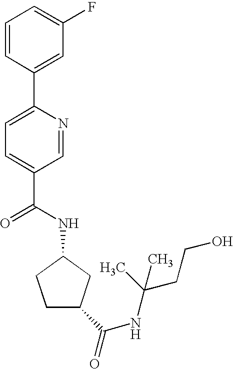

The title compound was prepared using analogous conditions to those described in Example 6 from (1R,3S)-3-({[6-(3-fluorophenyl)pyridin-3-yl]carbonyl}amino)cyclopentanecarboxylic acid (Example 11b) and 3-amino-3-methyl-1-butanol. The crude product was purified by HPLC method (B) (LCMS method (A) RT 2.97 min (100%) ES+m/z 414.21 [M+1]).

### EXAMPLE 173

### 6-(3-Fluorophenyl)-N-{(1S,3R)-3-[(4-methyl-3-oxopiperazin-1-yl)carbonyl]cyclopentyl}nicotinamide

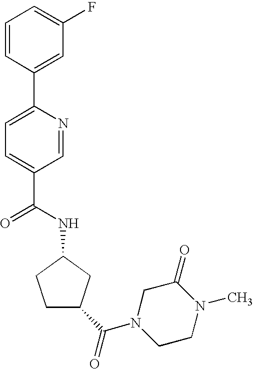

The title compound was prepared using analogous conditions to those described in Example 6 from (1R,3S)-3-({[6-(3-fluorophenyl)pyridin-3-yl]carbonyl}amino)cyclopentanecarboxylic acid (Example 11b) and 1-methyl-2-piperazinone. The crude product was purified by HPLC method (B) (LCMS method (A) RT 2.83 min (100%) ES+m/z 425.19 [M+1]).

### EXAMPLE 174

### 6-(3-Fluorophenyl)-N-{(1S,3R)-3-[(1-methylpiperidin-4-yl)carbamoyl]cyclopentyl}nicotinamide

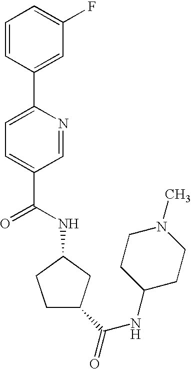

The title compound was prepared using analogous conditions to those described in Example 6 from (1R,3S)-3-({[6-(3-fluorophenyl)pyridin-3-yl]carbonyl}amino)cyclopentanecarboxylic acid (Example 11 band 1-methyl-4-amino-piperidine. The crude product was purified by HPLC method (B) (LCMS method (A) RT 2.24 min (100%) ES+m/z 425.22 [M+1]).

### EXAMPLE 175

### N-[(1S,3R)-3-Carbamoylcyclopentyl]-6-(3-fluorophenyl)nicotinamide

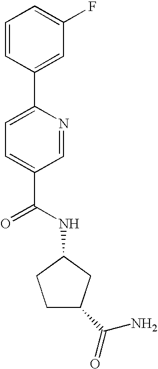

To a solution of (1R,3S)-3-({[6-(3-fluorophenyl)pyridin-3-yl]carbonyl}amino)cyclopentane carboxylic acid (Example 11b, 49.3 mg, 0.15 mmol) in N,N-dimethylformamide (2 ml) was added N,N-carbonyldiimidazole (27.9 mg, 0.172 mmol) and the mixture was stirred for 2 hours at room temperature. Ammonia in dioxane (0.5M, 1.0 ml) was added and the mixture was stirred at room temperature for 17 hours and then heated in a sealed vessel at 70° C. for 15 hours. The cooled reaction mixture was concentrated in vacuo and the residue was dissolved in DCM (10 ml). The solution was washed with water (7 ml), dried, concentrated and purified by HPLC method (B) (LCMS method (A) RT 2.91 min (100%) ES+m/z 328.22 [M+1]).

### EXAMPLE 176

### 6-(3-Fluorophenyl)-N-[(1S,3R)-3-(piperazin-1-ylcarbonyl)cyclopentyl]nicotinamide

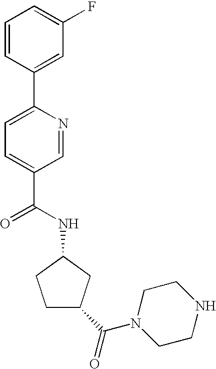

HBTU (78.2 mg, 0.2 mmol) was added to a solution of (1R,3S)-3-({[6-(3-fluorophenyl)pyridin-3-yl]carbonyl}amino)cyclopentane carboxylic acid (Example 11b, 59.3 mg, 0.18 mmol) and triethylamine (54.6 mg, 0.54 mmol) in N,N-dimethylformamide (2 ml) and the mixture was stirred at room temperature for one hour. Piperazine-1-carboxylic acid tent-butyl ester (42.8 mg, 0.23 mmol) was added and the reaction mixture was stirred at room temperature for a further 17 hours. The reaction mixture was diluted with ethyl acetate (35 ml), washed with water (2×30 ml) dried (MgSO₄) and concentrated. The residue was dissolved in HCl in dioxane (4N, 20 ml) and the resulting solution was stirred at room temperature for 6 hours and concentrated in vacuo. The crude product was purified by HPLC method (B) (LCMS method (A) RT 2.14 min (100%) ES+m/z 397.19 [M+1]).

### EXAMPLE 177

### N-{(1S,3R)-3-[(4-Aminopiperidin-1-yl)carbonyl]cyclopentyl}-6-(3-fluorophenyl)nicotinamide

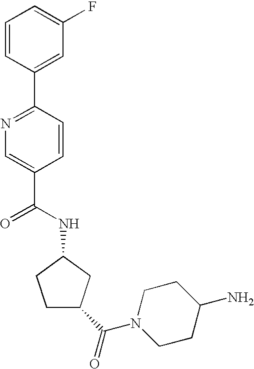

The title compound was prepared using analogous conditions to those described in Example 176 from (1R,3S)-3-({[6-(3-fluorophenyl)pyridin-3-yl]carbonyl}amino)cyclopentanecarboxylic acid (Example 11b) and 4-(tert-butoxycarbonyl)piperidine. The crude product was purified by HPLC method (B) (LCMS method (A) RT 2.16 min (100%) ES+m/z 411.21 [M+1]).

### EXAMPLE 178

### 6-(3-fluorophenyl)-N-{(1S,3R)-3-[(4-methyl-3-oxopiperazin-1-yl)carbonyl]cyclopentyl}nicotinamide

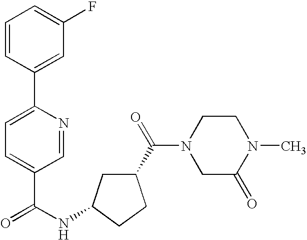

To a solution of (1R,3S)-3-({[6-(3-fluorophenyl)pyridine-3-yl]carbonyl}amino) cyclyopentanecarboxylic acid (Example 11b, 49.3 mg, 0.15 mmol) and triethylamine (68.2 mg, 0.675 mmol) in dimethylformamide (1.3 mL) was added HBTU (65.2 mg, 0.172 mmol) and the solution was stirred at room temperature for 1 hour. 1-Methylpiperazin-2-one (29.4 mg, 0.195 mmol) was added and the solution was stirred at room temperature overnight. The dimethylformamide was removed by evaporation in vacuo and the residue was partitioned between water (7 mL) and ethyl acetate (7 mL). The organic layer was separated and evaporated to give a red-brown gum which was purified by HPLC Method (B) to give 23.2 mg of the title compound (LCMS Method (A), RT 2.83 min, 100% area ES m/z [M+] 424.19).

### EXAMPLE 179

### 6-(3-Fluorophenyl)-N-[(1R,3S)-3-(morpholin-4-ylcarbonyl)cyclopentyl]nicotinamide

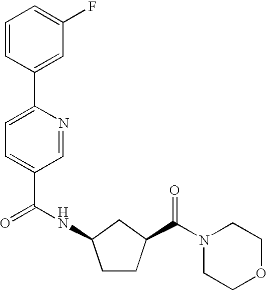

A solution of (1S,3R)-3-({6-(3-Fluorophenyl)pyridine-3-yl]carbonyl}amino)cyclopentane carboxylic acid (Example 11a, 40 mg, 0.122 mmol) in dimethylsulphoxide (0.5 mL) was treated with 1,1-carbonyldiimidazole (23.7 mg, 0.146 mmol) and stirred at room temperature for 1.5 hours. Morpholine (0.013 mL, 0.146 mmol) was added and the reaction mixture was stirred at room temperature overnight. The reaction was purified using reverse phase HPLC Method (B) to give 21.1 mg of the title compound (LCMS Method (A) RT 3.00 min 100% area, [M+] 397.18).

### EXAMPLE 180

### 6-(3-Fluorophenyl)-N-[(1R,3S)-3-{[(3R)-3-fluoropyrrolidin-1-yl]carbonyl}cyclopentyl]nicotinamide

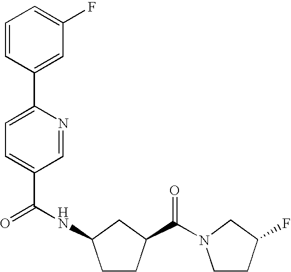

The title compound was prepared using analogous conditions to those described in Example 179 but using (3R)-3-fluoropyrrolidine (18.3 mg, 0.146 mmol) instead of morpholine. The title compound (29 mg) was isolated using reverse phase HPLC Method (B) (LCMS Method (A) RT 3.25 min 100% area, [M+] 399.18).

### EXAMPLE 181

### 6-(3-Fluorophenyl)-N-[(1R,3S)-3-{[(3S)-3-fluoropyrrolidin-1-yl]carbonyl}cyclopentyl]nicotinamide

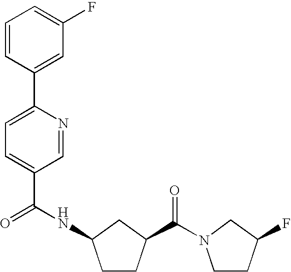

The title compound was prepared using analogous conditions to those described in Example 179 but using (3R)-3-fluoropyrrolidine (18.3 mg, 0.146 mmol) instead of morpholine. The title compound (28.2 mg) was isolated using reverse phase HPLC Method (B) (LCMS Method (A) RT 3.21 min 100% area, ES m/z [M+] 399.18).

### EXAMPLE 182

### 6-(3-Fluorophenyl)-N-[(1R,3S)-3-[(4-methyl-3-oxopiperazin-1-yl)carbonyl]cyclopentyl}nicotinamide

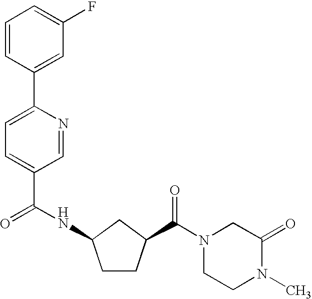

The title compound was prepared using analogous conditions to those described in Example 179 but using 1-methylpiperazin-2-one (13.9 mg, 0.122 mmol) instead of morpholine. The title compound (20.5 mg) was isolated using reverse phase HPLC Method (B) (LCMS Method (A) RT 2.74 min 100% area, [M+] 424.19).

### EXAMPLE 183

### N-[(1R,3S)-3-{[(2-Dimethylamino)ethylcarbamoyl}cyclopentyl]6-(3-fluorophenyl)nicotinamide

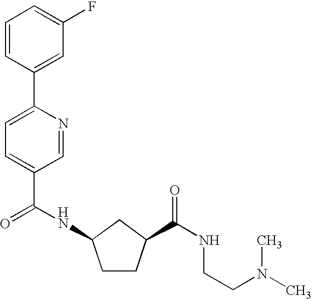

The title compound was prepared using analogous conditions to those described in Example 179 but using N,N-dimethylethylenediamine (16.1 mg, 0.183 mmol) instead of morpholine. The title compound (20.6 mg) was isolated using reverse phase HPLC Method (B) (LCMS Method (A) RT 2.24 min, 100% area, [M+] 398.21).

### EXAMPLE 184

### 6-(3-Fluorophenyl)-N-[(1R,3S)-3-{([4-(2-hydroxyethyl)piperazin-1-yl]carbonyl}cyclopentyl}nicotinamide

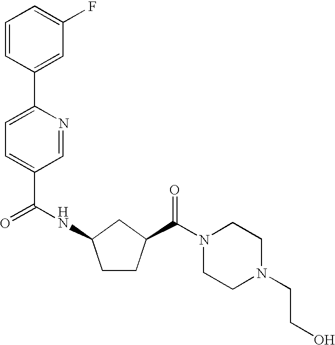

The title compound was prepared using analogous conditions to those described in Example 179 but using 2-piperazin-1-ylethanol (19.0 mg, 0.146 mmol) instead of morpholine. The title compound (23.3 mg) was isolated using reverse phase HPLC Method (B) (LCMS Method (A) RT 2.22 min 100% area, ES m/z [M+] 440.22).

### EXAMPLE 185

### 6-(3-Fluorophenyl)-N-[(1R,3S)-3-[2-methoxyethyl)carbamoyl]cyclopentyl}nicotinamide

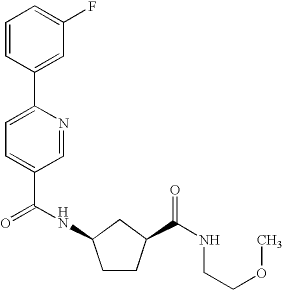

The title compound was prepared using analogous conditions to those described in Example 179 but using 2-methoxyethylamine (13.7 mg, 0.183 mmol) instead of morpholine. The title compound (22.3 mg) was isolated using reverse phase HPLC Method (B) (LCMS Method (A) RT 3.07 min 100% area, ES m/z [M+] 385.18).

### EXAMPLE 186

### N-[(1R,3S)-3-Carbamoylcyclopentyl]-6-(3-fluorophenyl)nicotinamide

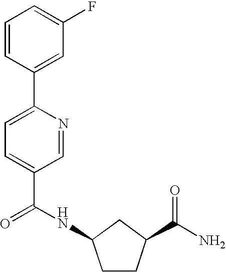

(1S,3R)-3-({6-(3-Fluorophenyl)pyridine-3-yl]carbonyl}amino)cyclopentanecarboxylic acid (Example 11a, 40 mg, 0.122 mmol) was dissolved in dimethylsulphoxide (0.5 mL) and the resulting solution was treated with 1,1-carbonyldiimidazole (23.7 mg, 0.146 mmol) and stirred at room temperature for 1.5 hours. A solution of ammonia in ethanol (2M, 0.122 mL, 0.244 mmol) was added and the reaction mixture was stirred at room temperature overnight. Further ammonia in ethanol (2M, 0.122 mL, 0.244 mmol) was added and the reaction mixture was heated at 50° C. for 4 hours then stirred overnight at room temperature. The title compound (6.1 mg) was isolated using reverse phase HPLC Method (B) (LCMS Method (A) RT 2.74 min, 100% area, ES m/z [M+] 327.14).

### EXAMPLE 187A

### tert-Butyl(1-{[(1S,3R)-3-({[6-(3-fluorophenyl)pyridine-3-yl]carbonyl}amino)cyclopentyl]carbonyl}piperidin-4-yl)carbamate

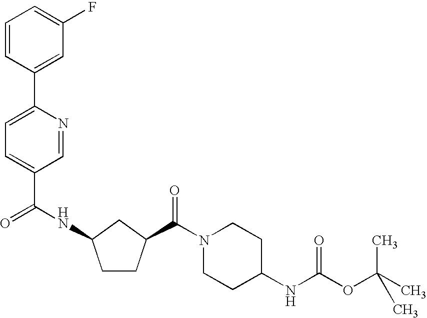

A solution of (1S,3R)-3-({6-(3-fluorophenyl)pyridine-3-yl]carbonyl}amino)cyclopentanecarboxylic acid (60 mg, 0.183 mmol) in dimethylformamide (0.5 mL) was treated with 1,1-carbonyldiimidazole (35.7 mg, 0.220 mmol) and stirred at room temperature for 1.5 hours. tert-Butyl piperidin-4-ylcarbamate (44.1 mg, 0.220 mmol) was added and the reaction mixture was stirred at room temperature for 60 hours. The reaction was partitioned between water (3 mL) and ethyl acetate (5 mL) and the organic layer was evaporated to give the title compound (85 mg) as a white solid (LRMS 511 [M+1] ES⁺ (obs) 511.610 [M+1] (calc)).

### EXAMPLE 187

### N-{(1R,3S)-3-[4-Aminopiperidin-1-yl)carbonyl]cyclopentyl}-6-(3-fluorophenyl)nicotinamide

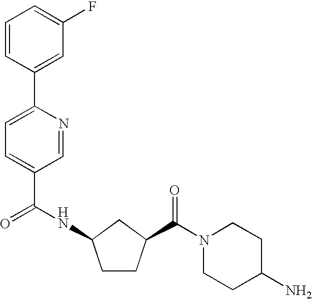

tert-Butyl(1-{[(1S,3R)-3-({[6-(3-fluorophenyl)pyridine-3-yl]carbonyl}amino)cyclopentyl]carbonyl}piperidin-4-yl)carbamate (80 mg, 0.157 mmol, Example 187A) was dissolved in dichloromethane (2.0 mL) and a solution of hydrogen chloride in 1,4-dioxane (4M, 0.589 mL, 2.36 mmol) was added dropwise. Methanol (0.2 mL) was added and the reaction mixture was stirred at room temperature overnight. The solvent was evaporated, the residue was dissolved in further dichloromethane and the solvent was re-evaporated to give a white foam. The crude product was purified by chromatography on an Isolute™ SCX-2 column eluting with methanol and then methanolic ammonia to give a gum and further purified using reverse phase HPLC Method (B) to give 46 mg of the title compound (LCMS Method (A) RT 2.38 min 100% area, ES m/z [M+] 410.21).

### EXAMPLE 188

### 6-(3-Fluorophenyl)-N-3-{[(1R)-2-hydroxy-1-methylethyl]carbamoyl}cyclohexyl]nicotinamide

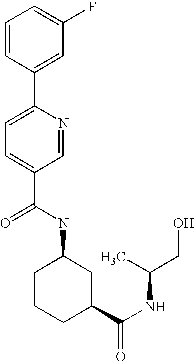

The title compound was prepared using analogous conditions to those described in Example 6 from 3-({[6-(3-fluorophenyl)pyridin-3-yl]carbonyl}amino)cyclopentanecarboxylic acid (Example 27) and (2S)-2-amino-1-propanol. The crude product was purified by HPLC method (B) (LCMS method (A) RT 2.82 min (100%) ES+m/z 400.19 [M+1]).

### EXAMPLE 189

### 6-(3-Fluorophenyl)-N-cis-3-{([2-(methylamino)ethyl]carbamoyl}cyclohexyl]nicotinamide

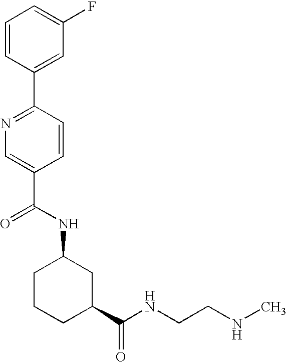

The title compound was prepared using analogous conditions to those described in Example 176 from cis-3-({[6-(3-Fluorophenyl)pyridin-3-yl]carbonyl}amino)cyclohexanecarboxylic acid (Example 27) and (2-amino-ethyl)-methyl-carbamic acid tert-butyl ester. The crude product was purified by HPLC method (B) (LCMS method (A) RT 2.24 min (100%) ES+m/z 399.21 [M+1]).

### EXAMPLE 190

### N-cis-3-{[(1R,5S,6S)-6-Amino-3-azabicyclo[3.1.0]hex-3-yl]carbonyl}cyclohexyl]-6-(3-fluorophenyl)nicotinamide hydrochloride

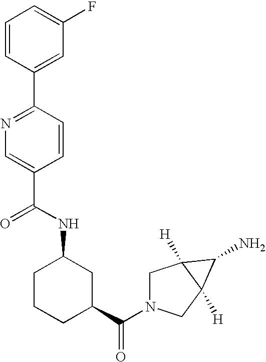

The title compound was prepared using analogous conditions to those described in Example 176 from cis-3-({[6-(3-Fluorophenyl)pyridin-3-yl]carbonyl}amino)cyclohexanecarboxylic acid (Example 27) and carbamic acid, N-(1α,5α,6α)-3-azabicyclo[3.1.0]hex-6-yl-1,1-dimethylethyl ester. LCMS Method (G) RT 0.96 min (100%) ES+m/z 423 [M+1].

### EXAMPLE 191

### 6-(3-Fluorophenyl)-N-cis-3-(piperidin-4-ylcarbamoyl)cyclohexyl]nicotinamide hydrochloride

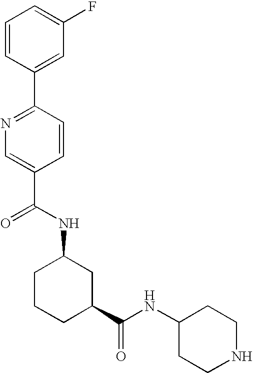

The title compound was prepared using analogous conditions to those described in Example 176 from cis-3-({[6-(3-Fluorophenyl)pyridin-3-yl]carbonyl}amino)cyclohexanecarboxylic acid (Example 27) and 4-amino-1-tert-butoxycarbonylpiperidine. LCMS Method (G) RT 0.96 min (100%) ES+m/z 425 [M+1].

### EXAMPLE 192

### 6-(3-Fluorophenyl)-N-{(1S,3R)-3-[methyl(piperidin-4-yl)carbamoyl]cyclohexyl}nicotinamide

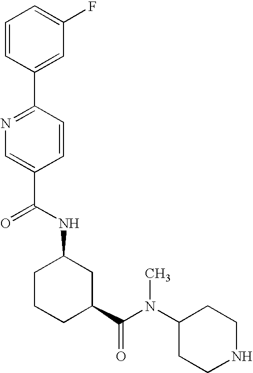

The title compound was prepared using analogous conditions to those described in Example 176 from cis-3-({[6-(3-fluorophenyl)pyridin-3-yl]carbonyl}amino)cyclohexanecarboxylic acid (Example 27) and 4-methylamino-1-tert-butoxycarbonylpiperidine. LCMS Method (G) RT 0.97 min (100%) ES+m/z 439 [M+1].

### EXAMPLE 193

### 6-(3-Fluorophenyl)-N-[(1S,3R)-3-{[trans-4-(methylamino)cyclohexyl]carbamoyl}cyclohexyl]nicotinamide

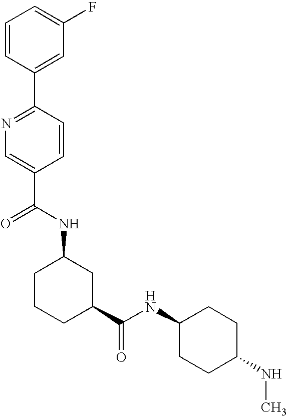

To a solution of cis-3-({[6-(3-fluorophenyl)pyridin-3-yl]carbonyl}amino)cyclohexanecarboxylic acid (Example 27, 78.7 mg, 0.23 mmol) and triethylamine (70 mg, 0.70 mmol) in DMF (4.0 ml) was added HBTU (100 mg, 0.265 mmol) and the mixture was stirred at room temperature for 30 minutes. N-(4-Amino-cyclohexyl)-2,2,2-trifluoro-N-methyl-acetamide (preparation 5) was added and the reaction mixture was stirred at room temperature for 17 hours. The reaction mixture was diluted with DCM (15 ml), washed with water (15 ml) dried and concentrated. The residue was dissolved in methanol (12.0 ml) and the resulting solution was heated at reflux while sodium hydroxide solution (2M 3.0 ml) was added dropwise. Following the addition, heating was continued for 1 hour. The cooled reaction mixture was poured into a mixture of DCM (15 ml), methanol (3.5 ml) and water (7.5 ml). The organic phase was separated, dried and concentrated to give a white solid.

HRMS: C₂₆H₃₄FN₄0₂ (MH⁺) requires 453.2665; found 453.2645.

¹H NMR (400 MHz, DMSO-d₆): δ ppm 0.89-1.59 (m, 7H) 1.59-1.90 (m, 9H) 2.10-2.27 (m, 5H) 3.34-3.50 (m, 1H) 3.75-3.89 (m, 1H) 7.26-7.37 (m, 1H) 7.52-7.67 (m, 2H) 7.90-8.03 (m, 2H) 8.09-8.15 (m, 1H) 8.25-8.31 (m, 1H) 8.46-8.54 (m, 1H) 9.04-9.09 (m, 1H).

### EXAMPLE 194

### 6-(3-Fluorophenyl)-N-cis-3-{[(1S)-2-hydroxy-1-methylethyl]carbamoyl}cyclohexyl]nicotinamide

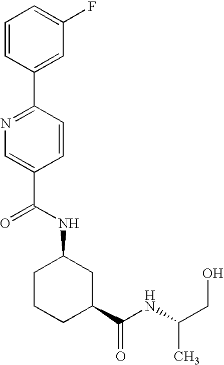

The title compound was prepared using general method (ii) (HBTU coupling) from cis-3-({[6-(3-fluorophenyl)pyridin-3-yl]carbonyl}amino)cyclohexanecarboxylic acid (Example 27) and (2S)-2-amino-1-propanol. The crude product was purified by HPLC method (B) (LCMS method (A) RT 2.75 min (100%) ES− m/z 398.19 [M+1]).

### EXAMPLE 195

### 6-(3-Fluorophenyl)-N-{(1S,3R)-3-[(2-hydroxy-2-methylpropyl)carbamoyl]cyclohexyl}nicotinamide

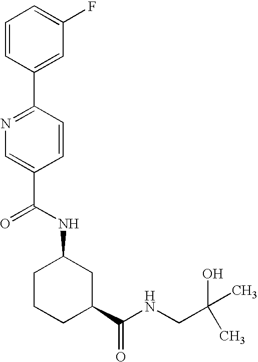

The title compound was prepared using general method (ii) (HBTU coupling) from cis-3-({[6-(3-fluorophenyl)pyridin-3-yl]carbonyl}amino)cyclohexanecarboxylic acid (Example 27) and 1-amino-2-methyl-2-propanol. The crude product was purified by HPLC method (A) (LCMS method (B) RT 2.77 min (100%) ES+m/z 414.21 [M+1]).

### EXAMPLE 196

### 6-(3-Fluorophenyl)-N-cis-3-[(2-hydroxy-1,1-dimethylethyl)carbamoyl]cyclohexyl}nicotinamide

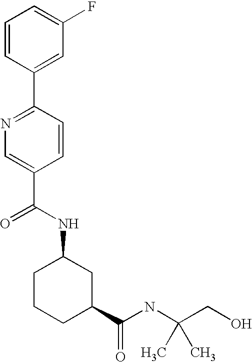

The title compound was prepared using general method (ii) (HBTU coupling) from cis-3-({[6-(3-fluorophenyl)pyridin-3-yl]carbonyl}amino)cyclohexanecarboxylic acid (Example 27) and 2-amino-2-methyl-1-propanol. The crude product was purified by HPLC method (B) (LCMS method (A) RT 2.83 min (100%) ES− m/z 414.21 [M+1]).

### EXAMPLE 197

### 6-(3-Fluorophenyl)-N-cis-3-[(2-hydroxybutyl)carbamoyl]cyclohexyl}nicotinamide

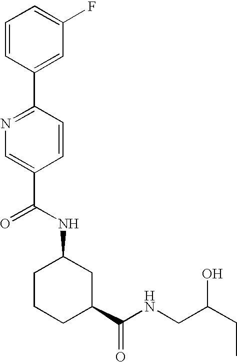

The title compound was prepared using general method (ii) (HBTU coupling) from cis-3-({[6-(3-fluorophenyl)pyridin-3-yl]carbonyl}amino)cyclohexanecarboxylic acid (Example 27) and 1-amino-2-butanol. The crude product was purified by HPLC method (A) (LCMS method (B) RT 2.83 min (100%) ES+m/z 414.21 [M+1]).

### EXAMPLE 198

### N-cis-3-{[2-(Dimethylamino)-2-oxoethyl]carbamoyl}cyclohexyl]-6-(3-fluorophenyl)nicotinamide

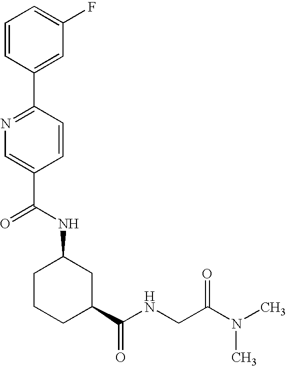

The title compound was prepared using general method (ii) (HBTU coupling) from cis-3-({[6-(3-fluorophenyl)pyridin-3-yl]carbonyl}amino)cyclohexanecarboxylic acid (Example 27) and 2-amino-N,N-dimethyl-acetamide. The crude product was purified by HPLC method (B) (LCMS method (A) RT 2.83 min (100%) ES+m/z 427.20 [M+1]).

### EXAMPLE 199

### 6-(3-Fluorophenyl)-N-[(cis)-3-(1-hydroxyethyl)cyclohexyl]nicotinamide

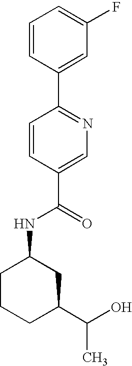

The product of Preparation 53 (57 mg, 0.4 mmol) was dissolved into DMF (1 ml) and 6-(3-fluorophenyl)nicotinic acid (86 mg, 0.4 mmol) was added. The reaction mixture was stirred at room temperature and DIPEA (0.1 g, 0.8 mmol) and HBTU (0.18 g, 0.48 mmol) were added. The reaction mixture was stirred at room temperature for 16 hours. The solvent was removed under reduced pressure and the residue was purified by flash chromatography on silica gel, eluting with DCM:methanol 95:5 by volume. The product-containing fractions were evaporated to give the title compound (53 mg) as a yellow oil (LCMS Method (H)RT 3.09 minutes (73%) area, ES m/z [M+1] 343.2).

### EXAMPLE 200A

### tert-Butyl[cis-3-({[6-(3-fluorophenyl)pyridine-3-yl]carbonyl}amino)cyclohexyl]carbamate

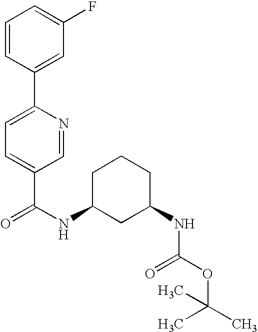

6-(3-Fluorophenyl)nicotinic acid (1025 mg. 4.67 mmol) was dissolved in dimethylformamide (5 mL), 1,1-carbonyl diimidazole (871 mg, 5.37 mmol) was added and the reaction mixture was stirred at room temperature for 1.5 hours. tert-Butyl(cis-3-aminocyclohexyl)carbamate (1000 mg, 4.67 mmol) was added and the reaction mixture was sonicated whereupon a thick precipitate formed. Further dimethyl formamide (5 mL) was added and the reaction mixture was heated to 50° C. for 18 hours with stirring. The dimethylformamide was evaporated in vacuo, water (20 mL) was added to the residue and the product was filtered off and dried in vacuo at 65° C. to give the title compound (1.90 g) as a beige coloured solid.

LRMS: [M+1] 414, [2M+1] 828.

¹H NMR (400 MHz, DMSO-d₆): δ ppm 1.07-1.16 (m, 1H) 1.23-1.37 (m, 4H) 1.39-1.40 (m, 9H) 1.75-1.85 (m, 3H) 2.00-2.03 (m, 1H) 3.81-3.87 (m, 1H) 6.84-6.86 (m, 1H) 7.31-7.36 (m, 1H) 7.56-7.70 (m, 1H) 7.96-8.04 (m, 2H) 8.14-8.16 (m, 1H) 8.29-8.32 (m, 1H) 8.51-8.52 (m, 1H) 9.09-9.10 (m, 1H).

### EXAMPLE 200

### N-[cis-3-Aminocyclohexyl]-6-(3-fluorophenyl)nicotinamide

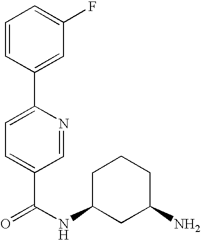

A solution of tert-butyl-cis-3-({[6-(3-fluorophenyl)pyridine-3-yl]carbonyl}amino)cyclohexyl] carbamate (Example 200A, 245 mg, 0.593 mmol) in dioxane (5 mL) was treated with a solution of hydrogen chloride in 1,4-dioxane (4M, 2.96 mL, 11.9 mmol) and the reaction mixture was stirred at room temperature overnight. The crude reaction mixture was partitioned between ethyl acetate (10 mL) and dilute sodium hydroxide solution (10 mL) and the organic layer was re-washed with dilute sodium hydroxide solution (10 mL). The organic layer was dried over magnesium sulphate, filtered and evaporated to give a white solid (43 mg) which was purified using Method (B) to give 20.4 mg of the title compound (LCMS Method (B) RT 2.90 min, 100% area ES m/z [M+] 313.16).

### EXAMPLE 201

### 6-(3-Fluorophenyl)-N-[trans-4-(piperidin-4-ylcarbamoyl)cyclohexyl]nicotinamide

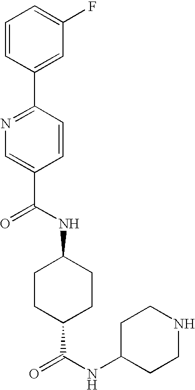

The title compound was prepared using analogous conditions to those described in Example 176 from trans-4-({[6-(3-fluorophenyl)pyridin-3yl]carbonyl}amino)cyclohexanecarboxylic acid (Example 84) and 4-amino-1-tert-butoxycarbonylpiperidine. LCMS Method (G)RT 0.93 min (100%) ES+m/z 425 [M+1].

### General Procedure for Examples 202-206

Examples 202-206 were prepared using the following general procedure. A solution of the appropriate acid (0.129 mmol) in dimethyl sulphoxide (0.5 mL) was treated with 1,1′-carbonyldiimidazole (0.129 mmol) and stirred at room temperature for 1.5 hours. N-[cis-3-aminocyclohexyl]-6-(3-fluorophenyl)nicotinamide (0.129 mmol, Example 200) along with N,N-diisopropylethylamine (0.067 mL) were added and the reaction mixture was stirred at room temperature overnight. The reaction was monitored by LCMS. Where necessary, further equivalents of the appropriate acid reactant which had been dissolved in dimethyl sulfoxide with the appropriate amount of 1,1′-carbonyldiimidazole for 1.5 hours were added. When the reactions were judged to have undergone sufficient conversion they were purified by reverse phase HPLC.

### EXAMPLE 202

### 6-(3-Fluorophenyl)-N-[cis-3-glycoloylamino}cyclohexyl]nicotinamide

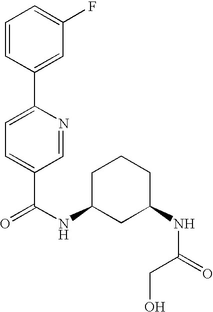

Using glycolic acid in the method described above for Examples 202-206, 18.7 mg of the title compound was isolated using HPLC Method (A). LCMS Method (B) RT 2.60 min, 100% area ES m/z [M+] 371.16.

### EXAMPLE 203

### 6-(3-Fluorophenyl)-N-cis-[3-(2-methoxyacetylamino)cyclohexyl]nicotinamide

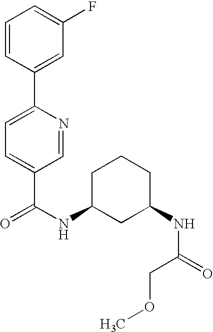

Using methoxyacetic acid in the method described above for Examples 202-206, 15.1 mg of the title compound was isolated using HPLC Method (A). LCMS Method (B) RT 2.96 min 100% area, ES m/z [M+] 385.18.

### EXAMPLE 204

### 6-(3-Fluorophenyl)-N-cis-3-{[(4-methylpiperazin-1-yl)acetyl]amino}cyclohexyl]nicotinamide

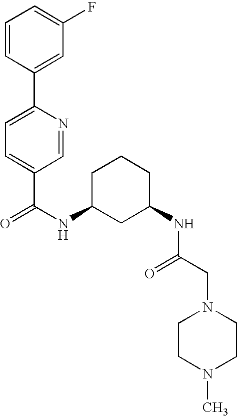

Using (4-methylpiperazin-1-yl)acetic acid in the method described above for Examples 202-206, 8 mg of the title compound was isolated using HPLC Method (B). LCMS Method (B) RT 2.17 min 100% area, ES m/z [M+] 453.25.

### EXAMPLE 205

### 6-(3-Fluorophenyl)-N-cis-[3-(2-hydroxy-2-methylpropionylamino)cyclohexyl]nicotinamide

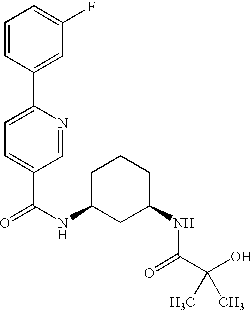

Using 2-hydroxy-2-methylpropanoic acid in the method described above for Examples 202-206, 20.4 mg of the title compound was isolated using HPLC Method (A). LCMS Method (A) RT 2.84 min 100% area, ES m/z [M+] 399.20.

### EXAMPLE 206

### 6-(3-Fluorophenyl)-N-{cis-3-{[(2S)-2-hydroxypropanoyl]amino}cyclohexyl]nicotinamide

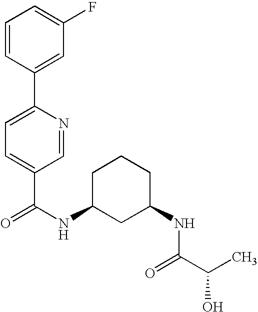

The title compound was prepared by the method described above for Examples 202-206, using (2S)-2-hydroxypropanoic acid, but in this case the reaction mixture was heated to 70° C. overnight and then at 80° C. for 4 hours. The title compound (15.3 mg) was isolated using HPLC Method (A). LCMS Method (A) RT 2.78 min 100% area, ES m/z [M+] 385.18.

### EXAMPLE 207

### 6-(3-Fluoro-phenyl)-N-cis-(3-methanesulfonylamino-cyclohexyl)-nicotinamide

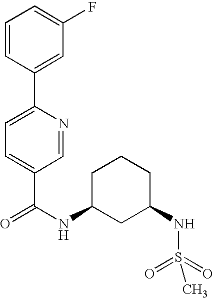

N-[cis-3-Aminocyclohexyl]-6-(3-fluorophenyl)nicotinamide (49.8 mg, 0.129 mmol, Example 200) was dissolved in dimethylsulphoxide (0.5 mL) and N,N-diisopropylethylamine (0.067 mL) was added. Methanesulphonyl chloride (0.015 mL. 0.194 mmol) was added and the reaction mixture was stirred at room temperature overnight. The reaction mixture was purified by reverse phase

HPLC Method (A) to give 15.5 mg of the title compound. LCMS Method (A) RT 2.97 min 100% area, ES m/z [M+] 391.14.

### EXAMPLES 208 AND 209

### 6-(3-Fluorophenyl)-N-{(1S,3R)-3-[4-methylpiperazin-1-yl]carbonyl]cyclohexyl}nicotinamide and 6-(3-fluorophenyl)-N-{(1S,3S)-3-[4-methylpiperazin-1-yl]carbonyl]cyclohexyl}nicotinamide

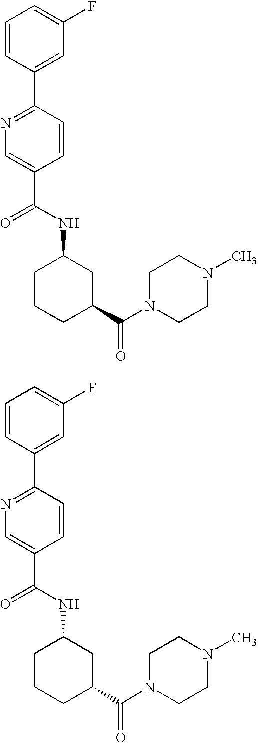

Cis-3-({[6-(3-fluorophenyl)pyridine-3-yl]carbonyl}amino)cyclohexanecarboxylic acid (Example 27, 71 mg, 0.207 mmol) was dissolved in dimethylformamide (1.0 mL) and the resulting solution was treated with 1,1′-carbonyl diimidazole (40.2 mg, 0.248 mmol) and stirred at room temperature for 1.5 hours. N-Methylpiperazine (21.7 mg, 0.217 mmol) was added and the reaction mixture was stirred at room temperature overnight. The dimethylformamide was evaporated, the residue was partitioned between ethyl acetate (5 mL) and water (5 mL) and the organic layer was separated and evaporated to give a gum which crystallised on scratching. Trituration with tert-butylmethylether gave a mixture of the two title compounds as a white solid (95 mg). The racemic mixture was separated into the two enantiomers by chiral preparative HPLC on a Chiracel OJ-H column eluting with a 1:1 (by volume) methanol:ethanol mixture, using a flow rate of 15 mL per min. Fraction 1 was >99.0% pure of the peak eluting at 4.7 min. Fraction 2 was 99.6% pure of the peak eluting at 6.3 min. The two fractions were evaporated and then re-evaporated from t-butyl-methyl ether to give the title compounds as white solids (26 mg of each obtained).

### EXAMPLE 210

### N-(trans-4-{[(1R,5S,6S)-6-Amino-3-azabicyclo[3.1.0]hex-3-yl]carbonyl}cyclohexyl)-6-(3-fluorophenyl)nicotinamide

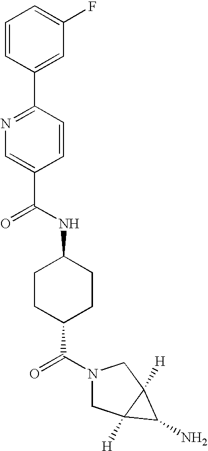

The title compound was prepared using analogous conditions to those described in Example 176 from trans-4-({[6-(3-Fluorophenyl)pyridin-3yl]carbonyl}amino)cyclohexanecarboxylic acid (Example 84) and carbamic acid, N-(1α,5α,6α)-3-azabicyclo[3.1.0]hex-6-yl-, 1,1-dimethylethyl ester. LCMS Method (G) RT 0.91 min (100%), ES+m/z 423 [M+1]

### EXAMPLE 211

### 6-(3-Fluorophenyl)-N-{trans-4-[methyl(piperidin-4-yl)carbamoyl]cyclohexyl}nicotinamide

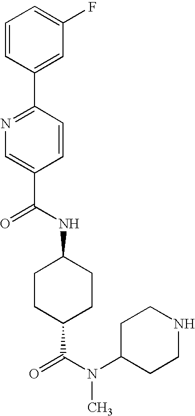

The title compound was prepared using analogous conditions to those described in Example 176 from trans-4-({[6-(3-Fluorophenyl)pyridin-3yl]carbonyl}amino)cyclohexanecarboxylic acid (Example 84) and 4-methylamino-piperidine-1-carboxylic acid tert-butyl ester. The crude product was purified by HPLC method (B) (LCMS method (A) RT 2.25 min (100%), ES+m/z 439.24 [M+])

### EXAMPLE 212

### N-[4-trans-((R)-Cyclopropylhydroxymethyl)cyclohexyl]-6-(3-fluorophenyl)nicotinamide

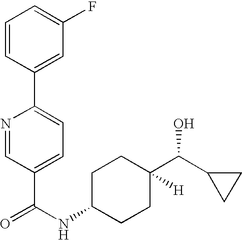

CDI (425 mg, 2.62 mmol) was added to a solution of 6-(3-fluorophenyl)nicotinic acid (Preparation 1, 475 mg, 2.19 mmol) in DMF (10 ml) and the mixture was stirred for 1 hour. The amine from Preparation 33 (370 mg, 2.19 mmol) was added as a solution in DMF (1 ml) and the reaction mixture was stirred for 18 hours at room temperature. The DMF was removed in vacuo to give a white solid which was added to a mixture of DCM (30 mL) and water (30 mL). The mixture was shaken vigorously but solid remained at interface between two layers. Methanol (3 ml) was added and vigorous shaking continued. The organic layer was separated and the aqueous layer was washed with 10% MeOH in DCM (2×30 mL). Some solid still remained. The aqueous phase was therefore reduced to half its original volume under reduced pressure and then extracted with ethyl acetate (2×20 mL). The majority of solid dissolved. The various organic phases were combined, dried over MgSO₄ and evaporated in vacuo to give a white solid (850 mg). Recrystallisation of the crude product from ethanol (20 mL) afforded a white solid which was collected by filtration, washed with ethanol (2×10 mL) and dried under reduced pressure to give the title compound (408 mg). Chiral HPLC on a Chiralpak IA column eluting with 80/20 heptane/isopropanol showed product to have an enentiomeric excess of 97.3%. The filtrate was concentrated in vacuo to give a further crop of title compound (397 mg) with reduced enantiomeric purity (87.8% enantiomeric excess).

LCMS Method (G): RT 1.51 min, m/z (ES+) [M+1] 369.

¹H NMR (400 MHz, DMSO-d6): δ ppm 0.21 (m, 2H), 0.36 (m, 2H), 0.80 (m, 1H), 1.20 (m, 2H), 1.33 (m, 3H), 1.83 (m, 1H), 1.94 (m, 2H), 2.61 (m, 1H), 3.74 (m, 1H), 4.28 (d, 1H), 7.32 (t, 1H), 7.58 (m, 1H), 7.94 (m, 1H), 8.00 (m, 1H), 8.12 (d, 1H), 8.30 (m, 1H), 8.43 (m, 1H), 9.08 (d, 1H).

### EXAMPLE 213

### 6-(3-Fluorophenyl)-N-(trans-4-pyrrolidin-1-ylcyclohexyl)nicotinamide

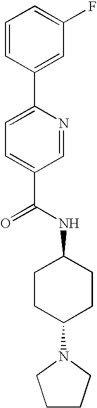

A suspension of 6-(3-fluorophenyl)nicotinic acid (135 mg, Preparation 1) in DCM was treated with oxalyl chloride (1 equivalent) and stirred for 5 minutes. DMF (1 drop) was added and the mixture was stirred for 4 hours at RT, during which time the heterogenous mixture/suspension formed a homogenous yellow solution. The solution was evaporated and the residue was azeotroped with toluene (3×50 ml) and dissolved in DCM (50 ml). 4-Pyrrolidin-1-yl-cyclohexylamine (150 mg) was added and the mixture was cooled to 0° C. Triethylamine (0.35 ml) was then added dropwise and the mixture was stirred overnight at room temperature. The reaction mixture was washed with 10% aqueous potassium carbonate (2×100 ml), dried (MgSO₄) and evaporated to give an amorphous light brown solid (200 mg). The solid was dissolved in DCM (100 ml) treated with 4N HCl in dioxane (20 ml) and evaporated. The residue was dried overnight in vacuo@ 60° C. to give crude product (238 mg) which was further purified by HPLC Method (B). LCMS Method (A) RT 2.15 min, [M+1] 368.2.

### EXAMPLE 214A

### 6-(3-Fluoro-phenyl)-N-(4-formyl-cyclohexyl)-nicotinamide

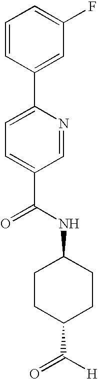

The product of Example 103 (1.78 g, 5.42 mmol) was dissolved in DCM (20 ml) and cooled to 0° C. Dess-Martin periodinane (15% in CH₂Cl₂, 13.5 ml) was added dropwise with stirring. The reaction mixture was allowed to warm to room temperature, THF (30 ml) was added and the solution was heated to reflux. After 3 hours, the solvents were removed in vacuo and the residued was dissolved in a mixture of ethyl acetate (100 ml) and MeOH (10 ml). The resulting solution was washed with saturated aqueous sodium hydrogen carbonate and brine, dried over Na₂SO₄ and evaporated to dryness. The resulting off-white powder was purified using flash column chromatography on silica eluting with MeOH/DCM 10/90 to obtain 755 mg of a fine white powder. LRMS: m/z 327.2 [M+1].

### EXAMPLE 214

### 6-(3-Fluorophenyl)-N-[trans-4-(1-hydroxypropyl)cyclohexyl]nicotinamide

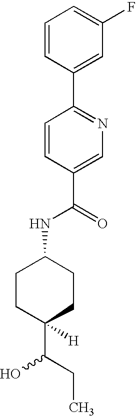

The title compound was prepared using analogous conditions to those described in Preparation 54 from 0.10 g of the product of Example 214A. The product was obtained as a white powder (40 mg). LCMS Method (Y): RT 3.31 mins, LRMS m/z [M+1]357.2.

### EXAMPLE 215

### 6-(3-Fluorophenyl)-N-[trans-4-(1-hydroxyethyl)cyclohexyl]nicotinamide

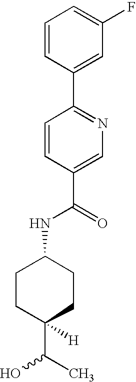

The title compound was prepared in a manner analogous to Preparation 54 using 0.15 g of the product of Example 214A. The product was obtained as a white powder (74 mg). LCMS Method (Y): RT, 3.15 min, [M+1] 343.2.

### EXAMPLE 216

### 6-(3-Fluorophenyl)-N-{cis-4-[(2-hydroxy-2-methylpropanoyl)amino]cyclohexyl}nicotinamide

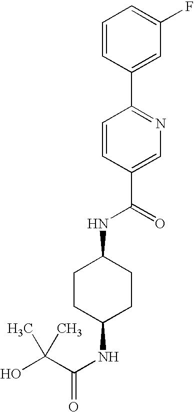

A solution of the product of Example 155 (100 mg, 0.32 mmol) in DMF (1 mL) was treated with HOAt (21.7 mg, 0.16 mmol), 2-hydroxy-2-methylpropanoic acid (47 mg, 0.45 mmol) and EDC (122 mg, 0.64 mmol). The reaction mixture was stirred at room temperature for 72 hours and then more EDC (60 mg, 0.32 mmol) and HOAt (10 mg, 0.08 mmol) were added. After stirring at room temperature for another 24 hours, water (20 mL) was added and the resulting suspension was extracted with ethyl acetate (2×20 mL). The combined organic layers were dried over anhydrous sodium sulphate and concentrated under reduced pressure to give a residue that was triturated from ether (20 mL) and water (20 mL). This gave the title compound (22 mg) as a white solid. LCMS Method (X): RT 2.91 minutes (95%) area, ES m/z [M+1] 400.2.

The following compounds 217-222 were prepared in an analogous manner to Example 216.

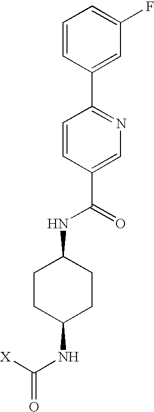

| | | | Purification, characterization and variation |
|---|---|---|---|
| Ex | X | Name | from Example 216 |
| 217 | 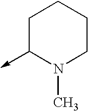 | 6-(3-fluorophenyl)-N-(cis-4-{[(1- methylpiperidin-2- yl)carbonyl]amino}cyclohexyl)nicotinamide | Not triturated. LCMS Method (X): RT 2.58 minutes (98%) area, ES m/z [M + 1] 439. |
| 218 | 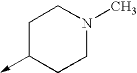 | 6-(3-fluorophenyl)-N-(cis-4-{[(1- methylpiperidin-4- yl)carbonyl]amino}cyclohexyl)nicotinamide | 1-Methylpiperidine-4-carboxylic acid hydrochloride salt and 1 equiv of DIPEA used as starting materials. The reaction was diluted with water (10 mL) and extracted with ethyl acetate (2 × 10 mL). Saturated aqueous sodium hydrogen carbonate solution (10 mL) was added to the aqueous phase and then it was extracted twice with ethyl acetate (2 × 10 mL). The combined organic layers were dried with anhydrous sodium sulphate and evaporated to give the title compound. LCMS Method (X): RT 2.56 minutes (99%) area, ES m/z [M + 1] 439.2. |
| 219 | 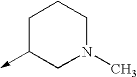 | 6-(3-fluorophenyl)-N-(cis-4-{[(1- methylpiperidin-3- yl)carbonyl]amino}cyclohexyl)nicotinamide | 1-Methylpiperidine-3-carboxylic acid hydrochloride salt and 1 equiv of DIPEA used as starting materials. The reaction was diluted with water (10 mL) and extracted with ethyl acetate (2 × 10 mL). Saturated aqueous sodium hydrogen carbonate solution (10 mL) was added to the aqueous phase and then it was extracted twice with ethyl acetate (2 × 10 mL). The combined organic layers were dried with anhydrous sodium sulphate and evaporated to give the title compound. LCMS Method (X): RT 2.52 minutes (97%) area, ES m/z [M + 1] 439. |
| 220 | 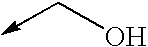 | 6-(3-fluorophenyl)-N-[cis-4- (glycoloylamino)cyclohexyl]nicotinamide | 1 equivalent of DIPEA was used in addition to the other starting materials. LCMS Method (X): RT 2.79 minutes (99%) area, ES m/z [M + 1] 372.2. |
| 221 | 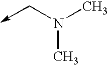 | N-{cis-4-[(N,N- dimethylglycyl)amino]cyclohexyl}-6-(3- fluorophenyl)nicotinamide | The reaction was diluted with water (10 mL) and extracted with ethyl acetate (2 × 10 mL). Saturated aqueous sodium hydrogen carbonate solution (10 mL) was added to the aqueous phase and then it was extracted twice with ethyl acetate (2 × 10 mL). The combined organic layers were dried with anhydrous sodium sulphate and evaporated to give the title compound. LCMS Method (X): RT 2.48 minutes (98%) area, ES m/z [M + 1] 399. |
| 222 | 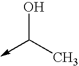 | 6-(3-fluorophenyl)-N-[cis-4- (lactoylamino)cyclohexyl]nicotinamide | The residue after final solvent evaporation was treated with 2 N aqueous sodium hydroxide solution (5 mL) and the mixture was heated to 50° C. for 20 minutes before being allowed to cool and extracted with ethyl acetate (10 mL). The solvent was removed under reduced pressure to give the title compound. LCMS Method (X): RT 2.85 minutes (97%) area, ES m/z [M + 1] 386.2. |

### EXAMPLE 223

### tert-Butyl(2-{[cis-4-({[6-(3-fluorophenyl)pyridin-3-yl]carbonyl}amino)cyclohexyl]amino}-2-oxoethyl)carbamate

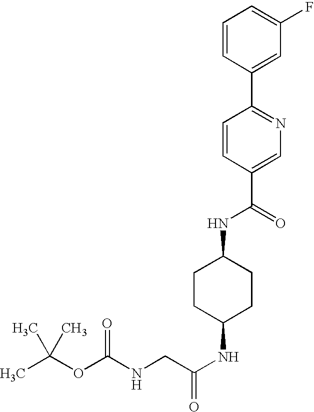

The product of Example 155 (150 mg, 0.48 mmol) was dissolved in DMF (3 mL) and N-(tert-butoxycarbonyl)glycine (0.11 g, 0.62 mmol), HOAt (33 mg, 0.24 mmol) and EDC (0.18 g, 0.96 mmol) were added. The reaction mixture was stirred for 48 hours. Saturated aqueous sodium hydrogen carbonate solution (20 mL) was and the mixture was extracted with ethyl acetate (2×20 mL). The combined organic layers were washed with brine (2×20 mL), dried over anhydrous sodium sulphate and evaporated to give the title compound (160 mg).

### EXAMPLE 224

### 6-(3-fluorophenyl)-N-[cis-4-(glycylamino)cyclohexyl]nicotinamide

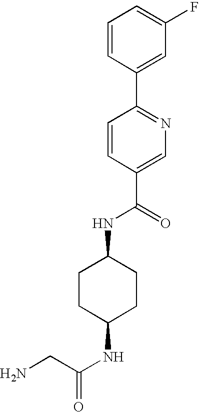

The product of Example 223 (0.16 g, 0.34 mmol) was treated with trifluoroacetic acid (2 mL, 26 mmol) and the resulting solution was stirred at room temperature for 3 hours. The solvent was then removed under reduced pressure and the residue was dissolved in water and extracted with ethyl acetate (20 mL). The pH of the aqueous phase was adjusted to 8 with saturated aqueous sodium hydrogen carbonate and the solution was extracted with further ethyl acetate (2×20 mL). The combined organic phases were washed with brine (20 mL), dried over anhydrous sodium sulphate and evaporated under reduced pressure. The residue was purified by flash chromatography on silica gel using DCM:methanol 90:10 by volume as eluant to give the title compound, 16 mg, as a yellow oil. LCMS Method (H): RT 2.61 minutes (98%) area, ES m/z [M+1] 371.2.

### EXAMPLE 225

### 6-(3-Fluorophenyl)-N-[4-(hydroxymethyl)-4-methoxycyclohexyl]nicotinamide

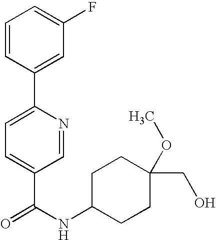

The title compound (35 mg, as a white powder) was prepared from the product of Preparation 39 (89 mg, 0.56 mmol) and 6-(3-fluorophenyl)nicotinic acid (127 mg, 0.587 mmol, Preparation 1), using general method (iii) for amide formation. LCMS Method (H): RT 2.88 min, [M+1] 359.

### EXAMPLE 226

### 6-(3-Fluorophenyl)-N-[4-hydroxy-4-(isopropoxymethyl)cyclohexyl]nicotinamide

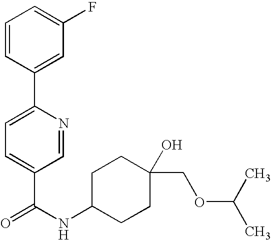

The title compound (36 mg of a white solid) was prepared in a manner similar to Example 225 starting from Preparation 41A and using analogous reactions and intermediates. LCMS Method (H): RT 3.23 min, [M+1] 387.

### EXAMPLE 227

### 6-(3-Fluorophenyl)-N-[4-hydroxy-4-(hydroxymethyl)cyclohexyl]nicotinamide

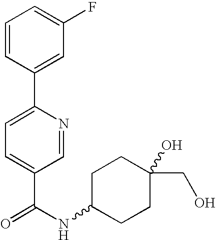

The title compound (36 mg of a white solid) was prepared in a manner similar to Example 225 starting from Preparation 91 and using analogous reactions and intermediates. LCMS Method (H): RT 3.15 min, [M+1] 343.2.

### EXAMPLE 228

### 6-(3-Fluorophenyl)-N-[4-hydroxy-4-(propoxymethyl)cyclohexyl]nicotinamide

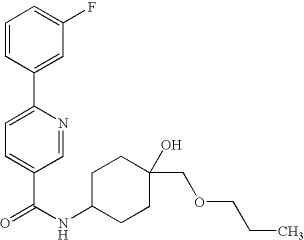

The title compound (111 mg as a white powered) was prepared in a manner similar to Example 225 starting from Preparation 93 and using analogous reactions and intermediates. LCMS Method (H): RT 3.28 min, [M+1] 387.

### EXAMPLE 229

### 1-[trans-4-({[6-(3-Fluorophenyl)pyridin-3-yl]carbonyl}amino)cyclohexyl]-1-methylethyl methylcarbamate

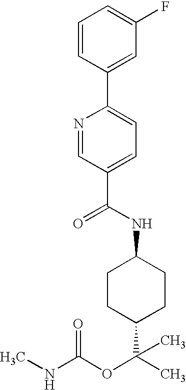

The product of Example 48 (0.050 g) was dissolved in 0.5 ml THF, and 0.12 g of methyl isocyanate was added. This resulting solution was heated to 150° C. in the microwave for 30 minutes. A second aliquot of 100 μl of methyl isocyanate was added and the reaction mixture was heated to 140° C. for 1 hour in the microwave. The reaction was quenched with 20 ml MeOH and concentrated in vacuo. The resulting white solid was purified using flash column chromatography eluting with a MeOH:DCM gradient of 2:98 to 6:94 by volume to give 60 mg of a white powder. The product was further purified using a second flash column eluting with an EtOAc:heptane gradient of 1:4 to 1:1 by volume to give the product (19 mg) as a clear oil that crystallized on standing. LCMS Method (H): RT 2.01 min, [M+1] 414.2.

### EXAMPLE 230

### 6-(3-Fluorophenyl)-N-{(1R,3S)-3-methyl-3-[(4-methylpiperazin-1-yl)carbonyl]cyclohexyl}nicotinamide

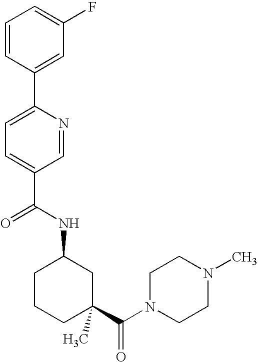

The title compound was prepared using analogous conditions to those described in Preparation 63 from 0.10 g of Example 231. The product (84 mg) was obtained as a colourless oil. LCMS (X): RT 2.65 min, [M+1] 439.2.

### EXAMPLE 231A

### Ethyl 3-({[6-(3-Fluorophenyl)pyridin-3-yl]carbonyl}amino)-1-methylcyclohexane carboxylate

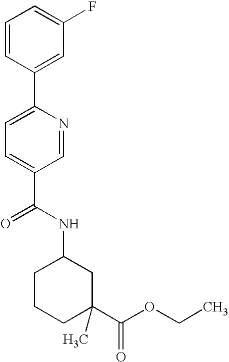

The title compound was prepared from the product of Preparation 45 using general method (iii) for amide formation. LCMS Method (H) RT 2.23 min, [M+1] 385.2.

### EXAMPLE 231

### (1S*,3R*)-3-({[6-(3-Fluorophenyl)pyridin-3-yl]carbonyl}amino)-1-methylcyclohexane carboxylic acid

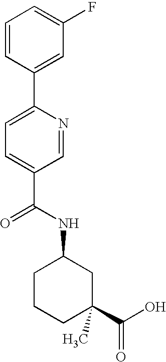

A solution of lithium hydroxide monohydrate (1.064 g) in water (25.0 ml) was added to a stirred solution of the product of Example 231A (1.95 g) in THF (25.0 ml) at room temperature. The resulting reaction mixture was stirred at 60° C. for 18 hours. Extra lithium hydroxide monohydrate (0.426 g, 2.0 equivalents) was added and the reaction mixture was stirred at 60° C. for a further 18 hours. The reaction mixture was cooled to room temperature and concentrated to remove the THF. The residue was diluted with water (20 ml) and acidified with 1M aq. HCl to pH 5. The precipitate which formed was collected by filtration and dried with a stream of air to give the crude product as a pink solid. The crude material was dissolved in methanol (5 ml) and silica 60-200 μm (2 g) was added. The solvent was carefully removed in vacuo and the adsorbed material was loaded on a flash column (silica 20-45 μm) and eluted with a gradient of CH₂Cl₂:MeOH 99:1 to 90:10 by volume to the product as a pink solid (350 mg). LCMS Method (H): RT 1.92 min, [M+1] 357.

### EXAMPLE 232

### 6-(3-Fluorophenyl)-N-[4-(1-hydroxyethyl)-4-methoxycyclohexyl]nicotinamide

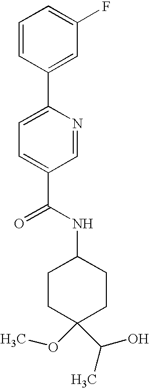

The title compound (212 mg of a colourless oil) was prepared in a manner similar to Example 225 starting from Preparation 48A (187 mg) and using analogous reactions and intermediates. LCMS Method (H): RT 1.85 min, [M+1] 373.

### EXAMPLE 233

### N-{(1R*,3S*)-3-[(4-Ethylpiperazin-1-yl)carbonyl]-3-methylcyclohexyl}-6-(3-fluorophenyl)nicotinamide

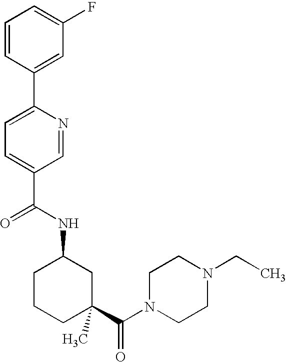

The title compound was prepared using analogous conditions to those described in Preparation 61 using 0.10 g of the product of Example 231. The product (79 mg) was obtained as a colourless oil. LCMS Method (X): RT 2.68 min, [M+1] 453.2.

### EXAMPLE 234

### N-{cis-4-[(Dimethylcarbamoyl)amino]cyclohexyl}-6-(3-fluorophenyl)nicotinamide

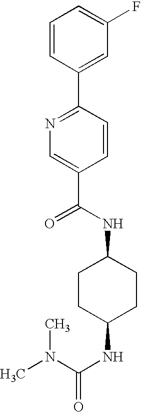

A solution of the product of Example 155 (0.15 g, 0.479 mmol) and DIEPA (0.167 ml, 0.957 mmol) in anhydrous N,N-dimethylformamide (3 ml) was added dropwise to a rapidly stirred solution of CDI (0.078 g, 0.479 mmol) in DCM (3 mL). After 1 hour at room temperature, the reaction mixture was treated with 2 ml of 2M dimethylamine in THF. After 2 further hours the solvents were removed in vacuo and the residue was dissolved in 20 ml of EtOAc. The resulting solution was washed with brine, dried over Na₂SO₄. and evaporated in vacuo to give 197 mg of a clear oil. The oil was stirred with 20 ml of diethylether and 2 ml of DCM and the resulting solid was collected by filtration and dried to give 37 mg of the product as a white powder. LCMS Method (X): RT 2.0 min, [M+1] 385.2.

### EXAMPLE 235

### 6-(3-Fluorophenyl)-N-[4-hydroxy-4-(methoxymethyl)cyclohexyl]nicotinamide

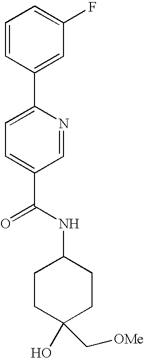

The title compound (21 mg of a pale yellow powder) was prepared using the method of Example 226 starting from Preparation 40 and using analogous reactions and intermediates. LCMS Method (X): RT 1.83 min, [M+1] 359.

### EXAMPLE 236

### 6-(3-Fluorophenyl)-N-{cis-4-[(methylcarbamoyl)amino]cyclohexyl}nicotinamide

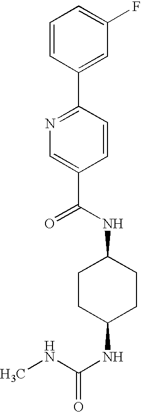

The title compound (33 mg of white flakes) was prepared from 75 mg of the product of Example 155 in a manner similar to Example 234. LCMS Method (X): RT 1.94 min, [M+1] 371.1.

### EXAMPLE 237

### N-[(1R*,3S*)-3-(Dimethylcarbamoyl)-3-methylcyclohexyl]-6-(3-fluorophenyl)nicotinamide

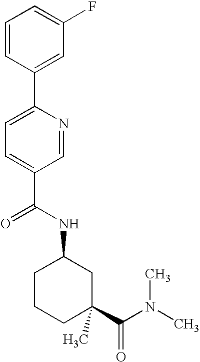

The title compound was prepared in a manner analogous to Preparation 61 using 0.93 g of the product of Example 231. The product (56 mg) was obtained as a colourless oil. LCMS Method (X): RT 3.20 min, [M+1] 384.2.

### EXAMPLE 238A

### trans-3-({[6-(3-Fluorophenyl)pyridin-3-yl]carbonyl}amino)cyclohexanecarboxylic acid

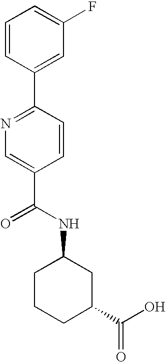

The title compound was prepared using analogous conditions to those described in Example 27 from 6-(3-fluorophenyl)nicotinic acid and trans-3-amino-cyclohexanecarboxylic acid methyl ester.

LCMS: RT 1.34 min (100%) ES+m/z 343 [M+1].

¹H NMR (400 MHz, DMSO-d₆): δ ppm 1.4-1.79 (m, 7H) 1.88-1.96 (m, 1H) 2.71-2.80 (m, 1H) 4.04-4.14 (m, 1H) 7.26-7.34 (m, 1H) 7.52-7.61 (m, 2H) 7.92-8.03 (m, 2H) 8.08-8.14 (m, 1H) 8.25-8.30 (m, 1H) 8.32-8.37 (m, 1H) 9.04-9.08 (m, 1H) 12.05-12.21 (br s, 1H).

### EXAMPLE 238

### N-trans-3-(Dimethylcarbamoyl)cyclohexyl]-6-(3-fluorophenyl)nicotinamide

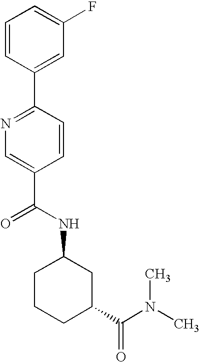

The title compound was prepared using general method (ii) for amide bond formation (HBTU coupling) from trans-3-({[6-(3-fluorophenyl)pyridin-3-yl]carbonyl}amino)cyclohexanecarboxylic acid (Example 238A) and dimethylamine. The product was purified by HPLC method (B). LCMS method (A): RT 2.97 min (100%) ES+m/z 370.18 [M+1].

### EXAMPLE 239

### 6-(3-Fluorophenyl)-N-trans-3-(pyrrolidin-1-ylcarbonyl)cyclohexyl]nicotinamide

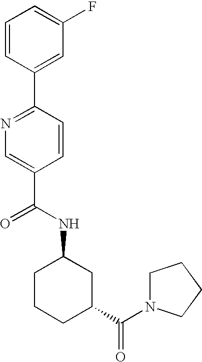

The title compound was prepared using general method (ii) (HBTU coupling) from trans-3-({[6-(3-fluorophenyl)pyridin-3-yl]carbonyl}amino)cyclohexanecarboxylic acid (Example 238A) and pyrrolidine. The product was purified by HPLC method (B). LCMS method (A): RT 3.13 min (100%) ES+m/z 396.20 [M+1].

### EXAMPLE 240

### 6-(3-Fluorophenyl)-N-trans-3-[(2-methoxyethyl)carbamoyl]cyclohexyl}nicotinamide

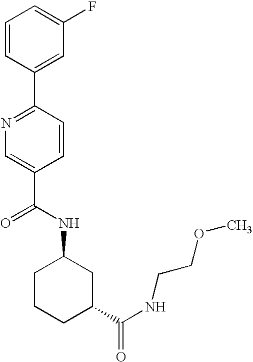

The title compound was prepared using general method (ii) (HBTU coupling) from trans-3-({[6-(3-fluorophenyl)pyridin-3-yl]carbonyl}amino)cyclohexanecarboxylic acid (Example 238A) and 2-methoxy-ethyl amine. The product was purified by HPLC method (B). LCMS method (A): RT 2.97 min (100%) ES+m/z 400.19 [M+1].

### EXAMPLE 241

### 6-(3-Fluorophenyl)-N-trans-3-(morpholin-4-ylcarbonyl)cyclohexyl]nicotinamide

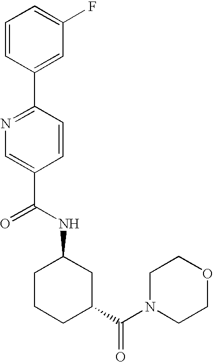

The title compound was prepared using general method (ii) (HBTU coupling) from trans-3-({[6-(3-fluorophenyl)pyridin-3-yl]carbonyl}amino)cyclohexanecarboxylic acid (Example 238A) and morpholine. The product was purified by HPLC method (B). LCMS method (A): RT 3.14 min (100%) ES+m/z 412.19 [M+1].

### EXAMPLE 242

### 6-(3-Fluorophenyl)-N-trans-3-[(3-hydroxypropyl)carbamoyl]cyclohexyl}nicotinamide

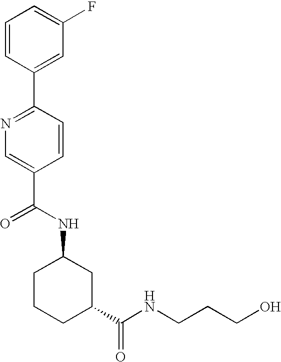

The title compound was prepared using general method (ii) (HBTU coupling) from trans-3-({[6-(3-fluorophenyl)pyridin-3-yl]carbonyl}amino)cyclohexanecarboxylic acid (Example 238A) and 3-amino-propan-1-ol. The product was purified by HPLC method (B). LCMS method (A): RT 2.77 min (100%) ES+m/z 400.19 [M+1].

### EXAMPLE 243

### 6-(3-Fluorophenyl)-N-trans-3-(piperazin-1-ylcarbonyl)cyclohexyl]nicotinamide

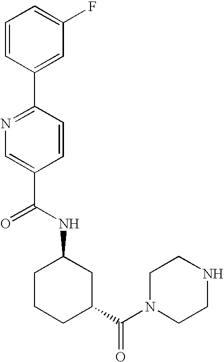

The title compound was prepared using analogous conditions to those described in Example 176 from trans-3-({[6-(3-fluorophenyl)pyridin-3-yl]carbonyl}amino)cyclohexanecarboxylic acid and piperazine-1-carboxylic acid tert-butyl ester. The product was purified by HPLC method (B). LCMS method (A): RT 2.31 min (100%) ES+m/z 411.21 [M+1].

### EXAMPLE 244A

### Tert-Butyl[tran-3-({[6-(3-fluorophenyl)pyridine-3-yl]carbonyl}amino)cyclohexyl]carbamate

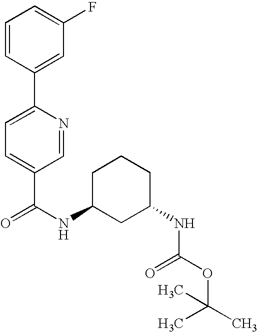

6-(3-Fluorophenyl)nicotinic acid (101 mg. 0.467 mmol) was dissolved in dimethylformamide (5 mL) along with HBTU (177 mg, 0.471 mmol), tert-butyl(trans-3-aminocyclohexyl)carbamate (Preparation 70, 100 mg, 0.467 mmol) and triethylamine (0.068 mL, 0.490 mmol) and the reaction mixture stirred at room temperature overnight. The reaction mixture was partitioned between ethyl acetate (10 mL) and water (10 mL) and the organic layer was separated, washed with saturated sodium carbonate solution, dried over magnesium sulphate and evaporated in vacuo to give the title compound as a white solid (173 mg). LRMS: [M+1] 414 AP⁺, 412 [M−1].

### EXAMPLE 244

### N-[trans-3-Aminocyclohexyl]-6-(3-fluorophenyl)nicotinamide

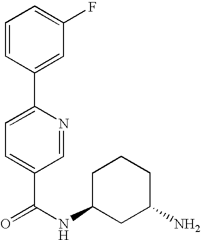

To a solution of tert-butyl-trans-3-({[6-(3-fluorophenyl)pyridine-3-yl]carbonyl}amino)cyclohexyl] carbamate (Example 244A, 173 mg, 0.593 mmol) in 1,4-dioxane (5 mL) was added a solution of 4M hydrogen chloride in 1,4-dioxane (0.504 mL, 2.02 mmol) and the reaction mixture was stirred at room temperature over the weekend. A further 1.08 mL of 4M hydrogen chloride in 1,4-dioxane was added and stirring was continued for 4 hours. The solvent was evaporated, the residue was dissolved in 4 M hydrogen chloride in 1,4-dioxane (2.02 mL, 8.06 mmol) and the reaction was stirred at room temperature overnight. The crude reaction mixture was partitioned between ethyl acetate (10 mL) and dilute sodium hydroxide solution (10 mL). The organic layer was re-washed with dilute sodium hydroxide solution (10 mL), dried over magnesium sulphate, filtered and evaporated to give a white solid (169 mg). A portion of the crude product (80 mg) was purified using HPLC Method (A) to give 19.1 mg of the title compound. LCMS Method (B): RT 2.93 min, 100% area, ES m/z [M+] 313.16.

### EXAMPLE 245

### 6-(3-Fluorophenyl)-N-[trans-3-glycoloylamino}cyclohexyl]nicotinamide

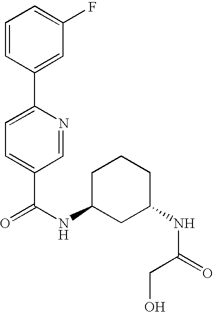

The title compound was prepared using analogous conditions to those described in Example 202. The crude product was purified using HPLC Method (A). LCMS Method (B): RT 2.61 min 100% area, ES m/z [M+] 371.16.

### EXAMPLE 246

### 6-(3-Fluorophenyl)-N-{trans-3-{[(2S)-2-hydroxypropanoyl]amino}cyclohexyl]nicotinamide

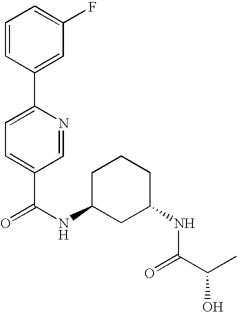

The title compound was prepared using analogous conditions to those described in Example 206. The crude product was purified using HPLC Method (A). LCMS Method (A): RT 2.78 min 100% area, ES m/z [M+] 385.18.

### EXAMPLE 247

### 6-(3-Fluorophenyl)-N-{trans-3-{[4-methylpiperazin-1-yl)acetyl]amino}cyclohexyl]nicotinamide

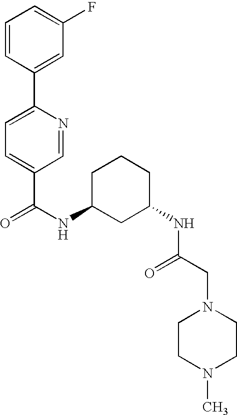

The title compound was prepared using analogous conditions to those described in Example 204. The crude product was purified using HPLC Method (A). LCMS Method (A): RT 2.20 min 100% area, ES m/z [M+] 453.25.

### EXAMPLE 248

### 6-(3-Fluorophenyl)-N-trans-[3-(2-hydroxy-2-methylpropionylamino)cyclohexyl]nicotinamide

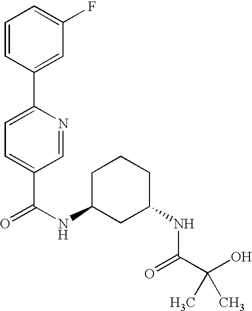

The title compound was prepared using analogous conditions to those described in Example 205. The crude product was purified by HPLC Method (A). LCMS Method (B): RT 2.75 min 100% area, ES m/z [M+] 399.20.

### EXAMPLE 249

### 6-(3-Fluoro-phenyl)-N-trans-(3-methanesulfonylamino-cyclohexyl)-nicotinamide

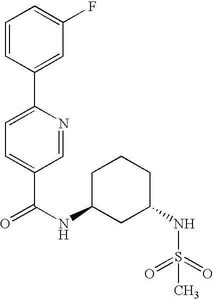

The title compound was prepared using analogous conditions to those described in Example 207. The crude product was purified by HPLC Method (A). LCMS Method (A): RT 2.98 min 100% area, [M+] 391.14.

### EXAMPLE 250

### N-[(1S*,3S*)-3-Aminocyclohexyl]-6-(3-fluorophenyl)nicotinamide

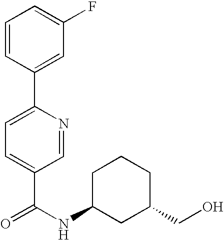

Triethylamine (0.057 mL, 0.406 mmol) was added to a solution of 6-(3-fluorophenyl)nicotinic acid (92.5 mg. 0.426 mmol), [1S*,3S*]-3-aminocyclohexyl]methanol (preparation 83)(50 mg, 0.387 mmol) and HBTU (154 mg, 0.406 mmol) in dimethylformamide 2.0 mL. The reaction was stirred at room temperature over night. The dimethyl formamide was evaporated off in vacuo and the resulting oil left to stand at room temperature over the weekend whereupon the oil started to crystallise. The oil was partitioned between dichloromethane (10 mL) and water (10 mL) and the organic layer separated. This was further washed with brine (10 mL) and then saturated potassium carbonate solution (10 mL), the organic layer was separated and evaporated in vacuo to give a colourless gum (129 mg) which crystallised on standing. 65 mg of this material was purified using reverse phase hplc Method (A) to give 25.6 mg of the title compound. LCMS Method (B) RT 2.97 min. 100% area ES m/z [M+] 328.16.

### EXAMPLE 251A

### 6-(3-Fluorophenyl)-N-cis-3-(hydroxymethyl)cyclohexyl]nicotinamide

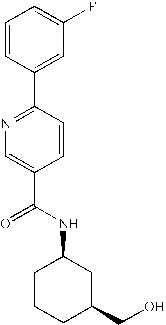

Lithium borohydride (2M, 11.2 ml, 22.4 mmol) was added to a solution of methyl cis-3-({[6-(3-fluorophenyl)pyridin-3-yl]carbonyl}amino) cyclohexane carboxylate (Example 133, 4.0 g, 11.2 mmol) in dry THF (100 ml) at room temperature and the reaction mixture was stirred at this temperature for 15 hours then heated under reflux for 3 hours. The reaction mixture was reduced in volume, cooled to 4° C. and diluted, first with (100 ml) and then by HCl (2N) until the pH of the aqueous was pH 2. The resulting mixture was stirred for 15 minutes. The pH was then adjusted to pH 9 with sodium carbonate and the mixture was extracted with ethyl acetate (2×150 ml). The combined organic phases were concentrated in vacuo to give a yellow solid which was purified by re-crystallisation from ethyl acetate to give the title compound (1.73 g). LCMS Method (G): RT 1.33 min (100%), ES+m/z 329 [M+1].

### EXAMPLE 251B

### cis-3-({[6-(3-Fluorophenyl)pyridin-3-yl]carbonyl}amino)cyclohexyl]methyl methanesulfonate

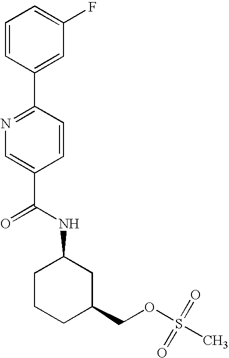

N,N-Diisopropylethyl amine (2.0 g 15.5 mmol) and methane sulphonic anhydride (1.5 g, 8.6 mmol) were added to a solution of 6-(3-fluorophenyl)-N-cis-3-(hydroxymethyl)cyclohexyl]nicotinamide (Example 251A, 1.7 g, 5.18 mmol) in DCM (40 ml) at room temperature and the reaction mixture was stirred for 17 hours. The reaction mixture was partitioned between water (300 ml) and EtOAc (300 ml) and the organic phase was separated, dried and concentrated in vacuo. The residue was trituated with a small volume of EtOAc to yield a white solid (1.7 g).

LCMS Method (G): RT 1.47 min (100%), ES+m/z 407 [M+1].

### EXAMPLE 251C

### N-cis-3-[(1,3-Dioxo-1,3-dihydro-2H-isoindol-2-yl)methyl]cyclohexyl}-6-(3-fluorophenyl)nicotinamide

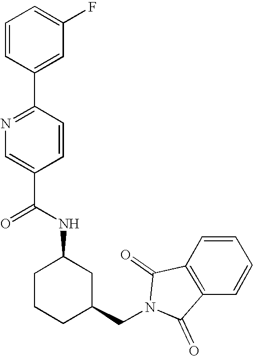

Potassium pthalimide (0.98 g, 6.7 mmol) was added to a solution of cis-3-({[6-(3-fluorophenyl)pyridin-3-yl]carbonyl}amino)cyclohexyl]methyl methanesulfonate (Example 251B, 1.13 g, 2.78 mmol) in NMP (22 ml) and the mixture was heated at 85° C. for 4 hours. The cooled reaction mixture was diluted with methanol (30 ml) and the solution was partitioned between water (100 ml) and EtOAc (100 ml). The organic phase was separated, washed with water (3×100 ml), dried and concentrated in vacuo. The residue was trituated with hot methanol and the resulting white solid (0.7 g) was isolated by filtration. MS (ES+): m/z 458 [M+1].

### EXAMPLE 251D

### N-cis-3-(Aminomethyl)cyclohexyl]-6-(3-fluorophenyl)nicotinamide

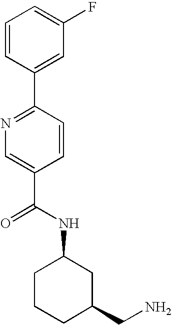

A suspension of N-cis-3-[(1,3-dioxo-1,3-dihydro-2H-isoindol-2-yl)methyl]cyclohexyl}-6-(3-fluorophenyl)nicotinamide (Example 251C, 0.7 g, 1.53 mmol) in ethanolic methylamine (33% in ethanol, 75 ml) was stirred at room temperature for 18 hours. The reaction mixture was concentrated and the resulting oil was re-dissolved in methanol (25 ml) and purified by chromatography on an Isolute®SCX-2 ion exchange column (20 g) eluting methanol (75 ml) then ammonia in methanol (2M, 300 ml) to give the product as a gum (400 mg). LCMS Method (G): RT 0.90 min (100%), ES+m/z 328 [M+1].

### EXAMPLE 251

### N-cis-3-(Acetamidomethyl)cyclohexyl]-6-(3-fluorophenyl)nicotinamide

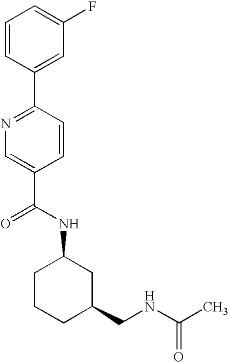

The title compound was prepared using analogous conditions to those described in Example 6 from N-cis-3-(aminomethyl)cyclohexyl]-6-(3-fluorophenyl)nicotinamide (Example 251D) and acetic acid. The crude product was purified by HPLC method (B). LCMS method (A): RT 2.92 min (100%), ES+m/z 370.18 [M+1].

### EXAMPLE 252

### 6-(3-Fluorophenyl)-N-cis-3-{[(methoxyacetyl)amino]methyl}cyclohexyl]nicotinamide

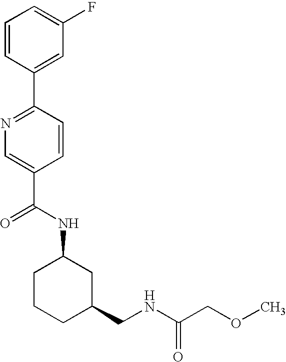

The title compound was prepared using analogous conditions to those described in Example 6 from N-cis-3-(aminomethyl)cyclohexyl]-6-(3-fluorophenyl)nicotinamide (Example 251D) and methoxyacetic acid. The crude product was purified by HPLC method (A). LCMS method (A): RT 2.84 min (100%), ES+m/z 400.19 [M+1].

### EXAMPLE 253

### N-cis-3-{[(N,N-Dimethylglycyl)amino]methyl}cyclohexyl]-6-(3-fluorophenyl)nicotinamide

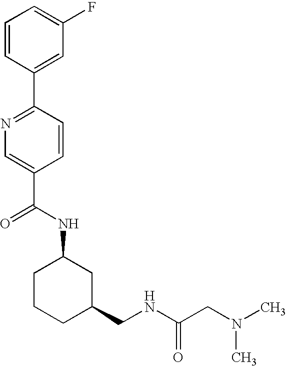

The title compound was prepared using analogous conditions to those described in example 6 from N-cis-3-(aminomethyl)cyclohexyl]-6-(3-fluorophenyl)nicotinamide (Example 251D) and N,N-dimethyl glycine. The crude product was purified by HPLC method (B). LCMS method (A): RT 2.33 min (100%), ES+m/z 413.19 [M+1].

### EXAMPLE 255

### 6-(3-Fluorophenyl)-N-cis-3-({[(1-methyl-1H-pyrazol-5-yl)carbonyl]amino}methyl)cyclohexyl]nicotinamide

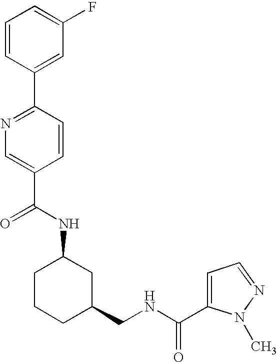

The title compound was prepared using analogous conditions to those described in Example 6 from N-cis-3-(aminomethyl)cyclohexyl]-6-(3-fluorophenyl)nicotinamide (Example 251D) and 1-methyl-1H-pyrazole-5-carboxylic acid. The crude product was purified by HPLC method (B). LCMS method (A): RT 3.06 min (100%), ES+m/z 436.27 [M+1].

### EXAMPLE 256

### 6-(3-Fluorophenyl)-N-{(1S,3R)-3-[(3-hydroxyazetidin-1-yl)methyl]cyclohexyl}nicotinamide

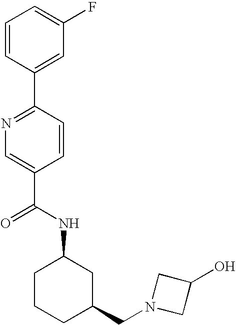

3-Azetidinol (20.2 mg, 0.184 mmol), triethylamine (100 mg, 1.0 mmol) and water (0.2 ml) were added to a solution of 6-(3-fluorophenyl)-N-cis-3-formylcyclohexyl]nicotinamide (Example 251D, 52.2 mg, 0.16 mmol) in methanol (2 ml) and the mixture was stirred for 10 minutes. Sodium triacetoxyborohydride (67.8 mg, 0.320 mmol) was added and the reaction mixture was stirred at room temperature for 17 hours. The reaction mixture was quenched by the addition of hydrochloric acid (2N, 1.0 ml) and, after stirring for 5 minutes, the pH was adjusted to 10 with 5% sodium carbonate solution. The resulting mixture was partitioned between DCM (7 ml) and water (7 ml) and the organic phase was separated, dried and concentrated in vacuo to give a gum. The crude product was purified by HPLC method (B). LCMS method (B): RT 2.90 min (100%), ES+m/z 384.21 [M+1].

### EXAMPLE 257

### N-cis-3-{[(1-Acetylazetidin-3-yl)amino]methyl}cyclohexyl]-6-(3-fluorophenyl)nicotinamide

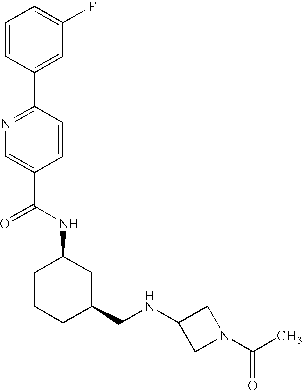

The title compound was prepared using analogous conditions to those described in Example 256 from 6-(3-fluorophenyl)-N-cis-3-formylcyclohexyl]nicotinamide (Example 251D) and 1-(3-amino-azetidin-1-yl)-ethanone. The crude product was purified by HPLC method (A). LCMS method (B): RT 2.77 min (100%), ES+m/z 425.22 [M+1].

### EXAMPLE 258

### N-cis-3-(Azetidin-1-ylmethyl)cyclohexyl]-6-(3-fluorophenyl)nicotinamide

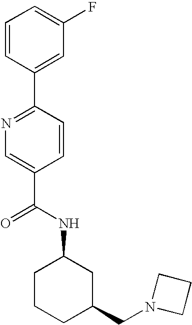

The title compound was prepared using analogous conditions to those described in Example 256 from 6-(3-fluorophenyl)-N-cis-3-formylcyclohexyl]nicotinamide (example 251D) and azetidine. The crude product was purified by HPLC method (B). LCMS method (B): RT 3.46 min (100%), ES+m/z 368.20 [M+1].

### EXAMPLE 259

### 6-(3-Fluorophenyl)-N-cis-3-[(L-prolylamino)methyl]cyclohexyl}nicotinamide

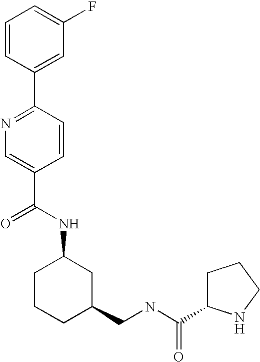

The title compound was prepared using analogous conditions to those described in Example 6 from N-cis-3-(aminomethyl)cyclohexyl]-6-(3-fluorophenyl)nicotinamide (Example 251D) and (2R)-1,2-pyrrolidinedicarboxylic acid, 1-(1,1-dimethylethyl) ester. The product was dissolved in 4M HCl in dioxane and the resulting solution was stirred for 6 hours. The solvent was evaporated to give a residue which was purified by HPLC method (B) to give 22.8 mg of the title compound. LCMS method (A): RT 2.23 min (100%), ES+m/z 425.27 [M+1].

### EXAMPLE 260

### 6-(3-Fluorophenyl)-N-{(1S,3R)-3-[(4-hydroxypiperidin-1-yl)carbonyl]cyclohexyl}nicotinamide

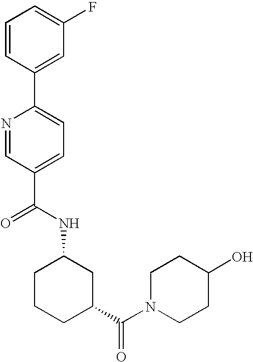

6-(3-Fluorophenyl)nicotinic acid (370 mg, 1.70 mmol) was dissolved in dimethylformamide (6.0 mL), 1,1-carbonyldiimidazole (332 mg, 2.04 mmol) was added and the reaction mixture was stirred at room temperature for 1.5 hours. 1-{[(1R,3S)-3-Aminocyclohexyl]carbonyl}piperidin-4-ol hydrochloride salt (Preparation 32, 498 mg, 1.70 mmol) was then added followed by N-ethyldiisopropylamine (0.594 mL, 3.41 mmol) and the reaction mixture was stirred at room temperature for 18 hours. The dimethylformamide was removed in vacuo and the residue was partitioned between water (15 ml) and ethyl acetate (50 mL). The organic layer was separated, washed with dilute sodium carbonate solution and evaporated to give the crude product which was triturated with ethyl acetate and recrystallised from ethanol (5 mL) to give the title compound as a white solid (390 mg).

LCMS Method (G): RT 1.24 min 100% area, [M+1] 426 obs, [M+1] 426.50 calculated.

¹H NMR (400 MHz, MeOD): δ ppm 1.39-1.58 (m, 6H) 1.70-1.75 (m, 1H) 1.82-2.03 (m, 5H) 2.87-2.93 (m, 1H) 3.11-3.15 (m, 1H) 3.32-3.35 (m, 1H) 3.84-3.89 (m, 2H) 3.99-4.05 (m, 2H) 7.19-7.23 (m, 1H) 7.49-7.55 (m, 1H) 7.81-7.89 (m, 2H) 7.97-7.99 (m, 1H) 8.27-8.29 (m, 1H) 9.05 (s, 1H).

### EXAMPLE 261A

### (4-{[6-Fluorophenyl)-pyridine-3-carbonyl]amino}cyclohexyl)acetic acid methyl ester

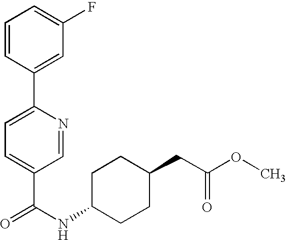

6-(3-Fluorophenyl)nicotinic acid (Preparation 1, 154 mg, 0.71 mmol), 1-hydroxybenzotriazole (118 mg, 0.771 mmol), EDC (148 mg, 0.77 mmol) and DIEA (0.68 mL, 4.14 mmol) were added to a solution of (4-aminocyclohexyl)acetic acid methyl ester hydrochloride (166 mg, 0.592 mmol) in DCM (3 ml) and the mixture was stirred at room temperature for 18 hours. The reaction mixture was partitioned between brine (10 mL) and DCM 1(0 mL) and the organic phase was separated, washed with brine (5×10 mL), dried over MgSO₄ and concentrated in vacuo. The crude product was washed with acetonitrile and filtered to give 218 mg of title compound as a white powder.

LRMS: m/z (ES+) [M+1] 371.

¹H NMR (400 MHz, CDCl₃): δ ppm 1.18 (m, 2H), 1.30 (m, 2H), 1/71 (m, 1H), 1.85 (m, 2H), 2.15 (m, 2H), 2.25 (d, 2H), 3.68 (s, 3H), 3.96 (m, 1H), 5.98 (d, 1H), 7.15 (m, 1H), 7.44 (m, 1H), 7.78 (m, 3H), 8.16 (m, 1H), 9.00 (m, 1H).

### EXAMPLE 261

### (4-{[6-Fluorophenyl)pyridine-3-carbonyl]amino}cyclohexyl)acetic acid

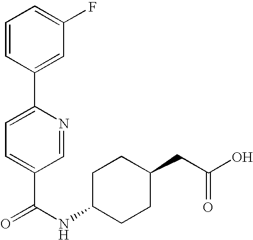

Lithium hydroxide (2M, 5.40 mL, 10.8 mmol) was added to a solution of (4-{[6-fluorophenyl)-pyridine-3-carbonyl]amino}cyclohexyl)acetic acid methyl ester (Example 261A, 80 mg, 0.22 mmol) in THF and the reaction was stirred for 2 hours at room temperature. The reaction mixture was acidified with 1M HCl_{(aq)} to pH 1-2 and extracted with DCM. The organic phase, on evaporation, gave 69 mg of the title compound as a white solid.

LRMS: m/z (ES+) [M+1] 357.

¹H NMR (400 MHz, DMSO-d₆): S ppm 1.06 (m, 2H), 1.34 (m, 2H), 1.62 (broad s, 1H), 1.75 (d, 2H), 1.86 (m, 2H), 2.11 (m, 2H), 3.73 (m, 1H), 7.30 (m, 1H), 7.55 (m, 1H), 7.96 (m, 2H), 8.11 (m, 1H), 8.25 (m, 1H), 8.45 (d, 1H), 9.05 (d, 1H), 12.08 (broad, 1H).

### EXAMPLE 261

### 6-(3-Fluorophenyl)-N-[trans 4-(3-methyl-[1,2,4]oxadiazol-5-ylmethyl)cyclohexyl]nicotinamide

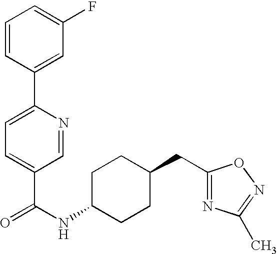

A solution of the product of Example 261B (69 mg, 0.19 mmol) in DMSO was treated with 1,1′-carbonyldiimidazole (47 mg, 0.291 mmol) and the reaction was stirred for 2 hours at room temperature. N-Hydroxyacetamidine (17 mg, 0.233 mmol) was then added and the reaction mixture was heated at 85° C. with stirring for 2 hours. The reaction temperature was then increased to 110° C. and stirring was continued for 72 hours. The crude product was purified by HPLC Method (A) giving 11 mg of the title compound. LCMS: RT 3.14 min, m/z 395 [M+1].

### EXAMPLE 262

### 6-(3-Fluorophenyl)-N-[1,3 cis-3-(2-hydroxy-2-methylpropyl)cyclohexyl]nicotinamide

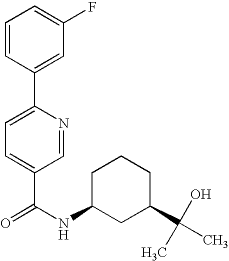

A solution of methyl magnesium chloride in THF (3M, 7.48 mL, 22.4 mmol) was added dropwise to a solution of the product of Example 133 (2.0 g, 5.61 mmol) in THF (20 mL) at 0° C. The reaction mixture was allowed to warm to room temperature and stirred for 18 hours. A further portion of methyl magnesium chloride (3M in THF, 1.87 mL, 5.6 mmol) was added followed by another (1.87 mL) after 1 hour and the reaction mixture was again left to stir for 18 hours. Methyl magnesium chloride (3M in THF, 1.87 mL, 5.6 mmol) was then added at hourly intervals for 5 hours and the reaction mixture was subsequently heated at 40° C. for 18 hours. The reaction mixture was cooled to room temperature, quenched by dropwise addition of water, concentrated in vacuo and extracted with ethyl acetate. Purification twice by silica chromatography eluting with a gradient of DCM to 98:2 DCM:MeOH by volume afforded the title compound as an off-white solid (60 mg). LCMS Method (G): RT 1.40 min, m/z (ES+) [M+1] 357.

### EXAMPLE 263

### N-{3-Cyano-3-[(4-methylpiperazin-1-yl)carbonyl]cyclohexyl}-6-(3-fluorophenyl)nicotinamide

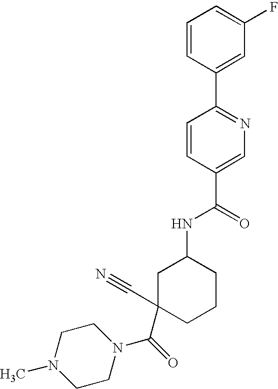

A suspension of 6-(3-fluorophenyl)nicotinic acid (61 mg, 0.28 mmol) in DCM (1 mL) was treated with DMF (1 mL). The solution was cooled to 0° C. and stirred vigorously as EDC (59 mg, 0.31 mmol) and HOAt (4 mg, 0.028 mmol) were added. Preparation 62 (70 mg, 0.28 mmol) was then added and the solution was stirred for 1 hour at 0° C. and then 48 hours at room temperature. The solvent was removed under reduced pressure and the residue was purified by flash chromatography on silica gel eluting with DCM:methanol 95:5 by volume. Once all the organic material had been removed using this solvent system the column was eluted with DCM:methanol 1:1 by volume, the product containing fractions were evaporated and the residue was stirred with diethylether (20 mL). The solid was filtered off and repurified by flash chromatography on silica gel eluting with DCM:methanol 9:1 y volume. The product containing fractions were evaporated and the residue was stirred with diethylether (10 mL) for 16 hours. The solid was filtered off and dried to give the title compound, 20 mg, as a grey solid. LCMS

Method (I): RT 3.13 minutes (56%) and 3.28 minutes (40%) area, ES m/z [M+1] 450.2.

### EXAMPLE 264

### N-{3-Cyano-3-[(4-ethylpiperazin-1-yl)carbonyl]cyclohexyl}-6-(3-fluorophenyl)nicotinamide

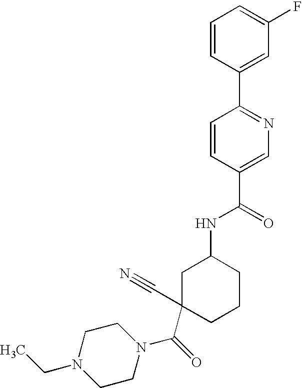

A mixture of 6-(3-Fluorophenyl)nicotinic acid (64 mg, 0.3 mmol), DMF (1 mL) and Preparation 64 (78 mg, 0.3 mmol) was cooled to 0° C. and DIPEA (38 mg, 0.295 mmol) and HBTU (0.145 g, 0.384 mmol) were added. The reaction mixture was stirred at 0° C. for 1 hour and then at room temperature for 16 hours. The solvent was removed under reduced pressure and the residue was stirred with di-isopropyl ether (20 mL) for 16 hours. The di-isopropyl ether was decanted and the remaining solid was purified by flash chromatography on silica gel eluting with DCM:methanol 96:4 by volume to give the title compound, 41 mg, as a yellow solid. LCMS Method (H): RT 2.70 minutes (88%) area, ES m/z [M+1] 464.2.

### EXAMPLE 265

### N-[3-Cyano-3-(morpholin-4-ylcarbonyl)cyclohexyl]-6-(3-fluorophenyl)nicotinamide

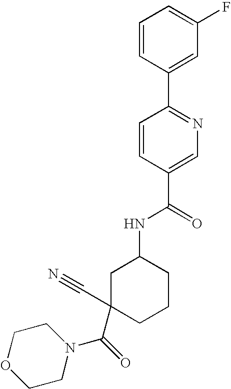

A suspension of 6-(3-fluorophenyl)nicotinic acid (69 mg, 0.316 mmol) in DMF (1 mL) was cooled to 0° C. and stirred vigorously as HBTU (156 mg, 0.411 mmol) was added. Preparation 66 (75 mg, 0.316 mmol) was then added and the solution was stirred for 1 hour at 0° C. and then 72 hours at room temperature. The solvent was removed and the residue was stirred with diisopropylether (20 mL) for 16 hours. The diisopropylether was decanted and the solid was stirred with DCM (20 mL). The combined DCM and diisopropylether portions were evaporated under reduced pressure and the residue was purified by flash chromatography on silica gel eluting with DCM:methanol 96:4 by volume. The product containing fractions were combined and evaporated to give a solid. The solid was stirred with DCM (20 mL), the remaining solid was filtered off and the mother liquor evaporated to give a yellow oil. The oil was repurified by flash chromatography on silica gel eluting with DCM:methanol 96:4 by volume. The product containing fractions were evaporated and the material was repurified by flash chromatography on silica gel eluting with ethyl acetate:n-heptane 2:1 by volume. The product containing fractions were evaporated to give the title compound (17 mg). LCMS Method (H): RT 3.26 minutes (97%) area, ES m/z [M+1].

### EXAMPLE 266

### N-[3-Cyano-3-(dimethylcarbamoyl)cyclohexyl]-6-(3-fluorophenyl)nicotinamide

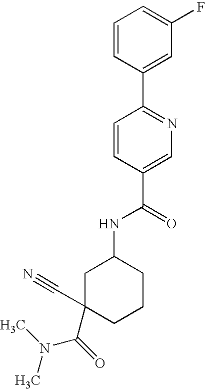

A suspension of 6-(3-fluorophenyl)nicotinic acid (125 mg, 0.574 mmol) in DMF (1 mL) was cooled to 0° C. and stirred vigorously as HBTU (239 mg, 0.631 mmol) was added. Example 68 (112 mg, 0.574 mmol) and DIPEA (74 mg, 0.574 mmol) were then added and the reaction mixture was stirred for 1 hour at 0° C. and then 72 hours at room temperature. The solvent was removed and the residue was stirred in diisopropylether (20 mL) for 16 hours. The diisopropylether was decanted and the solid was purified by flash chromatography on silica gel eluting with DCM:methanol 96:4 by volume. The product containing fractions were combined and evaporated to give a solid. The solid was stirred with diethylether (20 mL) for 6 hours, filtered off and dried to give the title compound (8 mg). LCMS Method (H): RT 3.15 minutes (93%) area, ES m/z [M+1] 395.2

### EXAMPLES 267

### N-cis-3-({[6-(3-Fluorophenyl)pyridine-3-yl]carbonyl}amino)cyclohexyl]morpholine-4-carboxamide

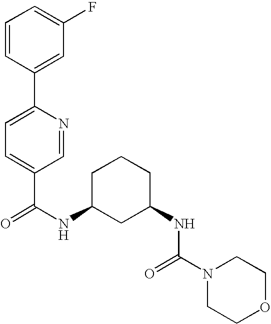

N-[cis-3-Aminocyclohexyl]-6-(3-fluorophenyl)nicotinamide (49.8 mg, 0.129 mmol, Example 200), N,N-diisopropylethylamine (0.045 mL, 0.258 mmol) and 1,1-carbonyldiimidazole (20.9 mg, 0.129 mmol) were stirred together with dimethylsulphoxide for 1.5 hours. Morpholine (0.017 mL, 0.194 mmol) was then added and the reaction mixture was stirred at room temperature overnight. The reaction mixture was purified by reverse phase HPLC Method (A) to give 11.7 mg of the title compound. LCMS Method (A): RT 2.76 min 100% area, ES m/z [M+] 426.21.

### EXAMPLE 268

### N-(cis-3-({[6-(3-Fluorophenyl)pyridine-3-yl]carbonyl}amino)cyclohexyl]-4-methylpiperazine-1-carboxamide

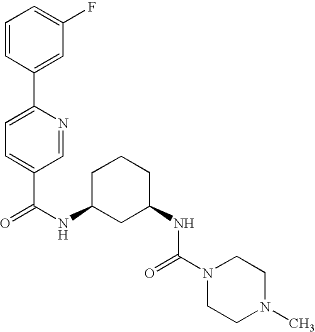

N-[cis-3-Aminocyclohexyl]-6-(3-fluorophenyl)nicotinamide (Example 200, 49.8 mg, 0.129 mmol), N,N-diisopropylethylamine (0.045 mL, 0.258 mmol) and 1,1-carbonyldiimidazole (20.9 mg, 0.129 mmol) were stirred together with dimethylsulphoxide for 1.5 hours. N-Methylpiperazine (0.021 mL, 0.194 mmol) was then added and the reaction mixture was stirred at room temperature for 60 hours. The crude product was purified HPLC Method (B). LCMS Method (B): RT 2.80 min 100% area, ES m/z [M+] 439.24.

### EXAMPLE 269

### N-[trans-3-({[6-(3-Fluorophenyl)pyridine-3-yl]carbonyl}amino)cyclohexyl]-4-methylpiperazine-1-carboxamide

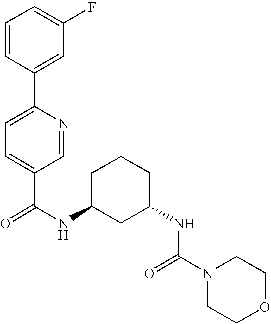

N-[trans-3-Aminocyclohexyl]-6-(3-fluorophenyl)nicotinamide (49.8 mg, 0.129 mmol, Example 244), N,N-diisopropylethylamine (0.045 mL, 0.258 mmol) and 1,1-carbonyldiimidazole (20.9 mg, 0.129 mmol) were stirred together with dimethylsulphoxide for 1.5 hours. Morpholine (0.017 mL, 0.194 mmol) was added and the reaction mixture was stirred at room temperature overnight. The reaction mixture was purified on reverse phase HPLC Method (A). LCMS Method (B): RT 2.85 min 100% area, ES m/z [M+] 426.21.

### EXAMPLE 270

### 6-(3-Fluorophenyl)-N-{trans-4-(methylcarbamoyl)amino}cyclohexyl}nicotinamide

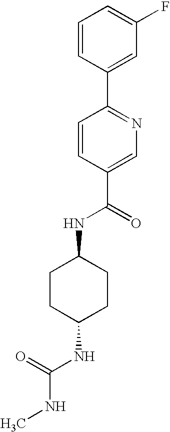

N-(trans-4-Aminocyclohexyl)-6-(3-fluorophenyl)nicotinamide (50 mg, 0.129 mmol, Example 142B) was dissolved in dimethylsulphoxide (1.0 mL). The resulting solution was treated with N,N-diisopropylamine (0.135 mL, 0.774 mmol) and 1,1′-carbonyldiimidazole (25.1 mg, 0.155 mmol) and the reaction mixture was stirred at room temperature for 1.5 hours. Methylamine hydrochloride (10.5 mg, 0.155 mmol) was then added along with further N,N-diisopropylethylamine (0.067 mL, 0.387 mmol) and the reaction mixture was stirred at room temperature for 2 hours, then heated to 50° C. for 18 hours. The crude product was purified using reverse phase HPLC Method (B) to give 11.5 mg of the title compound. LCMS Method (B): RT 2.69 min 100% area, ES m/z [M+] 370.18.

### EXAMPLE 271

### 6-N-{trans-4-[Dimethylcarbamoyl)amino]cyclohexyl}-6-(3-fluorophenyl)nicotinamide

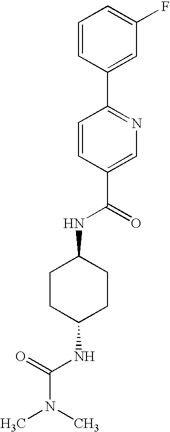

N-(trans-4-Aminocyclohexyl)-6-(3-fluorophenyl)nicotinamide (49.8 mg, 0.129 mmol, Example 142B) was dissolved in dimethylsulphoxide (1.0 mL). N,N-Diisopropylamine (0.135 mL, 0.774 mmol) was added followed by 1,1′-carbonyldiimidazole (25.1 mg, 0.155 mmol) and the reaction mixture was stirred at room temperature for 1.5 hours. Dimethylamine hydrochloride (12.6 mg, 0.155 mmol) was then added along with further N,N-diisopropylethylamine (0.067 mL, 0.387 mmol) and the reaction mixture was stirred at room temperature for 2 hours, then heated to 50° C. for 18 hours. The reaction was purified using reverse phase HPLC Method (A) to give 14.9 mg of the title compound. LCMS Method (A): RT 2.83 min 100% area, ES m/z [M+] 384.20.

### EXAMPLE 272

### 6-(3-Fluorophenyl)-N-{trans-4-[methyl(methylcarbamoyl)amino]cyclohexyl}nicotinamide

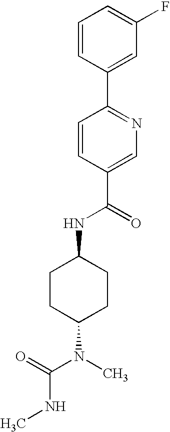

Methylamine hydrochloride (10.5 mg, 0.155 mmol), N,N-diisopropylethylamine (0.135 mL, 0.774 mmol) and 1,1-carbonyldiimidazole (25.1 mg, 0.155 mmol) were stirred together with dimethylsulphoxide (1 mL) for 1.5 hours. 6-(3-Fluorophenyl)-N-[trans-4-(methylamino)cyclohexyl]-nicotinamide (51.6 mg, 0.155 mmol, Example 94) and further N,N-diisopropylethylamine (0.067 mL, 0.387 mmol) were added and the reaction mixture was stirred at room temperature for 2 hours, then warmed to 50° C. for 18 hours. The crude product was purified using reverse phase HPLC Method (A) to give 23.6 mg of the title compound. LCMS Method (B): RT 2.76 min 100% area, ES m/z [M+] 384.20.

### EXAMPLE 273A

### Methyl [cis-4-({[6-(3-fluorophenyl)pyridine-3-yl]carbonyl}amino)cyclohexyl]acetate

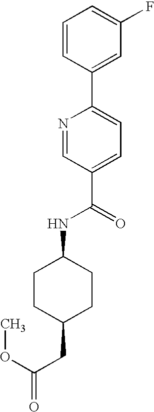

6-(3-Fluorophenyl)nicotinic acid (1056 mg. 4.81 mmol) was dissolved in dimethylformamide (5 mL), 1,1-carbonyl di-imidazole (898 mg, 5.54 mmol) was added and the reaction mixture was stirred at room temperature for 1.5 hours. Methyl (cis-4-aminocyclohexyl)acetate hydrochloride salt (1.0 g, 4.81 mmol) was then added followed N,N-diisopropylethylamine (1.26 mL, 7.22 mmol) and the reaction mixture was stirred at room temperature for 18 hours. The dimethylformamide was evaporated and the residue was partitioned between ethyl acetate and water. The organic layer was separated and evaporated to give a gum which crystallised on scratching. The gum was triturated with t-butylmethylether and the resulting was solid filtered off and dried to give the title compound (1.3 g).

LRMS: [M+1] 371 (obs), [M+1] 371.424 (calc).

¹H NMR (400 MHz, DMSO-d₆): δ ppm, 1.49-1.75 (m, 8H), 1.91-2.01 (m, 1H) 2.35-2.37 (m, 2H) 3.62 (s, 3H) 3.97-4.03 (m, 1H) 7.31-7.36 (m, 1H) 7.56-7.60 (m, 1H) 7.96-8.04 (m, 2H) 8.14-8.16 (m, 1H) 8.28-8.33 (m, 2H) 9.09 (s, 1H).

### EXAMPLE 273B

### [Cis-4-({[6-(3-Fluorophenyl)pyridine-3-yl]carbonyl}amino)cyclohexyl]acetic acid

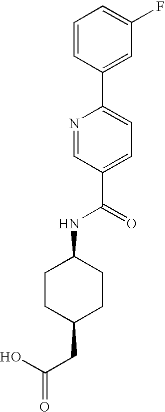

Aqueous sodium hydroxide (1M, 2.97 mL) was added to a suspension of methyl [cis-4-({[6-(3-fluorophenyl)pyridine-3-yl]carbonyl}amino)cyclohexyl]acetate (Example 273A) (1.00 g, 2.70 mmol) in methanol (10 mL) and the resulting mixture was stirred at room temperature for 3 h and then at 40° C. overnight. Approximately half of the methanol was evaporated and the residue was acidified to pH 2 with 2N hydrochloric acid. A gummy precipitate formed which started to crystallise on scratching. The gum and solid was broken up with a spatula until a white crystalline solid resulted. The white solid was then filtered off and dried in vacuo at 65° C. to give the title compound (0.95 g).

LRMS: [M+1] 357 (obs), [M+1] 356.397 (calc).

¹H NMR (400 MHz, DMSO-d₆): δ ppm 1.47-1.69 (m, 8H) 1.90-1.93 (m, 1H) 3.95-4.01 (m, 1H) 7.29-7.32 (m, 1H) 7.54-7.59 (m, 1H) 7.94-8.02 (m, 2H) 8.11-8.12 (m, 1H) 8.26-8.32 (m, 2H) 9.06-9.07 (m, 1H) 11.95 (broad s, 1H).

### EXAMPLE 273

### 6-(3-Fluorophenyl)-N-{cis-4-[(5-methyl-1,3,4-oxadiazolyl-2-yl)methyl]cyclohexyl}nicotinamide

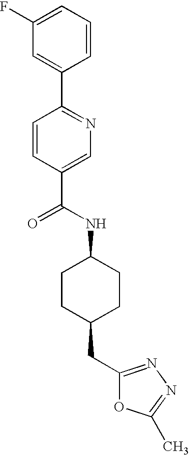

[Cis-4-({[6-(3-Fluorophenyl)pyridine-3-yl]carbonyl}amino)cyclohexyl]acetic acid (50.0 mg, 0.140 mmol, Example 273B) was dissolved in dimethylsulphoxide (0.5 mL), 1,1-carbonyldiimidazole (24.2 mg, 0.149 mmol) was added and the resulting mixture was stirred at room temperature for 1.5 hours. N-Hydroxyacetamidine (11.0 mg, 0.1479 mmol) was then added and the reaction mixture was stirred at room temperature for 60 hours. Further N-hydroxyacetamidine (6.00 mg, 0.081 mmol) was then added and the reaction mixture was heated at 85° C. overnight. The reaction was purified by reverse phase HPLC using Method (B) to give 26.6 mg of the title compound. LCMS Method (A): RT 3.34 min 100% area, ES m/z [M+] 394.18.

### EXAMPLE 274

### 6-(3-Fluorophenyl)-N-{cis-4-[2-(-methylpiperazin-1-yl)-2-oxoethyl]cyclohexyl}nicotinamide

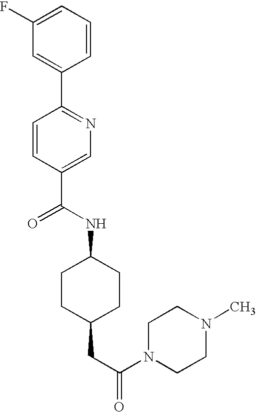

1,1-Carbonyldiimidazole (25.0 mg, 0.154 mmol) was added to a solution of [cis-4-({[6-(3-fluorophenyl)pyridine-3-yl]carbonyl}amino)cyclohexyl]acetic acid (50.0 mg, 0.140 mmol, Example 273B) in dimethylsulphoxide (0.75 mL) and the reaction mixture was stirred at room temperature for 1.5 hours. N-Methylpiperazine (0.016 mL, 0.147 mmol) was then added and the reaction mixture was stirred at room temperature overnight. The crude product was purified by reverse phase HPLC using Method (B) to give 35.6 mg of the title compound. LCMS Method (A): RT 2.23 min 100% area, ES m/z [M+] 438.24.

### EXAMPLE 275

### 6-(3-Fluorophenyl)-N-[cis-4-(1-hydroxy-1-methylethyl)cyclohexyl]nicotinamide

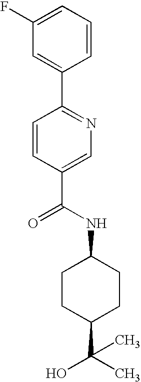

1,1-Carbonyldiimidazole (44.8 mg, 0.276 mmol) was added to a solution of 6-(3-fluorophenyl)nicotinic acid (50.0 mg, 0.230 mmol) in dimethylformamide (1.5 mL) and the reaction mixture was stirred at room temperature for 1.5 hours. 2-(cis-4-Aminocyclohexyl)propan-2-ol (36.2 mg, 0.230 mmol, Preparation 78) was then added and the reaction mixture was stirred at room temperature overnight. The reaction mixture was partitioned between water (3 mL) and ethyl acetate (5 mL) and the organic layer was evaporated in vacuo to give a gum. The crude product was purified by reverse phase HPLC using Method (A) to give 24.8 mg of the title compound. LCMS Method (A): RT 3.00 min 100% area, ES m/z [M+] 356.19.

### EXAMPLE 276

### Ethyl (1S,3R)-3-({[6-(3-fluorophenyl)pyridin-3yl]carbonyl}amino)cyclopentanecarboxylate

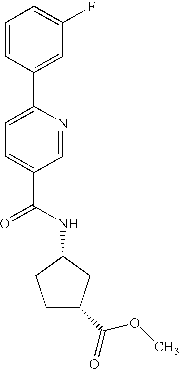

The title compound was prepared using analogous conditions to those described in Example 131 starting from methyl (1R,3S)-3-aminocyclopentane carboxylate.

¹H NMR (400 MHz, DMSO-d₆): δ ppm 1.62-1.69 (m, 1H) 1.79-1.97 (m, 4H) 2.19-2.27 (m, 1H) 2.83-2.89 (m, 1H) 3.61 (s, 3H) 4.25-4.32 (m, 1H) 7.23-7.27 (m, 1H) 7.54-7.59 (m, 1H) 7.92-7.97 (m, 1H) 7.98-8.01 (m, 1H) 8.10-8.14 (m, 1H) 8.26-8.30 (m, 1H) 8.57-8.61 (m, 1H) 9.08-9.09 (m, 1H).

### EXAMPLE 277

### 6-(3-Fluorophenyl)-N-cis-3-formylcyclohexyl]nicotinamide

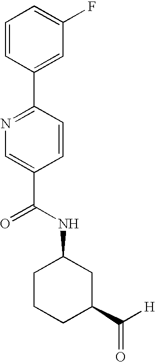

Dess-Martin periodinane (2.8 g, 6.6 mmol) was added to a solution of 6-(3-fluorophenyl)-N-cis-3-(hydroxymethyl)cyclohexyl]nicotinamide (Example 251A, 1.0 g, 3.05 mmol) in acetonitrile (150 ml) and the reaction mixture was stirred at room temperature for 48 hours. The reaction mixture was filtered, reduced in volume to 40 ml by evaporation and diluted with DCM (150 ml). The resulting solution was poured into saturated aqueous sodium bicarbonate (100 ml) and stirred rapidly. Aqueous sodium thiosulfate solution (5%, 50 ml) was added and the mixture was stirred for 10 minutes. The organic phase was separated, washed with sodium bicarbonate solution (50 ml) and water (50 ml), dried and concentrated in vacuo to give the title compound as a white solid (460 mg). LRMS (ES+): m/z 327 [M+1].

### EXAMPLE 278

### 6-(3-Fluorophenyl)-N-{(1R,3S)-3-[(4-hydroxypiperidin-1-yl)carbonyl]cyclohexyl}nicotinamide

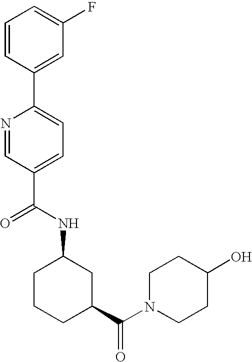

The title compound was prepared by separation of the mixture of enantiomers produced in Example 28 (440 mg). The following HPLC conditions were used:

| | |
|---|---|
| Column prep (250 * 21.2 mm id) | Chiralpak AD-H |
| Mobile phase: | MeOH/EtOH (50:50) |
| Flow rate (ml/min) | 15 |
| Detection (nm) | 225 nm and 254 nm |
| Temperature: | Ambient |
| Sample dissolution(mg/ml): | 440 mg in 4 ml MeOH = 110 mg/ml |
| Maximum injection volume (μl): | 200 ul |

This separation gave 118 mg of the title compound with retention time of 15.49 minutes and 98.5% ee in the analytical system described below and 160 mg of Example 260 which had a retention time of 15.51 minutes and 83.1% ee in the analytical system described below. The NMR and mass spectrum of the title compound were identical to Example 260.

### HPLC Analytical Conditions:

| | |
|---|---|
| Column analytical (250 * 4.6 mm id) | Chiralpak AD-H |
| Mobile phase: | MeOH/EtOH (50:50) |
| Flow rate (ml/min) | 1.0 |
| Detection (nm) | 225 nm and 254 nm |
| Temperature: | Ambient |
| injection volume (μl): | 20 ul |

### EXAMPLE 279

### N-{trans-4-[Cyclopropyl(hydroxy)methyl]cyclohexyl}-6-(3-fluorophenyl)nicotinamide

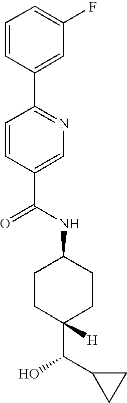

N′N′-Carbonyl-diimidazole (220 mg, 1.28 mmol) was added to a solution of 6-(3-fluorophenyl)nicotinic acid (231 mg, 1.10 mmol) in DMF (15 ml) at RT and the reaction mixture was stirred for 2 hours. Preparation 96 (0.18 g, 1.06 mmol) and triethylamine (0.215 g, 2.13 mmol) were then added and the mixture was stirred for 72 hours at room temperature. The reaction mixture was diluted with water (50 ml) and extracted with EtOAc (3×50 ml) and the combined organic phases were washed with brine (3×40 ml), dried over MgSO₄, and concentrated in vacuo. The residue was purified by flash chromatography on silica gel eluting with a gradient of heptane to heptane:ethyl acetate 20:80 by volume to give the title compound as a solid (98 mg). ¹H NMR and mass spectral data were identical to those obtained with the product of Example 212.

The following section describes the synthesis of intermediates which were used in the preparation of the foregoing examples.

### PREPARATION 1

### 6-(3-Fluorophenyl)nicotinic acid

3-Fluorophenylboronic acid (39.5 g, 0.282 mol), a solution of K₂CO₃ (150 g) in water (700 mL), [Bu₄N]Br (3.5 g, 0.0107 mol), and Pd(PPh₃)₄ (12.4 g, 0.0107 mol) were added to a solution of 6-chloronicotinic acid (37.0 g, 0.235 mol) in toluene. The reaction mixture was stirred under reflux for 20 hours. After cooling, the reaction mixture was filtered and acidified with 2 M HCl to pH 3. The resulting precipitate was separated by filtration and dried to give 6-(3-fluorophenyl)nicotinic acid (49.9 g).

¹H NMR (400 MHz, DMSO-d₆) δ ppm 7.29 (td, J=8.46, 2.42 Hz, 1H) 7.50-7.56 (m, 1H) 7.93 (dd, J=10.47, 2.15 Hz, 1H) 7.97 (d, J=7.79 Hz, 1H) 8.11 (d, J=8.06 Hz, 1H) 8.30 (dd, J=8.32, 2.15 Hz, 1H) 9.11 (d, J=1.88 Hz, 1H), 13.48 (bs, 1H).

### PREPARATION 2

### 5-Chloro-6-(3-fluorophenyl)nicotinic acid

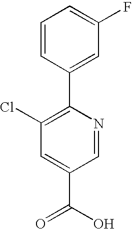

To a round bottom flask was added 5,6-dichloronicotinic acid (500 mg, 2.60 mmol), 3-fluorophenylboronic acid (364 mg, 2.60 mmol), DMF (25 mL), 2M Cs₂CO₃ (6 mL) and Pd(PPh₃)₄ (30.1 mg, 0.026 mmol). The reaction mixture was heated to 90° C. for 3 h and then allowed to cool to room temperature. The mixture was diluted with ethyl acetate/water and the layers were separated. The organic layer was washed with brine, dried (MgSO₄) evaporated to give a solid, which was purified by chromatography (silica, DCM/MeOH) to give the desired product, 5-chloro-6-(3-fluorophenyl)nicotinic acid (623 mg, 95%).

LRMS: observed 252 [M+H], calculated 252.02 [M+H].

### PREPARATION 3

### 6-(3,5-Difluorophenyl)-nicotinic acid

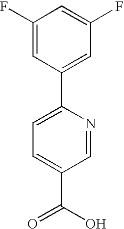

Step A: Preparation of tert-butyl 6-bromonicotinate To a round bottom flask containing a solution of 2-bromo-5-pyridinecarboxylic acid (10.0 g, 49 mmol) in DCM (500 mL) were added oxalyl bromide (7.4 mL) and 5 drops of DMF. After some gas evolution, the reaction mixture was stirred at reflux for approximately 6 hours, then cooled to room temperature, diluted with heptane (100 mL) and concentrated. The mixture was then suspended in THF (400 mL) and cooled to 0° C. t-BuOK (5.8 g, 52 mmol) was added and the reaction was allowed to warm to room temperature and stirred for 2 hours. The mixture was poured into EtOAc, washed with 1 N NaOH, water and brine, dried over MgSO₄, filtered and concentrated. The residue was purified by silica gel chromatography (Biotage 40S, Heptane:EtOAc 0-80%, 3 L) to afford the title compound 4.2 g (36%) as a white solid. ¹H NMR (400 MHz, DMSO-d₆) δ ppm 8.78-8.86 (1H, m), 8.14 (1H, dd, J=8.4, 2.4 Hz), 7.81 (1H, d, J=8.4 Hz), 1.56 (9H, s).

Step B: Preparation of tert-butyl 6-(3,5-difluorophenyl)nicotinate To a round-bottom flask was added 3,5-difluoro phenylboronic acid (1.84 g, 11.6 mmol), palladium tetrakis(triphenylphosphine) (89.5 mg, 0.08 mmol) and tert-butyl 6-bromonicotinate (2.0 g, 7.75 mmol) and the mixture was evacuated 3 times with nitrogen. The solids were dissolved in DMF (50 mL), followed by addition of 2M cesium carbonate (11 mL). The resulting mixture was heated to ˜90° C. until no starting bromide material was apparent by HPLC. The mixture was cooled to room temperature and then poured into a separating funnel, followed by addition of EtOAc and water (1×200 mL). The layers were separated and the organic extract was washed with brine (1×200 mL), dried over MgSO₄, filtered and concentrated to afford an orange oil. The crude mixture was purified by silica gel column chromatography (Biotage, 2-10% EtOAc in Heptane, approximately 2.5 L) to afford the title compound (2.1 g, 93%) as a white solid. ¹H NMR (400 MHz, DMSO-d₆) δ ppm 9.10-9.14 (1H, m), 8.29-8.35 (1H, m), 8.20-8.25 (1H, m), 7.90 (2H, dd, J=9.0, 1.5 Hz), 7.42 (1H, s), 1.59 (9H, s).

Step C: Preparation of 6-(3,5-difluoro-phenyl)-nicotinic acid To tert-butyl 6-(3,5-difluorophenyl)nicotinate in DCM (80 mL) was added trifluoroacetic acid (20 mL). After stirring at room temperature overnight, toluene was added (100 mL) and the solvent was removed to give the crude product as a white powder. The solid was re-crystallized from MeOH to afford the title compound 1.269 g (74%) as a white solid. ¹H NMR (400 MHz, DMSO-d₆) δ ppm 9.16 (1H, d, J=1.7 Hz), 8.37 (1H, dd, J=8.2, 2.0 Hz), 8.23 (1H, d, J=8.2 Hz), 7.86-7.95 (2H, m), 7.36-7.47 (1H, m).

### PREPARATION 5

### N-trans-(4-aminocyclohexyl)-2,2,2-trifluoro-N-methyl-acetamide hydrochloride

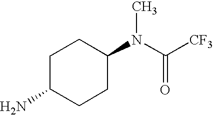

4M HCl in dioxan (15 mL) was added to the compound of Preparation 6 (324 mg, 1.0 mmol) and the solution was stirred at room temperature for 3 hours after which time a white precipitate had formed. The reaction mixture was evaporated to give 255 mg of the title compound as the hydrochloride salt. ¹H NMR (400 MHz, DMSO-d₆) δ ppm 1.42-1.51 (m, 2H) 1.61-1.87 (m, 4H) 1.99-2.09 (m, 2H) 2.88, 2.97 (2 singlets, together 3H) 2.99 (m, 1H) 3.54-4.62, 4.07-4.13 (multiplets, together 1H) 8.02-8.13 (s broad, 3H).

### PREPARATION 6

### tert-Butyl{trans-4-[methyl(trifluoroacetyl)amino]-cyclohexyl}carbamate

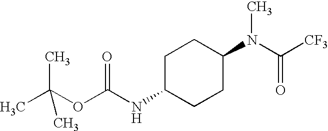

A solution of tert-butyl{trans-4-[(trifluoroacetylamino)]-cyclohexyl}carbamate (2.05 g, 6.61 mmol, prepared using the method described in WO-A-2000/055162) was dissolved in dimethylformamide (25 mL) by warming to 50° C. The solution was cooled to room temperature, caesium carbonate (3.23 g, 9.91 mmol) was added, and methyl-paratoluene sulphonate (1.48 g, 7.93 mmol) was then added in portions. The reaction was heated to 75° C. for 72 hours. Further caesium carbonate (815 mg, 2.5 mmol) and further methyl-paratoluene sulphonate (372 mg, 2 mmol) were added. After a further 17 hours at 75° C., the reaction was cooled to room temperature, concentrated in vacuo and partitioned between ethyl acetate (150 mL) and water (150 mL). The aqueous layer was adjusted to pH 7 with 2 molar aqueous hydrochloric acid and the mixture was re-partitioned. The combined organic layers were washed with water (2×150 mL), dried over MgSO₄, filtered and concentrated in vacuo. The residue was triturated with ether and the resulting white solid was filtered to give 1.4 g of the title compound. ¹H NMR (400 MHz, DMSO-d₆) a ppm 1.21-1.30 (m, 2H) 1.39 (s, 9H) 1.57-0.67 (m, 3H) 1.75-1.90 (m, 3H) 2.87+2.97 (2 singlets, together 3H) 3.23-3.36, 3.58-3.64, 4.00-4.10 (3 multiplets, together 2H). 6.74-6.76 (m, 1H).

### PREPARATION 7

### tert-Butyl-{trans-4-[2-(benzyloxyethoxy)cyclohexyl}carbamate

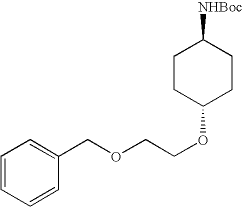

trans-(4-Hydroxy-cyclohexyl)-carbamic acid tert-butyl ester (200 mg, 0.929 mmol) was dissolved in dimethylacetamide (2 mL), sodium hydride (60% dispersion in oil, 37.2 mg, 0.929 mmol) was added and the reaction was stirred at room temperature for 30 minutes. [(2-Bromoethoxymethyl]benzene (147 μL, 0.9 mmol) was added. The reaction mixture was stirred at room temperature for 60 hours and then partitioned between ethyl acetate and dilute aqueous sodium hydrogen carbonate solution. The organic layer was separated and evaporated to give 300 mg of the title compound. LRMS (ES): observed 250 (loss of BOC group), [M+1] calc 350.2 [M+1].

### PREPARATION 8

### trans-4-[2-(Benzyloxy)ethoxy]cyclohexylamine, hydrochloride salt

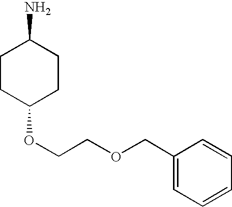

A solution of hydrogen chloride in 1,4-dioxane (4 M), 0.858 mL, was added to a solution of the compound of Preparation 7 (300 mg, 0.858 mmol) in dichloromethane (1 mL). The reaction was stirred at room temperature for 18 hours. The reaction was evaporated to dryness to give 100 mg of the title compound.

LRMS: observed APCI-250 [M+1], calculated 250.2 [M+1].

### PREPARATION 9

### N-{trans-4-[(2-Benzyloxy)ethyoxy]cyclohexyl}-6-(3-fluorophenyl)nicotinamide

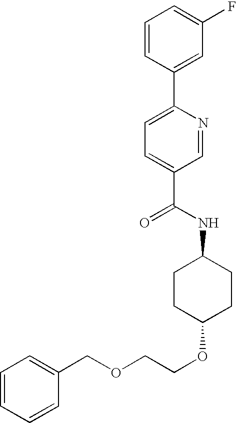

The title compound was prepared according to the general amide coupling conditions using HBTU with trans-4-[2-(benzyloxy)ethoxy]cyclohexylamine hydrochloride salt (Preparation 8). LRMS (ES): observed 449 [M+1], calculated 449.2 [M+1].

### PREPARATION 12

### Benzyl (2R)-4-{[trans-4-({[6-(3-fluorophenyl)pyridine-3-yl]carbonyl}amino)cyclohexyl]carbonyl}-2-methylpiperazine-1-carboxylate

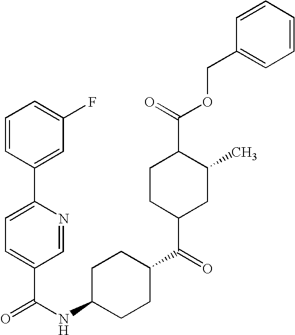

The title compound was prepared using the method of Example 138 starting from benzyl (2R)-2-methylpiperazine-1-carboxylate.

LRMS (ES): observed 560 [M+1], calculated 559.65 [M+1].

### PREPARATION 13

### Benzyl (2S)-4-{[trans-4-({[6-(3-fluorophenyl)pyridine-3-yl]carbonyl}amino)cyclohexyl]carbonyl}-2-methylpiperazine-1-carboxylate

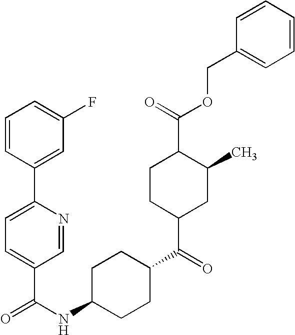

The title compound was prepared using the method of Preparation 12, starting from benzyl (2S)-2-methylpiperazine-1-carboxylate.

LRMS (ES): observed 560 [M+1], calculated 559.65 [M+1].

### PREPARATION 14

### Methyl cis-4-[(tert-butoxycarbonyl)amino]-1-methylcyclohexanecarboxylate (A) and methyl trans-4-[(tert-butoxycarbonyl)amino]-1-methylcyclohexanecarboxylate (B)

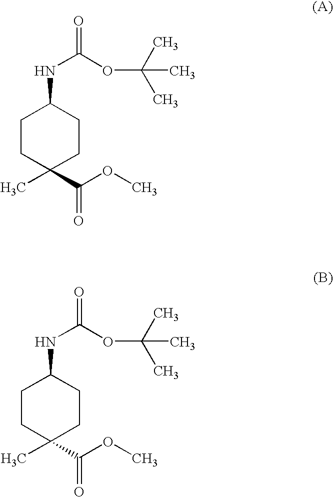

Diisopropylamine (2.92 g, 9.65 mmol) was dissolved in tetrahydrofuran (8 mL) and cooled to 0° C. n-Butyllithium (3.9 mL of a 2.5M solution in hexane, 9.65 mmol) was added slowly. The reaction was stirred at 0° C. for 15 min and then cooled to −78° C. Methyl 4-[(tert-butoxycarbonyl)amino]cyclohexanecarboxylate (1.08 g, 4.2 mmol, prepared as described in Heterocycles, 471-504, 58, 2002) dissolved in tetrahydrofuran (2 mL) was added over 5 min. The temperature was then allowed to rise to −30° C. and the reaction was stirred for 30 min. The thick suspension was diluted with dimethoxyethane (5 ml) and stirred for another 45 min at −60° C. Methyl iodide (596 mg, 4.20 mmol) was then added and the solution was stirred for 1 hour at −60° C. The reaction was then quenched by the addition of an aqueous solution of citric acid (10% w/w, 20 ml). The solution was extracted twice with ethyl acetate (40 mL) and dichloromethane (20 mL). The organic phases were combined and dried over anhydrous MgSO₄. The solvents were removed under reduced pressure and the residue was purified by chromatography on silica eluting with a mixture of heptane:ethyl acetate 100:0, 80:20 and 70:30.

Methyl cis-4-[(tert-butoxycarbonyl)amino]-1-methylcyclohexanecarboxylate (A) (245 mg) was collected first as a colourless oil. ¹H NMR (400 MHz CDCl₃) δ ppm 1.07-1.28 (m, 7H), 1.35-1.48 (m, 9H), 1.82-1.91 (m, 2H), 2.12-2.21 (m, 2H), 3.33-3.44 (m, 1H), 3.66 (s, 3H), 4.28-4.39 (m, 1H).

Methyl trans-4-[(tert-butoxycarbonyl)amino]-1-methylcyclohexanecarboxylate (B) was then collected as a 3:2 mixture with starting material (50 mg).

### PREPARATION 15

### Methyl cis-4-amino-1-methylcyclohexanecarboxylate hydrochloride salt.

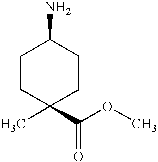

Methyl cis-4-[(tert-butoxycarbonyl)amino]-1-methylcyclohexanecarboxylate (250 mg, 0.92 mmol) was dissolved in a solution of 4N HCl in 1,4-dioxane (10 ml) and the reaction mixture was stirred at room temperature for 3 hours. The solvent was removed under reduced pressure to give a colourless solid (200 mg). ¹H NMR (400 MHz, DMSO-d₆) δ ppm 1.05 (s, 3H), 1.15-1.34 (m, 4H), 1.76-1.86 (m, 2H), 2.01-2.12 (m, 2H), 2.95 (s, 1H), 3.63 (s, 3H), 7.90 (bs, 3H).

### PREPARATION 16

### Methyl trans-4-amino-1-methylcyclohexanecarboxylate hydrochloride salt

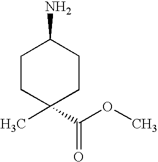

A mixture of methyl trans-4-[(tert-butoxycarbonyl)amino]-1-methylcyclohexanecarboxylate and methyl 4-[(tert-butoxycarbonyl)amino]cyclohexanecarboxylate (50 mg, 0.18 mmol) was dissolved in a solution of 4N HCl in 1,4-dioxane (10 mL) and the reaction mixture stirred at room temperature for 3 hours. The solvent was removed under reduced pressure to give a colourless solid (41 mg).

### PREPARATION 18

### Tert-Butyl [4-(4,5-dihydro-1H-imidazol-2-yl)cyclohexyl]carbamate

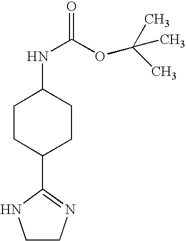

tert-Butyl (4-formylcyclohexyl)carbamate (850 mg, 3.74 mmol) was dissolved in tert-butanol (20 mL) and ethylenediamine (247 mg, 4.11 mmol) was added. The reaction mixture was stirred at room temperature under nitrogen for 30 minutes and then potassium carbonate (1.55 g, 11.2 mmol) and iodine (1.19 g, 4.68 mmol) were added. The reaction mixture was stirred at 70° C. for 3 hours, whereupon the reaction had changed from dark brown to light yellow. The reaction was quenched with 5% w/w aqueous sodium metabisulphite solution (20 mL) and then extracted using dichloromethane (50 mL). The aqueous phase was extracted again with dichloromethane (20 mL). The organic layers were combined, washed with saturated aqueous sodium hydrogen carbonate (10 mL), dried over anhydrous MgSO₄, filtered and evaporated to give the product as a yellow gum (800 mg).

LRMS (ES): observed 268, calculated 268.2 [M+1].

### PREPARATION 19

### tert-Butyl [4-(1H-imidazol-2-yl)cyclohexyl]carbamate

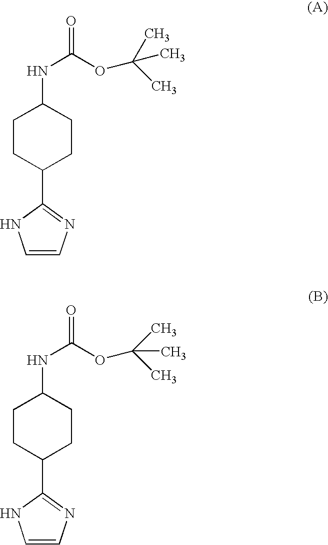

tert-Butyl [4-(4,5-dihydro-1H-imidazol-2-yl)cyclohexyl]carbamate (800 mg, 2.99 mmol) was added to a suspension of diacetoxyiodobenzene (1.06 g, 3.29 mmol) and potassium carbonate (454 mg, 2.99 mmol) in DMSO (5 mL). The reaction mixture was stirred at room temperature overnight. The reaction mixture was diluted with saturated aqueous sodium hydrogen carbonate (20 mL) and ethyl acetate (20 mL) and stirred for 5 min. The organic layer was separated and the aqueous phase was extracted again with ethyl acetate (20 mL). The organic layers were combined, dried with anhydrous MgSO₄, evaporated under reduced pressure and purified by chromatography on silica using dichloromethane to dichloromethane:methanol:0.88 aqueous ammonia 70:30:3 to give a higher running product, assigned as (A) (70 mg) and a lower running spot assigned as (B) (40 mg).

For compound assigned as (A)—LRMS (ES): observed 264 (M−1), calculated 264.18 [M−1].

For compound assigned as (B)—LRMS (ES): observed 264 (M−1), calculated 264.18 [M−1].

### PREPARATION 20

### cis and trans isomers of 4-(1H-Imidazol-2-yl)cyclohexylamine

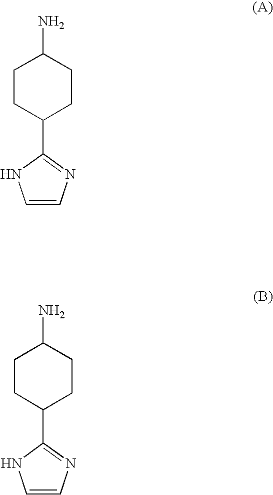

tert-Butyl [4-(1H-imidazol-2-yl)cyclohexyl]carbamate diastereomer (A) (70 mg, 0.26 mmol) (Preparation 19) was dissolved in a 4M solution of hydrogen chloride in dioxane (10 mL). The reaction mixture was stirred at room temperature for 2 hours. The solvent was then removed under reduced pressure. The residue was dissolved in methanol and eluted through an SCX-2 cartridge with firstly methanol and then a 0.5M solution of ammonia in methanol. The solvent was evaporated to give the title compound (29 mg) as a brown gum.

LRMS (APCI): observed 166 (M+1), calculated 166.24 [M+1].

tert-Butyl [4-(1H-imidazol-2-yl)cyclohexyl]carbamate diastereomer (B) (40 mg, 0.15 mmol) (Preparation 19) was dissolved in a 4M solution of hydrogen chloride in dioxane (10 mL). The reaction mixture was stirred at room temperature for 2 hours. The solvent was then removed under reduced pressure. The residue was dissolved in methanol and eluted through an SCX-2 cartridge with firstly methanol and then a 0.5M solution of ammonia in methanol. The solvent was evaporated to give the title compound (27 mg) as a brown gum.

LRMS (APCI): observed 166 (M+1), calculated 166.24 [M+1].

### PREPARATION 21

### N-1,4-Dioxaspiro[4.5]dec-8-yl-6-(3-fluorophenyl)nicotinamide

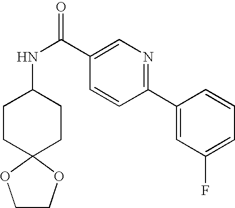

6-(3-Fluorophenyl)nicotinic acid (746 mg, 3.44 mmol) and 1,4-dioxaspiro[4.5]decan-8-amine (540 mg, 3.44 mmol) were dissolved in DMF (5 mL). Triethylamine (1.73 g, 17.2 mmol) and HBTU (1.63 g, 4.29 mmol) were added. The reaction was stirred at 50° C. for 16 hours. The solvent was removed under reduced pressure and the residue was partitioned between dichloromethane (10 mL) and semi-saturated aqueous sodium hydrogen carbonate solution (5 mL). The dichloromethane layer was filtered through a phase separation tube and evaporated under reduced pressure. The residue was purified by chromatography on silica eluting with heptane:ethyl acetate 90:10 to 0:100. This gave the title compound as a solid (1.45 g).

LRMS (ES): observed 357 [M+1], calculated 357.15 [M+1].

¹H NMR (400 MHz CDCl₆) δ ppm 1.58-1.87 (m, 6H), 2.02-2.13 (m, 2H), 3.95 (s, 4H), 4.04-4.16 (m, 1H), 6.03-6.12 (m, 1H), 7.09-7.19 (m, 1H), 7.39-7.48 (m, 1H), 7.72-7.83 (m, 3H), 8.09-8.20 (m, 1H), 8.95-9.01 (m, 1H).

### PREPARATION 23

### cis-3-(Dibenzylamino)-N,N-dimethylcyclobutanecarboxamide

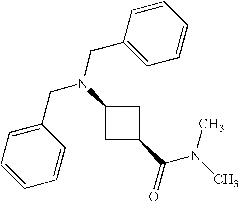

N,N-Dimethyl-3-oxocyclobutanecarboxamide (7.33 g, 51 mmol) and dibenzylamine (10.95 mL, 56.9 mmol) were stirred together in dichloroethane (200 mL) for 1 hour. Sodium triacetoxyborohydride (15.3 g, 72.4 mmol) and acetic acid (2.96 g, 51 mmol) were then added and the reaction mixture was stirred at room temperature for 5 days. The reaction was quenched with sodium bicarbonate solution and extracted with dichloromethane. The organic layer was separated, dried over anhydrous Na₂SO₄, filtered and evaporated to give 16.7 g of residue. This was purified on silica eluting with heptane:ethyl acetate 50:50, then ethyl acetate 100% to give (15.9 g) of the title compound.

### PREPARATION 24

### cis-N,N-Dibenzyl-3-[(dimethylamino)methyl]cyclobutanamine

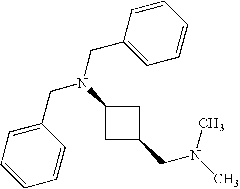

To an ice-cooled solution of the compound of Preparation 23 (15.9 g, 48.9 mmol) in THF was added a solution of lithium aluminium hydride (1 M in THF, 48.9 mL) dropwise. After complete addition, the reaction was warmed to room temperature and stirred for 1 hour. The reaction was cooled in ice and quenched by the sequential dropwise addition of water (0.88 ml), 15% sodium hydroxide aqueous (0.88 mL) and water (2.64 mL). The reaction was stirred for 1 hour and filtered through Celite®. The filter pad was washed with ethyl acetate and the filtrate was evaporated. The residue was columned on silica eluting with 2-methyltetrahydrofuran containing methanol (3%) and aqueous ammonia (5%), followed by 2-methyltetrahydrofuran containing methanol (20%) and aqueous ammonia (20%). This gave 14.2 g of the title compound.

### PREPARATION 25

### cis-3-[(Dimethylamino)methyl]cyclobutanamine

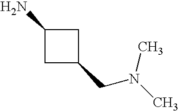

A solution of the compound of Preparation 24 (1.35 g) in methanol (90 mL) was hydrogenated using standard conditions for debenzylation at 50° C. After evaporation of the solvent, a solution of hydrogen chloride in dioxane (4M, 4 mL) was added to form the hydrochloride salt. After evaporation this gave 940 mg of the title compound as a yellow solid.

LRMS: observed 129 [M+1], calculated 129.13 [M+1].

¹H NMR (400 MHz MeOD-d₄) δ ppm 2.05-2.09 (m, 2H) 2.58-2.64 (m, 3H) 2.86 (s, 6H) 3.27-3.32 (m, 3H) 3.72-3.78 (m, 1H).

### PREPARATION 26

### 1-Isopropyl-1,5,6,7-tetrahydro-4H-indazol-4-one

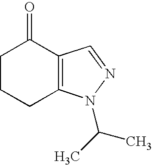

2-[(Dimethylamino)methylene]cyclohexane-1.3-dione (3 g, 17.9 mmol), isopropyl hydrazine (1.98 g, 17.9 mmol) and sodium hydroxide (718 mg, 17.9 mmol) were mixed in methanol (50 mL) at 0° C. The reaction mixture was allowed to warm to room temperature and stirred overnight. The solvent was evaporated and the residue was purified on silica eluting with ethyl acetate/methanol solvent mixtures to give 2.2 g of the title product.

LRMS: observed [M]+178, calculated 178.2 [M]+.

### PREPARATION 27

### 1-Isopropyl-1,5,6,7-tetrahydro-4H-indazol-4-one oxime

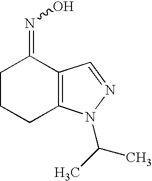

To a solution of the compound of Preparation 26 (1.63 g, 9.15 mmol) in THF (20 mL) and ethanol (20 mL) were added hydroxylamine hydrochloride (3.18 g, 45.7 mmol) and sodium acetate (3.75 g, 45.7 mmol). The reaction was heated under reflux for 6 hours, allowed to cool and evaporated. The residue was diluted with water and extracted with ethyl acetate. The organic extracts were washed with brine, dried over anhydrous Na₂SO₄, filtered and concentrated to give 1.0 g of the title compound as a solid.

LRMS (ES): observed 194 [M+1], calculated 193.1.

### PREPARATION 28

### 1-Isopropyl-1,5,6,7-tetrahydro-4H-indazol-4-ylamine

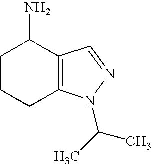

To a solution of lithium aluminium hydride (216 mg, 5.69 mmol) in THF (20 mL) was added the compound of Preparation 27 (1.1 g, 5.69 mmol). After complete addition, the reaction mixture was refluxed overnight and then allowed to cool to room temperature. The reaction mixture was diluted with water and 2.5 M aqueous sodium hydroxide solution. The mixture was stirred for 30 min and then filtered through Celite® and the filter pad was washed with ethyl acetate. The filtrate was dried over anhydrous Na₂SO₄, filtered and evaporated to give 0.98 g of the title compound which was used without further purification.

### PREPARATION 29

### 1-Ethyl-4,5,6,7-tetrahydro-1H-benzoimidazol-5-yl)-(4-methoxy-benzyl)-amine

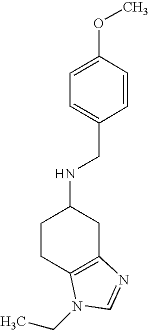

1-Ethyl-1,4,6,7-tetrahydrobenzoimidazolone (50 mg, 0.3 mmol) and p-methoxybenzylamine (63 mg, 0.456 mmol) were mixed with dichloromethane (1 mL). To this was added acetic acid (27 mg, 0.456 mmol) and sodium triacetoxyborahydride (96 mg, 0.456 mmol). The above reagents were stirred for 18 hours. The reaction was basified to pH 8-9 with saturated aqueous sodium bicarbonate, diluted with dichloromethane (10 mL) and passed through a phase separator and the organic phase was evaporated. The crude product was purified using chromatography on silica eluting with a mixture of dichloromethane:methanol:aqueous ammonia 100:0:0 to 90:10:1. The product fractions were combined and evaporated to give the desired product as an oil (72 mg).

LRMS (ES+): observed 286 [M+1], calculated 286.39 [M+1].

¹H NMR (400 MHz MeOD-d₄) δ ppm 0.84-0.94 (m, 3H) 1.67-1.81 (m, 1H) 2.15-2.25 (m, 1H) 2.39-2.48 (m, 1H) 2.50-2.61 (m, 1H) 2.64-2.74 (m, 1H) 2.86-2.95 (m, 1H) 3.00-3.09 (m, 1H) 3.77-3.93 (m, 8H) 6.87-6.93 (m, 2H) 7.28-7.34 (m, 2H) 7.47-7.51 (m, 1H).

### PREPARATION 30

### 1-Ethyl-4,5,6,7-tetrahydro-1H-benzoimidazol-5-ylamine

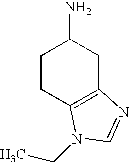

A mixture of the compound of Preparation 29 (72 mg, 0.25 mmol), ethanol (3 mL), 20% palladium hydroxide on carbon (42 mg, 0.302 mmol) and ammonium formate (159 mg, 2.52 mmol) was heated at reflux for 1 hour under nitrogen. The reaction mixture was allowed to cool to room temperature and filtered through arbocel. The filtrate was concentrated in vacuo to give a yellow oil which was purified using chromatography on silica eluting with a mixture of dichloromethane:methanol:aqueous ammonia 100:0:0 to 90:10:1. The product fractions were combined and evaporated to give the desired product as a yellow oil (28 mg).

LRMS (ES): observed 166 [M+1], calculated 166.24 [M+1].

¹H NMR (400 MHz CDCl₃) δ ppm 1.33-1.41 (m, 3H) 1.66-1.80 (m, 1H) 1.97-2.08 (m, 1H) 2.37-2.47 (m, 1H) 2.49-2.66 (m, 2H) 2.86-2.95 (m, 1H) 3.20-3.30 (m, 1H) 3.78-3.87 (m, 2H) 7.33-7.39 (m, 1H).

### PREPARATION 31

### tert-Butyl{(1S,3R)-3-[(4-hydroxypiperidin-1-yl)carbonyl]cyclohexyl]carbamate

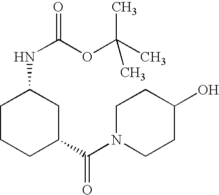

(1R,3S)-3-[(tert-Butoxycarbonyl)amino]cyclohexanecarboxylic acid (500 mg, 2.06 mmol) was dissolved in dimethylformamide (2.0 mL), 1,1-carbonyldiimidazole (433 mg, 2.67 mmol) was added and the reaction mixture was stirred at room temperature for 1.5 hours, whereupon a precipitate formed. 4-Hydroxypiperidine (270 mg, 2.67 mmol) was then added and the reaction mixture was stirred at room temperature for 2 hours. The reaction mixture was partitioned between water (10 mL) and ethyl acetate (20 mL) and the organic layer was separated and evaporated to give the title compound as a white foam (671 mg).

LRMS: (AP+) [M+1] 327 obs, [M+1] 327.43 calc.

¹H NMR (400 MHz, CDCl₃): δ ppm 1.08-1.12 (m, 1H) 1.40-1.49 (m, 13H) 1.69-1.70 (m, 1H) 1.79-1.89 (m, 3H) 1.96-2.00 (m, 3H) 2.58-2.64 (m, 1H) 3.15-3.28 (m, 2H) 3.44-3.54 (m, 2H) 3.74-3.83 (m, 1H) 3.93-3.97 (m, 1H) 4.05-4.11 (m, 1H) 4.44-4.51 (m, 1H).

### PREPARATION 32

### 1-{[(1R,3S)-3-Aminocyclohexyl]carbonyl}piperidin-4-ol hydrochloride salt

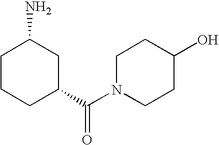

To a solution of tert-butyl{(1S,3R)-3-[(4-hydroxypiperidin-1-yl)carbonyl]cyclohexyl]carbamate (670 mg, 1.80 mmol) in dichloromethane (10 mL) was added a solution of 4M hydrogen chloride in 1,4-dioxane (6.93 mL) and the reaction mixture was stirred at room temperature overnight. The solvent was evaporated in vacuo and the residue was dissolved in dichloromethane and the solvent was re-evaporated to give the title compound as a foam (500 mg).

¹H NMR (400 MHz, DMSO-d₆): δ ppm 1.12-1.51 (m, 6H) 1.61-1.75 (m, 4H) 1.86-1.97 (m, 2H) 2.74-2.78 (m, 1H) 2.94-3.08 (m, 2H) 3.19-3.25 (m, 1H) 3.64-3.74 (m, 3H) 3.87-3.92 (m, 1H) 8.12 (bs, 3H).

### PREPARATION 33

### (1R)-(4-Aminocyclohexyl)cyclopropylmethanol

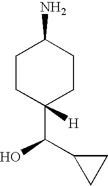

Step (a): To a mixture of trans-4-aminocyclohexanecarboxylic acid (18.0 g, 130 mmol) and potassium carbonate (52.3 g, 378 mmol) in acetonitrile (314 mL) was added benzyl bromide (45.6 mL, 383 mmol). The reaction mixture was stirred at 90° C. for 24 hours, quenched with water (200 mL) and extracted with ethyl acetate (2×200 mL). The combined organic phases were dried with MgSO₄ and concentrated in vacuo to give 46 g of 4-dibenzylaminocyclohexanecarboxylic acid benzyl ester as a white solid.

Step (b): O,N-Dimethylhydroxylamine HCl (4.86 g, 36.3 mmol) was dissolved in THF (50 mL) and the resulting solution was cooled to −10° C. A solution of isopropyl magnesium chloride in THF (2M, 36.3 mL, 72.5 mmol) was added dropwise over 30 minutes followed by the product of step (a) (3.0 g, 7.25 mmol) as a solution in THF (40 mL). The reaction mixture was stirred at −10° C. for 2 hours and then warmed to room temperature and stirred for 18 hours. The reaction was quenched by dropwise addition of saturated aqueous NH₄Cl and then partitioned between ethyl acetate (200 mL) and aqueous NH₄Cl. The organic phase was dried over MgSO₄ and evaporated in vacuo to give 3.42 g of a pale yellow oil. The crude product was combined with an earlier batch (2.02 g crude) and purified by silica column chromatography eluting with a gradient of heptane to heptane:ethyl acetate 3:1 by volume to give the product (4.16 g) as a colourless oil which began to convert to a waxy solid on standing.

LRMS: m/z (ES+) [M+1] 367.

¹H NMR (400 MHz, CDCl3): δ ppm 1.38-1.47 (m, 4H); 1.82-1.86 (m, 2H); 1.94-1.99 (m, 2H); 2.55-2.63 (m, 2H); 3.15 (s, 3H); 3.63 (s, 4H); 3.68 (s, 3H); 7.18-7.22 (m, 2H); 7.25-7.29 (m, 4H); 7.35-7.37 (m, 4H).

Step (c): A solution of cyclopropyl bromide (3.61 mL, 45.3 mmol) in THF (60 ml) was cooled to −78° C. A solution of tert-butyl lithium in pentane (1.7M, 26.6 mL, 45.3 mmol) was added at −78° C. and the mixture was stirred at −78° C. for 30 minutes. The product from step (b) (4.15 g, 11.32 mmol) was then added as a solution in THF (30 mL). The reaction mixture was allowed to warm to room temperature and left to stir for 18 hours. The reaction was quenched by the addition of saturated aqueous NH₄Cl (150 mL) and extracted with ethyl acetate (150 mL). The organic phase was washed with brine (100 mL), dried over MgSO₄ and evapourated in vacuo to give 3.938 g of cyclopropyl-(4-dibenzylaminocyclohexyl)-methanone as an orange gum.

LC-MS (6 min): 348−MH+, 1.97 mins; 100% ELSD.

Step (d): A solution of the product from step (c) (1.50 g, 4.317 mmol) and (R)-2-methyl-CBS-oxazaborolidine (1.26 g, 4.53 mmol) in toluene (30 mL) was cooled to −78° C. A solution of borane in THF (1M, 4.53 mL, 4.53 mmol) was added dropwise over 10 minutes. The reaction mixture was allowed to warm to room temperature over 18 hours, quenched by the addition of MeOH and stirred for 4 hours. Solvents were removed in vacuo to give a cloudy pale yellow gum which was taken up in 70 ml of 10% MeOH in ethyl acetate to give a cloudy solution which was washed with 40 ml of 0.880 aqueous ammonia. The aqueous layer was washed with further ethyl acetate (30 mL) and the combined organic phases were dried over MgSO₄ and concentrated in vacuo to give 2.57 g of a pale yellow gum. The crude product was purified by silica chromatography eluting with DCM then 98/2/0.2 and finally 90/10/1 DCM/MeOH/NH₃ by volume to give 1.132 g of (1R)-cyclopropyl-(trans-4-dibenzylaminocyclohexyl)methanol as a colourless gum. Chiral HPLC (reverse phase) showed the material to have a 92% enantiomeric excess in favour of the required enantiomer.

LCMS: (preAP3): RT1.85 mins, m/z [M+1]349.

Step (e): To a solution of the product of step (d) (0815 g, 2.33 mmol) in ethanol (20 mL) was added palladium hydroxide (98 mg; 0.70 mmol) and ammonium formate (1.47 g; 23.3 mmol) under an atmosphere of nitrogen. The resulting suspension was heated under reflux for 2 hours. The reaction mixture was cooled to room temperature and through Arbocel under a stream of nitrogen. The eluant was loaded onto an SCX-2 cartridge and eluted with ethanol (50 mL) and then 2M methanolic NH₃ solution (60 mL) to yield, after evaporation in vacuo, the title compound (373 mg) as a pale yellow gum.

¹H NMR (400 MHz, CDCl₃): δ ppm 0.20-0.28 (m, 2H); 0.45-0.60 (m, 2H); 0.88-0.97 (m, 1H); 1.05-1.23 (m, 4H); 1.42-1.50 (m, 1H); 1.86-1.98 (m, 4H); 2.58-2.66 (m, 2H).

### PREPARATION 34

### (4-Pyrrolidin-1-yl-cyclohexyl)-carbamic acid tert-butyl ester

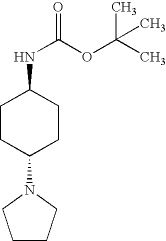

(4-Amino-cyclohexyl)-carbamic acid tert-butyl ester (4.9 g, 23 mmol) and sodium hydrogencarbonate (5.8 g) were added to toluene followed by 1,4-dibromobutane (5.0 g, 23 mmol). The heterogenous mixture was then heated at reflux with a Dean-Stark trap to remove water, under a nitrogen atmosphere, for 18 hours. The mixture was cooled to room temperature, filtered and evaporated. The crude residue was dissolved in ethyl acetate, loaded onto a pad of silica (120 g) and eluted with ethyl acetate (˜300 ml) and then 90/10/2 ethyl acetate/MeOH/0.880 ammonia (400 ml). The eluate was evaporated to give the title compound as an amorphous solid (5.1 g). LRMS: m/z [M+1] 269.

### PREPARATION 35

### 4-Pyrrolidin-1-yl-cyclohexylamine

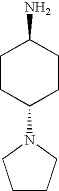

The title compound (502 mg) was prepared in an analogous manner to Example 44 starting from 800 mg of the product of Preparation 34.

### PREPARATION 36

### Dibenzyl-(1-oxa-spiro[2.5]oct-6-yl)-amine

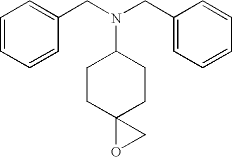

Sodium hydride (2.121 g, 53.0 mmol) and trimethylsulfoxonium iodide (11.21 g, 50.9 mmol) were stirred in dimethylsulphoxide (100 ml) at room temperature for 1 hour. A solution of 4-(dibenzylamino)cyclohexanone (12.45 g, 42.4 mmol) in 50 ml dimethylsulfoxide was then added dropwise and stirring was continued for 1 hour. Ethyl acetate (200 ml) and water (100 ml) were added and the phases were separated. The organic layer was washed with water and brine, dried over Na₂SO₄ and evaporated to dryness. This yielded 12.96 g of a light orange oil, which crystallized on standing and was purified by flash column chromatography on silica, eluting with a gradient of heptane:ethyl acetate 95:5 to 85:15 by volume, to yield 4.213 g of the title compound as a white solid. LRMS: m/z 307 [M+].

### PREPARATIONS 37 AND 38

### 4-Dibenzylamino-1-methoxymethyl-cyclohexanol and PEB4 (4-Dibenzylamino-1-methoxy-cyclohexyl)-methanol

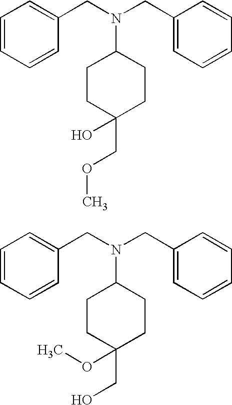

A solution of 5.51 g (17.9 mmol) of the product of Preparation 36 in MeOH (55 ml) was treated with concentrated H₂SO₄ (478 μl) and heated at reflux for 3 hours. The reaction mixture was diluted with 100 ml of water and basified by addition of 50 ml saturated aqueous NaHCO₃. A white suspension was formed. The suspension was extracted with ethyl acetate (2×200 ml) and the combined organic phases were washed with brine (150 ml), dried over Na₂SO₄ and concentrated in vacuo to give the crude product as a clear oil. The crude product was purified by flash column chromatography on silica eluting with a gradient of heptane:ethyl acetate 75:25 to 30:70 by volume. Product-containing fractions were combined and to give 2 products:

4-Dibenzylamino-1-methoxymethyl-cyclohexanol (Preparation 37): 1.832 g as a white solid

LCMS Method (X): RT 1.50 min, [M+1] 340.2.

¹H NMR (DMSO-d₆, 400 MHz): δ 1.20 (m, 2H), 1.51 (m, 4), 1.71 (m, 2H), 2.33 (m, 1H), 3.02 (s, 2H), 3.20 (s, 3H), 3.59 (s, 4H), 4.58 (s, 1H), 7.21 (m, 2H), 7.30 (m, 4H) 7.36 (m, 4H).

(4-Dibenzylamino-1-methoxy-cyclohexyl)-methanol (Preparation 38): 0.934 g as a colourless oil that solidified on standing.

LCMS Method (X): RT 1.46 min, [M+1] 340.2.

¹H NMR (DMSO-d₆, 400 MHz): δ 1.09 (m, 2H), 1.41 (m, 2H), 1.69 (m, 2H), 1.84 (m, 2H), 2.47 (m, 2H), 3.06 (s, 3H), 3.48 (m, 2H), 4.37 (m, 2H), 7.20 (m, 2H), 7.31 (m, 8H).

### PREPARATION 39

### (4-Amino-1-methoxy-cyclohexyl)-methanol

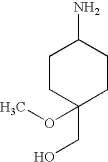

The material from Preparation 37 (175 mg) was treated as per Preparation 53 to deliver 89 mg of the title compound.

### PREPARATION 40

### 6-(3-Fluorophenyl)-N-[4-(hydroxymethyl)-4-methoxycyclohexyl]nicotinamide

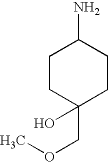

The material from Preparation 38 (0.934 g) was treated as per Preparation 53 to deliver the title compound.

### PREPARATION 41

### 4-Dibenzylamino-1-isopropoxymethyl-cyclohexanol

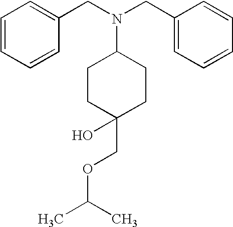

A lump of sodium (115 mg) was added to 2-propanol (5.0 ml) and the mixture was stirred until all the sodium had reacted. Then, 0.6 g of the product of Preparation 36 was added. The reaction was stirred at 50° C. for 2 hours and overnight at ambient temperature. The reaction mixture was partitioned between water (25 ml) and EtOAc (25 ml) and the aqueous phase was extracted again with EtOAc (25 ml). The combined organic phases were washed with brine (25 ml), dried over Na₂SO₄ and evapourated to give the crude product as a pale solid. The crude product was purified by flash column chromatography on silica using gradient elution with heptane:EtOAc 95:5 to 75:25 by volume to give the desired product as a white solid (351 mg). LCMS Method (X): RT 1.58 min, [M+1] 368.

### PREPARATION 41A

### 4-Amino-1-isopropoxymethyl-cyclohexanol

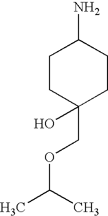

The product of Preparation 41 (345 mg, 0.939 mmol) was treated in a similar manner to Preparation 53 to deliver 164 mg of the title compound which was used crude without further characterization.

### PREPARATION 42

### 1-Methyl-3-oxo-cyclohexanecarbonitrile

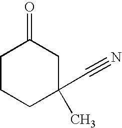

3-Methylcyclohexenone (10 g) was heated at 105° C. with KCN (1.2 equivalents) and NH₄Cl (1.2 equivalents) in 15% H₂O/DMF (100 ml) for 16 hours. The resulting mixture was cooled to ambient temperature and concentrated in vacuo. Water (100 ml) was added and the resulting mixture was extracted twice with DCM (75 ml). The combined organic extracts were washed with water (25 ml) and brine (2×30 ml) and dried over Na₂SO₄. The solvent was removed in vacuo to give a brown oil which was purified by flash column chromatography on silica gel to give the title compound (2.78 g) of an orange oil. GCMS: [M+] 137.

### PREPARATION 43

### 1-Methyl-3-oxo-cyclohexanecarboxylic acid ethyl ester

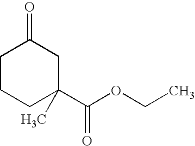

Acetyl chloride (15.4 ml) was added dropwise to 30 ml EtOH with cooling. The product of Preparation 42 was added as a solution in EtOH (15 ml) and the reaction mixture was heated to 68° C. for 72 hours. The reaction mixture was cooled to ambient temperature and the solids were removed by filtration. The filtrate was concentrated in vacuo, co-evaporated twice with toluene (30 ml) and DCM (20 ml) to give 3.92 g of a brown oil. The product was purified by flash chromatography of silica gel eluting with 2:1 Hept-EtOAc. 1.72 g of product was obtained as a yellow oil. GCMS: [M+] 184.

### PREPARATION 44

### Ethyl 3-Benzylamino-1-methylcyclohexanecarboxylate

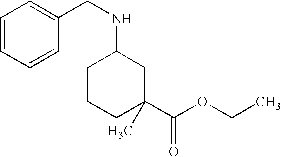

Sodium triacetoxyborohydride (1.5 eq) was added portionwise to a solution of the product of Preparation 43 and benzylamine in DCM (15 ml). The resulting mixture was stirred at 20° C. for 16 hours. The reaction mixture was quenched with saturated aqueous NaHCO₃ until the pH was basic. The layers were separated and the aqueous layer was extracted with DCM (20 mL). The combined organic layers were dried over sodium sulfate, filtered and evapourated in vacuo to yield the product (2.334 g). LCMS Method (Y): [M+1] 276.2.

### PREPARATION 45

### Ethyl 3-amino-1-methylcyclohexanecarboxylate

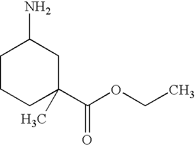

The title compound was prepared in a manner analogous to the method of Preparation 53 from 2.3 g of the product of Preparation 44. The title compound (1.71 g) was obtained as a colourless oil. GCMS: [M+] 185.

### PREPARATION 47

### 4-Dibenzylamino-1-methoxy-cyclohexanecarbaldehyde

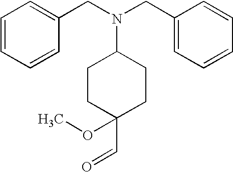

The title compound was prepared in a manner analogous to the method of Preparation 51 using 0.656 g of (4-dibenzylamino-1-methoxy-cyclohexyl)methanol from Preparation 39. The title compound (523 mg) was obtained as a colourless oil. LCMS Method (X): RT 1.55 min, [M+1] 338.

### PREPARATION 48

### 1-(4-Dibenzylamino-1-methoxy-cyclohexyl)-ethanol

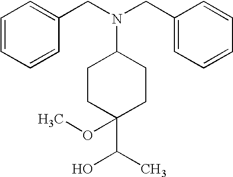

The title compound was prepared in a manner analogous to the method of Preparation 52 from 0.47 g of the product of Preparation 47. The title compound (0.388 g) was obtained as a white solid. LCMS Method (H): RT 1.46 min, m/z 338 [M+1].

### PREPARATION 48A

### 1-(4-Amino-1-methoxy-cyclohexyl)-ethanol

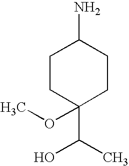

The product of Preparation 48 (380 mg, 1.08 mmol) was treated as per Preparation 53 to deliver 187 mg of the title compound which was used crude without further characterization.

### PREPARATION 49

### Benzyl (1R,3S)-3-(dibenzylamino)cyclohexanecarboxylate

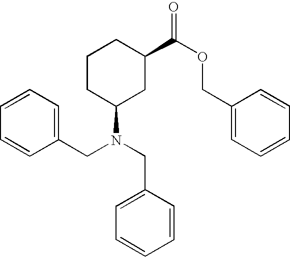

Potassium carbonate (14.48 g, 105 mmol) was added to a vigourously stirred suspension of cis-3-aminocyclohexanecarboxylic acid (5 g, 34.9 mmol) in acetonitrile (50 mL) followed by benzyl bromide (14.6 mL, 122 mmol). The suspension was stirred at room temperature for 16 hours. The reaction mixture was filtered and the solvent was removed under reduced pressure. The residue was diluted with heptane (60 mL) and stirred at 0° C. for 1 hour. The resulting suspension was filtered and redissolved into heptane (60 mL) and stirred at room temperature for 72 hours. The remaining solid was dried to give the title compound (8.64 g) as a solid.

LCMS Method (X): RT 3.14 minutes (100%) area, ES m/z [M+1] 414.2.

### PREPARATION 50

### [(cis)-3-(Dibenzylamino)cyclohexyl]methanol

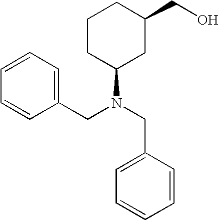

The product of Preparation 49 (4 g, 9.67 mmol) was dissolved in dry THF (40 mL) and the solution was put under an atmosphere of nitrogen gas and cooled to 0° C. A 2.4 M solution of lithium aluminium hydride in hexanes (8 ml, 19.3 mmol) was then added slowly to the stirred solution. The reaction mixture was stirred at room temperature for 30 minutes then cooled to 0° C. Sodium sulphate decahydrate was added until effervescence ceased. An extra 50 mL of THF was added and the suspension was stirred for 5 minutes. The suspension was filtered through Celite® and the filter cake was washed with DCM (2×15 mL). The combined organic eluants were evaporated and the residue was purified by flash chromatography on silica gel eluting with a mixture of ethyl acetate:heptane 25:75 to give a mixture of products after evaporation. Heptane (60 mL) was added and the mixture was stored at 4° C. overnight. The heptane was decanted off from the product oil and the residue was dissolved in a mixture of 0.5 M aqueous hydrochloric acid (20 mL) and DCM (20 mL). The aqueous layer was removed and 0.5 M aqueous sodium hydroxide solution (20 mL) was added to it. The product was then extracted with DCM (20 mL). The organic phase was dried over anhydrous sodium sulphate, filtered and then evaporated to give the title compound (1.47 g) as a yellow oil.

ES: m/z [M+1] 309.5.

### PREPARATION 51

### (cis)-3-(Dibenzylamino)cyclohexanecarbaldehyde

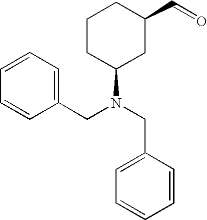

Dimethylsulphoxide (1.88 mL, 26.6 mmol) was added to a solution of Preparation 50 (1.37 g, 4.43 mmol) in DCM (10 mL). Triethylamine (3.7 mL, 26.6 mmol) was then added followed by sulphur trioxide pyridine complex (2.1 g, 26.6 mmol). The reaction mixture was stirred at room temperature for 1 hour. The reaction mixture was then washed with saturated aqueous sodium hydrogen carbonate (3×50 mL) and the combined aqueous layers were extracted with DCM (10 mL). The combined organic layers were washed with water (50 mL) and brine (2×50 mL), dried over anhydrous sodium sulphate and evapourated to give a residue that was purified by flash chromatography on silica gel eluting with ethyl acetate:heptane 8:1. This gave the title compound (0.71 g) as an impure liquid. The material was used crude in subsequent experiments.

ES: m/z [M+1] 308.2.

### PREPARATION 52

### 1-[(cis)-3-(Dibenzylamino)cyclohexyl]ethanol

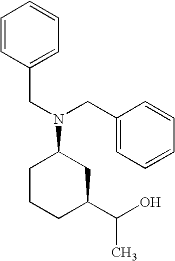

The product of Preparation 51 (0.35 g, 1.1 mmol) was dissolved in dry THF (5 mL) and the resulting solution was cooled to −10° C. A 22% w/w solution of methylmagnesium chloride in THF (1 mL, 3 mmol) was added dropwise and the reaction mixture was stirred at −10° C. The reaction was quenched by the addition of water (5 mL) after 90 minutes. The solvents were removed under reduced pressure and the residue was stirred for 1 hour with ethyl acetate (30 mL). The ethyl acetate was evaporated to give the title compound as an oil (0.42 g).

ES: m/z [M+1] 324.2.

### PREPARATION 53

### 1-[(cis)-3-Aminocyclohexyl]ethanol

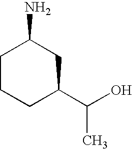

The product of Preparation 52 (0.14 g, 0.43 mmol) was dissolved into ethanol (2 mL) and 10% palladium on carbon (46 mg) was added. The reaction mixture was placed under an atmosphere of hydrogen (1 atmosphere pressure) and stirred for 16 hours at room temperature.

The reaction mixture was filtered through Celite® and the filter cake washed with ethanol (10 mL) and DCM (20 mL). The combined organic portions were evaporated to give the title compound, 57 mg, as a colourless oil.

ES: m/z [M+1] 144.2.

### PREPARATION 54

### 1-[(cis)-3-(Dibenzylamino)cyclohexyl]propan-1-ol

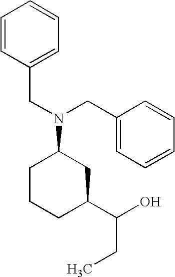

The title compound was prepared in an analogous manner to Preparation 52 using 0.35 g of Preparation 51 (1.14 mmol) and was obtained as an oil (214 mg).

ES: m/z [M+1] 338.2.

### PREPARATION 55

### 1-[(cis)-3-Aminocyclohexyl]propan-1-ol

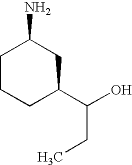

The title compound was prepared in an analogous manner to Preparation 53 using 157 mg of preparation 54 (0.47 mmol) and was obtained as an oil (38 mg).

ES: m/z [M+1] 158.2.

### PREPARATION 56

### Dibenzyl N,N-dibenzylglutamate

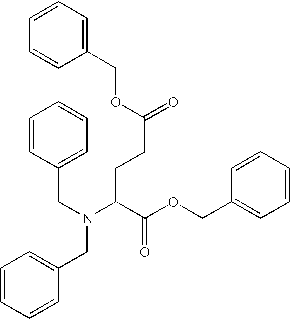

Glutamic acid hydrochloride salt (15 g, 91 mmol) was dissolved in a mixture of dioxane (75 mL) and water (75 mL). The resulting solution was stirred while sodium hydroxide (11.8 g, 295 mmol) was added at room temperature. Potassium carbonate (28.2 g, 204 mmol) and benzyl bromide (69.9 ml, 409 mmol) were then added and the reaction mixture was heated under reflux for 16 hours. The reaction mixture was allowed to cool to room temperature and was concentrated until a white gel formed. The gel was partitioned between water (1 L) and DCM (500 mL). Methanol (250 mL) was added and the organic layer was removed, dried with anhydrous sodium sulphate, and evaporated to give the title compound, 42 g, as a colourless oil.

ES: m/z [M+1] 508.2.

### PREPARATION 57

### 2-(Dibenzylamino)pentane-1,5-diol

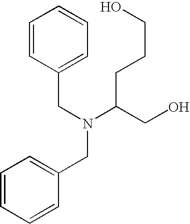

Lithium aluminium hydride (41.5 ml of a 2.4 M solution in THF, 100 mmol) was added to a solution of the product of Preparation 56 (42.1 g, 83 mmol) in anhydrous THF (400 mL). The reaction was then quenched with water (30 mL) after two hours. The solvent was removed from the reaction until the THF had been evaporated and the residue was extracted with DCM (400 mL) and methanol (400 mL). The organic layers were combined, washed with brine (200 mL), dried over anhydrous sodium sulphate and evaporated to give a residue that was purified by flash chromatography on silica gel using ethyl acetate:heptane1:1 as eluant to give the title compound (20.88 g) as a colourless oil.

ES: m/z [M+1] 300.2.

### PREPARATION 58

### N,N-Dibenzyl-1,5-dichloropentan-2-amine

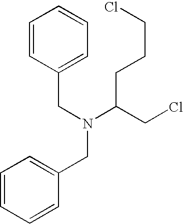

Thionyl chloride (0.6 g, 5 mmol) was added to a solution of Preparation 57 (0.5 g, 1.67 mmol) in toluene (5 mL) at room temperature. The reaction mixture was then heated to 77° C. for 30 minutes. The solvent was removed under reduced pressure and the residue was redissolved in a mixture of diethylether (10 mL) and saturated aqueous sodium hydrogen carbonate solution (10 mL). The organic phase was removed and the aqueous phase was extracted with another portion of diethylether (10 mL). The combined organic phases were washed with water (10 mL) and brine (10 mL), dried over anhydrous sodium sulphate, filtered and evaporated under reduced pressure. This gave the title compound (449 mg) as a yellow oil which was used without any further purification.

ES: m/z [M] 336.2.

### PREPARATION 59

### Ethyl 1-cyano-3-(dibenzylamino)cyclohexanecarboxylate

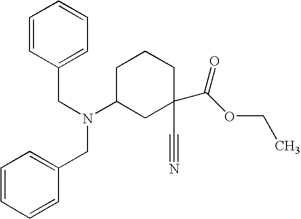

Ethyl cyanoacetate (3.46 g, 30.6 mmol) and caesium carbonate (29.9 g, 92 mmol) were added to a solution of the product of Preparation 58 (10.3 g, 30.6 mmol) in DMF (70 mL). The reaction mixture was stirred at 79° C. for 16 hours and the solvent was removed under reduced pressure. The residue was purified by flash chromatography on silica gel eluting with heptane:ethyl acetate 6:1. The product containing fractions were evaporated to give 6.83 g of oil which solidified on standing.

ES: m/z [M+1] 377.2.

### PREPARATION 60

### 1-Cyano-3-(dibenzylamino)cyclohexanecarboxylic acid

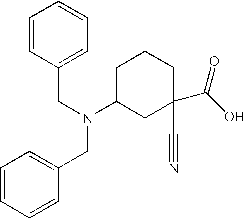

Lithium hydroxide monohydrate (3.34 g, 80 mmol) was added to a solution of the product of Preparation 59 in a mixture of THF (35 mL) and water (35 mL) and the resulting mixture was stirred at 52° C. for 16 hours. The reaction mixture was then allowed to cool and the THF was removed under reduced pressure. The pH of the resulting white suspension was adjusted to 6 with 1M aqueous hydrochloric acid and it was extracted with DCM (2×50 mL). The combined organic layers were washed with brine (25 mL) and water (25 mL), dried with anhydrous magnesium sulphate, filtered and then evaporated under reduced pressure to give the title compound (4.62 g) as a white solid.

ES: m/z [M+1] 349.2.

### PREPARATION 61

### 3-(Dibenzylamino)-1-[(4-methylpiperazin-1-yl)carbonyl]cyclohexanecarbonitrile

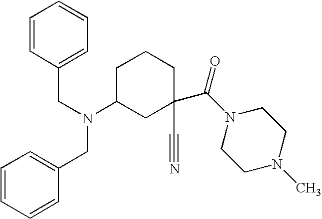

The product of preparation 60 (0.5 g, 1.435 mmol) and 1-methylpiperazine (4 g, 40 mmol) were dissolved in the DMF (5 mL) and the resulting solution was stirred at 0° C. as HATU (0.6 g, 1.6 mmol) was added. The reaction mixture was stirred at 0° C. for 50 minutes and then at room temperature for 48 hours. The reaction mixture was evaporated under reduced pressure and the residue was partitioned between brine (30 mL) and ethyl acetate (2×20 mL). The combined organic layers were dried over anhydrous sodium sulphate, filtered and evaporated. The residue was purified by flash chromatography on silica gel eluting with DCM:methanol 9:1 to give the title compound (0.285 g) as a yellow oil.

ES: m/z [M+1] 431.4.

### PREPARATION 62

### 3-Amino-1-[(4-methylpiperazin-1-yl)carbonyl]cyclohexanecarbonitrile

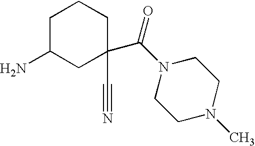

The product of Preparation 61 (0.26 g, 0.59 mmol) was dissolved in ethanol (3 mL) and 10% palladium on carbon (0.19 g, 0.18 mmol) was added. The reaction mixture was stirred under an atmosphere of hydrogen (1 atmosphere) for 24 hours and then further 10% palladium on carbon (0.19 g, 0.18 mmol) was added). The reaction mixture was stirred for a further 24 hours at room temperature and then filtered through Celite®. The filter cake was washed with ethanol (20 mL) and DCM (20 mL). The combined organic eluants were evaporated to give the title compound, 0.147 g, as an oil. This material was used crude in subsequent experiments

### PREPARATION 63

### 3-(Dibenzylamino)-1-[(4-ethylpiperazin-1-yl)carbonyl]cyclohexanecarbonitrile

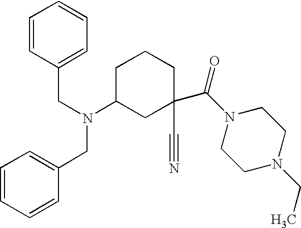

The product of Preparation 60 (0.5 g, 1.44 mmol) was dissolved into DMF (5 mL) and the resulting solution was stirred and cooled to 0° C. before being treated with HATU (0.818 g, 2.15 mmol) and then N-ethylpiperazine (4.59 g, 40.2 mmol). The reaction mixture was stirred for 50 minutes at 0° C. and then for 48 hours at room temperature. An extra portion of HATU (0.414 g, 1.1 mmol) was added and the solution was stirred for another 88 hours. The solvent was removed under reduced pressure and the residue was purified by flash chromatography on silica gel eluting with DCM:methanol 9:1. The product containing fractions were evaporated under reduced pressure and the residue stirred with diethylether (20 mL) for 2 hours. The resulting solid was filtered off and the mother liquor was evaporated to give the title compound, 213 mg, as a brown oil.

ES: m/z [M+1] 445.4.

### PREPARATION 64

### 3-Amino-1-[(4-ethylpiperazin-1-yl)carbonyl]cyclohexanecarbonitrile

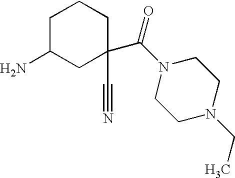

The product of Preparation 63 (0.21 g, 0.48 mmol) was dissolved in ethanol (2 mL) and 10% palladium on carbon (2 mg) was added. The reaction mixture was placed under an atmosphere of hydrogen gas (1 atmosphere) and stirred for 16 hours at room temperature. The reaction mixture was filtered through Celite® and the filter cake was washed with DCM (20 mL) and ethanol (20 mL). The combined organic eluants were evaporated to give the title compound (78 mg) as a brown oil.

ES: m/z [M+1] 265.2.

### PREPARATION 65

### 3-(Dibenzylamino)-1-(morpholin-4-ylcarbonyl)cyclohexanecarbonitrile

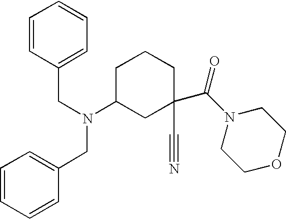

The product of Preparation 60 (0.5 g, 1.44 mmol) was dissolved in DMF (5 mL) and the solution was cooled to 0° C. The reaction mixture was stirred while HATU (0.818 g, 2.15 mmol) and then morpholine (3.54 g, 40.2 mmol) were added. The reaction mixture was then stirred for 50 minutes at 0° C. and for 48 hours at room temperature. An extra portion of HATU (0.414 g, 1.1 mmol) was added and the solution was stirred for another 88 hours. The solvent was removed under reduced pressure and the residue was purified by flash chromatography on silica gel eluting with DCM:methanol 95:5. The product containing fractions were evaporated under reduced pressure to give the title compound (161 mg) as a brown oil.

ES: m/z [M+1] 418.2.

### PREPARATION 66

### 3-Amino-1-(morpholin-4-ylcarbonyl)cyclohexanecarbonitrile

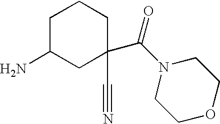

Preparation 65 (0.16 g, 0.48 mmol) was dissolved in ethanol (2 mL) and 10% palladium on carbon (2 mg) was added. The reaction mixture was placed under an atmosphere of hydrogen gas (1 atmosphere) and stirred for 16 hours at room temperature. The reaction mixture was filtered through Celite® and the filter cake was washed with DCM (20 mL) and ethanol (20 mL). The combined organic fractions were evaporated to give the title compound (75 mg) as a brown oil. This material was used crude in subsequent reactions.

### PREPARATION 67

### 1-Cyano-3-(dibenzylamino)-N,N-dimethylcyclohexanecarboxamide

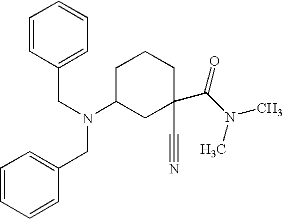

Preparation 60 (0.5 g, 1.44 mmol) was dissolved into DMF (5 mL) and the solution was cooled to 0° C. The reaction mixture was stirred while HATU (0.818 g, 2.15 mmol) and then dimethylamine (1.8 g, 40.2 mmol) were added. The reaction mixture was then stirred for 50 minutes at 0° C. and then for 48 hours at room temperature. An extra portion of HATU (0.414 g, 1.1 mmol) was added and the solution was stirred for another 88 hours. The solvent was removed under reduced pressure and the residue was purified by flash chromatography on silica gel eluting with DCM:methanol 95:5. The product containing fractions were evaporated under reduced pressure and the residue was stirred with ether (20 mL) for 2 hours. The resulting solid was filtered off and the mother liquor was evaporated to give the title compound (0.139 g) as an oil.

ES: m/z [M+1] 376.2.

### PREPARATION 68

### 3-Amino-1-cyano-N,N-dimethylcyclohexanecarboxamide

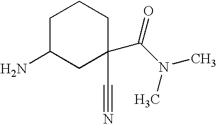

The product of Preparation 67 (0.227 g, 0.605 mmol) was dissolved in ethanol (2 mL) and 10% palladium on carbon (2 mg) was added. The reaction mixture was placed under an atmosphere of hydrogen gas (1 atmosphere) and stirred for 16 hours at room temperature. The reaction mixture was filtered through Celite® and the filter cake was washed with DCM (20 mL) and ethanol (20 mL). The combined organic fractions were evaporated to give the title compound (112 mg) as a brown oil. This material was used crude in subsequent experiments.

### PREPARATION 69

### tert-Butyl(cis-3-aminocyclohexyl)carbamate

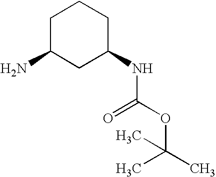

cis-1,3-Cyclohexanediamine dihydrochloride (1.80 g, 9.64 mmol) was dissolved in methanol (25 mL) at room temperature and 1M aqueous sodium hydroxide (9.63 mL, 9.64 mmol) was added.

The reaction mixture was stirred for 30 minutes at room temperature and then cooled in ice and treated with a solution of di-tert butyldicarbonate (2.10 g, 9.64 mmol) drop wise over 15 minutes. The resulting stirred solution was allowed to warm to room temperature and stirred at room temperature for 1 hour. The reaction was basified with 1M sodium hydroxide solution (10 mL) and the methanol was removed by evaporation in vacuo. The reaction mixture was extracted with dichloromethane (2×50 mL) and the combined organic phases were washed with brine (30 mL), dried over magnesium sulphate, filtered and evaporated to give the title compound as a fawn coloured solid (2.2 g).

LRMS: 215 [M+1] (obs), [M+1] 214.3 (calc).

¹H NMR (400 MHz, DMSO-d₆): δ ppm 0.82-1.00 (m, 3H) 1.13-1.19 (m, 1H) 1.39 (s, 9H) 1.61-1.66 (m, 3H) 1.83-1.86 (m, 1H) 3.04-3.10 (m, 1H) 3.29-3.41 (m, 1H) 6.68-6.72 (m, 1H).

### PREPARATION 70

### tert-Butyl(trans-3-aminocyclohexyl)carbamate

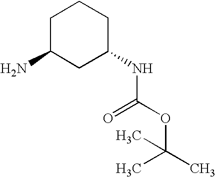

Trans-1,3-cyclohexanediamine tartrate salt (2.55 g, 9.64 mmol) was dissolved in methanol (25 mL) at room temperature and 1M sodium hydroxide (9.63 mL, 9.64 mmol) was added. The reaction mixture was stirred for 30 min at room temperature and then cooled in ice and treated with a solution of di-tert-butyldicarbonate (2.10 g, 9.64 mmol) drop wise over 15 minutes. The resulting mixture was allowed to warm to room temperature and stirred at room temperature for 1 hour. The reaction was basified with 1M aqueous sodium hydroxide solution (10 mL) and the methanol was removed by evaporation. The reaction mixture was extracted with dichloromethane (2×50 mL) and the combined organic phases were washed with brine (30 mL), dried over magnesium sulphate, filtered and evaporated to give the title compound as a fawn coloured solid (1.6 g).

LRMS: 215 [M+1] (obs), 214.3 [M+1] (calc).

¹H NMR (400 MHz, DMSO-d₆): δ ppm 0.82-1.00 (m, 3H) 1.13-1.19 (m, 1H) 1.39 (s, 9H) 1.61-1.66 (m, 3H) 1.83-1.86 (m, 1H) 3.04-3.10 (m, 1H) 3.29-3.41 (m, 1H) 6.70-6.74 (m, 1H).

### PREPARATION 71

### Trans-1,3-diaminocyclohexane tartrate salt

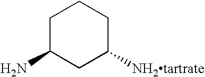

A cis/trans mixture of 1,3-diaminocyclohexane (5.00 g, 43.8 mmol) was dissolved in methanol (60 mL) and stirred at room temperature. To this solution was added D-tartaric acid (6.57 g, 43.8 mmol) in warm methanol (60 mL). This resulting solid white mass was heated under reflux with stirring for 5 hour and then stood at room temperature over night. The resulting mixture was filtered to give 11 g of solid which was a 3:1 trans:cis mixture by NMR. This was suspended in methanol (200 mL) and heated to reflux. Large amounts of precipitate remained. Water was added until material was almost in solution and then the suspension was allowed to cool overnight. Since no product had crystallized, the solution was evaporated to a clear oil. Addition of methanol resulted in a precipitate and evaporation gave a white solid. This solid was suspended/dissolved in water (15 mL) and methanol (300 mL) was added. The resulting mixture was heated under reflux for 1 hour and then allowed to cool overnight. The liquors were decanted off from the crystalline solid and the solid dried to give 10.9 g of product. NMR indicated cis:trans isomers in 1:4 ratio. This solid was dissolved in water (20 mL), methanol (50 mL) was added and the resulting mixture was heated to 65° C. Further methanol (150 mL) was added and the mixture was heated under reflux for a further 1 hour, then allowed to cool slowly to room temperature overnight. The precipitated solid was filtered and dried to give 8.8 g of a product which was shown to be cis:trans 1.0:6.5. The recrystallisation was repeated using water (35 mL) and methanol (100 mL). After cooling to room temperature the precipitated solid was filtered off and dried to give 8.06 g of the title compound as a white solid. NMR showed a cis:trans ratio of 1:24. ¹H NMR (400 MHz, D₂O-d₆): δ ppm 1.23-1.31 (m, 3H) 1.63-1.43 (m, 2H) 1.87-1.99 (m, 3H) 2.29-2.32 (m, 1H) 3.19-3.26 (m, 2H).

### PREPARATION 72

### Methyl trans-4-(dibenzylamino)cyclohexanecarboxylate hydrochloride salt

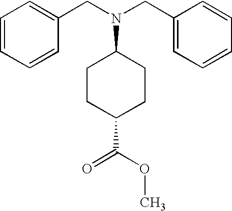

Potassium carbonate (14.5 g, 105 mmol) and benzyl bromide (10.4 mL, 88.0 mmol) were added to a suspension of methyl trans-4-aminocyclohexanecarboxylate hydrochloride salt (6.785 g, 35.0 mmol) in acetonitrile (100 mL) and the reaction mixture was stirred at room temperature for 3 hours. The salts were filtered off and the solvent was evaporated off to give a clear oil (15.3 g). This oil was dissolved in tetrahydrofuran (15 mL) and 1M HCl in diethyl ether (50 mL) was added dropwise. The resulting sticky suspension was stirred at room temperature for 1 hour and the resulting fine white powder was filtered off and dried to give the title compound (13.2 g).

LRMS: [M+1] 338 (obs), [M+1] 338.42 (calc).

¹H NMR (400 MHz, DMSO-d₆): δ ppm 1.18-1.28 (m, 2H) 1.70-1.83 (m, 2H) 2.00-2.09 (m, 2H) 2.21-2.90 (m, 2H) 2.30-2.38 (m, 2H) 3.05-3.11 (m, 1H) 3.55-3.62 (m, 3H) 4.11-4.20 (m, 2H) 4.42-4.50 (m, 2H) 7.39-7.45 (m, 6H) 7.52-7.55 (m, 4H) 10.24 (bs, 1H).

### PREPARATION 73

### trans-4-(Dibenzylamino)cyclohexanecarboxylic acid hydrochloride salt

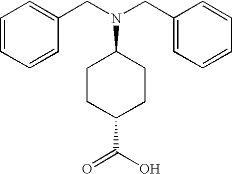

Methyl trans-4-(dibenzylamino)cyclohexanecarboxylate hydrochloride salt (10 g, 26.7 mmol) was dissolved in 6M hydrochloric acid (50 mL) and the resulting solution was heated under reflux for 15 minutes. The reaction mixture was cooled to room temperature and the solvent was evaporated in vacuo. The residue was suspended in ethanol (100 mL) and evaporated to give the title compound as a white solid (9.74 g).

LRMS [M+1] 324 (obs), [M+1] 324.39 (calc).

¹H NMR (400 MHz, DMSO-d₆): δ ppm 1.14-1.23 (m, 2H) 1.70-1.79 (m, 2H) 2.00-2.09 (m, 2H) 2.17-2.29 (m, 3H) 3.00-3.03 (m, 1H) 4.11-4.16 (m, 2H) 4.43-4.48 (m, 2H) 7.39-7.42 (m, 6H) 7.59-7.62 (m, 4H) 10.64 (bs, 1H) 12.25 (bs, 1H).

### PREPARATION 74

### trans-4-(Dibenzylamino)cyclohexanecarbonitrile

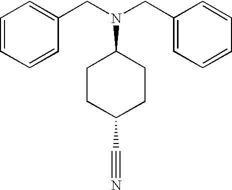

Methyl trans-4-(dibenzylamino)cyclohexanecarboxylate hydrochloride salt (Preparation 73, 9.7 g, 27.0 mmol) was dissolved in tetrahydrofuran (100 mL) and the resulting solution was cooled to 0° C. Triethylamine (26.3 mL, 189 mmol) and ammonium chloride (4.33 g, 81 mmol) were added followed by 1-propanephosphonic acid cyclic anhydride (24.1 mL, 81 mmol) dropwise. The reaction mixture was heated under reflux overnight, then cooled to room temperature and the evaporated in vacuo. Ethyl acetate (250 mL) was added along with saturated sodium hydrogen carbonate solution (200 mL) and the phases were separated. The organic layer was washed with brine, dried over sodium sulphate, filtered and evaporated to dryness. The residue was purified on silica eluting with a gradient of ethyl acetate:heptane 1:9 to 9:1. This gave the title compound as a clear oil (3.34 g) which was re-purified on silica eluting with ethyl acetate:heptane 1:9 to 1:2 in a gradient elution. This gave the title compound as a white crystalline powder (2.19 g). ¹H NMR (400 MHz, DMSO-d₆): δ ppm 1.30-1.50 (m, 4H) 1.81-1.89 (m, 2H) 1.99-2.08 (m, 2H) 2.37-2.46 (m, 1H) 2.56-2.63 (m, 1H) 7.17-7.20 (m, 2H) 7.28-7.35 (m, 8H).

### PREPARATION 75

### N-{1-[trans-4-(Dibenzylamino)cyclohexyl]ethyl}acetamide

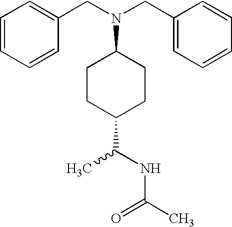

Trans-4-(dibenzylamino)cyclohexanecarbonitrile (Preparation 74, 100 mg, 0.328 mmol) was dissolved in tetrahydrofuran (5 mL) and the resulting solution was cooled to 0° C. A solution of methylmagnesium chloride in tetrahydrofuran (1.6M, 0.821 mL, 1.314 mmol) was added dropwise and the reaction mixture was heated under reflux for 2 hours. The reaction mixture was cooled to 0° C., treated with a solution of sodium borohydride in tetraglyme (0.046 mL, 1.314 mmol) and stirred at room temperature overnight. Acetic anhydride (0.5 mL, 5.30 mmol) was then added and the reaction mixture was stirred at room temperature for 2 hours. The reaction was quenched with saturated sodium hydrogen carbonate (5 mL) and stirred vigorously for 30 minutes. Ethanol (10 mL) was added and the solvents were evaporated. The resulting residue was stirred with dichloromethane:ethanol 1:1 (10 mL) and the salts were filtered off. The salts were re-washed with dichloromethane:ethanol 1:1 (10 mL). The filtrate was evaporated to give a white powder (125 mg) which was purified on silica eluting with a gradient of methanol:dichloromethane 5:95 to 10:90. LRMS: [M+1] 365 (obs), [M+1] 365.52 (calc). Another product was present by mass spec indicating 70% purity of desired product. This crude product was used without further purification in Preparation 76.

### PREPARATION 76

### N-[1-trans-4-Aminocyclohexyl)ethyl]acetamide

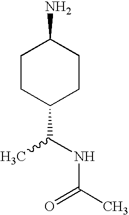

N-{1-[trans-4-(Dibenzylamino)cyclohexyl]ethyl}acetamide (Preparation 75, 0.129 g, 0.354 mmol) was dissolved in ethanol (5 mL) and the resulting solution was hydrogenated over 10% palladium on carbon (0.038 g, 0.035 mmol) at 1 atmosphere pressure for 2 hours. The reaction mixture was filtered through Celite®, and the filter pad was washed with ethanol and dichloromethane. The combined eluants were evaporated to give the title compound as a clear oil (72 mg).

LRMS: [M+1] 185 (obs), [M+1] 185.28 (calc).

¹H NMR (400 MHz, CDCl₃): δ ppm 1.02-1.12 (m, 7H) 1.69-1.81 (m, 2H) 1.89-1.94 (m, 2H) 2.57-2.63 (m, 1H) 3.80-3.89 (m, 1H) 5.28-5.34 (m, 1H).

### PREPARATION 77

### 2-[Cis-4-(Dibenzylamino)cyclohexyl]propan-2-ol

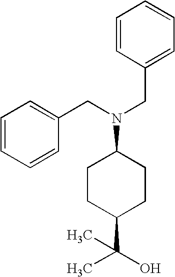

Benzyl cis-4-(dibenzylamino)cyclohexanecarboxylate (US-2008/021048, 0.7 g, 1.69 mmol) was dissolved in 2-methyl tetrahydrofuran (40 mL) and the resulting solution was cooled in an icebath. A solution of methyl magnesium bromide in diethyl ether (3M, 4.52 mL) was added drop wise and the reaction mixture was allowed to warm slowly to room temperature. The reaction was stirred at room temperature for 16 hours. The reaction mixture was then cooled in ice and quenched by dropwise addition of ammonium chloride solution. The organic layer was separated, dried over magnesium sulphate, filtered and evaporated to give an oil. The crude oil was purified by silica gel chromatography eluting with a gradient of heptane:ethyl acetate 90:10 to 80:20, and the product-containing fractions were combined and evaporated to give the title compound as a gum (510 mg).

LRMS: [M+1] 338 (obs), [M+1] 338.5 (calc).

¹H NMR (400 MHz, CDCl₃): δ ppm 1.20 (s, 6H) 1.40-1.58 (m, 7H) 2.10-2.19 (m, 2H) 2.79-2.82 (m, 1H) 7.18-7.71 (m, 2H) 7.25-7.30 (m, 8H).

### PREPARATION 78

### 2-(cis-4-Aminocyclohexyl)propan-2-ol

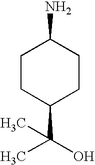

2-[cis-4-(Dibenzylamino)cyclohexyl]propan-2-ol (Preparation 77, 500 mg, 1.48 mmol) was dissolved in ethanol (15 mL) and the resulting solution was hydrogenated over 20% palladium hydroxide on carbon (100 mg) at room temperature and 30 p.s.i. pressure for 20 hours. The reaction mixture was filtered through Arbocel®, the filter pad was washed with methanol and the filtrate was evaporated to give the title compound as a gum. ¹H NMR (400 MHz, CDCl₃): δ ppm 1.18 (s, 6H) 1.20-1.30 (m, 1H) 1.58-1.62 (m, 3H) 1.68-1.72 (m, 5H) 3.20-3.21 (m, 1H).

### PREPARATION 79

### Methyl {cis-4-[(tert-butoxycarbonyl)amino]cyclohexyl}acetate

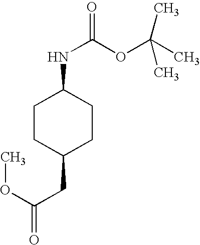

Methyl iodide (1.16 mL, 18.7 mmol) was added to a suspension of {cis-4-[(tert-butoxycarbonyl)amino]cyclohexyl}acetic acid (4.0 g, 16.0 mmol) and caesium carbonate (2.53 g, 7.77 mmol) in dimethylformamide (20 mL) and the reaction mixture was stirred at room temperature overnight. The reaction mixture was diluted with water (100 mL) and extracted with ethyl acetate (3×70 mL). The combined organic extracts were washed with water (3×50 mL), dried over magnesium sulphate, filtered and evaporated in vacuo to give the title compound as a white solid (3.8 g). ¹H NMR (400 MHz, CDCl₃): δ ppm 1.43 (s, 9H) 1.54-1.64 (m, 7H) 1.86-1.93 (m, 1H) 2.23-2.25 (m, 2H) 3.65 (s, 3H) 3.67-3.71 (m, 1H) 4.55-4.60 (m, 1H).

### PREPARATION 80

### Methyl (cis-4-aminocyclohexyl)acetate hydrochloride salt

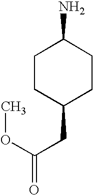

Methyl {cis-4-[(tert-butoxycarbonyl)amino]cyclohexyl}acetate (Preparation 79, 3.8 g, 14 mmol) was dissolved in a 4N solution of hydrogen chloride in 1,4-dioxane (35 mL) and the reaction mixture was stirred at room temperature overnight. The reaction mixture was evaporated to give the title compound as a white solid (1.9 g). ¹H NMR (400 MHz, DMSO-d₆): δ ppm 1.67-1.74 (m, 4H) 1.87-1.92 (m, 4H) 2.12-2.17 (m, 1H) 2.73-2.74 (m, 2H) 3.36-3.40 (m, 1H) 3.86 (s, 3H) 8.36 (bs, 3H).

### PREPARATION 81

### Benzyl-trans-[3-(hydroxymethyl)cyclohexyl]carbamate

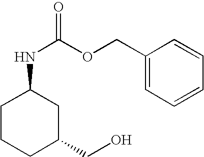

tert-Butyl-trans-(3-hydroxymethyl)cyclohexylcarbamate (Preparation 80, 5.0 g, 20 mmol) was treated with a solution of 4M hydrogen chloride in 1,4-dioxane (4.94 mL) at room temperature. The solvent was evaporated and the remaining oil was triturated with acetonitrile to leave a colourless but cloudy thick oil. This material was dissolved in a mixture of tetrahydrofuran (100 mL) and water (25 mL) and potassium carbonate (9.32 g, 65.4 mmol) was added followed by benzyl chloroformate (4.94 mL, 32.7 mmol). The reaction mixture was stirred at room temperature for 2 hours, diluted with diethyl ether, washed with saturated sodium bicarbonate solution and then brine, dried over sodium sulphate, filtered and evaporated to give the title compound as a clear, colourless liquid (6.0 g). This crude product was purified on silica eluting with a gradient of dichloromethane to methanol:dichloromethane 5:95. This gave the title compound (4.2 g) as a clear, colourless oil. LCMS: [M+1] 264 (obs), [M+1] 264.34 (calc) ES+, RT 2.57 min 91% area.

### PREPARATION 82

### Benzyl [(1S,3S)-3-(hydroxymethyl)cyclohexyl]carbamate

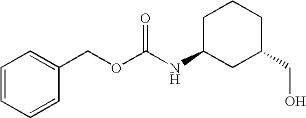

Racemic benzyl-trans-[3-(hydroxymethyl)cyclohexyl]carbamate, Preparation 81, was separated into the two enantiomers by chromatography on an AD-H (Chiral Technologies) 50×250 mm column eluting with 25% ethanol/75% CO₂ using a flow rate of 200 mL/min. The fractions were analysed on an AD-H column (Chiral Technologies), elutant 50% ethanol/50% CO₂: the title compound is the second peak which elutes at 1.77 minutes.

### PREPARATION 83

### [(1S,3S)-3-Aminocyclohexyl]methanol

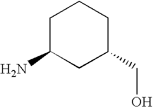

Benzyl [(1S,3S)-3-(hydroxymethyl)cyclohexyl]carbamate (Preparation 82, 5.70 g, 22.0 mmol) was dissolved in ethanol (70 mL) and the resulting solution was hydrogenated over 10% palladium on carbon (0.57 g) at room temperature and 50 p.s.i pressure for 4 hours. The catalyst was filtered through Celite® and the filter pad was washed with ethanol. The filtrate was concentrated to give the title compound as a clear oil (3.0 g). After cooling in the refrigerator overnight a sticky solid was formed. This was dissolved in ethanol (50 mL) and ethyl acetate (100 mL) was added. The resulting clear solution was concentrated to give an oil. Further ethyl acetate (100 mL) was added, the solution was re-evaporated and this process was repeated until the title compound solidified (2.8 g). LCMS: [M+1] 130 (obs) [M+1] 130.20 (calc), RT 0.13 min 100% area.

### PREPARATION 84

### (4-Aminocyclohexyl)acetic acid methyl ester hydrochloride

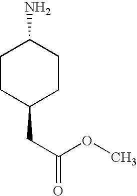

Step (a): Methyl iodide (0.87 mL, 14.0 mmol) was slowly added to a suspension of (4-tert butoxycarbonylaminocyclohexyl)acetic acid (3.0 g, 11.7 mmol) and cesium carbonate (1.9 g, 5.83 mmol) in DMF (30 mL) and the reaction mixture was stirred at room temperature for 18 hours. The reaction mixture was diluted with water (25 ml) and extracted with ethyl acetate (3×100 ml) and the combined organic phases were washed with brine (25 mL), dried over MgSO₄ and concentrated to afford 4-tert-butoxycarbonylaminocyclohexanecarboxylic acid methyl ester as a white solid 2.87 g (11.7 mmol).

LRMS: m/z (AP+) [M+1-CO₂tBu] 172.

¹H NMR (400 Mhz, CDCl₃): δ ppm 1.09 (m. 4H), 1.41 (s, 9H), 1.72 (m, 1H), 1.75 (m, 2H), 1.97 (m, 2H), 2.17 (d, 2H), 3.35 (broad s, 1H), 3.64 (s, 3H), 4.39 (broad s, 1H).

Step (b): To a solution of the product of step (a) in DCM under nitrogen was carefully added 4M HCl in 1,4 dioxane. The reaction mixture was stirred for 1 hour and then evaporated in vacuo to give the title compound as a white solid 2.2 g.

LRMS: m/z (AP+) [M+1] 172.

¹H NMR (400 MHz, DMSO-d6): δ ppm 1.029 (m, 2H), 1.30 (m, 2H), 1.60 (m, 1H), 1.70 (m, 2H), 1.90 (m, 2H), 2.18 (d, 2H), 2.87 (broad s, 1H), 3.31 (s, 3H), 7.97 (broad s, 2H).

### PREPARATION 85

### 4-Amino-1-trifluoromethlycyclohexanol

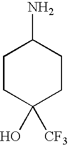

Step (a): Dibenzylaminocyclohexanone [Ger. Offen., 4326344, 1.0 g, 3.41 mmol) was dissolved in THF (5 mL) and trimethyl(trifluoromethyl)silane (533 mg, 5.75 mmol) was added dropwise to the resulting solution. The reaction mixture was cooled to 0° C., a solution of tetrabutylammonium fluoride in THF (1 M, 0.5 mL, 0.05 mmol) was added and the reaction mixture was then allowed to warm to room temp and stirred at that temperature for 18 hours. A 6M aqueous solution of HCl (5 ml) was added to the reaction mixture and stirring was continued for 24 hours. The THF was removed in vacuo, and the remaining mixture was extracted with ethyl acetate. The organic extract was passed through a phase separation cartridge and concentrated in vacuo. The resulting oil was then purified by silica chromatography eluting with a gradient of DCM to 95:5 DCM:MeOH to give 4-dibenzylamino-1-trifluormethylcycohexanol (230 mg) as pale yellow crystals.

¹H NMR (400 MHz, DMSO-d₆): [symbol] ppm 1.33 (m, 2H), 1.72 (m, 4H), 1.94 (m, 2H), 2.62 (m, 1H), 3.60 (s, 4H), 7.28 (m, 10H).

¹⁹F NMR (376 MHz, DMSO-d₆): [symbol] ppm −79.97 ppm.

Step (b): the product from step (a) (230 mg, 0.633 mmol) was dissolved in ethanol (5 mL) and the resulting solution was treated with Pd(OH)₂ on carbon (107 mg, 0.760 mmol) and ammonium formate (399 mg, 6.33 mmol) under an atmosphere of N₂. The reaction mixture was heated to reflux for 2 hours, cooled, filtered through Arbocel® and concentrated to give an oil. This crude product was loaded onto an SCX-2 column eluting with methanol (100 ml) then 10% 880 NH₃ (aq) in methanol. Removal of the solvent afforded the title compound as a yellow oil (60 mg).

¹H NMR (CDCl₃, 400 MHz); [symbol] ppm 1.46-1.58 (m, 4H), 1.85-2.22 (m, 5H), 3.18 (m, 1H).

LRMS: m/z (ES+) [M+1]184.

### PREPARATION 86

### 2-((1S,3R)-3-Aminocyclophentyl)propan-2-ol

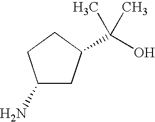

Step (a): (+)-(1S,3R)—N-Boc-3-Aminocyclopentane carboxylic acid (355 mg, 1.55 mmol) and cesium carbonate (252 mg, 0.774 mmol) were dissolved in methanol (3 ml) and the resulting solution was evaporated in vacuo. The residue was suspended in dry toluene and concentrated in vacuo twice. The residue was then dissolved in dry DMF (5 ml), iodomethane (290 mL, 4.64 mmol) was added and the resulting solution was stirred at room temperature for 18 hours. The reaction mixture was concentrated in vacuo and partitioned between DCM and water and the organic phase was dried, concentrated and purified by silica column chromatography eluting with a gradient of DCM to 5% MeOH:DCM to give 224 mg of (1S,3R)-3-tert-butoxycarbonlyamino-cyclopentanecarboxylic acid methyl ester as a pale yellow oil.

¹H NMR (CDCl₃, 400 MHz); [symbol] ppm 1.44 (s, 9H), 1.62 (m, 1H), 1.70 (m, 1H), 1.92 (m, 3H), 2.21 (m, 1H), 2.83 (m, 1H), 3.69 (s, 3H), 4.04 (broad 1H), 4.91 (broad, 1H).

Step (b): A solution of methyl magnesium chloride in THF (3M, 1.20 mL, 3.62 mmol) was added to a solution of the product of step (a) in THF (4 mL) cooled to 0° C. The reaction mixture was allowed to warm to room temperature and stirred overnight. The reaction was quenched by dropwise addition of water and concentrated in vacuo. The residue was extracted with ethyl acetate, filtered, evaporated and purified by silica column chromatography eluting with a gradient of DCM to 2% MeOH in DCM to give 150 mg of ((1R,3S)-3-acetylcyclopentyl)carbamic acid tert-butyl ester.

¹H NMR (CDCl₃, 400 MHz); [symbol] ppm 1.43 (s, 9H), 1.62 (m, 1H), 1.68 (m, 1H), 1.88 (m, 3H), 2.11 (m, 1H), 2.18 (s, 3H), 3.00 (m, 1H), 4.02 (m, 1H), 4.83 (m, 1H).

Step (c): A solution of methyl magnesium chloride solution in THF (3M, 0.88 mL, 2.64 mmol) was added to a solution of the product of step (b) (200 mg, 0.88 mmol) in THF (3.5 mL) at 0° C. and the reaction mixture was allowed to warm to room temperature and stirred for 24 hours. The reaction was quenched by the dropwise addition of water and extracted with ethyl acetate and the organic phase was dried and evaporated to give 113 mg crude [(1R,3S)-3-(1-hydroxy-1-methylethyl)cyclopentyl]carbamic acid tert butyl ester which was used as such in step (d).

Step (d): A 4N solution of HCl in 1,4 dioxane (2.23 mL, 9.28 mmol) was added to the product of step (c) the resulting mixture was stirred at room temperature for 3 hours. The reaction mixture was concentrated in vacuo, dissolved in methanol and eluted through an SCX cartridge with methanol and then 10% NH₃/methanol. The solvent was removed in vacuo to give 38 mg of the title compound which was used as such in subsequent experiments.

### PREPARATION 87

### 6-(3-Fluorophenyl)-N-(1-oxaspiro[2.5]oct-6-yl)nicotinamide

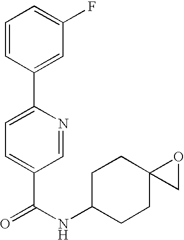

Step (a): Triethylamine (0.22 mL, 1.59 mmol) was added to a solution of the product of Example 104 (100 mg, 0.318 mmol) in dry DMSO (3 mL) and the resulting solution was stirred for 5 minutes. Pyridine sulphur trioxide complex (202 mg, 1.27 mmol) was added and the reaction mixture was stirred for 30 minutes. The reaction was quenched by the addition of water to precipitate the product as a white solid which was collected by filtration (80 mg, 90% purity)

MS: m/z (ES+) [M+1] 313.

Step (b): A suspension of sodium hydride (56 mg, 1.41 mmol) in THF (3 mL) was treated with trimethylsulfoxonium iodide (310 mg, 1.41 mmol) and heated under reflux for 4 hours. The resulting solution was then cooled to 0° C. and stirred for 10 minutes before the product of step (a) (220 mg, 0.704 mmol) was added as a solution THF (2 mL). This resulting mixture was stirred at room temp for 72 hours. Water was added, and the reaction mixture was stirred for 30 minutes and extracted with EtOAc four times. The combined organic phases were washed with water, dried over sodium sulfate and concentrated in vacuo to give 215 mg of the title compound as an off-white solid.

MS: m/z (ES+) [M+1] 327.

### PREPARATION 88

### [3-(4-Aminocyclohexyl)-3-hydroxypropyl]carbamic acid tert butyl ester

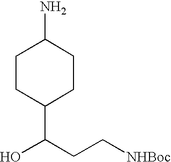

Step (a): A mixture of acetonitrile (0.535 mL, 10.2 mmol) and THF (5 mL) was cooled to −78° C., treated dropwise with a solution of lithium bis(trimethylsilyl)amide solution in THF (1M, 10.6 mL, 10.6 mmol) and stirred for 5 minutes. Methyl 4-(dibenzylamine)cyclohexane carboxylate (EP-A-537696, 1.78 g, 5.275 mmol) was then added in one portion and the reaction mixture was allowed to stir at −78° C. for 2 hours before the cooling bath was removed and the mixture was stirred at room temperature for 72 hours. The reaction mixture was concentrated in vacuo and partitioned between ethyl acetate and water (acidified to pH 1 using 2M aqueous hydrochloric acid). The organic phase was washed with brine, dried using a phase separation cartridge and concentrated in vacuo to give 1.33 g of 3-(4-aminocyclohexyl)-3-oxopropionitrile as a solid.

MS: m/z (ES+) [M+1] 347.

Step (b): The product from the previous step (1.33 g, 3.839 mmol) was dissolved in THF (11 ml), under N₂ and the resulting solution was cooled to 0° C. A solution of LiAlH₄ in THF (2M, 1.94 mL, 3.88 mmol) was added dropwise and the mixture was then allowed to warm to room temperature and stirred for 18 hours. The mixture was then heated to 60° C. for 30 minutes, cooled to 0° C., treated with a further portion of LiAlH₄ (3.83 mL, 7.67 mmol), stirred at room temperature for 30 mins and then heated at 60° C. for 1 hour. The reaction was cautiously quenched with water (0.45 mL), 2M aqueous NaOH (0.45 mL) and further water (1.35 mL), then diluted with diethyl ether and left to stir for 18 hours. The precipitated aluminium salts were filtered off and the filter cake was washed with diethyl ether and ethyl acetate. The combined organic phases were washed with water and concentrated in vacuo. Purification of the residue by silica chromatography eluting with 95:5 DCM:MeOH gave 3-amino-1-(4-aminocyclohexyl)propan-1-ol 350 mg as a yellow oil which crystallised on standing.

LRMS: m/z (ES+) [M+1] 349.

Step (c): Di-tert-butyl dicarbonate (217 mg, 0.993 mmol) was added in portions to a solution of the product of step (b) (350 mg, 0.993 mmol.) in DCM (2 mL) cooled in an ice bath. The reaction mixture was allowed to warm to room temperature and stirred for 18 hours. The solvent was removed in vacuo to give [3-(4-aminocyclohexyl)-3-hydroxypropyl]carbamic acid tert butyl ester (485 mg) as a white solid.

MS: m/z (ES+) [M+1] 453.

Step (d): The product from step (c) (486 mg, 1.07 mmol) was dissolved in ethanol and palladium hydroxide on carbon (181 mg, 1.29 mmol) was added. Under a stream of nitrogen, ammonium formate (677 mg, 10.7 mmol) was added and the reaction mixture was heated to reflux under N₂ for 2 hours. The reaction mixture was cooled and filtered through Arbocel® to remove catalyst and the filter was washed with further ethanol. The solvent was evapourated and an attempt to partition the residue showed the desired product to be water soluble. Therefore, all phases were combined and concentrated in vacuo to give an oil. The oil was heated with methanol to effect complete dissolution and the resulting solution was allowed to cool. The resulting solid was removed by filtration. Evaporation in vacuo of the filtrate gave the title compound (394 mg) as an oil which solidified on standing and was used crude in subsequent experiments.

### PREPARATION 89

### N-(4-Aminocyclohexylmethyl)-methanesulfonamide

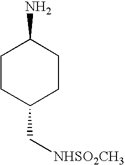

DIEA (0.66 ml; 3.78 mmol) was added to a solution of tert-butyl-trans-4-aminocyclohexyl carbamate (500 mg; 1.89 mmol) in dry DCM (10 mL) under nitrogen. The solution was cooled to 0° C. and methanesulphonyl chloride (0.18 mL, 2.27 mmol) was added dropwise. The reaction mixture was allowed to warm to room temperature and stirred for 18 hours. LCMS showed 75% conversion of the SM to the expected product. The starting material was not entirely soluble in DCM and so DMF (3 mL) was added followed by further DIEA (0.66 mL, 3.87 mmol) and methanesulfonyl chloride (0.18 mL, 2.27 mmol) at 0° C. The reaction mixture was then stirred at room temperature for 5 hours. Water (20 mL) was added and the reaction mixture was extracted with DCM (20 mL). The organic phase was washed sequentially with 1 M aqueous sodium hydroxide (2×10 mL), 1M aqueous HCl (10 mL) and brine (20 mL), dried over MgSO₄ and evaporated to afford 450 mg of the title compound as an off white powder.

LRMS: m/z (ES−) [M−1] 305.

### PREPARATION 90

### 4-Dibenzylamino-1-hydroxymethyl-cyclohexanol

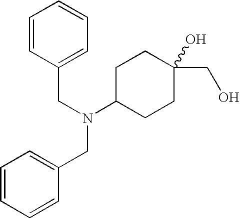

The product of Preparation 36 (256 mg, 0.833 mmol) was treated using the method of Preparation 37 to deliver 142 mg of the title compound which was used crude without further characterization.

### PREPARATION 91

### 4-Dibenzylamino-1-hydroxymethyl-cyclohexanol

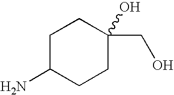

The product of Preparation 90, 4-dibenzylamino-1-hydroxymethyl-cyclohexanol (128 mg, 0.393 mmol) was treated using the method of Preparation 53 to deliver 47 mg of the title compound which was used crude without further characterization.

### PREPARATION 92

### 4-Dibenzylamino-1-n-propoxymethyl-cyclohexanol

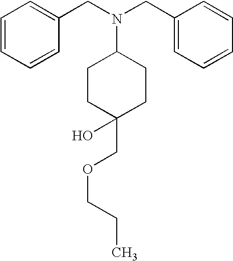

The material from Preparation 36 (600 mg, 1.95 mmol) was treated as per Preparation 41 to deliver 685 mg of the title compound which was used crude without further characterization.

### PREPARATION 93

### 4-Amino-1-n-propoxymethyl-cyclohexanol

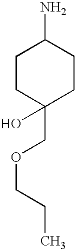

The product from Preparation 92, 4-dibenzylamino-1-n-propoxymethyl-cyclohexanol (620 mg, 1.687 mmol) was treated as per Preparation 53 to deliver 342 mg of the title compound which was used crude without further characterization.

### PREPARATION 94

### trans-4-(Dibenzylamino)cyclohexanecarbaldehyde

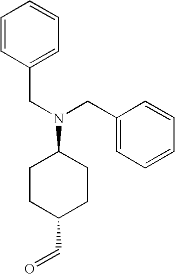

The title compound was prepared in a manner analogous to Preparation 51 using [trans-4-(dibenzylamino)cyclohexyl]nethanol (WO-2008/051493, 3.0 g, 9.69 mmol). The material was used crude in subsequent reactions. ES: m/z [M+1] 308.2.

### PREPARATION 95

### Cyclopropyl[trans-4-(dibenzylamino)cyclohexyl]methanol

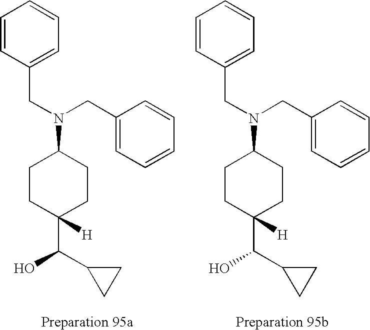

The product of Preparation 94 (3.38 g, 11 mmol) was dissolved into tetrahydrofuran (100 ml) and the solution was cooled to 0° C. Cyclopropyl magnesium bromide (0.5M solution in tetrahydrofuran, 26.3 mL, 13.2 mmol) was added and the reaction mixture was allowed to warm to room temperature. After one hour of stirring at room temperature the reaction mixture was cooled to 0° C. and more cyclopropyl magnesium bromide was added (22 mL, 11 mmol). The reaction was stirred for another hour at 0° C. The solvent was removed under reduced pressure and the residue was purified by flash chromatography on silica gel eluting with a gradient of ethyl acetate:heptane 1:4 to 1:2 by volume. This gave the title compound (0.7 g) as an oil. The crude product, containing enantiomers Preparation 95a and Preparation 95b was analysed by HPLC using the following conditions:

| | |
|---|---|
| Column analytical (250 * 4.6 mm id) | Chiralpak AD-1A |
| Mobile phase: | MeCN |
| Flow rate (ml/min) | 1.0 (analytical) |
| Detection (nm) | 225 nm and 254 nm |
| Temperature: | Ambient |
| injection volume (μl): | 20 ul |

The two enantiomers had retention times of 7.6 minutes and 10.5 minutes in the above system. The reaction product was separated into its individual enantiomers using the following preparative HPLC conditions:

| Column prep (250 * 21.2 mm id) | Chiralpak IA |
|---|---|
| Mobile phase: | 100% MeCN |
| Flow rate (ml/min) | 15 15 min run |
| Detection (nm) | 225 nm |
| Temperature: | ambient |
| Sample dissolution(mg/ml): | 1000 in 10 ml MeCN |
| Maximum injection volume (μl): | 450 |

This gave 320 mg of Preparation 95a (retention time 7.6 minutes) and 310 mg of Preparation 95b (retention time 10.5 minutes).

¹H NMR (400 MHz, CDCl₃): δ ppm 0.18-0.22 (m, 2H), 0.42-0.58 (m, 2H) 0.03-0.91 (m, 1H) 1.00-1.12 (m, 2H) 1.35-148 (m, 4H) 1.90-2.00.

### PREPARATION 96

### (S)-(trans-4-Aminocyclohexyl)(cyclopropyl)methanol

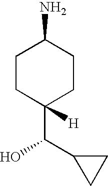

The title compound was prepared in an analogous manner to Preparation 53 using 350 mg of Preparation 95b (1.0 mmol). The product was used crude in subsequent experiments without characterization.

### Biological Data

Fluoresecence Intensity h-PGDSTBA Enzyme Assay

Prostaglandin D Synthase (PGDS) converts the substrate prostaglandin H₂ (PGH₂) to prostaglandin D₂. The depletion of PGH₂ was measured via an Fe(II) reduction of the remaining PGH₂ to malondialdehyde (MDA) and 12-HHT. The enzyme assay is based on the quantitative formation of a fluorescent complex from the non-fluorescent compounds MDA and 2-thiobarbituric acid (TBA), substantially as described in U.S. patent application publication US-2004/152148 by Lombardt.

The enzyme assay (31 μls) contained 100 mM Tris base pH 8.0, 100 μM MgCl₂, 0.1 mg/ml IgG Rabbit serum, 5.0 μM PGH2 (Cayman; ethanol solution, #17020), 2.5 mM L-Glutathione (Sigma; reduced form. #G4251), 1:175,000 human recombinant H-PGDS (from 1 mg/ml), 0.5% DMSO and inhibitor (varying concentration). Three μls of diluted inhibitor (dissolved in DMSO) was plated into a 384-well assay plate followed by a 25 μl addition of an enzyme solution containing h-PGDS, Tris, MgCl₂, IgG and L-Glutathione. After preincubation of inhibitor and enzyme solution for 10 minutes at room temperature, the reaction was initiated with a 3 μl addition of substrate solution in 10 mM HCl. The reaction was terminated after 42 second by the addition (3 μl) of stop buffer containing FeCl₂ and citric acid. After addition of 45.5 μls of TBA plates were heated for one hour in a 70° C. oven. Plates were cooled at room temperature overnight and read on a plate reader the next day with excitation @ 530 nm and emission @ 565 nm.

IC₅₀' s of inhibitors were calculated with a 4-parameter fit using 11 inhibitor concentrations in duplicate with 3-fold serial dilutions. Controls on each plate included no inhibitor (zero % effect) and an inhibitor 10-fold in excess of its' IC₅₀ (100% effect). The highest inhibitor concentration tested was typically 1 μM.

Examples 6-8, 11a-16, 23, 26-47, 49-103, 105-113, 115-118 and 126-128 were tested in a slightly modified assay: The enzyme assay (30 μls during biological process) contained 100 mM Trizma pH 8.0, 100 μM MgCl₂, 0.1 mg/ml IgG Rabbit serum, 5.0 μM PGH2 (Cayman; ethanol solution, #17020), 2.5 mM L-Glutathione (Sigma; reduced form #G4251), 1:40,000 human recombinant H-PGDS (from 1 mg/ml), 0.5% DMSO and inhibitor (varying concentration). 3 μls of diluted inhibitor (dissolved in DMSO) was plated into a 384-well assay plate followed by a 24 μl addition of an enzyme solution containing h-PGDS, Trizma, MgCl₂, IgG and L-Glutathione. After pre-incubation of inhibitor and enzyme solution for 10 minutes at room temperature, the reaction was initiated with a 3 μl addition of substrate solution in 10 mM HCl. The reaction was terminated after 40 second by the addition of 3 μl stop buffer containing FeCl₂ and citric acid. After addition of 45 μls of TBA plates were heated for one hour in a 70° C. oven. Plates were cooled at room temperature overnight and read on a plate reader the next day with excitation @ 530 nm and emission @ 560 nm. IC₅₀' s of inhibitors were calculated with a 4-parameter fit using 11 inhibitor concentrations in duplicate with ½ log serial dilutions. Controls on each plate included no inhibitor (zero % effect) and an inhibitor 500-fold in excess of its' IC₅₀ (100% effect). The highest inhibitor concentration tested was typically 10 μM.

The following table shows the IC₅₀ values thus obtained.

| | IC₅₀ |
|---|---|
| Example | (nM) |
| 1 | 64.9 |
| 2 | 319 |
| 3 | 246 |
| 4 | 1000 |
| 5 | 1000 |
| 6 | 159 |
| 7 | 292 |
| 8 | 1720 |
| 9 | 7.11 |
| 10 | 94.3 |
| 11a | 125 |
| 11b | 13.7 |
| 12 | 11.2 |
| 13 | 16.5 |
| 14 | 7.48 |
| 15 | 10.3 |
| 16 | 29.1 |
| 17 | 2.87 |
| 18 | 7.72 |
| 19 | 10.2 |
| 20 | 105 |
| 21 | 1.66 |
| 22 | 40.8 |
| 23 | 17.4 |
| 24 | 20.1 |
| 25 | 22.3 |
| 26 | 18.2 |
| 27 | 14.2 |
| 28 | 3.72 |
| 29 | 5.02 |
| 30 | 25.6 |
| 31 | 10.8 |
| 32 | 37.9 |
| 33 | 10 |
| 34 | 8.32 |
| 35 | 8.29 |
| 36 | 8.34 |
| 37 | 25.4 |
| 38 | 8.43 |
| 39 | 4.26 |
| 40 | 9.00 |
| 41 | 7.18 |
| 41a | 3.8/171 |
| 41b | 3.8/171 |
| 42 | 10.2 |
| 43 | 14.4 |
| 44 | 67.5 |
| 45 | 10.5 |
| 46 | 10.7 |
| 47 | 23.0 |
| 48 | 0.614 |
| 49 | 69.2 |
| 50 | 5.76 |
| 51 | 172 |
| 52 | 39.1 |
| 53 | 9.26 |
| 54 | 87.2 |
| 55 | 18.5 |
| 56 | 30.6 |
| 57 | 32.0 |
| 58 | 307 |
| 59 | 30.5 |
| 60 | 111 |
| 61 | 45.0 |
| 62 | 137 |
| 63 | 13.0 |
| 64 | 6.20 |
| 64A | 8.3 |
| 64B | 64.6 |
| 64C | 47 |
| 64D | 14.7 |
| 64E | 9.3 |
| 64F | 16.8 |
| 64G | 17.2 |
| 64H | 13 |
| 64I | 11.1 |
| 64J | 19.6 |
| 64K | 10.9 |
| 64L | 255 |
| 64M | 509 |
| 65 | 15.7 |
| 66 | 97.4 |
| 67 | 118 |
| 68 | 13.2 |
| 69 | 14.7 |
| 70 | 4.62 |
| 71 | 22.0 |
| 72 | 23.2 |
| 73 | 14 |
| 74 | 10.8 |
| 75 | 20.6 |
| 76 | 31.8 |
| 77 | 6.42 |
| 78 | 7.89 |
| 79 | 15.9 |
| 80 | 16.6 |
| 81 | 53.4 |
| 82 | 12.6 |
| 83 | 6.93 |
| 84 | 39.8 |
| 85 | 2.75 |
| 86 | 25.6 |
| 87 | 2.07 |
| 88 | 14.7 |
| 89 | 3.93 |
| 90 | 12.6 |
| 91 | 3.94 |
| 92 | 2.71 |
| 93 | 15.3 |
| 94 | 51.9 |
| 95 | 6.28 |
| 96 | 23.7 |
| 97 | 2.90 |
| 98 | 21.6 |
| 99 | 11.2 |
| 100 | 12.9 |
| 101 | 15.8 |
| 102 | 9.74 |
| 103 | 1.43 |
| 104 | 2.29 |
| 105 | 14.7 |
| 106 | 14.2 |
| 107 | 31.1 |
| 108 | 21.8 |
| 109 | 16.7 |
| 110 | 5.90 |
| 111 | 23.0 |
| 112 | 15.8 |
| 113 | 9.76 |
| 113A | 21.1 |
| 113B | 7 |
| 113C | 7.4 |
| 113D | 10.8 |
| 113E | 8.8 |
| 113F | 60 |
| 113G | 4.5 |
| 113H | 46 |
| 113I | 10.5 |
| 113J | 6.6 |
| 113K | 9.6 |
| 113L | 6.1 |
| 113M | 7.6 |
| 113N | 11.5 |
| 113O | 7.3 |
| 113P | 9.1 |
| 113Q | 9.7 |
| 113R | 13.8 |
| 114 | 4.41 |
| 115 | 8.74 |
| 116 | 139 |
| 117 | 7.17 |
| 118 | 51.2 |
| 119 | 1.28 |
| 120 | 1.77 |
| 121 | 88.0 |
| 122 | 4.16 |
| 123 | 30.0 |
| 124 | 22.7 |
| 125 | 6.66 |
| 126 | 4.07 |
| 127 | 5.03 |
| 128 | 179 |
| 129 | 194 |
| 130 | 15.2 |
| 130A | 52.3 |
| 131 | |
| 132 | |
| 133 | |
| 134 | |
| 135 | |
| 136 | |
| 137 | |
| 138 | |
| 139 | |
| 140 | |
| 141 | |
| 142A | |
| 142B | 16.4 |
| 143 | 7.2 |
| 144 | 7.5 |
| 145 | 11.6 |
| 146 | 21 |
| 146A | |
| 147 | 5.7 |
| 148 | 1.5/5.6 |
| 149 | 1.5/5.6 |
| 150 | 8.9 |
| 151 | 10.3 |
| 152 | 2.2 |
| 153 | 13.2 |
| 154 | 40.3 |
| 155 | 97.8 |
| 155A | |
| 156 | 11.5 |
| 157 | 5.8 |
| 158 | 68.9 |
| 159 | 4.9 |
| 160 | 11.2 |
| 161 | 4.1 |
| 162 | 22.7 |
| 163 | 6.1 |
| 164 | 16.1 |
| 165 | 16.9 |
| 166 | 35.5 |
| 167 | 28.5 |
| 168 | 4.4 |
| 169 | 15.1 |
| 170 | 4.3 |
| 171 | 6.3 |
| 172 | 6.4 |
| 173 | 21.6 |
| 174 | 1860 |
| 175 | 20.2 |
| 176 | 9.0 |
| 177 | 21.4 |
| 178 | 21.6 |
| 179 | 5.6 |
| 180 | 8.9 |
| 181 | 5.8 |
| 182 | 17.8 |
| 183 | 75.2 |
| 184 | 20.3 |
| 185 | 25.4 |
| 186 | 23.9 |
| 187 | 30.7 |
| 187A | |
| 188 | 17.1 |
| 189 | 13.7 |
| 190 | 17.5 |
| 191 | 29.2 |
| 192 | 15.3 |
| 193 | 10.2 |
| 194 | 14.7 |
| 195 | 11.8 |
| 196 | 9.6 |
| 197 | 10.2 |
| 198 | 19.1 |
| 199 | 21.8 |
| 200 | 189 |
| 200A | |
| 201 | 31 |
| 202 | 17.1 |
| 203 | 17.1 |
| 204 | 68.7 |
| 205 | |
| 206 | 13.5 |
| 207 | 81.1 |
| 208 | 78.7/2.6 |
| 209 | 78.7/2.6 |
| 210 | 9.6 |
| 211 | 13 |
| 212 | 2.3 |
| 213 | 36 |
| 214 | 3 |
| 214A | |
| 215 | 5.9 |
| 216 | 31.8 |
| 217 | 67.1 |
| 218 | 79.7 |
| 219 | 32.7 |
| 220 | 32.7 |
| 221 | 52.7 |
| 222 | 50.8 |
| 223 | |
| 224 | 74 |
| 225 | 5.3 |
| 226 | 13.2 |
| 227 | 23.8 |
| 228 | 17.5 |
| 229 | 2.5 |
| 230 | 37.3 |
| 231 | 13.8 |
| 232 | 31.2 |
| 233 | 4.1 |
| 234 | 11 |
| 235 | 11.8 |
| 236 | 5.7 |
| 237 | 5.1 |
| 238 | 47.1 |
| 238A | 27.4 |
| 239 | 34.9 |
| 240 | 26.5 |
| 241 | 15.4 |
| 242 | 19.8 |
| 243 | 29.1 |
| 244 | 192 |
| 244A | |
| 245 | 21.6 |
| 246 | 16.2 |
| 247 | 57 |
| 248 | 10.4 |
| 249 | 13.9 |
| 250 | 26.4 |
| 251A | 13.3 |
| 251B | |
| 251C | |
| 251D | |
| 252 | 17.1 |
| 253 | 50.9 |
| 254 | 40.5 |
| 255 | 12.3 |
| 256 | 44.1 |
| 257 | 34.5 |
| 258 | 29.2 |
| 259 | 44.1 |
| 260 | 59.9 |
| 261 | 4.0 |
| 261A | |
| 261B | |
| 262 | 81 |
| 263 | 5.0 |
| 264 | 9.2 |
| 265 | 2.8 |
| 266 | 10.6 |
| 267 | 11 |
| 268 | 24.6 |
| 269 | 15.4 |
| 270 | 9.1 |
| 271 | 18 |
| 272 | 11 |
| 273 | 5.4 |
| 273A | |
| 274 | 13.7 |
| 275 | 81.8 |
| 276 | |
| 277 | |

In the case of Examples 41a/41b, 148/149 and 208/209, two possible assay results are given, since in each case the two enantiomers have not been structurally assigned.

1. A compound of formula (I): 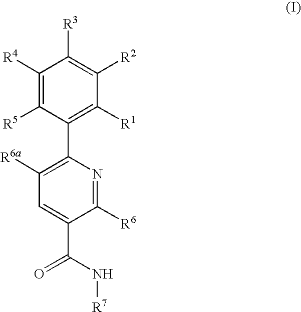
or a pharmaceutically acceptable salt thereof, wherein:
R¹, R², R³, R⁴ and R⁵ are each independently H, F, Cl, —CN, —NH₂, —CH₃, —CH₂F, —CHF₂, —CF₃, —OH, —OCH₃, —OCH₂F, —OCHF₂ or —OCF₃;
R⁶ is H, —NH₂, —OH or —CH₃;
R^{6a} is H, F or Cl;
R⁷ is C₃-C₈ cycloalkyl or C₅-C₁₂ bicycloalkyl, said C₃-C₈ cycloalkyl being optionally fused to a phenyl ring or a 5- or 6-membered aromatic heterocyclic ring; said group R⁷ being (a) optionally substituted by 1-3 substituents selected from R^{a}, —OR^{b}, —S(O)ₙR^{b}, —COR^{b}, —NR^{x}R^{b}, —OCOR^{b}, —COOR^{b}, —NR^{x}COR^{b}, —CONR^{x}R^{b}, —NR^{x}SO₂R^{b}, SO₂NR^{x}R^{b}, —NR^{x}SO₂NR^{x}R^{b}, —NHCOOR^{b}, NHCONR^{x}R^{b}, —OCONR^{x}R^{b}, —OCOOR^{b}, —CONHSO₂R^{b}, oxo and —CN, and (b) optionally substituted by one or more halo atoms;
R^{a} is in each instance independently selected from C₁-C₈ alkyl, C₃-C₈ cycloalkyl, C₆-C₁₂ bicycloalkyl, Aryl¹, Het¹, Het², Het³ and Het⁴, said C₁-C₈ alkyl, C₃-C₈ cycloalkyl, C₆-C₁₂ bicycloalkyl, Aryl¹, Het¹, Het², Het³ and Het⁴ each being optionally substituted by 1-3 substituents selected from R^{c}, —OR^{d}, —S(O)ₙR^{d}, —COR^{d}, —NR^{x}R^{d}, —OCOR^{d}, —COOR^{d}, —NR^{x}COR^{d}, —CONR^{x}R^{d}, —NR^{x}SO₂R^{d}, SO₂NR^{x}R^{d}, —NR^{x}SO₂NR^{x}R^{d}, —NHCOOR^{d}, NHCONR^{x}R^{d}, —OCONR^{x}R^{d}, —OCOOR^{d}, —CONHSO₂R^{d}, and —CN, and one or more halo atoms;
R^{b} is in each instance independently selected from H, C₁-C₈ alkyl, C₃-C₈ cycloalkyl, C₆-C₁₂ bicycloalkyl, Aryl¹, Het¹, Het², Het³ and Het⁴, said C₁-C₆ alkyl, C₃-C₈ cycloalkyl, C₆-C₁₂ bicycloalkyl, Aryl¹, Het¹, Het², Het³ and Het⁴ each being optionally substituted by 1-3 substituents selected from R^{c}, —OR^{d}, —S(O)ₙR^{d}, —COR^{d},
—NR^{x}R^{d}, —OCOR^{d}, —COOR^{d}, —NR^{x}COR^{d}, —CONR^{x}R^{d}, —NR^{x}SO₂R^{d}, SO₂NR^{x}R^{d}, —NR^{x}SO₂NR^{x}R^{d}, —NHCOOR^{d}, NHCONR^{x}R^{d}, —OCONR^{x}R^{d}, —OCOOR^{d}, —CONHSO₂R^{d}, and —CN, and one or more halo atoms;
n is 0, 1 or 2;
R^{x} is in each instance independently H, C₁-C₈ alkyl or C₃-C₈ cycloalkyl, said C₁-C₈ alkyl or C₃-C₈ cycloalkyl being optionally substituted by one or more halo atoms;
Aryl¹ is phenyl or naphthyl;
Het¹ is a 3 to 8-membered saturated or partially unsaturated monocyclic heterocycle, containing 1 or 2 heteroatoms selected from O and N;
Het² is a 6 to 12-membered saturated or partially unsaturated multicyclic heterocycle containing 1 or 2 heteroatoms selected from O and N;
Het³ is (i) a 6-membered aromatic heterocycle containing 1-3 N atoms or (ii) a 5-membered aromatic heterocycle containing either (a) 1-4 N atoms or (b) 1 O or S atom and 0-3 N atoms;
Het⁴ is (i) a 10-membered bicyclic aromatic heterocycle containing 1-4 N atoms or (ii) a 9-membered bicyclic aromatic heterocycle containing either (a) 1-4 N atoms or (b) 1 O or S atom and 0-3 N atoms;
R^{c} is in each instance independently selected from C₁-C₈ alkyl, C₃-C₈ cycloalkyl, C₆-C₁₂ bicycloalkyl, Aryl², Het⁵, Het⁶, Het⁷ and Het⁸, said C₁-C₈ alkyl, C₃-C₈ cycloalkyl, C₆-C₁₂ bicycloalkyl, Aryl², Het⁵, Het⁶, Het⁷ and Het⁸ each being optionally substituted by 1-3 substituents selected from R^{e} and one or more halo atoms;
R^{d} is in each instance independently selected from H, C₁-C₆ alkyl, C₃-C₈ cycloalkyl, C₆-C₁₂ bicycloalkyl, Aryl², Het⁵, Het⁶, Het⁷ and Het⁸, said C₁-C₆ alkyl, C₃-C₈ cycloalkyl, C₆-C₁₂ bicycloalkyl, Aryl², Het⁵, Het⁶, Het⁷ and Het⁸ each being optionally substituted by 1-3 substituents selected from R^{e} and one or more halo atoms;
Aryl² is phenyl or naphthyl;
Het⁵ is a 3 to 8-membered saturated or partially unsaturated monocyclic heterocycle, containing 1 or 2 heteroatoms selected from O and N;
Het⁶ is a 6 to 12-membered saturated or partially unsaturated multicyclic heterocycle containing 1 or 2 heteroatoms selected from O and N;
Het⁷ is (i) a 6-membered aromatic heterocycle containing 1-3 N atoms or (ii) a 5-membered aromatic heterocycle containing either (a) 1-4 N atoms or (b) 1 O or S atom and 0-3 N atoms;
Het⁸ is (i) a 10-membered bicyclic aromatic heterocycle containing 1-4 N atoms or (ii) a 9-membered bicyclic aromatic heterocycle containing either (a) 1-4 N atoms or (b) 1 O or S atom and 0-3 N atoms; and
R^{e} is —OR^{x}, —S(O)ₙR^{x}, —COR^{x}, —NR^{x}R^{x}, —OCOR^{x}, —COOR^{x}, —NR^{x}COR^{x}, —CONR^{x}R^{x}, —NR^{x}SO₂R^{x}, —SO₂NR^{x}R^{x}, —NR^{x}SO₂NR^{x}R^{x}, —NHCOOR^{x}, NHCONR^{x}R^{x}, —OCONR^{x}R^{x}, —OCOOR^{x}, —CONHSO₂R^{x}, or —CN;
with the proviso that the compound of formula (I) is not:
N-cyclohexyl-2-methyl-6-phenyl-3-pyridinecarboxamide,
N-(2-methylcyclohexyl)-2-methyl-6-(3-bromophenyl)-3-pyridinecarboxamide,
N-{2-[(hydroxyamino)carbonyl]cyclopentyl}-6-(2-methylphenyl)-3-pyridinecarboxamide, or N-{2-[hydroxyamino)carbonyl]cyclopentyl}-6-(2-methoxyphenyl)-3-pyridinecarboxamide.

2. A compound of claim 1, or a pharmaceutically acceptable salt thereof, wherein R¹, R², R³, R⁴ and R⁵ are each independently H, F, —CH₃, or —OCH₃.

3. A compound of claim 1, or a pharmaceutically acceptable salt thereof, wherein R¹ and R⁵ are H; and R², R³ and R⁴ are each independently H, F, —CH₃ or —OCH₃.

4. A compound of claim 1, or a pharmaceutically acceptable salt thereof, wherein R¹, R³, R⁴ and R⁵ are H and R² is F; or R¹, R³, R⁴ and R⁵ are H and R² is —CH₃; or R¹, R³, R⁴ and R⁵ are H and R² is —OCH₃; or R¹, R², R⁴ and R⁵ are H and R³ is F; or R¹, R³ and R⁵ are H and R² and R⁴ are both F; or R¹, R², R³, R⁴ and R⁵ are each H.

5. A compound of claim 1, or a pharmaceutically acceptable salt thereof, wherein R⁶ is H.

6. A compound of claim 1, or a pharmaceutically acceptable salt thereof, wherein R^{6a} is H or Cl.

7. A compound of claim 1, or a pharmaceutically acceptable salt thereof, wherein R⁷ is C₃-C₆ cycloalkyl, said C₃-C₆ cycloalkyl being optionally fused to a phenyl ring or a 5- or 6-membered aromatic heterocyclic ring; said group R⁷ being optionally substituted by 1-3 substituents selected from R^{a}, —OR^{b}, —COR^{b}, —NR^{x}R^{b}, —COOR^{b}, —NR^{x}COR^{b}, —CONR^{x}R^{b}, oxo and one or more halo atoms.

8. A compound of claim 1, or a pharmaceutically acceptable salt thereof, wherein R⁷ is C₃-C₆ cycloalkyl, said C₃-C₆ cycloalkyl being optionally fused to a phenyl ring or a 5- or 6-membered aromatic heterocyclic ring; said group R⁷ being optionally substituted by 1-2 substituents selected from —COON, —COO(C₁-C₆ alkyl), Het³, —(C₁-C₆ alkylene)Het¹, —COHet¹, Het¹, —OHet³, —OH, —O(C₁-C₆ alkyl), —O(C₁-C₆ alkylene)OH, —O(C₁-C₆alkylene)OR^{x}, —(C₁-C₆ alkylene)OH, C₁-C₆ alkyl, —(C₁-C₆ alkylene)CONR^{x}R^{x}, —(C₁-C₆ alkylene)NR^{x}R^{x}, —O(C₁-C₆ alkylene)CONR^{x}R^{x}, —CONR^{x}R^{x},
—CONR^{x}(C₁-C₆ alkylene)Ph, —CONR^{x}(C₁-C₆ alkylene)NR^{x}R^{x}, —NR^{x}R^{x}, —NR^{x}COR^{x}, —O(C₁-C₆ alkyl), oxo or one or more halo atoms, each C₁-C₆ alkyl being optionally substituted by one or more halo atoms and said Het³, —(C₁-C₆ alkylene)Het¹, —COHet¹, Het¹,
—NR^{x}Het¹ and —OHet³ being optionally substituted by 1-2 substituents selected from C₁-C₆ alkyl, C₃-C₆ cycloalkyl, OR^{x}, NR^{x}R^{x}, —COO(C₁-C₆ alkyl) and —S(C₁-C₆ alkyl).

9. A compound of claim 1, or a pharmaceutically acceptable salt thereof, wherein R⁷ is C₃-C₆ cycloalkyl, said C₃-C₆ cycloalkyl being optionally fused to a phenyl, imidazolyl, pyridyl or pyrazolyl ring; said group R⁷ being optionally substituted by 1-2 substituents selected from pyridyl, imidazolyl, (C₁-C₆ alkyl)imidazolyl, (C₁-C₆ alkyl)thioimidazolyl, (C₁-C₆ alkyl)tetrazolyloxy, piperazinylcarbonyl, (C₁-C₆ alkyl)piperazinylcarbonyl, (C₁-C₆ cycloalkyl)piperazinylcarbonyl, (C₁-C₆ alkyl)piperazinyl, [(C₁-C₆ alkyl)-OCO][C₁-C₆ alkyl]piperazinylcarbonyl, aminoazetidinylcarbonyl, pyrrolidinylcarbonyl, hydroxypyrrolidinylcarbonyl, hydroxypyrrolidinyl, aminopyrrolidinylcarbonyl, hydroxypiperidinylcarbonyl, hyydroxypiperidinyl, morpholinyl, morpholinylcarbonyl, morpholinyl(C₁-C₆ alkyl), (C₁-C₆ alkyl)piperazinyl(C₁-C₆ alkyl)carboxy, amino, (C₁-C₆ alkyl)amino, furanylamino, (C₁-C₆ haloalkyl)carbonylamino, hydroxy, hydroxy(C₁-C₆ alkyl), hydroxy(C₁-C₆ alkoxy), C₁-C₆ alkoxy, (C₁-C₆ alkoxy)C₁-C₆ alkoxy, [(C₁-C₆ alkoxy)C₁-C₆ alkyl]amino, [(C₁-C₆ alkoxy)C₁-C₆ alkyl][C₁-C₆ alkyl]aminophenyl(C₁-C₆ alkyl)aminocarbonyl, (phenyl(C₁-C₆alkyl))(C₁-C₆ alkylaminocarbonyl, di-(C₁-C₆ alkyl)aminocarbonyl, (di-(C₁-C₆ alkyl)aminocarbonyl) C₁-C₆ alkoxy, oxo, (di-(C₁-C₆ alkyl)aminocarbonyl)C₁-C₆ alkyl, (di-(C₁-C₆ alkyl)amino) C₁-C₆ alkyl, (C₁-C₆ alkyl)oxycarbonyl, carboxy, oxazepinyl, C₁-C₆ alkyl, (C₃-C₈ cycloalkyl)aminocarbonyl, ((C₁-C₆ alkylamino) C₁-C₆ alkyl)(C₁-C₆ alkyl)aminocarbonyl, (C₁-C₆ alkyl)carbonylamino and fluoro.

10. A compound of claim 1, which is:
6-(3-fluorophenyl)-N-{cis-3-[(4-hydroxypiperidin-1-yl)carbonyl]cyclohexyl}nicotinamide;
N-[trans-4-(dimethylcarbamoyl)cyclohexyl]-6-(3-fluorophenyl)nicotinamide;
N-[4-trans-(cyclopropylhydroxymethyl)cyclohexyl]-6-(3-fluorophenyl)nicotinamide; or
N-{trans-4-[acetamidoethyl]cyclohexyl}-6-(3-fluorophenyl)nicotinamide;
or a pharmaceutically acceptable salt thereof.

11. 6-(3-Fluorophenyl)-N-{(1S,3R)-3-[(4-hydroxypiperidin-1-yl)carbonyl]cyclohexyl}nicotinamide or a pharmaceutically acceptable salt thereof.

12. A pharmaceutical composition comprising a compound of claim 1, or a pharmaceutically acceptable salt thereof, and a pharmaceutically acceptable excipient.

13. A method of treating a disease or condition mediated at least in part by prostaglandin D₂ produced by H-PGDS in a subject in need of such treatment, which method comprises administering to said subject a therapeutically effective amount of a compound of claim 1 or a pharmaceutically acceptable salt thereof.

14. The method of claim 11 wherein the disease or condition is asthma.
